SEQUENCE LISTING (189 sequences)

SEQ ID NO: 1 - RNA, 45 nt, synthetic construct
  source 1..45: mol_type = other RNA
gtggaatagtataacaatatgctaaatgttgttatagtatcccac

SEQ ID NO: 2 - RNA, 55 nt, synthetic construct
  source 1..55: mol_type = other RNA
ggtgtcgagaagaggagaacaatatgctaaatgttgttctcgtctcctcgacacc

SEQ ID NO: 3 - RNA, 55 nt, synthetic construct
  source 1..55: mol_type = other RNA
ggtgtcgagaagaggagaacaatatctttcatgttgttctcctctcctcgacacc

SEQ ID NO: 4 - RNA, 45 nt, synthetic construct
  source 1..45: mol_type = other RNA
gtggaatagtataacaatatctttcatgttgttatactatcccac

SEQ ID NO: 5 - DNA, 102 bp, synthetic construct
  source 1..102: mol_type = other DNA
ggtgtcgagaagaggagaacaatatgctactgccatcagtcggcgtggactgtagaaccatgccgactgatggcagaatgttgttctcgtctcctcgacacc

SEQ ID NO: 6 - DNA, 102 bp, synthetic construct
  source 1..102: mol_type = other DNA
ggtgtcgagaagaggagaacaatatctttctgccatcagtcggcgtggactgtagaaccatgccgactgatggcagcatgttgttctcgtctcctcgacacc

SEQ ID NO: 7 - DNA, 102 bp, synthetic construct
  source 1..102: mol_type = other DNA
ggtgtcgagaagaggagaacaatatctttctgccatcagtcggcgtggactgtagaaccatgccgactgatggcagcatgttgttctcctctcctcgacacc

SEQ ID NO: 8 - DNA, 100 bp, synthetic construct
  source 1..100: mol_type = other DNA
ggtgtcgagaagaggagaacaatatgctaaatgttgttctcgtctcctcgacacccagtagattcaacatgctgtaaaaaacagcatgttgaatctactg

SEQ ID NO: 9 - DNA, 100 bp, synthetic construct
  source 1..100: mol_type = other DNA
ggtgtcgagaagaggagaacaatatgctaaatgttgttctcgtctcctcgacacccagtaggttcaacatgctgtaaaaaacagcatgttgaatctactg

SEQ ID NO: 10 - DNA, 100 bp, synthetic construct
  source 1..100: mol_type = other DNA
ggtgtcgagaagaggagaacaatatgctaaatgttgttctcgtctcctcgacacccagtagcttcaacatgctgtaaaaaacagcatgttgaagctactg

SEQ ID NO: 11 - DNA, 100 bp, synthetic construct
  source 1..100: mol_type = other DNA
ggtgtcgagaagaggagaacaatatgctaaatgttgttctcgtctcctcgacacccagtagtttcaacatgctgtaaaaaacagcatgttgaagctactg

SEQ ID NO: 12 - DNA, 100 bp, synthetic construct
  source 1..100: mol_type = other DNA
ggtgtcgagaagaggagaacaatatgctaaatgttgttctcgtctcctcgacacccagtagttacaacatgctgtaaaaaacagcatgttgtaactactg

SEQ ID NO: 13 - DNA, 100 bp, synthetic construct
  source 1..100: mol_type = other DNA
ggtgtcgagaagaggagaacaatatgctaaatgttgttctcgtctcctcgacacccagtagttgcaacatgctgtaaaaaacagcatgttgtaactactg

SEQ ID NO: 14 - DNA, 100 bp, synthetic construct
  source 1..100: mol_type = other DNA
ggtgtcgagaagaggagaacaatatgctaaatgttgttctcgtctcctcgacacccagtagatacaacatgctgtaaaaaacagcatgttgtatctactg

SEQ ID NO: 15 - DNA, 100 bp, synthetic construct
  source 1..100: mol_type = other DNA
ggtgtcgagaagaggagaacaatatgctaaatgttgttctcgtctcctcgacacccagtagatgcaacatgctgtaaaaaacagcatgttgtatctactg

SEQ ID NO: 16 - DNA, 100 bp, synthetic construct
  source 1..100: mol_type = other DNA
ggtgtcgagaagaggagaacaatatgctaaatgttgttctcgtctcctcgacacccagtaggtacaacatgctgtaaaaaacagcatgttgtatctactg

SEQ ID NO: 17 - DNA, 100 bp, synthetic construct
  source 1..100: mol_type = other DNA
ggtgtcgagaagaggagaacaatatgctaaatgttgttctcgtctcctcgacacccagtaggtgcaacatgctgtaaaaaacagcatgttgtatctactg

SEQ ID NO: 18 - DNA, 100 bp, synthetic construct
  source 1..100: mol_type = other DNA
ggtgtcgagaagaggagaacaatatgctaaatgttgttctcgtctcctcgacacccagtagctacaacatgctgtaaaaaacagcatgttgtagctactg

SEQ ID NO: 19 - DNA, 100 bp, synthetic construct
  source 1..100: mol_type = other DNA
ggtgtcgagaagaggagaacaatatgctaaatgttgttctcgtctcctcgacacccagtagctgcaacatgctgtaaaaaacagcatgttgtagctactg

SEQ ID NO: 20 - DNA, 100 bp, synthetic construct
  source 1..100: mol_type = other DNA
ggtgtcgagaagaggagaacaatatgctaaatgttgttctcgtctcctcgacacccagtagttacaacatgctgtaaaaaacagcatgttgtagctactg

SEQ ID NO: 21 - DNA, 100 bp, synthetic construct
  source 1..100: mol_type = other DNA
ggtgtcgagaagaggagaacaatatgctaaatgttgttctcgtctcctcgacacccagtagttgcaacatgctgtaaaaaacagcatgttgtagctactg

SEQ ID NO: 22 - DNA, 100 bp, synthetic construct
  source 1..100: mol_type = other DNA
ggtgtcgagaagaggagaacaatatgctaaatgttgttctcgtctcctcgacacccagtaggtacaacatgctgtaaaaaacagcatgttgtacctactg

SEQ ID NO: 23 - DNA, 100 bp, synthetic construct
  source 1..100: mol_type = other DNA
ggtgtcgagaagaggagaacaatatgctaaatgttgttctcgtctcctcgacacccagtaggtgcaacatgctgtaaaaaacagcatgttgtacctactg

SEQ ID NO: 24 - DNA, 100 bp, synthetic construct
  source 1..100: mol_type = other DNA
ggtgtcgagaagaggagaacaatatgctaaatgttgttctcgtctcctcgacacccagtagttccaacatgctgtaaaaaacagcatgttggaactactg

SEQ ID NO: 25 - DNA, 100 bp, synthetic construct
  source 1..100: mol_type = other DNA
ggtgtcgagaagaggagaacaatatgctaaatgttgttctcgtctcctcgacacccagtagtttcaacatgctgtaaaaaacagcatgttggaactactg

SEQ ID NO: 26 - DNA, 100 bp, synthetic construct
  source 1..100: mol_type = other DNA
ggtgtcgagaagaggagaacaatatgctaaatgttgttctcgtctcctcgacacccagtagatccaacatgctgtaaaaaacagcatgttggatctactg

SEQ ID NO: 27 - DNA, 100 bp, synthetic construct
  source 1..100: mol_type = other DNA
ggtgtcgagaagaggagaacaatatgctaaatgttgttctcgtctcctcgacacccagtagattcaacatgctgtaaaaaacagcatgttggatctactg

SEQ ID NO: 28 - DNA, 100 bp, synthetic construct
  source 1..100: mol_type = other DNA
ggtgtcgagaagaggagaacaatatgctaaatgttgttctcgtctcctcgacacccagtaggtccaacatgctgtaaaaaacagcatgttggatctactg

SEQ ID NO: 29 - DNA, 100 bp, synthetic construct
  source 1..100: mol_type = other DNA
ggtgtcgagaagaggagaacaatatgctaaatgttgttctcgtctcctcgacacccagtaggttcaacatgctgtaaaaaacagcatgttggatctactg

SEQ ID NO: 30 - DNA, 100 bp, synthetic construct
  source 1..100: mol_type = other DNA
ggtgtcgagaagaggagaacaatatgctaaatgttgttctcgtctcctcgacacccagtagctccaacatgctgtaaaaaacagcatgttggagctactg

SEQ ID NO: 31 - DNA, 100 bp, synthetic construct
  source 1..100: mol_type = other DNA
ggtgtcgagaagaggagaacaatatgctaaatgttgttctcgtctcctcgacacccagtagcttcaacatgctgtaaaaaacagcatgttggagctactg

SEQ ID NO: 32 - DNA, 100 bp, synthetic construct
  source 1..100: mol_type = other DNA
ggtgtcgagaagaggagaacaatatgctaaatgttgttctcgtctcctcgacacccagtagttccaacatgctgtaaaaaacagcatgttggagctactg

SEQ ID NO: 33 - DNA, 100 bp, synthetic construct
  source 1..100: mol_type = other DNA
ggtgtcgagaagaggagaacaatatgctaaatgttgttctcgtctcctcgacacccagtagtttcaacatgctgtaaaaaacagcatgttggagctactg

SEQ ID NO: 34 - DNA, 100 bp, synthetic construct
  source 1..100: mol_type = other DNA
ggtgtcgagaagaggagaacaatatgctaaatgttgttctcgtctcctcgacacccagtaggtccaacatgctgtaaaaaacagcatgttggacctactg

SEQ ID NO: 35 - DNA, 100 bp, synthetic construct
  source 1..100: mol_type = other DNA
ggtgtcgagaagaggagaacaatatgctaaatgttgttctcgtctcctcgacacccagtaggttcaacatgctgtaaaaaacagcatgttggacctactg

SEQ ID NO: 36 - DNA, 100 bp, synthetic construct
  source 1..100: mol_type = other DNA
ggtgtcgagaagaggagaacaatatgctaaatgttgttctcgtctcctcgacacccagtagatgcaacatgctgtaaaaaacagcatgttgcatctactg

SEQ ID NO: 37 - DNA, 100 bp, synthetic construct
  source 1..100: mol_type = other DNA
ggtgtcgagaagaggagaacaatatgctaaatgttgttctcgtctcctcgacacccagtaggtgcaacatgctgtaaaaaacagcatgttgcatctactg

SEQ ID NO: 38 - DNA, 100 bp, synthetic construct
  source 1..100: mol_type = other DNA
ggtgtcgagaagaggagaacaatatgctaaatgttgttctcgtctcctcgacacccagtagctgcaacatgctgtaaaaaacagcatgttgcagctactg

SEQ ID NO: 39 - DNA, 100 bp, synthetic construct
  source 1..100: mol_type = other DNA
ggtgtcgagaagaggagaacaatatgctaaatgttgttctcgtctcctcgacacccagtagttgcaacatgctgtaaaaaacagcatgttgcagctactg

SEQ ID NO: 40 - DNA, 100 bp, synthetic construct
  source 1..100: mol_type = other DNA
ggtgtcgagaagaggagaacaatatgctaaatgttgttctcgtctcctcgacacccagtagagtcaacatgctgtaaaaaacagcatgttgaatctactg

SEQ ID NO: 41 - DNA, 100 bp, synthetic construct
  source 1..100: mol_type = other DNA
ggtgtcgagaagaggagaacaatatgctaaatgttgttctcgtctcctcgacacccagtagcgtcaacatgctgtaaaaaacagcatgttgaagctactg

SEQ ID NO: 42 - DNA, 100 bp, synthetic construct
  source 1..100: mol_type = other DNA
ggtgtcgagaagaggagaacaatatgctaaatgttgttctcgtctcctcgacacccagtagtgacaacatgctgtaaaaaacagcatgttgtaactactg

SEQ ID NO: 43 - DNA, 100 bp, synthetic construct
  source 1..100: mol_type = other DNA
ggtgtcgagaagaggagaacaatatgctaaatgttgttctcgtctcctcgacacccagtagagacaacatgctgtaaaaaacagcatgttgtatctactg

SEQ ID NO: 44 - DNA, 100 bp, synthetic construct
  source 1..100: mol_type = other DNA
ggtgtcgagaagaggagaacaatatgctaaatgttgttctcgtctcctcgacacccagtagcgacaacatgctgtaaaaaacagcatgttgtagctactg

SEQ ID NO: 45 - DNA, 100 bp, synthetic construct
  source 1..100: mol_type = other DNA
ggtgtcgagaagaggagaacaatatgctaaatgttgttctcgtctcctcgacacccagtagggacaacatgctgtaaaaaacagcatgttgtacctactg

SEQ ID NO: 46 - DNA, 100 bp, synthetic construct
  source 1..100: mol_type = other DNA
ggtgtcgagaagaggagaacaatatgctaaatgttgttctcgtctcctcgacacccagtagtgccaacatgctgtaaaaaacagcatgttggaactactg

SEQ ID NO: 47 - DNA, 100 bp, synthetic construct
  source 1..100: mol_type = other DNA
ggtgtcgagaagaggagaacaatatgctaaatgttgttctcgtctcctcgacacccagtagagccaacatgctgtaaaaaacagcatgttggatctactg

SEQ ID NO: 48 - DNA, 100 bp, synthetic construct
  source 1..100: mol_type = other DNA
ggtgtcgagaagaggagaacaatatgctaaatgttgttctcgtctcctcgacacccagtagcgccaacatgctgtaaaaaacagcatgttggagctactg

SEQ ID NO: 49 - DNA, 100 bp, synthetic construct
  source 1..100: mol_type = other DNA
ggtgtcgagaagaggagaacaatatgctaaatgttgttctcgtctcctcgacacccagtagggccaacatgctgtaaaaaacagcatgttggacctactg

SEQ ID NO: 50 - DNA, 100 bp, synthetic construct
  source 1..100: mol_type = other DNA
ggtgtcgagaagaggagaacaatatgctaaatgttgttctcgtctcctcgacacccagtagaggcaacatgctgtaaaaaacagcatgttgcatctactg

SEQ ID NO: 51 - DNA, 100 bp, synthetic construct
  source 1..100: mol_type = other DNA
ggtgtcgagaagaggagaacaatatgctaaatgttgttctcgtctcctcgacacccagtagcggcaacatgctgtaaaaaacagcatgttgcagctactg

SEQ ID NO: 52 - DNA, 95 bp, synthetic construct
  source 1..95: mol_type = other DNA
ccatatatattaagttatcgggtgtcgagaagaggagaacaatatgctaaatgttgttctcgtctcctcgacacccgataacctaatatatatgg

SEQ ID NO: 53 - DNA, 95 bp, synthetic construct
  source 1..95: mol_type = other DNA
ccatatatattaagttatcgggtgtcgagaagaggagaacaatatgctaaatgttgttctcgtctcctcgacacccgataacctgatatatatgg

SEQ ID NO: 54 - DNA, 95 bp, synthetic construct
  source 1..95: mol_type = other DNA
ccatatatattaagttatcgggtgtcgagaagaggagaacaatatgctaaatgttgttctcgtctcctcgacacccgataacctagtatatatgg

SEQ ID NO: 55 - DNA, 95 bp, synthetic construct
  source 1..95: mol_type = other DNA
ccatatatattaagttatcgggtgtcgagaagaggagaacaatatgctaaatgttgttctcgtctcctcgacacccgataaccgaatatatatgg

SEQ ID NO: 56 - DNA, 95 bp, synthetic construct
  source 1..95: mol_type = other DNA
ccatatatatttaattatcgggtgtcgagaagaggagaacaatatgctaaatgttgttctcgtctcctcgacacccgataatcaaatatatatgg

SEQ ID NO: 57 - DNA, 95 bp, synthetic construct
  source 1..95: mol_type = other DNA
ccatatatatttaattatcgggtgtcgagaagaggagaacaatatgctaaatgttgttctcgtctcctcgacacccgatagtcaaatatatatgg

SEQ ID NO: 58 - DNA, 95 bp, synthetic construct
  source 1..95: mol_type = other DNA
ccatatatatttaattatcgggtgtcgagaagaggagaacaatatgctaaatgttgttctcgtctcctcgacacccgatgatcaaatatatatgg

SEQ ID NO: 59 - DNA, 95 bp, synthetic construct
  source 1..95: mol_type = other DNA
ccatatatatttaattatcgggtgtcgagaagaggagaacaatatgctaaatgttgttctcgtctcctcgacacccgataagcaaatatatatgg

SEQ ID NO: 60 - DNA, 95 bp, synthetic construct
  source 1..95: mol_type = other DNA
ccatatatatataaatatcgggtgtcgagaagaggagaacaatatgctaaatgttgttctcgtctcctcgacacccgatattcatatatatatgg

SEQ ID NO: 61 - DNA, 95 bp, synthetic construct
  source 1..95: mol_type = other DNA
ccatatatatataaatatcgggtgtcgagaagaggagaacaatatgctaaatgttgttctcgtctcctcgacacccgatgttcatatatatatgg

SEQ ID NO: 62 - DNA, 95 bp, synthetic construct
  source 1..95: mol_type = other DNA
ccatatatatataaatatcgggtgtcgagaagaggagaacaatatgctaaatgttgttctcgtctcctcgacacccgatagtcatatatatatgg

SEQ ID NO: 63 - DNA, 95 bp, synthetic construct
  source 1..95: mol_type = other DNA
ccatatatataaagatatcgggtgtcgagaagaggagaacaatatgctaaatgttgttctcgtctcctcgacacccgatatccttatatatatgg

SEQ ID NO: 64 - DNA, 95 bp, synthetic construct
  source 1..95: mol_type = other DNA
ccatatatataaagatatcgggtgtcgagaagaggagaacaatatgctaaatgttgttctcgtctcctcgacacccgatatccttgtatatatgg

SEQ ID NO: 65 - DNA, 95 bp, synthetic construct
  source 1..95: mol_type = other DNA
ccatatatataaagatatcgggtgtcgagaagaggagaacaatatgctaaatgttgttctcgtctcctcgacacccgatatcctgatatatatgg

SEQ ID NO: 66 - DNA, 95 bp, synthetic construct
  source 1..95: mol_type = other DNA
ccatatattaataaaattcgggtgtcgagaagaggagaacaatatgctaaatgttgttctcgtctcctcgacacccgaatttcattaatatatgg

SEQ ID NO: 67 - DNA, 95 bp, synthetic construct
  source 1..95: mol_type = other DNA
ccatatattaataaaattcgggtgtcgagaagaggagaacaatatgctaaatgttgttctcgtctcctcgacacccgagtttcattaatatatgg

SEQ ID NO: 68 - DNA, 95 bp, synthetic construct
  source 1..95: mol_type = other DNA
ccatatattaataaaattcgggtgtcgagaagaggagaacaatatgctaaatgttgttctcgtctcctcgacacccgaagttcattaatatatgg

SEQ ID NO: 69 - DNA, 95 bp, synthetic construct
  source 1..95: mol_type = other DNA
ccatatattaaaagaattcgggtgtcgagaagaggagaacaatatgctaaatgttgttctcgtctcctcgacacccgaattcctttaatatatgg

SEQ ID NO: 70 - DNA, 95 bp, synthetic construct
  source 1..95: mol_type = other DNA
ccatatattaaaagaattcgggtgtcgagaagaggagaacaatatgctaaatgttgttctcgtctcctcgacacccgaattcctttgatatatgg

SEQ ID NO: 71 - DNA, 95 bp, synthetic construct
  source 1..95: mol_type = other DNA
ccatatattaaaagaattcgggtgtcgagaagaggagaacaatatgctaaatgttgttctcgtctcctcgacacccgaattccttgaatatatgg

SEQ ID NO: 72 - DNA, 111 bp, synthetic construct
  source 1..111: mol_type = other DNA
gtgaacgtcaaactgtcggaccaatatggcagaatcttctctcatctcaactttccatatccgtatcatggaatcatagcatcctgtaactactagctctcttacagctgg

SEQ ID NO: 73 - DNA, 111 bp, synthetic construct
  source 1..111: mol_type = other DNA
gtgaacgtcaaactgtcggaccaatgtggcagaatcttctctcatctcaactttccatgtccgtgtcatggaatcatggcatcctgtgactgctggctctcttgcagctgg

SEQ ID NO: 74 - DNA, 111 bp, synthetic construct
  source 1..111: mol_type = other DNA
gtgaacgtcaaactgtcggaccaagagggcagaatcttctctcatctcaactttccagatccggatcatggaatcagagcatcctggaacgacgagctctctgacagctgg

SEQ ID NO: 75 - DNA, 111 bp, synthetic construct
  source 1..111: mol_type = other DNA
gtgaacgtcaaactgtcggaccaagagggcagaatcgtcgcgcatcgcaacggtccagatccggatcatggaatcagagcatccgggaacgacgagctctcggacagctgg

SEQ ID NO: 76 - DNA, 111 bp, synthetic construct
  source 1..111: mol_type = other DNA
gtgaacgtcaaactgtcggaccaatgtggcagaatcgtcgcgcatctcagctttccatgtccgtgtcatggaatcatggcatccggtgactgctggctctcgtgcagctgg

SEQ ID NO: 77 - DNA, 111 bp, synthetic construct
  source 1..111: mol_type = other DNA
gtgaagataagccagtcctctagtaacagaatgagcaagacggcaagagcttacccagtcacttgtgtggagacttaaatacttgcataaagatccattgggatagtactc

SEQ ID NO: 78 - DNA, 111 bp, synthetic construct
  source 1..111: mol_type = other DNA
gtgaagataagccagtcctctggtgacagaatgagcaagacggcaagagcttgcccagtcacttgtgtggagacttgaatgcttgcatgaagatccgttgggatggtgctc

SEQ ID NO: 79 - DNA, 111 bp, synthetic construct
  source 1..111: mol_type = other DNA
gtgaagataagccagtcctcgaggaacagaatgagcaagacggcaagagctgacccagtcacttgtgtggagactgaaagacttgcagaaagatccagtgggagaggactc

SEQ ID NO: 80 - DNA, 111 bp, synthetic construct
  source 1..111: mol_type = other DNA
gtgaagataagccagtcctcgaggaacagaatgagcaagacggcaagagcggacccagtcacgggtgtggagacggaaagacgggcagaaagatccagtgggagaggactc

SEQ ID NO: 81 - DNA, 111 bp, synthetic construct
  source 1..111: mol_type = other DNA
gtgaagataagccagtcctctggtgacagaatgagcaagacggcaagagcggacccagtcacgggtgtggagacgtgaatgcgggcatgaagatccgttgggatggtgctc

SEQ ID NO: 82 - DNA, 111 bp, synthetic construct
  source 1..111: mol_type = other DNA
catattacagaataccttgatagcatccaatttgcatccttggttagggtcaacccagtattctccactcttgagttcaggatggcagaatttcaggtctctgcagtttct

SEQ ID NO: 83 - DNA, 111 bp, synthetic construct
  source 1..111: mol_type = other DNA
catattacagaataccttggtagcatccagtttgcatccttggttagggtcaacccagtgttctccactcttgagttcagggtggcagagtttcaggtctctgcagtttct

SEQ ID NO: 84 - DNA, 111 bp, synthetic construct
  source 1..111: mol_type = other DNA
catattacagaataccttgagagcatccaagttgcatccttggttagggtcaacccaggagtctccactcttgagttcaggagggcagaagttcaggtctctgcagtttct

SEQ ID NO: 85 - DNA, 111 bp, synthetic construct
  source 1..111: mol_type = other DNA
catattacagaataccttgagagcatccaaggtgcatccttggttagggtcaacccaggagtctccactcgtgagttcaggagggcagaaggtcaggtctcggcaggttct

SEQ ID NO: 86 - DNA, 111 bp, synthetic construct
  source 1..111: mol_type = other DNA
catattacagaataccttggtagcatccagtgtgcatccttggttagggtcaacccagtgttctccactcgtgagttcagggtggcagagtgtcaggtctctgcaggttct

SEQ ID NO: 87 - DNA, 111 bp, synthetic construct
  source 1..111: mol_type = other DNA
gtctttgataaaaggcgtacataattcttgtgtctactgtacagaatactgccgccagctggatttcccaattctgagtaacactctgcaatccaaacagggttcaaccct

SEQ ID NO: 88 - DNA, 111 bp, synthetic construct
  source 1..111: mol_type = other DNA
gtctttgatgaaaggcgtgcgtagttcttgtgtctgctgtgcagaatgctgccgccagctgggtttcccagttctgagtgacactctgcaatccaaacagggttcaaccct

SEQ ID NO: 89 - DNA, 111 bp, synthetic construct
  source 1..111: mol_type = other DNA
gtctttgagaaaaggcggacagaagtcttgtgtcgactggacagaagactgccgccagctggagttcccaagtctgaggaacactctgcaatccaaacagggttcaaccct

SEQ ID NO: 90 - DNA, 111 bp, synthetic construct
  source 1..111: mol_type = other DNA
gtctttgagaaaaggcggacagaagtctggtgtcgacgggacagaagacggccgccagctggagggcccaaggcggaggaacactcggcaatccaaacagggggcaaccct

SEQ ID NO: 91 - DNA, 111 bp, synthetic construct
  source 1..111: mol_type = other DNA
gtctttgatgaaaggcgtgcgtagttcgggtgtctgctgtgcagaatgcggccgccagctgggtggcccagtgcggagtgacacgcggcaatccaaacagggggcaaccct

SEQ ID NO: 92 - DNA, 111 bp, synthetic construct
  source 1..111: mol_type = other DNA
tacatcatactttccagcagatcggccagacttcctttgtttaagctttcgtgtccaaccttcaggcaaggtggggtcatcatacataggtccccggtcacggataatgga

SEQ ID NO: 93 - DNA, 158 bp, synthetic construct
  source 1..158: mol_type = other DNA
aaccatgccgactgatggcagtacatcatactttccagcagatcggccagacttcctttgtttaagctttcgtgtccaaccttcaggcaaggtggggtcatcatacataggtccccggtcacggataatggactgccatcagtcggcgtggactgtag

SEQ ID NO: 94 - DNA, 158 bp, synthetic construct
  source 1..158: mol_type = other DNA
aaccatgccgactgatggcagtacatcatactttccagcagatcggccagacttccggtgttgaagcggtcgtgtccaaccgtcaggcaaggtgggggcatcagacagaggtccccggtcacggagaatggactgccatcagtcggcgtggactgtag

SEQ ID NO: 95 - DNA, 158 bp, synthetic construct
  source 1..158: mol_type = other DNA
aaccatgccgactgatggcagtacatcatactttccagcagatcggccagacttccggtgtttgagcggtcgtgtccaaccgtcaggcaaggtgggggcatcatgcatgggtccccggtcacggatgatggactgccatcagtcggcgtggactgtag

SEQ ID NO: 96 - DNA, 83 bp, synthetic construct
  source 1..83: mol_type = other DNA
gcagaatcttggccctgaaaaagggcctcatctcaactttccatatccgtatcatgggccctgaaaaagggcctcatagcatc

SEQ ID NO: 97 - DNA, 83 bp, synthetic construct
  source 1..83: mol_type = other DNA
gcagaatcttggccctgaaaaagggcctcatctcaactttccatgtccgtgtcatgggccctgaaaaagggcctcatagcatc

SEQ ID NO: 98 - DNA, 83 bp, synthetic construct
  source 1..83: mol_type = other DNA
gcagaatcttggccctgaaaaagggcctcatctcaactttccagatccggatcatgggccctgaaaaagggcctcatagcatc

SEQ ID NO: 99 - DNA, 83 bp, synthetic construct
  source 1..83: mol_type = other DNA
gcagaatcttggccctgaaaaagggcctcatcgcaacggtccagatccggatcatgggccctgaaaaagggcctcatagcatc

SEQ ID NO: 100 - DNA, 83 bp, synthetic construct
  source 1..83: mol_type = other DNA
gcagaatcttggccctgaaaaagggcctcatcgcaacggtccatgtccgtgtcatgggccctgaaaaagggcctcatagcatc

SEQ ID NO: 101 - DNA, 87 bp, synthetic construct
  source 1..87: mol_type = other DNA
ctctcatctcaactttccatatccgtatcatggaatcatagcatcctgtaagttgtggaaggtccagttttgaggggctattacaac

SEQ ID NO: 102 - DNA, 87 bp, synthetic construct
  source 1..87: mol_type = other DNA
ctctcatctcaactttccatgtccgtgtcatggaatcatagcatcctgtaagttgtggaaggtccagttttgaggggctattacaac

SEQ ID NO: 103 - DNA, 87 bp, synthetic construct
  source 1..87: mol_type = other DNA
ctctcatctcaactttccagatccggatcatggaatcatagcatcctgtaagttgtggaaggtccagttttgaggggctattacaac

SEQ ID NO: 104 - DNA, 87 bp, synthetic construct
  source 1..87: mol_type = other DNA
ctctcatctcaacggtccagatccggatcatggaatcatagcatcctgtaagttgtggaaggtccagttttgaggggctattacaac

SEQ ID NO: 105 - DNA, 87 bp, synthetic construct
  source 1..87: mol_type = other DNA
ctctcatctcaacggtccatgtccgtgtcatggaatcatagcatcctgtaagttgtggaaggtccagttttgaggggctattacaac

SEQ ID NO: 106 - DNA, 144 bp, synthetic construct
  source 1..144: mol_type = other DNA
ggtgtcgagaagaggagaacaatatctttcatgttgttctcctctcctcgacaccgcttacccagtcacttgtgtaaaggacgctcatccaaggagccaaacggccaatcagctccaacagaaaccagggcacccgccagggcc

SEQ ID NO: 107 - DNA, 144 bp, synthetic construct
  source 1..144: mol_type = other DNA
ggtgtcgagaagaggagaacaatatctttcatgttgttctcctctcctcgacaccgcttacccagtcacttgtgtaaagggatagtactccatcacaaaaaaatattccatgcgtccatctaaaatctccaactataaagcggg

SEQ ID NO: 108 - DNA, 144 bp, synthetic construct
  source 1..144: mol_type = other DNA
ggtgtcgagaagaggagaacaatatctttcatgttgttctcctctcctcgacaccgcttacccagtcacttgtgtaaaggggtagtgctccatcacaaaaaaatgttccgtgcgtccatctaaaatctccaactgtaaagcggg

SEQ ID NO: 109 - DNA, 144 bp, synthetic construct
  source 1..144: mol_type = other DNA
ggtgtcgagaagaggagaacaatatctttcatgttgttctcctctcctcgacaccgcttacccagtcacttgtgtaaagggagaggactccatcacaaaaaaagagtccaggcgtccatctaaaatctccaacgagaaagcggg

SEQ ID NO: 110 - DNA, 144 bp, synthetic construct
  source 1..144: mol_type = other DNA
ggtgtcgagaagaggagaacaatatgctaaatgttgttctcgtctcctcgacaccccaccgcctaagagaataggaaatgctgccatccaggacatttaaactgaaagatccagagaattaaaagaacaccctgtgcaagaaca

SEQ ID NO: 111 - DNA, 144 bp, synthetic construct
  source 1..144: mol_type = other DNA
ggtgtcgagaagaggagaacaatatgctaaatgttgttctcgtctcctcgacaccccaccgcctgagagaataggaaatgctgccatccaggacatttaaactgaaagatccagagaattaaaagaacaccctgtgcaagaaca

SEQ ID NO: 112 - DNA, 144 bp, synthetic construct
  source 1..144: mol_type = other DNA
ggtgtcgagaagaggagaacaatatgctaaatgttgttctcgtctcctcgacaccccaccgccgaagagaataggaaatgctgccatccaggacatttaaactgaaagatccagagaattaaaagaacaccctgtgcaagaaca

SEQ ID NO: 113 - DNA, 143 bp, synthetic construct
  source 1..143: mol_type = other DNA
ggtgtcgagaagaggagaacaatatgctaaatgttgttctcgtctcctcgacaccccaccgccaagagaataggaaatgctgccatccaggacatttaaactgaaagatccagagaattaaaagaacaccctgtgcaagaaca

SEQ ID NO: 114 - DNA, 144 bp, synthetic construct
  source 1..144: mol_type = other DNA
ggtgtcgagaagaggagaacaatatgctaaatgttgttctcgtctcctcgacacctgtccatcaacagacttgagaaaacggggcaaaaccgccagctaaatcacctccaatcccagcaataaactccaacaccaccacagcaa

SEQ ID NO: 115 - DNA, 144 bp, synthetic construct
  source 1..144: mol_type = other DNA
ggtgtcgagaagaggagaacaatatgctaaatgttgttctcgtctcctcgacacctgtccgtcaacagacttgagaaaacggggcaaaaccgccagctaaatcacctccaatcccagcaataaactccaacaccaccacagcaa

SEQ ID NO: 116 - DNA, 144 bp, synthetic construct
  source 1..144: mol_type = other DNA
ggtgtcgagaagaggagaacaatatgctaaatgttgttctcgtctcctcgacacctgtccagcaacagacttgagaaaacggggcaaaaccgccagctaaatcacctccaatcccagcaataaactccaacaccaccacagcaa

SEQ ID NO: 117 - DNA, 143 bp, synthetic construct
  source 1..143: mol_type = other DNA
ggtgtcgagaagaggagaacaatatgctaaatgttgttctcgtctcctcgacacctgtccacaacagacttgagaaaacggggcaaaaccgccagctaaatcacctccaatcccagcaataaactccaacaccaccacagcaa

SEQ ID NO: 118 - DNA, 144 bp, synthetic construct
  source 1..144: mol_type = other DNA
ggtgtcgagaagaggagaacaatatctttcatgttgttctcctctcctcgacacccagtagccacaacatgctgtaaaggcactcggtcaccagtcagaaaaaaccctccatcagagtccaaaaaggcagcggtcgccagcggg

SEQ ID NO: 119 - DNA, 144 bp, synthetic construct
  source 1..144: mol_type = other DNA
ggtgtcgagaagaggagaacaatatctttcatgttgttctcctctcctcgacacccagtagtcacaacatgctgtaaaggcactcggtcaccagtcagaaaaaaccctccatcagagtccaaaaaggcagcggtcgccagcggg

SEQ ID NO: 120 - DNA, 144 bp, synthetic construct
  source 1..144: mol_type = other DNA
ggtgtcgagaagaggagaacaatatctttcatgttgttctcctctcctcgacacccagtagttacaacatgctgtaaaggcactcggtcaccagtcagaaaaaaccctccatcagagtccaaaaaggcagcggtcgccagcggg

SEQ ID NO: 121 - DNA, 144 bp, synthetic construct
  source 1..144: mol_type = other DNA
ggtgtcgagaagaggagaacaatatctttcatgttgttctcctctcctcgacacccagattccaggtgggacgcaaaagccacagaccacatcacgacaaacacgccgggacactcatcgaaaagcaccaagccagcgaagcag

SEQ ID NO: 122 - DNA, 144 bp, synthetic construct
  source 1..144: mol_type = other DNA
ggtgtcgagaagaggagaacaatatctttcatgttgttctcctctcctcgacacccagatttcaggtgggacgcaaaagccacagaccacatcacgacaaacacgccgggacactcatcgaaaagcaccaagccagcgaagcag

SEQ ID NO: 123 - DNA, 144 bp, synthetic construct
  source 1..144: mol_type = other DNA
ggtgtcgagaagaggagaacaatatctttcatgttgttctcctctcctcgacacccagattttaggtgggacgcaaaagccacagaccacatcacgacaaacacgccgggacactcatcgaaaagcaccaagccagcgaagcag

SEQ ID NO: 124 - DNA, 144 bp, synthetic construct
  source 1..144: mol_type = other DNA
ggtgtcgagaagaggagaacaatatctttcatgttgttctcctctcctcgacaccacgcaaccaagtcatagtccaaaagccgccgatccacacggagaaacctcagggcagcggaaccgcaaagctcgaagtccagggcgacg

SEQ ID NO: 125 - DNA, 144 bp, synthetic construct
  source 1..144: mol_type = other DNA
ggtgtcgagaagaggagaacaatatctttcatgttgttctcctctcctcgacaccacgcaatcaagtcatagtccaaaagccgccgatccacacggagaaacctcagggcagcggaaccgcaaagctcgaagtccagggcgacg

SEQ ID NO: 126 - DNA, 144 bp, synthetic construct
  source 1..144: mol_type = other DNA
ggtgtcgagaagaggagaacaatatctttcatgttgttctcctctcctcgacaccacgcaattaagtcatagtccaaaagccgccgatccacacggagaaacctcagggcagcggaaccgcaaagctcgaagtccagggcgacg

SEQ ID NO: 127 - DNA, 35 bp, synthetic construct
  source 1..35: mol_type = other DNA
gcggatccaccatggctagcaaaggagaagaactc

SEQ ID NO: 128 - DNA, 20 bp, synthetic construct
  source 1..20: mol_type = other DNA
ctagaaggcacagtcgaggc

SEQ ID NO: 129 - DNA, 28 bp, synthetic construct
  source 1..28: mol_type = other DNA
gctggcaactagaaggcacagtcgaggc

SEQ ID NO: 130 - DNA, 29 bp, synthetic construct
  source 1..29: mol_type = other DNA
gacacgtgctgaagtcaagtttgaaggtg

SEQ ID NO: 131 - DNA, 27 bp, synthetic construct
  source 1..27: mol_type = other DNA
cagcagatgtggatcagcaagcaggag

SEQ ID NO: 132 - DNA, 25 bp, synthetic construct
  source 1..25: mol_type = other DNA
ggaagggggggcacgaaggctcatc

SEQ ID NO: 133 - DNA, 21 bp, synthetic construct
  source 1..21: mol_type = other DNA
gccgtatcccaaacaaacacc

SEQ ID NO: 134 - DNA, 21 bp, synthetic construct
  source 1..21: mol_type = other DNA
ccaaacaacaatcacccctgt

SEQ ID NO: 135 - DNA, 20 bp, synthetic construct
  source 1..20: mol_type = other DNA
ccttggatgagcgtccagtt

SEQ ID NO: 136 - DNA, 21 bp, synthetic construct
  source 1..21: mol_type = other DNA
tgctgggattggaggtgaaaa

SEQ ID NO: 137 - DNA, 20 bp, synthetic construct
  source 1..20: mol_type = other DNA
caacaagcatgaggatgcgg

SEQ ID NO: 138 - DNA, 20 bp, synthetic construct
  source 1..20: mol_type = other DNA
gtgcccgtagtcgtgatacc

SEQ ID NO: 139 - DNA, 21 bp, synthetic construct
  source 1..21: mol_type = other DNA
tgtgtgcaacaacccagaaat

SEQ ID NO: 140 - DNA, 23 bp, synthetic construct
  source 1..23: mol_type = other DNA
gtactgcttcttcctccttggtg

SEQ ID NO: 141 - DNA, 20 bp, synthetic construct
  source 1..20: mol_type = other DNA
gccccattacaggctcacac

SEQ ID NO: 142 - DNA, 20 bp, synthetic construct
  source 1..20: mol_type = other DNA
ttgaagtgtggagcaggggg

SEQ ID NO: 143 - DNA, 20 bp, synthetic construct
  source 1..20: mol_type = other DNA
ccagacgattgcacggaatg

SEQ ID NO: 144 - DNA, 20 bp, synthetic construct
  source 1..20: mol_type = other DNA
aaccttcagcccaccattct

SEQ ID NO: 145 - DNA, 151 bp, synthetic construct
  source 1..151: mol_type = other DNA
gggcttcgactggccgctgctggtgtttttcctgcttcgctggcttggtgccttttttcgatgagtgtcccggcgtgggcctggcgctgccgctttttccccgcggtcgtgatgtggtctgtggccttttttgcgtcccacctcgaatgcc

SEQ ID NO: 146 - DNA, 87 bp, synthetic construct
  source 1..87: mol_type = other DNA
gttggagaggatggtcgatttttctcctgagattctttttttctgactggtgaccgagtgcctttttacagcatgttgtcgctagcc

SEQ ID NO: 147 - DNA, 18 bp, synthetic construct
  source 1..18: mol_type = other DNA
ctgctgcggccgctttcg

SEQ ID NO: 148 - DNA, 89 bp, synthetic construct
  source 1..89: mol_type = other DNA
cgtcgccctggacttcgagctttgcggttccgctgccctgaggtttctccgtgtggatcggcggcttttggactatgacttcgttggca

SEQ ID NO: 149 - DNA, 27 bp, synthetic construct
  source 1..27: mol_type = other DNA
acaacaagtttcccagggctcttctcc

SEQ ID NO: 150 - DNA, 25 bp, synthetic construct
  source 1..25: mol_type = other DNA
ctagcgctaccggactcagatctcg

SEQ ID NO: 151 - DNA, 6226 bp, synthetic construct
  source 1..6226: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattccaccatggccgccgctgccgccaccgccgccgccgccgccgcgccgagcggaggaggaggaggaggcgaggaggagagactggaggaaaagtcagaagaccaggatctccagggcctcagagacaagccactgaagtttaagaaggcgaagaaagacaagaaggaggacaaagaaggcaagcatgagccactacaaccttcagcccaccattctgcagagccagcagaggcaggcaaagcagaaacatcagaaagctcaggctctgccccagcagtgccagaagcctcggcttcccccaaacagcggcgctccattatccgtgaccggggacctatgtatgatgaccccaccttgcctgaaggttagacacgaaagcttaaacaaaggaagtctggccgatctgctggaaagtatgatgtatatttgatcaatccccagggaaaagcttttcgctctaaagtagaattgattgcatactttgaaaaggtgggagacacctccttggaccctaatgattttgacttcacggtaactgggagagggagcccctccaggagagagcagaaaccacctaagaagcccaaatctcccaaagctccaggaactggcaggggtcggggacgccccaaagggagcggcactgggagaccaaaggcagcagcatcagaaggtgttcaggtgaaaagggtcctggagaagagccctgggaaacttgttgtcaagatgcctttccaagcatcgcctgggggtaagggtgagggaggtggggctaccacatctgcccaggtcatggtgatcaaacgccctggcagaaagcgaaaagctgaagctgacccccaggccattcctaagaaacggggtagaaagcctgggagtgtggtggcagctgctgcagctgaggccaaaaagaaagccgtgaaggagtcttccatacggtctgtgcatgagactgtgctccccatcaagaagcgcaagacccgggagacggtcagcatcgaggtcaaggaagtggtgaagcccctgctggtgtccacccttggtgagaaaagcgggaagggactgaagacctgcaagagccctgggcgtaaaagcaaggagagcagccccaaggggcgcagcagcagtgcctcctccccacctaagaaggagcaccatcatcaccaccatcactcagagtccacaaaggcccccatgccactgctcccatccccacccccacctgagcctgagagctctgaggaccccatcagcccccctgagcctcaggacttgagcagcagcatctgcaaagaagagaagatgccccgaggaggctcactggaaagcgatggctgccccaaggagccagctaagactcagcctatggtcgccaccactaccacagttgcagaaaagtacaaacaccgaggggagggagagcgcaaagacattgtttcatcttccatgccaaggccaaacagagaggagcctgtggacagccggacgcccgtgaccgagagagttagcggtaccgcgggcccgggatccatcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 152 - DNA, 8941 bp, synthetic construct
  source 1..8941: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcatgcctacagctgagagtgaagcaaaagtaaaaaccaaagttcgctttgaagaattgcttaagacccacagtgatctaatgcgtgaaaagaaaaaactgaagaaaaaacttgtcaggtctgaagaaaacatctcacctgacactattagaagcaatcttcactatatgaaagaaactacaagtgatgatcccgacactattagaagcaatcttccccatattaaagaaactacaagtgatgatgtaagtgctgctaacactaacaacctgaagaagagcacgagagtcactaaaaacaaattgaggaacacacagttagcaactgaaaatcctaatggtgatgctagtgtagaggaagacaaacaaggaaagccaaataaaaaggtgataaagacggtgccccagttgactacacaagacctgaaaccggaaactcctgagaataaggttgattctacacaccagaaaacacatacaaagccacagccaggcgttgatcatcagaaaagtgagaaggcaaatgagggaagagaagagactgatttagaagaggatgaagaattgatgcaagcatatcagtgccatgtaactgaagaaatggcaaaggagattaagaggaaaataagaaagaaactgaaagaacagttgacttactttccctcagatactttattccatgatgacaaactaagcagtgaaaaaaggaaaaagaaaaaggaagttccagtcttctctaaagctgaaacaagtacattgaccatctctggtgacacagttgaaggtgaacaaaagaaagaatcttcagttagatcagtttcttcagattctcatcaagatgatgaaataagctcaatggaacaaagcacagaagacagcatgcaagatgatacaaaacctaaaccaaaaaaaacaaaaaagaagactaaagcagttgcagataataatgaagatgttgatggtgatggtgttcatgaaataacaagccgagatagcccggtttatcccaaatgtttgcttgatgatgaccttgtcttgggagtttacattcaccgaactgatagacttaagtcagattttatgatttctcacccaatggtaaaaattcatgtggttgatgagcatactggtcaatatgtcaagaaagatgatagtggacggcctgtttcatcttactatgaaaaagagaatgtggattatattcttcctattatgacccagccatatgattttaaacagttaaaatcaagacttccagagtgggaagaacaaattgtatttaatgaaaattttccctatttgcttcgaggctctgatgagagtcctaaagtcatcctgttctttgagattcttgatttcttaagcgtggatgaaattaagaataattctgaggttcaaaaccaagaatgtggctttcggaaaattgcctgggcatttcttaagcttctgggagccaatggaaatgcaaacatcaactcaaaacttcgcttgcagctatattacccacctactaagcctcgatccccattaagtgttgttgaggcatttgaatggtggtcaaaatgtccaagaaatcattacccatcaacactgtacgtaactgtaagaggactgaaagttccagactgtataaagccatcttaccgctctatgatggctcttcaggaggaaaaaggtaaaccagtgcattgtgaacgtcaccatgagtcaagctcagtagacacagaacctggattagaagagtcaaaggaagtaataaagtggaaacgactccctgggcaggcttgccgtatcccaaacaaacacctcttctcactaaatgcaggagaacgaggatgtttttgtcttgatttctcccacaatggaagaatattagcagcagcttgtgccagccgggatggatatccaattattttatatgaaattccttctggacgtttcatgagagaattgtgtggccacctcaatatcatttatgatctttcctggtcaaaagatgatcactacatccttacttcatcatctgatggcactgccaggatatggaaaaatgaaataaacaatacaaatactttcagagttttacctcatccttcttttgtttacacggctaaattccatccagctgtaagagagctagtagttacaggatgctatgattccatgatacggatatagaaagttgagatgagagaagattctgccatattggtccgacagtttgacgttcacaaaagttttatcaactcactttgttttgatactgaaggtcatcatatgtattcaggagattgtacaggggtgattgttgtttggaatacctatgtcaagattaatgatttggaacattcagtgcaccactggactataaataaggaaattaaagaaactgagtttaagggaattccaataagttatttggagattcatcccaatggaaaacgtttgttaatccataccaaagacagtactttgagaattatggatctccggatattagtagcaaggaagtttgtaggagcagcaaattatcgggagaagattcatagtactttgactccatgtgggacttttctgtttgctggaagtgaggatggtatagtgtatgtttggaacccagaaacaggagaacaagtagccatgtattctgacttgccattcaagtcacccattcgagacatttcttatcatccatttgaaaatatggttgcattctgtgcatttgggcaaaatgagccaattcttctgtatatttacgatttccatgttgcccagcaggaggctgaaatgttcaaacgctacaatggaacatttccattacctggaatacaccaaagtcaagatgccctatgtacctgtccaaaactaccccatcaaggctcttttcagattgatgaatttgtccacactgaaagttcttcaacgaagatgcagctagtaaaacagaggcttgaaactgtcacagaggtgatacgttcctgtgctgcaaaagtcaacaaaaatctctcatttacttcaccaccagcagtttcctcacaacagtctaagttaaagcagtcaaacatgctgaccgctcaagagattctacatcagtttggtttcactcagaccgggattatcagcatagaaagaaagccttgtaaccatcaggtagatacagcaccaacggtagtggctctttatgactacacagcgaatcgatcagatgaactaaccatccatcgcggagacattatccgagtgtttttcaaagataatgaagactggtggtatggcagcataggaaagggacaggaaggttattttccagctaatcatgtggctagtgaaacactgtatcaagaactgcctcctgagataaaggagcgatcccctcctttaagccctgaggaaaaaactaaaatagaaaaatctccagctcctcaaaagcaatcaatcaataagaacaagtcccaggacttcagactaggctcagaatctatgacacattctgaaatgagaaaagaacagagccatgaggaccaaggacacataatggatacacggatgaggaagaacaagcaagcaggcagaaaagtcactctaatagagtagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 153 - DNA, 8468 bp, synthetic construct
  source 1..8468: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcatgacttcctcgctgcagcggccctggcgggtgccctggctaccatggaccatcctgctggtcagcgctgcggctgcttcgcagaatcaagaacggctatgtgcgtttaaagatccgtatcagcaagaccttgggataggtgagagtagaatctctcatgaaaatgggacaatattatgctcgaaaggtagcacctgctatggcctttgggagaaatcaaaaggggacataaatcttgtaaaacaaggatgttggtctcacattggagatccccaagagtgtcactatgaagaatgtgtagtaactaccactcctccctcaattcagaatggaacataccgtttctgctgttgtagcacagatttatgtaatgtcaactttactgagaattttccacctcctgacacaacaccactcagtccacctcattcatttaaccgagatgagacaataatcattgctttggcatcagtctctgtattagctgttttgatagttgccttatgctttggatacagaatgttgacaggagaccgtaaacaaggtcttcacagtatgaacatgatggaggcagcagcatccgaaccctctcttgatctagataatctgaaactgttggagctgattggccgaggtcgatatggagcagtatataaaggctccttggatgagcgtccagttgctgtaaaagtgttttcctttgcaaaccgtcagaattttatcaacgaaaagaacatttacagagtgcctttgatggaacatgacaacattgcccgctttatagttggagatgagagagtcactgcagatggacgcatggaatatttgcttgtgatggagtactatcccaatggatctttatgcaagtatttaagtctccacacaagtgactaggtaagctcttgccgtcttgctcattctgttactagaggactggcttatcttcacacagaattaccacgaggagatcattataaacctgcaatttcccatcgagatttaaacagcagaaatgtcctagtgaaaaatgatggaacctgtgttattagtgactttggactgtccatgaggctgactggaaatagactggtgcgcccaggggaggaagataatgcagccataagcgaggttggcactatcagatatatggcaccagaagtgctagaaggagctgtgaacttgagggactgtgaatcagctttgaaacaagtagacatgtatgctcttggactaatctattgggagatatttatgagatgtacagacctcttcccaggggaatccgtaccagagtaccagatggcttttcagacagaggttggaaaccatcccacttttgaggatatgcaggttctcgtgtctagggaaaaacagagacccaagttcccagaagcctggaaagaaaatagcctggcagtgaggtcactcaaggagacaatcgaagactgttgggaccaggatgcagaggctcggcttactgcacagtgtgctgaggaaaggatggctgaacttatgatgatttgggaaagaaacaaatctgtgagcccaacagtcaatccaatgtctactgctatgcagaatgaacgcaacctgtcacataataggcgtgtgccaaaaattggtccttatccagattattcttcctcctcatacattgaagactctatccatcatactgacagcatcgtgaagaatatttcctctgagcattctatgtccagcacacctttgactataggggaaaaaaaccgaaattcaattaactatgaacgacagcaagcacaagctcgaatccccagccctgaaacaagtgtcaccagcctctccaccaacacaacaaccacaaacaccacaggactcacgccaagtactggcatgactactatatctgagatgccatacccagatgaaacaaatctgcataccacaaatgttgcacagtcaattgggccaacccctgtctgcttacagctgacagaagaagacttggaaaccaacaagctagacccaaaagaagttgataagaacctcaaggaaagctctgatgagaatctcatggagcactctcttaaacagttcagtggcccagacccactgagcagtactagttctagcttgctttacccactcataaaacttgcagtagaagcaactggacagcaggacttcacacagactgcaaatggccaagcatgtttgattcctgatgttctgcctactcagatctatcctctccccaagcagcagaaccttcccaagagacctactagtttgcctttgaacaccaaaaattcaacaaaagagccccggctaaaatttggcagcaagcacaaatcaaacttgaaacaagtcgaaactggagttgccaagatgaatacaatcaatgcagcagaacctcatgtggtgacagtcaccatgaatggtgtggcaggtagaaaccacagtgttaactcccatgctgccacaacccaatatgccaatgggacagtactatctggccaaacaaccaacatagtgacacatagggcccaagaaatgttgcagaatcagtttattggtgaggacacccggctgaatattaattccagtcctgatgagcatgagcctttactgagacgagagcaacaagctggccatgatgaaggtgttctggatcgtcttgtggacaggagggaacggccactagaaggtggccgaactaattccaataacaacaacagcaatccatgttcagaacaagatgttcttgcacagggtgttccaagcacagcagcagatcctgggccatcaaagcccagaagagcacagaggcctaattctctggatctttcagccacaaatgtcctggatggcagcagtatacagataggtgagtcaacacaagatggcaaatcaggatcaggtgaaaagatcaagaaacgtgtgaaaactccctattctcttaagcggtggcgcccctccacctgggtcatctccactgaatcgctggactgtgaagtcaacaataatggcagtaacagggcagttcattccaaatccagcactgctgtttaccttgcagaaggaggcactgctacaaccatggtgtctaaagatataggaatgaactgtctgtgagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 154 - DNA, 9752 bp, synthetic construct
  source 1..9752: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcatgatgagctttgtgcaaaaggggagctggctacttctcgctctgcttcatcccactattattttggcacaacaggaagctgttgaaggaggatgttcccatcttggtcagtcctatgcggatagagatgtctggaagccagaaccatgccaaatatgtgtctgtgactcaggatccgttctctgcgatgacataatatgtgacgatcaagaattagactgccccaacccagaaattccatttggagaatgttgtgcagtttgcccacagcctccaactgctcctactcgccctcctaatggtcaaggacctcaaggccccaagggagatccaggccctcctggtattcctgggagaaatggtgaccctggtattccaggacaaccagggtcccctggttctcctggcccccctggaatctgtgaatcatgccctactggtcctcagaactattctccccagtatgattcatatgatgtcaagtctggagtagcagtaggaggactcgcaggctatcctggaccagctggccccccaggccctcccggtccccctggtacatctggtcatcctggttcccctggatctccaggataccaaggaccccctggtgaacctgggcaagctggtccttcaggccctccaggacctcctggtgctataggtccatctggtcctgctggaaaagatggagaatcaggtagacccggacgacctggagagcgaggattgcctggacctccaggtatcaaaggtccagctgggatacctggattccctggtatgaaaggacacagaggcttcgatggacgaaatggagaaaagggtgaaacaggtgctcctggattaaagggtgaaaatggtcttccaggcgaaaatggagctcctggacccatgggtccaagaggggctcctggtgagcgaggacggccaggacttcctggggctgcaggtgctcggggtaatgacggtgctcgaggcagtgatggtcaaccaggccctcctggtcctcctggaactgccggattccctggatcccctggtgctaagggtgaagttggacctgcagggtctcctggttcaaatggtgcccctggacaaagaggagaacctggacctcagggacacgctggtgctcaaggtcctcctggccctcctgggattaatggtagtcctggtggtaaaggcgaaatgggtcccgctggcattcctggagctcctggactgatgggagcccggggtcctccaggaccagccggtgctaatggtgctcctggactgcgaggtggtgcaggtgagcctggtaagaatggtgccaaaggagagcccggaccacgtggtgaacgcggtgaggctggtattccaggtgttccaggagctaaaggcgaagatggcaaggatggatcacctggagaacctggtgcaaatgggcttccaggagctgcaggagaaaggggtgcccctgggttccgaggacctgctggaccaaatggcatcccaggagaaaagggtcctgctggagagcgtggtgctccaggccctgcagggcccagaggagctgctggagaacctggcagagatggcgtccctggaggtccaggaatgaggggcatgcccggaagtccaggaggaccaggaagtgatgggaaaccagggcctcccggaagtcaaggagaaagtggtcgaccaggtcctcctgggccatctggtccccgaggtcagcctggtgtcatgggcttccccggtcctaaaggaaatgatggtgctcctggtaagaatggagaacgaggtggccctggaggacctggccctcagggtcctcctggaaagaatggtgaaactggacctcagggacccccagggcctactgggcctggtggtgacaaaggagacacaggaccccctggtccacaaggattacaaggcttgcctggtacaggtggtcctccaggagaaaatggaaaacctggggaaccaggtccaaagggtgatgccggtgcacctggagctccaggaggcaagggtgatgctggtgcccctggtgaacgtggacctcctggattggcaggggccccaggacttagaggtggagctggtccccctggtcccgaaggaggaaagggtgctgctggtcctcctgggccacctggtgctgctggtactcctggtctgcaaggaatgcctggagaaagaggaggtcttggaagtcctggtccaaagggtgacaagggtgaaccaggcggtccaggtgctgatggtgtcccagggaaagatggcccaaggggtcctactggtcctattggtcctcctggcccagctggccagcctggagataagggtgaaggtggtgcccccggacttccaggtatagctggacctcgtggtagccctggtgagagaggtgaaactggccctccaggacctgctggtttccctggtgctcctggacagaatggtgaacctggtggtaaaggagaaagaggggctccgggtgagaaaggtgaaggaggccctcctggagttgcaggaccccctggaggttctggacctgctggtcctcctggtccccaaggtgtcaaaggtgaacgtggcagtcctggtggacctggtgctgctggcttccctggtgctcgtggtcttcctggtcctcctggtagtaatggtaacccaggacccccaggtcccagcggttctccaggcaaggatgggcccccaggtcctgcgggtaacactggtgctcctggcagccctggagtgtctggaccaaaaggtgatgctggccaaccaggagagaagggatcgcctggtgcccagggcccaccaggagctccaggcccacttgggattgctgggatcactggagcacggggtcttgcaggaccaccaggcatgccaggtcctaggggaagccctggccctcagggtgtcaagggtgaaagtgggaaaccaggagctaacggtctcagtggagaacgtggtccccctggaccccagggtcttcctggtctggctggtacagctggtgaacctggaagagatggaaaccctggatcagatggtcttccaggccgagatggatctcctggtggcaagggtgatcgtggtgaaaatggctctcctggtgcccctggcgctcctggtcatccaggcccacctggtcctgtcggtccagctggaaagagtggtgacagaggagaaagtggccctgctggccctgctggtgctcccggtcctgctggttcccgaggtgctcctggtcctcaaggcccacgtggtgacaaaggtgaaacaggtgaacgtggagctgctggcatcaaaggacatcgaggattccctggtaatccaggtgccccaggttctccaggccctgctggtcagcagggtgcaatcggcagtccaggacctgcaggccccagaggacctgttggacccagtggacctcctggcaaagatggaaccagtggacatccaggtcccattggaccaccagggcctcgaggtaacagaggtgaaagaggatctgagggctccccaggccacccagggcaaccaggccctcctggacctcctggtgcccctggtccttgctgtggtggtgttggagccgctgccattgctgggattggaggtgaaaaagctggcggttttgccccgtattatggagatgaaccaatggatttcaaaatcaacaccgatgagattatgacttcactcaagtctgttaatggacaaatagaaagcctcattagtcctgatggttctcgtaaaaaccccgctagaaactgcagagacctgaaattctgccatcctgaactcaagagtggagaatactaggttgaccctaaccaaggatgcaaattggatgctatcaaggtattctgtaatatggaaactggggaaacatgcataagtgccaatcctttgaatgttccacggaaacactggtggacagattctagtgctgagaagaaacacgtttggtttggagagtccatggatggtggttttcagtttagctacggcaatcctgaacttcctgaagatgtccttgatgtgcagctggcattccttcgacttctctccagccgagcttcccagaacatcacatatcactgcaaaaatagcattgcatacatggatcaggccagtggaaatgtaaagaaggccctgaagctgatggggtcaaatgaaggtgaattcaaggctgaaggaaatagcaaattcacctacacagttctggaggatggttgcacgaaacacactggggaatggagcaaaacagtctttgaatatcgaacacgcaaggctgtgagactacctattgtagatattgcaccctatgacattggtggtcctgatcaagaatttggtgtggacgttggccctgtttgctttttataagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 155 - DNA, 9849 bp, synthetic construct
  source 1..9849: mol_type = other DNA
cactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccaccatgaacatccccgctctggtggaaaaccagaagaagtactttggcacctacagcgtgatggccatgctgaacgctcagaccgtgctggaccacatccagaaggtggccgatattgagggcgagcagaacgagaacaacgagaatctgtggtttcaccccgtgatgagccacctgtacaacgccaagaacggctacgacaagcagcccgagaaaaccatgttcatcatcgagcggctgcagagctacttcccattcctgaagatcatggccgagaaccagagagagtacagcaacggcaagtacaagcagaaccgcgtggaagtgaacagcaacgacatcttcgaggtgctgaagcgcgccttcggcgtgctgaagatgtacagggacctgaccaacgcatacaagacctacgaggaaaagctgaacgacggctgcgagttcctgaccagcacagagcaacctctgagcggcatgatcaacaactactacacagtggccctgcggaacatgaacgagagatacggctacaagacagaggacctggccttcatccaggacaagcggttcaagttcgtgaaggacgcctacggcaagaaaaagtcccaagtgaataccggattcttcctgagcctgcaggactacaacggcgacacacagaagaagctgcacctgagcggagtgggaatcgccctgctgatctgcctgttcctggacaagcagtacatcaacatctttctgagcaggctgcccatcttctccagctacaatgcccagagcgaggaacggcggatcatcatcagatccttcggcatcaacagcatcaagctgcccaaggaccggatccacagcgagaagtccaacaagagcgtggccatggatatgctcaacgaagtgaagcggtgccccgacgagctgttcacaacactgtctgccgagaagcagtcccggttcagaatcatcagcgacgaccacaatgaagtgctgatgaagcggagcagcgacagattcgtgcctctgctgctgcagtatatcgattacggcaagctgttcgaccacatcaggttccacgtgaacatgggcaagctgagatacctgctgaaggccgacaagacctgcatcgacggccagaccagagtcagagtgatcgagcagcccctgaacggcttcggcagactggaagaggccgagacaatgcggaagcaagagaacggcaccttcggcaacagcggcatccggatcagagacttcgagaacatgaagcgggacgacgccaatcctgccaactatccctacatcgtggacacctacacacactacatcctggaaaacaacaaggtcgagatgtttatcaacgacaaagaggacagcgccccactgctgcccgtgatcgaggatgatagatacgtggtcaagacaatccccagctgccggatgagcaccctggaaattccagccatggccttccacatgtttctgttcggcagcaagaaaaccgagaagctgatcgtggacgtgcacaaccggtacaagagactgttccaggccatgcagaaagaagaagtgaccgccgagaatatcgccagcttcggaatcgccgagagcgacctgcctcagaagatcctggatctgatcagcggcaatgcccacggcaaggatgtggacgccttcatcagactgaccgtggacgacatgctgaccgacaccgagcggagaatcaagagattcaaggacgaccggaagtccattcggagcgccgacaacaagatgggaaagagaggcttcaagcagatctccacaggcaagctggccgacttcctggccaaggacatcgtgctgtttcagcccagcgtgaacgatggcgagaacaagatcaccggcctgaactaccggatcatgcagagcgccattgccgtgtacgatagcggcgacgattacgaggccaagcagcagttcaagctgatgttcgagaaggcccggctgatcggcaagggcacaacagagcctcatccatttctgtacaaggtgttcgcccgcagcatccccgccaatgccgtcgagttctacgagcgctacctgatcgagcggaagttctacctgaccggcctgtccaacgagatcaagaaaggcaacagagtggatgtgcccttcatccggcgggaccagaacaagtggaaaacacccgccatgaagaccctgggcagaatctacagcgaggatctgcccgtggaactgcccagacagatgttcgacaatgagatcaagtcccacctgaagtccctgccacagatggaaggcatcgacttcaacaatgccaacgtgacctatctgatcgccgagtacatgaagagagtgctggacgacgacttccagaccttctaccagtggaaccgcaactaccggtacatggacatgcttaagggcgagtacgacagaaagggctccctgcagcactgcttcaccagcgtggaagagagagaaggcctctggaaagagcgggcctccagaacagagcggtacagaaagcaggccagcaacaagatccgcagcaaccggcagatgagaaacgccagcagcgaagagatcgagacaatcctggataagcggctgagcaacagccggaacgagtaccagaaaagcgagaaagtgatccggcgctacagagtgcaggatgccctgctgtttctgctggccaaaaagaccctgaccgaactggccgatttcgacggcgagaggttcaaactgaaagaaatcatgcccgacgccgagaagggaatcctgagcgagatcatgcccatgagcttcaccttcgagaaaggcggcaagaagtacaccatcaccagcgagggcatgaagctgaagaactacggcgacttctttgtgctggctagcgacaagaggatcggcaacctgctggaactcgtgggcagcgacatcgtgtccaaagaggatatcatggaagagttcaacaaatacgaccagtgcaggcccgagatcagctccatcgtgttcaacctggaaaagtgggccttcgacacataccccgagctgtctgccagagtggaccgggaagagaaggtggacttcaagagcatcctgaaaatcctgctgaacaacaagaacatcaacaaagagcagagcgacatcctgcggaagatccggaacgccttcgatgcaaacaattaccccgacaaaggcgtggtggaaatcaaggccctgcctgagatcgccatgagcatcaagaaggcctttggggagtacgccatcatgaagggaagcctgcagctgcctccacttgaaagactgacactgggatcccagctgcatttaccgcaggttttagctgacgctgtctcacgcctggtcctgggtaagtttggtgacctgaccgacaacttctcctcccctcacgctcgcagaaaagtgctggctggagtcgtcatgacaacaggcacagatgttaaagatgccaaggtgataagtgtttctacaggaacaaaatgtattaatggtgaatacatgagtgatcgtggccttgcattaaatgactgccatgcagaaataatatctcggagatccttgctcagatttctttatacacaacttgagctttacttaaataacaaagatgatcaaaaaagatccatctttcagaaatcagagcgaggggggtttaggctgaaggagaatgtccagtttcatctgtacatcagcacctctccctgtggagatgccagaatcttctcaccacatgagccaatcctggaagaaccagcagatagacacccaaatcgtaaagcaagaggacagctacggaccaaaatagagtctggtcaggggacgattccagtgcgctccaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcagtggcatcagcaatgcagaagcacggcagccagggaaggcccccaacttcagtgtcaactggacggtaggcgactccgctattgaggtcatcaacgccacgactgggaaggatgagctgggccgcgcgtcccgcctgtgtaagcacgcgttgtactgtcgctggatgcgtgtgcacggcaaggttccctcccacttactacgctccaagattaccaagcccaacgtgtaccatgagtccaagctggcggcaaaggagtaccaggccgccaaggcgcgtctgttcacagccttcatcaaggcggggctgggggcctgggtggagaagcccaccgagcaggaccagttctcactcacgtaagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaa

SEQ ID NO: 156 - DNA, 7070 bp, synthetic construct
  source 1..7070: mol_type = other DNA
tatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattcgccaccatggactacaaggacgacgatgacaaactagtaaatgcccgtacgcgccggcgcgaacggcgagcagaaaagcaggcacagtggaaggcagctaatggcggagggggaagcgggggcggtggaagtgggggtggaggcagcaatgcgcggacccgccgacgtgaacggcgagcagaaaaacaggcgcaatggaaagcagccaacggcggcggtggaagtggaggtggtggaagcggaggcggtggtagtaacgccagaacccgacgcagagagaggagggcagagaaacaggcccagtggaaagccgctaacggaggagggggatccggtggaggcggcagtggaggcgggggatctaacgcacgaacacgacgacgtgagcgtcgcgctgagaaacaagctcaatggaaagctgcaaacttgcacttggatcagacgccatctcgccagcctattcccagtgagggtcttcagctgcatttaccgcaggttttagctgacgctgtctcacgcctggtcctgggtaagtttggtgacctgaccgacaacttctcctcccctcacgctcgcagaaaagtgctggctggagtcgtcatgacaacaggcacagatgttaaagatgccaaggtgataagtgtttctacaggaacaaaatgtattaatggtgaatacatgagtgatcgtggccttgcattaaatgactgccatgcagaaataatatctcggagatccttgctcagatttctttatacacaacttgagctttacttaaataacaaagatgatcaaaaaagatccatctttcagaaatcagagcgaggggggtttaggctgaaggagaatgtccagtttcatctgtacatcagcacctctccctgtggagatgccagaatcttctcaccacatgagccaatcctggaagaaccagcagatagacacccaaatcgtaaagcaagaggacagctacggaccaaaatagagtctggtcaggggacgattccagtgcgctccaatgcgagcatccaaacgtgggacggggtgctgcaaggggagcggctgctcaccatgtcctgcagtgacaagattgcacgctggaacgtggtgggcatccagggatccctgctcagcattttcgtggagcccatttacttctcgagcatcatcctgggcagcctttaccacggggaccacctttccagggccatgtaccagcggatctccaacatagaggacctgccacctctctacaccctcaacaagcctttgctcagtggcatcagcaatgcagaagcacggcagccagggaaggcccccaacttcagtgtcaactggacggtaggcgactccgctattgaggtcatcaacgccacgactgggaaggatgagctgggccgcgcgtcccgcctgtgtaagcacgcgttgtactgtcgctggatgcgtgtgcacggcaaggttccctcccacttactacgctccaagattaccaaacccaacgtgtaccatgagtccaagctggcggcaaaggagtaccaggccgccaaggcgcgtctgttcacagccttcatcaaggcggggctgggggcctgggtggagaagcccaccgagcaggaccagttctcactcacgccactagtccaccatcaccatcaccattgagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccaga

SEQ ID NO: 157 - DNA, 6158 bp, synthetic construct
  source 1..6158: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagcttggtaccgagctcggatccaccatggctagcaaaggagaagaactcttcactggagttgtcccaattcttgttgaattagatggtgatgttaacggccacaagttctctgtcagtggagagggtgaaggtgatgcaacatacggaaaacttaccctgaagttcatctgcactactggcaaactgcctgttccgtggccgacactagtgacgacgctctgctatggcgtccagtgcttttcaagatacccggatcacatgaaacggcatgactttttcaagagtgccatgcccgaaggttatgtacaggaaaggaccatcttcttcaaagatgacggcaactacaagacacgtgctgaagtcaagtttgaaggtgatacccttgttaatagaatcgagttaaaaggtattgacttcaaggaagatggcaacattctgggacacaaattggaatacaactataactcacacaatgtatacatcatggcagacaaacaaaagaatggaatcaaagtgaacttcaagacccgccacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaatccggctctagagggcccttcgaacaaaaactcatctcagaagaggatctgaatatgcataccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 158 - DNA, 4902 bp, synthetic construct
  source 1..4902: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttatcgactacggaagactaggtgtcgaggagacgagaacaacatttagcatattgttctcctcttctcgacaccggatcccgcgtcctttccacaagatatataaacccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattctaattatctctctaacagccttgtatcgtatatgcaaatatgaaggaatcatgggaaataggccctcttcctgcccgaccttggcgcgcgctcggcgcgcggtcacgctccgtcacgtggtgcgttttgcctgcgcgtctttccactggggaattcatgcttctcctccctttagtgagggtaattctctctctctccctatagtgagtcgtattaattccttctcttctatagtgtcacctaaatcgttgcaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaaaaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattcaggcttacctttatgcttccggctcgtatgttgtgtggaattgtgagcggataacaatttcacacaggaaacagctatgaccatgattacgccaagctctagctagaggtcgacggtatacagacatgataagatacattgatgagtttggacaaaccacaactagaatgcagtgaaaaaaatgctttatttgtgaaatttgtgatgctattgctttatttgtaaccattataagctgcaataaacaagttggggtgggcgaagaactccagcatgagatccccgcgctggaggatcatccagccggcgtcccggaaaacgattccgaagcccaacctttcatagaaggcggcggtggaatcgaaatctcgtagcacgtgctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatcacgtgctgatcagatccgaaaatggatatacaagctcccgggagctttttgcaaaagcctaggcctccaaaaaagcctccccactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctaactaattgagatgcatgctttgcatacttctgcctgctggaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 159 - DNA, 2551 bp, synthetic construct
  source 1..2551: mol_type = other DNA
gctagcgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgctcgcttcggcagcacatatactagtcgacgccatcagtcgccggtcccaagcccggataaaatgggagggggcgggaaaccgcctaaccatgccgactgatggcagtagtcttcactggtaccgagctcggatccactggaattcgccatgcatctagggccctagaagactactgccatcagtcggcgtggactgtagaacactgccaatgccggtcccaagcccggataaaagtggagggtacagtccacgctctagagcggacttcggtccgctttttactaggacctgcaggcatgcaagcttgacgtcggttaccgatatccatatggcggccgcatcgatctcgagccgcggactagtaacttgtttattgcagcttataatggttacaaataaagcaatagcatcacaaatttcacaaataaagcatttttttcactgcattctagttgtggtttgtccaaactcatcaatgtatcttaaccggtggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaacggccggatttcttgtcacgctttgatgcatcgttggtggttgatggatatctgacgaacatgtgcacagtggtacaaggtattcctggttg

SEQ ID NO: 160 - DNA, 83 bp, synthetic construct
  source 1..83: mol_type = other DNA
actagctctcggccctgaaaaagggcccagctggatggaatctagccgtgtaaacaggccctgaaaaagggccgaaggatgag

SEQ ID NO: 161 - DNA, 83 bp, synthetic construct
  source 1..83: mol_type = other DNA
actagctctcggccctgaaaaagggcccagctggatggaagcgagccgtgtaaacaggccctgaaaaagggccgaaggatgag

SEQ ID NO: 162 - DNA, 83 bp, synthetic construct
  source 1..83: mol_type = other DNA
actagctctcggccctgaaaaagggcccagctggatggagtctggccgtgtaaacaggccctgaaaaagggccgaaggatgag

SEQ ID NO: 163 - DNA, 83 bp, synthetic construct
  source 1..83: mol_type = other DNA
atgacccaggggccctgaaaaagggccaggggcgccaccgcctaagagaatagggaggccctgaaaaagggccttcacacgtt

SEQ ID NO: 164 - DNA, 83 bp, synthetic construct
  source 1..83: mol_type = other DNA
atgacccaggggccctgaaaaagggccaggggcgccaccgccgaagagaatagggaggccctgaaaaagggccttcacacgtt

SEQ ID NO: 165 - DNA, 83 bp, synthetic construct
  source 1..83: mol_type = other DNA
atgacccaggggccctgaaaaagggccaggggcgccaccgcctgagagaatagggaggccctgaaaaagggccttcacacgtt

SEQ ID NO: 166 - DNA, 84 bp, synthetic construct
  source 1..84: mol_type = other DNA
gcttgttcttcggccctgaaaaagggccatccgtgtatccatcatgtgtccttggtcggccctgaaaaagggccatggctctgt

SEQ ID NO: 167 - DNA, 84 bp, synthetic construct
  source 1..84: mol_type = other DNA
gcttgttcttcggccctgaaaaagggccatccgtgtatccagcatgtgtccttggtcggccctgaaaaagggccatggctctgt

SEQ ID NO: 168 - DNA, 84 bp, synthetic construct
  source 1..84: mol_type = other DNA
gcttgttcttcggccctgaaaaagggccatccgtgtatccgtcatgtgtccttggtcggccctgaaaaagggccatggctctgt

SEQ ID NO: 169 - DNA, 84 bp, synthetic construct
  source 1..84: mol_type = other DNA
gtggaatgaacggccctgaaaaagggcccctgttactgccatcattgttgacttcacggccctgaaaaagggccccagcgattc

SEQ ID NO: 170 - DNA, 84 bp, synthetic construct
  source 1..84: mol_type = other DNA
gtggaatgaacggccctgaaaaagggcccctgttactgccagcattgttgacttcacggccctgaaaaagggccccagcgattc

SEQ ID NO: 171 - DNA, 84 bp, synthetic construct
  source 1..84: mol_type = other DNA
gtggaatgaacggccctgaaaaagggcccctgttactgccgtcattgttgacttcacggccctgaaaaagggccccagcgattc

SEQ ID NO: 172 - DNA, 83 bp, synthetic construct
  source 1..83: mol_type = other DNA
gactaatgagggccctgaaaaagggccttctatttgtccatcaacagacttgagtgggccctgaaaaagggcctcataatctc

SEQ ID NO: 173 - DNA, 83 bp, synthetic construct
  source 1..83: mol_type = other DNA
gactaatgagggccctgaaaaagggccttctatttgtccagcaacagacttgagtgggccctgaaaaagggcctcataatctc

SEQ ID NO: 174 - DNA, 83 bp, synthetic construct
  source 1..83: mol_type = other DNA
gactaatgagggccctgaaaaagggccttctatttgtccgtcaacagacttgagtgggccctgaaaaagggcctcataatctc

SEQ ID NO: 175 - DNA, 220 bp, synthetic construct
  source 1..220: mol_type = other DNA
aaccatgccgactgatggcagaatgttgttctcgtctcctcgacacctcgtgtccaaccttcaggcaaaaagtcatcatacataggtccccaaacacggataatggagcgccgcaaagatcaaatatacatcatactaaatccagcagatcggccagactaaaaaggtgtcgagaagaggagaacaatatgctactgccatcagtcggcgtggactgtag

SEQ ID NO: 176 - DNA, 220 bp, synthetic construct
  source 1..220: mol_type = other DNA
aaccatgccgactgatggcagcatgttgttctcgtctcctcgacacctcgtgtccaaccttcaggcaaaaagtcatcatacataggtccccaaacacggataatggagcgccgcaaagatcaaatatacatcatactaaatccagcagatcggccagactaaaaaggtgtcgagaagaggagaacaatatctttctgccatcagtcggcgtggactgtag

SEQ ID NO: 177 - DNA, 220 bp, synthetic construct
  source 1..220: mol_type = other DNA
aaccatgccgactgatggcagcatgttgttctcgtctcctcgacacctcgtgtccaaccttcaggcaaaaagtcatcatgcataggtccccaaacacggatgatggagcgccgcaaagatcaaatatacatcgtactaaatccagcagatcggccagactaaaaaggtgtcgagaagaggagaacaatatctttctgccatcagtcggcgtggactgtag

SEQ ID NO: 178 - DNA, 220 bp, synthetic construct
  source 1..220: mol_type = other DNA
aaccatgccgactgatggcagcatgttgttctcctctcctcgacacctcgtgtccaaccttcaggcaaaaagtcatcatgcataggtccccaaacacggatgatggagcgccgcaaagatcaaatatacatcgtactaaatccagcagatcggccagactaaaaaggtgtcgagaagaggagaacaatatctttctgccatcagtcggcgtggactgtag

SEQ ID NO: 179 - RNA, 59 nt, synthetic construct
  source 1..59: mol_type = other RNA
  modified_base 1..3: mod_base = OTHER; note = 2prime-O-Methyl RNA Base
  modified_base 57..59: mod_base = OTHER; note = 2prime-O-Methyl RNA Base
  modified_base 1..26: mod_base = OTHER; note = phosphorothioate bond
  modified_base 34..59: mod_base = OTHER; note = phosphorothioate bond
tgagtatctgcctaacttcatgtaacccacgaatcttgtcccaacccaggtctctaggt

SEQ ID NO: 180 - RNA, 59 nt, synthetic construct; identical to SEQ ID NO: 179
  source 1..59: mol_type = other RNA
  modified_base 1..3: mod_base = OTHER; note = 2prime-O-Methyl RNA Base
  modified_base 54..59: mod_base = OTHER; note = 2prime-O-Methyl RNA Base
  modified_base 22..24: mod_base = OTHER; note = 2prime-O-Methyl RNA Base
  modified_base 35..37: mod_base = OTHER; note = 2prime-O-Methyl RNA Base
  modified_base 1..26: mod_base = OTHER; note = phosphorothioate bond
  modified_base 34..59: mod_base = OTHER; note = phosphorothioate bond

SEQ ID NO: 181 - RNA, 59 nt, synthetic construct
  source 1..59: mol_type = other RNA
  modified_base 1..26: mod_base = OTHER; note = phosphorothioate bond
  modified_base 34..59: mod_base = OTHER; note = phosphorothioate bond
  modified_base 1..3: mod_base = OTHER; note = 2prime-O-Methyl RNA Base
  modified_base 57..59: mod_base = OTHER; note = 2prime-O-Methyl RNA Base
  modified_base 35..37: mod_base = OTHER; note = 2prime-O-Methyl RNA Base
tgagtatctgcctaacttcatgtgacccacgaatcttgtcccaacccaggtctctgggt

SEQ ID NO: 182 - DNA, 220 bp, synthetic construct; identical to SEQ ID NO: 177
  source 1..220: mol_type = other DNA

SEQ ID NO: 183 - DNA, 23 bp, synthetic construct
  source 1..23: mol_type = other DNA
ggaagctttgtcagagccctacc

SEQ ID NO: 184 - DNA, 20 bp, synthetic construct
  source 1..20: mol_type = other DNA
aacccgtccggaaaatggcc

SEQ ID NO: 185 - DNA, 20 bp, synthetic construct
  source 1..20: mol_type = other DNA
tgtacaagaaagctgggtcg

SEQ ID NO: 186 - DNA, 19 bp, synthetic construct
  source 1..19: mol_type = other DNA
ctttgcgaagtgtcaacct

SEQ ID NO: 187 - DNA, 20 bp, synthetic construct
  source 1..20: mol_type = other DNA
ttccaggcggaccatacaac

SEQ ID NO: 188 - DNA, 22 bp, synthetic construct
  source 1..22: mol_type = other DNA
catgagccactacaaccttcag

SEQ ID NO: 189 - DNA, 21 bp, synthetic construct
  source 1..21: mol_type = other DNA
ctcctggaggggctccctctc

(19) United States
(12) Patent Application Publication
(10) Pub. No.: US 2026/0174903 A1
(43) Pub. Date: Jun. 25, 2026

(54) ARTIFICIAL NUCLEIC ACIDS FOR SITE-DIRECTED EDITING OF A TARGET RNA

(71) Applicant: Eberhard Karls Universität Tübingen, Tübingen (DE)

(72) Inventors: Thorsten Stafforst, Tübingen (DE); Philipp Reautschnig, Tübingen (DE); Charlotte Pauline Wiegand, Tübingen (DE); Annika Eva Schulz, Albstadt (DE)

(73) Assignee: Eberhard Karls Universität Tübingen, Tübingen (DE)

(21) Appl. No.: 19/128,740

(22) PCT Filed: Nov. 10, 2023
(86) PCT No.: PCT/EP2023/081421

§ 371 Date: May 9, 2025

(30) Foreign Application Priority Data
Nov. 11, 2022 (WO) PCT/EP2022/081611

(51) Int. Cl. A61K 48/00; A61P 25/14; C12N 9/22; C12N 9/78; C12N 15/11; C12N 15/90

(52) U.S. Cl. CPC A61K 48/0058; A61P 25/14; C12N 9/226; C12N 9/78; C12N 15/11; C12N 15/907; C12N 2310/20; C12N 2310/321

(57) ABSTRACT

The present invention relates to artificial nucleic acids for site-directed editing of a target RNA. In particular, the present invention relates to artificial nucleic acids which provide for enhanced editing specificity and avoid undesirable off-target editing. The invention also relates to a method for providing said artificial nucleic acids. Furthermore, the present invention provides a vector encoding said artificial nucleic acid, as well as a cell, a composition and a kit comprising said artificial nucleic acid. Moreover, the invention provides the use of the artificial nucleic acid, the vector, the cell, the composition or the kit for site-directed editing of a target RNA or for in vitro diagnosis. In addition, the artificial nucleic acid, the vector, the cell, the composition or the kit of the present invention are provided for use as a medicament or for use in diagnosis of a disease or disorder.

The present invention relates to artificial nucleic acids for site-directed editing of a target RNA. In particular, the present invention relates to artificial nucleic acids which provide for enhanced editing specificity and avoid undesirable off-target editing. The invention also relates to a method for providing said artificial nucleic acids. Furthermore, the present invention provides a vector encoding said artificial nucleic acid, as well as a cell, a composition and a kit comprising said artificial nucleic acid. Moreover, the invention provides the use of the artificial nucleic acid, the vector, the cell, the composition or the kit for site-directed editing of a target RNA or for in vitro diagnosis. In addition, the artificial nucleic acid, the vector, the cell, the composition or the kit of the present invention are provided for use as a medicament or for use in diagnosis of a disease or disorder.

### BACKGROUND OF THE INVENTION

In conventional gene therapy, the genetic information is typically manipulated at the DNA level, thus permanently altering the genome. Depending on the application, the persistent modification of the genome may be either advantageous or imply serious risks. In this respect, the targeting of RNA instead of DNA represents an attractive alternative approach. When treating a subject on the RNA level, the change in gene expression is usually reversible, tunable and very frequently also more efficient. On the one hand, the limited duration of the effect will also limit the risks related to harmful side-effects. In addition, the possibility to finely tune the effect allows for continuously adjusting the therapy and to control the adverse effects in a time and dose-dependent manner. Furthermore, many manipulations of gene expression are not feasible or ineffective at the genome level, e.g. when the gene loss is either lethal or readily compensated by redundant processes. For example, it appears particularly attractive to target signaling networks at the RNA level. Many signaling cues are either essential, or they are strongly redundant so that a knockout sometimes does not result in a clear phenotype while a knockdown does.

Accordingly, there is an increasing interest in the engineering of RNA targeting strategies. One such strategy is RNA editing. (A)denosine-to-(I)nosine RNA editing is a natural enzymatic mechanism to diversify the transcriptome. Since inosine is biochemically interpreted as guanosine, A-to-I editing formally introduces A-to-G mutations, which can result in the recoding of amino acid codons, START and STOP codons, alteration of splicing, and alteration of miRNA activity, amongst others. Targeting such enzyme activities to specific sites at selected transcripts, a strategy called site-directed RNA editing, holds great promise for the treatment of disease and the general study of protein and RNA function.

Site-directed adenosine-to-inosine (A-to-I) RNA base editing is a very promising novel technology with a clear path for clinical application. Hydrolytic deamination of adenosine at its C6 position by enzymes of the ADAR family (adenosine deaminases acting on RNA) results in inosine, which is biochemically interpreted as guanosine (G) in many cellular processes like splicing or translation and in consequence functionally substitutes A by G on the RNA-level. In the past, the ADAR deaminase domain has been engineered into various artificial editing approaches that enable the efficient and highly programmable editing of any given target adenosine in the transcriptome by applying guide RNAs and simple Watson-Crick base pairing rules. Typical examples are the SNAP-ADAR, the λN-ADAR and the Cas13-ADAR approaches. However, even after several rounds of optimization, major limitations of such systems remain: a guide RNA plus a protein component needs to be delivered, non-human protein parts are included, and global off-target editing hampers their clinical development. A promising solution to the all three limitations could be the harnessing of the ubiquitously expressed endogenous ADAR enzyme for RNA base editing. It was shown recently that endogenous ADAR can be recruited by either chemically modified antisense oligonucleotides (Merkle, T., et al., Precise RNA editing by recruiting endogenous ADARs with antisense oligonucleotides. Nat Biotechnol, 2019, 37(2): p. 133-138) or by genetically encoded guide RNAs (Reautschnig, P., et al., CLUSTER guide RNAs enable precise and efficient RNA editing with endogenous ADAR enzymes in vivo. Nat Biotechnol, 2022. 40(5): p. 759-768; Yi, Z., et al., Engineered circular ADAR-recruiting RNAs increase the efficiency and fidelity of RNA editing in vitro and in vivo. Nat Biotechnol, 2022, 40: p. 946-955; Qu, L., et al., Programmable RNA editing by recruiting endogenous ADAR using engineered RNAs. Nat Biotechnol, 2019, 37: p 1059-1069).

Genetically encoded guide RNAs are particularly desired for the long-lasting correction of disease-causing G-to-A point mutations by viral delivery of the guide RNA component. However, genetically encoded guide RNAs currently suffer from massive off-target editing (also referred to as “bystander editing”) in the guide RNA/mRNA duplex. This is on one hand due to the large size guide RNA/mRNA duplex (70-200 bp) and on the other hand due to the ubiquitous presence of highly editable adenosine bases all over the duplex.

Both ADAR isoforms, ADAR1 and ADAR2, prefer similar “nearest neighbors”, e.g. U>A>C>G at the 5′ position relative to the target adenosine and G>C˜A>U at the 3′ position (Eggington, J. M., T. Greene, and B. L. Bass, Predicting sites of ADAR editing in double-stranded RNA. Nat Commun, 2011. 2: p. 319). Consequently, bystander editing is dominated by a handful of preferred nucleotide triplets, in particular all four 5′-UAN triplets (N=A, T, G, C), 5′-AAG and 5′-CAG. Bystander editing can lead to unwanted recoding events in the target and might even cause ribosome stalling.

Thus the avoidance of bystander editing represents an essential engineering problem. Today, a limited number of strategies have been suggested. A recent approach called CLUSTER (Reautschnig, P. et al., supra) minimizes the presence of editable adenosines in the guide RNA/mRNA duplex by fragmenting the guide RNA into several parts which bind the target transcript in areas selected for the absence of editable adenosine bases. In another approach called LEAPER (Yi, Z. et al., supra; Qu, L. et al, supra) bystander editing was regularly suppressed by mismatching some or even all adenosine bases prone for bystander editing with guanosine. The rationale behind this is the preference of the ADAR deaminase for a specific counter base (C>U>A/G) opposite the targeted adenosine (Wong, S. K., S. Sato, and D. W. Lazinski, Substrate recognition by ADAR1 and ADAR2. RNA, 7(6): p. 846-58 (2001)). However, the latter strategy is not optimal as it tends to fail depending on the sequence context and it can dramatically reduce editing efficiency for targets that are rich in editable triplets or that comprise the targeted adenosine within an adenosine-rich sequence context, like 5′-UAAG, 5′-UAAU, etc. Thus, precise editing can cost notable editing efficiency. In principle, the same challenge holds true for natural editing sites which are guided in cis by intronic editing guiding sequences (EGS). Interestingly, such natural editing sites are typically not found in perfect RNA helices but rather contain bulges, mismatches, and wobble base pairs. This structural layer that makes an RNA a good or poor substrate for ADAR is still underexplored, however, several recent works have addressed this issue (Schneider, M. F., et al., Optimal guide RNAs for re-directing deaminase activity of hADAR1 and hADAR2 in trans. Nucleic Acids Res, 2014. 42(10): p. e87; Uzonyi, A., et al., Deciphering the principles of the RNA editing code via large-scale systematic probing. Mol Cell, 2021; Liu, X., et al., Learning cis-regulatory principles of ADAR-based RNA editing from CRISPR-mediated mutagenesis. Nat Commun, 2021. 12(1): p. 2165).

In LEAPER 2.0 guide RNAs (Yi, Z., et al., supra), bystander/off-target editing is suppressed by removing the uridine bases opposite of off-target edited adenosine bases. This depletion of uridines leads to the bulging out of the unpaired adenosine base in the target and reduces its editing yield. While uridine depletion seems to suppress bystander editing slightly better than the G-A mismatch, it also lowers the hybridization strength and perturbs the substrate duplex structure. This can have a negative influence on the on target editing efficiency, in particular if an off-target A is close to the target A.

The G·U wobble base pair does not only occur in many naturally edited substrates but it is the most abundant type of non-Watson-Crick base pair in the transcriptome (Varani, G. and W. H. McClain, The G×U wobble base pair. A fundamental building block of RNA structure crucial to RNA function in diverse biological systems. EMBO Rep, 2000. 1(1): p. 18-23). Surrounding the G·U wobble base, the RNA helix structure is perturbed affecting the groove width, base stacking, and the electrostatic profile (Xu, D., et al., The electrostatic characteristics of G·U wobble base pairs. Nucleic Acids Res, 2007. 35(11): p. 3836-47). Specific structural effects induced by G·U wobble bases have been shown important for the interaction of dsRNA-binding proteins with dsRNA substrates, including ADAR (Stefl, R., et al., Structure and specific RNA binding of ADAR2 double-stranded RNA binding motifs. Structure, 2006. 14(2): p. 345-55).

From the aforesaid it becomes clear that there is an urgent need of RNA editing strategies that allow for high editing yields and high specificity which do not result in off-target editing. In particular, compounds are required that are suitable for recruiting (endogenous) deaminases and which can be expressed by the individual itself based on vectors encoding the guide nucleic acids.

It is thus an objective of the present invention to provide a compound that is capable of recruiting a deaminase, preferably an endogenous deaminase, e.g. an adenosine deaminase, to an RNA target to be edited. A particular objective of the present invention is the provision of a compound suitable for editing an RNA target with high efficiency and high specificity, in particular with a reduced rate of off-target editing. Improved RNA editing approaches shall thus be provided, which allow for high yields of RNA editing at a specifically targeted site in a target RNA, preferably without or with reduced unspecific editing at other transcriptomic sites.

The solution of said object is achieved by the embodiments described herein and defined by the claims.

### DETAILED DESCRIPTION OF THE INVENTION

In a first aspect, the present invention concerns novel artificial nucleic acids for site-directed editing of a target RNA which comprises a target sequence comprising an adenosine as a target nucleotide to be edited and at least one off-target sequence comprising an adenosine that is not to be edited. In particular, an artificial nucleic acid is provided herein, which comprises:
a first nucleic acid sequence comprising or consisting of 3 nucleotides that is complementary or partially complementary to a nucleic acid sequence in the target sequence, which comprises a target adenosine nucleotide to be edited and the nucleotides immediately 5′ and 3′ of said target adenosine nucleotide, and a second nucleic acid sequence comprising at least 3 nucleotides that is complementary or partially complementary to a nucleic acid sequence in the at least one off-target sequence, which comprises an adenosine nucleotide not to be edited and the nucleotides immediately 5′ and 3′ of said adenosine nucleotide, wherein the target sequence comprises at least one nucleotide triplet selected from the group consisting of 5′-UAG-3′, 5′-GAU-3′, 5′-GAG-3′, 5′-GAC-3′, 5′-GAA-3′, 5′-CAG-3′, and 5′-AAG-3′, and/or the off-target sequence comprises at least one nucleotide triplet selected from the group consisting of 5′-UAU-3′, 5′-UAG-3′, 5′-UAC-3′, 5′-UAA-3′, 5′-GAU-3′, 5′-CAU-3′, and 5′-AAU-3′, and/or a third nucleic acid sequence comprising or consisting of 4 or 5 nucleotides that is complementary or partially complementary to a nucleic acid sequence in the target RNA which comprises an adenosine nucleotide to be edited, at least one neighboring adenosine that is not to be edited and the nucleotides immediately 5′ and 3′ of said adenosine nucleotides, wherein the target RNA comprises at least one nucleotide sequence selected from the group consisting of 5′-UAAA-3′, 5′-UAAU-3′, 5′-UAAC-3′, 5′-UAAG-3′, 5′-AAAU-3′, 5′-CAAU-3′, 5′-GAAU-3′, and 5′-UAAAU-3′, wherein
the first nucleic acid sequence of the artificial nucleic acid
   consists of a nucleotide triplet selected from the group consisting of 5′-UUA-3′, and 5′-UCA-3′, preferably if the target adenosine nucleotide to be edited in the target sequence is the central adenosine nucleotide in a nucleotide triplet 5′-UAG-3′;
   consists of a nucleotide triplet selected from the group consisting of 5′-AUU-3′, and 5′-ACU-3′, preferably if the target adenosine nucleotide to be edited in the target sequence is the central adenosine nucleotide in a nucleotide triplet 5′-GAU-3′;
   consists of a nucleotide triplet selected from the group consisting of 5′-CUU-3′, 5′-UUC-3′, 5′-UUU-3′, 5′-UUG-3′, 5′-GUU-3′, 5′-UUA-3′, 5′-AUU-3′, 5′-CCU-3′, 5′-UCC-3′, 5′-UCU-3′, preferably if the target adenosine nucleotide to be edited in the target sequence is the central adenosine nucleotide in a nucleotide triplet 5′-GAG-3′;
   consists of a nucleotide triplet selected from the group consisting of 5′-GUU-3′, and 5′-GCU-3′, preferably if the target adenosine nucleotide to be edited in the target sequence is the central adenosine nucleotide in a nucleotide triplet 5′-GAC-3′;
   consists of a nucleotide triplet selected from the group consisting of 5′-UUU-3′, 5′-AUU-3′, 5′-UCU-3′, and 5′-ACU-3′, preferably if the target adenosine nucleotide to be edited in the target sequence is the central adenosine nucleotide in a nucleotide triplet 5′-GAA-3′;
   consists of a nucleotide triplet selected from the group consisting of 5′-UUG-3′, 5′-UCG-3′, 5′-UUA-3′, and 5′-UCA-3′, preferably if the target adenosine nucleotide to be edited in the target sequence is the central adenosine nucleotide in a nucleotide triplet 5′-CAG-3′; and/or
   consists of a nucleotide triplet selected from the group consisting of 5′-UUU-3′, and 5′-UCU-3′, preferably if the target adenosine nucleotide to be edited in the target sequence is the central adenosine nucleotide in a nucleotide triplet 5′-AAG-3′, and/or
the second nucleic acid sequence of the artificial nucleic acid
   does not comprise a nucleotide triplet 5′-AUA-3′ and comprises one or more of a nucleotide triplet selected from the group consisting of 5′-GUG-3′, 5′-AUG-3′, 5′-GUA-3′, 5′-GGG-3′, 5′-AGG-3′, and 5′-GGA-3′, preferably if an adenosine nucleotide not to be edited in the off-target sequence is the central adenosine nucleotide in a nucleotide triplet 5′-UAU-3′;
   does not comprise a nucleotide triplet 5′-CUA-3′ and comprises one or more of a nucleotide triplet selected from the group consisting of 5′-CUG-3′, 5′-UUG-3′, 5′-CGG-3′, and 5′-UGG-3′, preferably if an adenosine nucleotide not to be edited in the off-target sequence is the central adenosine nucleotide in a nucleotide triplet 5′-UAG-3′;
   does not comprise a nucleotide triplet 5′-GUA-3′ and comprises one or more of a nucleotide triplet selected from the group consisting of 5′-GUG-3′, and 5′-GGG-3′, preferably if an adenosine nucleotide not to be edited in the off-target sequence is the central adenosine nucleotide in a nucleotide triplet 5′-UAC-3′;
   does not comprise a nucleotide triplet 5′-UUA-3′ and comprises one or more of a nucleotide triplet selected from the group consisting of 5′-UUG-3′, 5′-UGG-3′, 5′-GUG-3′, and 5′-GGG-3′, preferably if an adenosine nucleotide not to be edited in the off-target sequence is the central adenosine nucleotide in a nucleotide triplet 5′-UAA-3′;
   does not comprise a nucleotide triplet 5′-AUC-3′ and comprises one or more of a nucleotide triplet selected from the group consisting of 5′-GUC-3′, 5′-GUU-3′, 5′-GGC-3′, and 5′-GGU-3′, preferably if an adenosine nucleotide not to be edited in the off-target sequence is the central adenosine nucleotide in a nucleotide triplet 5′-GAU-3′;
   does not comprise a nucleotide triplet 5′-AUG-3′ and comprises one or more of a nucleotide triplet selected from the group consisting of 5′-GUG-3′, and 5′-GGG-3′, preferably if an adenosine nucleotide not to be edited in the off-target sequence is the central adenosine nucleotide in a nucleotide triplet 5′-CAU-3′; and/or
   does not comprise a nucleotide triplet 5′-AUU-3′ and comprises one or more of a nucleotide triplet selected from the group consisting of 5′-GUU-3′, 5′-GGU-3′, 5′-GUG-3′, and 5′-GGG-3′, preferably if an adenosine nucleotide not to be edited in the off-target sequence is the central adenosine nucleotide in a nucleotide triplet 5′-AAU-3′, and/or
the third nucleic acid sequence of the artificial nucleic acid
   consists of a nucleotide sequence 5′-UCUG-3′, or of a nucleotide sequence 5′-GCUG-3′, preferably if the adenosine nucleotides in the target sequence are present in the context of the nucleic acid sequence 5′-UAAA-3′;
   consists of a nucleotide sequence 5′-ACUG-3′, or of a nucleotide sequence 5′-GUCA-3′, preferably if the adenosine nucleotides in the target sequence are present in the context of the nucleic acid sequence 5′-UAAU-3′;
   consists of a nucleotide sequence 5′-GCUG-3′, preferably if the adenosine nucleotides in the target sequence are present in the context of the nucleic acid sequence 5′-UAAC-3′;
   consists of a nucleotide sequence 5′-CCUG-3′, or of a nucleotide sequence 5′-UCUG-3′, or of a nucleotide sequence 5′-UUUG-3′, preferably if the adenosine nucleotides in the target sequence are present in the context of the nucleic acid sequence 5′-UAAG-3′;
   consists of a nucleotide sequence 5′-GUCU-3′, or of a nucleotide sequence 5′-GUCG-3′, preferably if the adenosine nucleotides in the target sequence are present in the context of the nucleic acid sequence 5′-AAAU-3′;
   consists of a nucleotide sequence 5′-GUCG-3′, preferably if the adenosine nucleotides in the target sequence are present in the context of the nucleic acid sequence 5′-CAAU-3′;
   consists of a nucleotide sequence 5′-GUCC-3′, or of a nucleotide sequence 5′-GUCU-3′, or of a nucleotide sequence 5′-GUUU-3′, preferably if the adenosine nucleotides in the target sequence are present in the context of the nucleic acid sequence 5′-GAAU-3′; and/or
   consists of a nucleotide sequence 5′-GUCUG-3′, preferably if the adenosine nucleotides in the target sequence are present in the context of the nucleic acid sequence 5′-UAAAU-3′.

The inventors surprisingly found that RNA editing at a specifically targeted site can be improved, and unspecific editing at other transcriptomic sites can be reduced by using specific U·G wobble base pairs and/or G·U wobble base pairs 3′ and 5′ to the target adenosine nucleotide and to the off-target adenosine nucleotide, respectively. The artificial nucleic acid described herein is preferably designed accordingly.

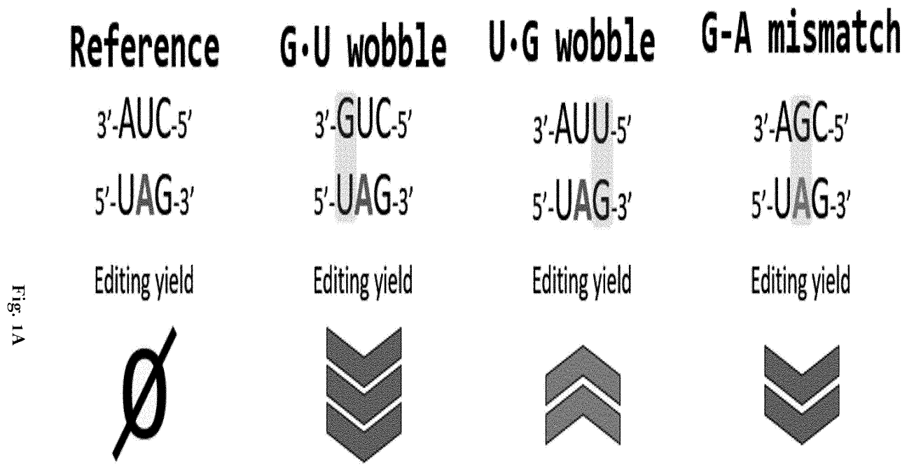

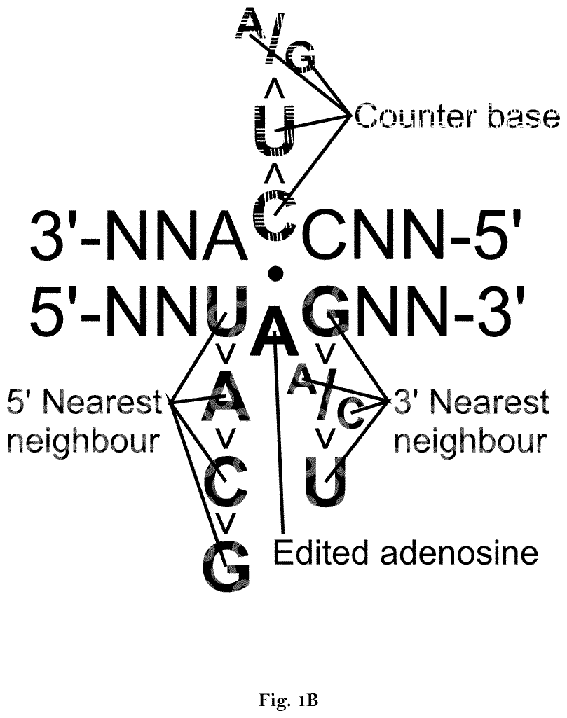

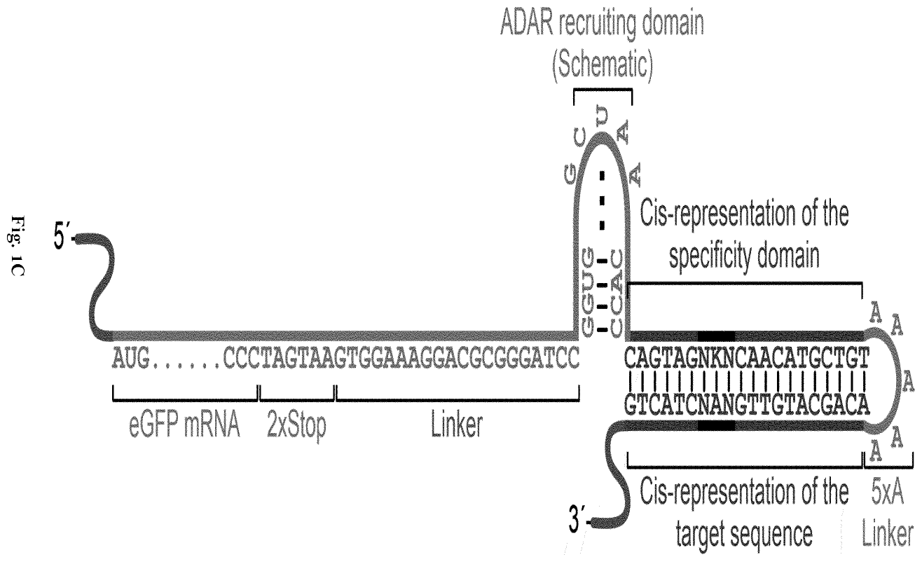

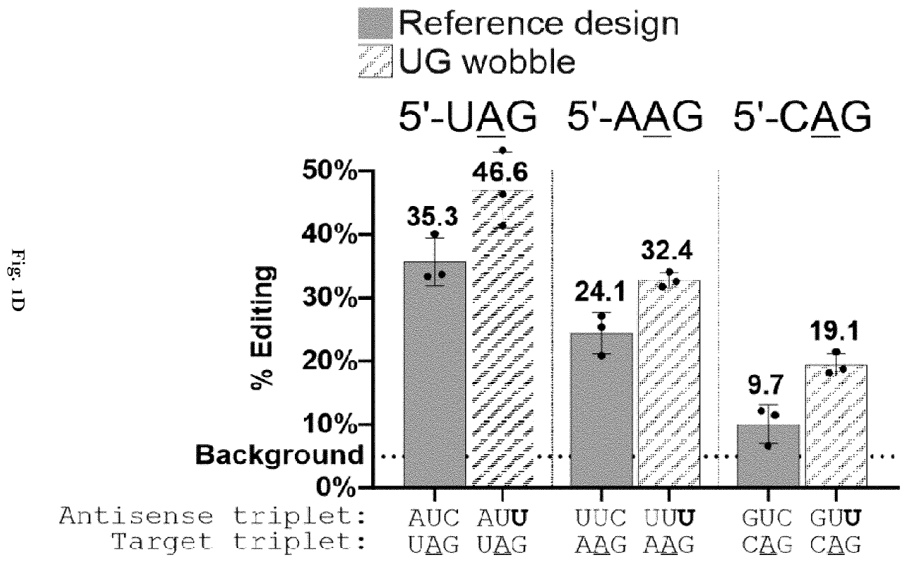

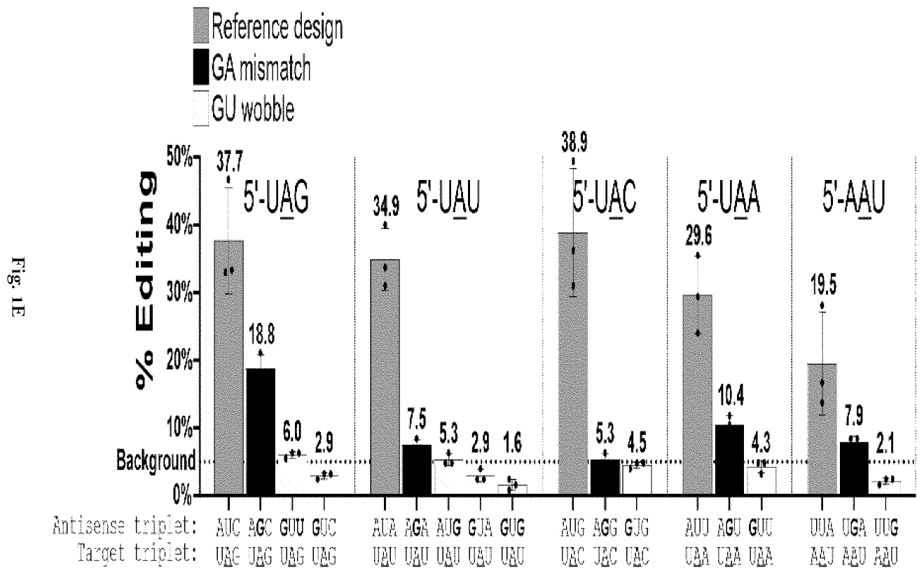

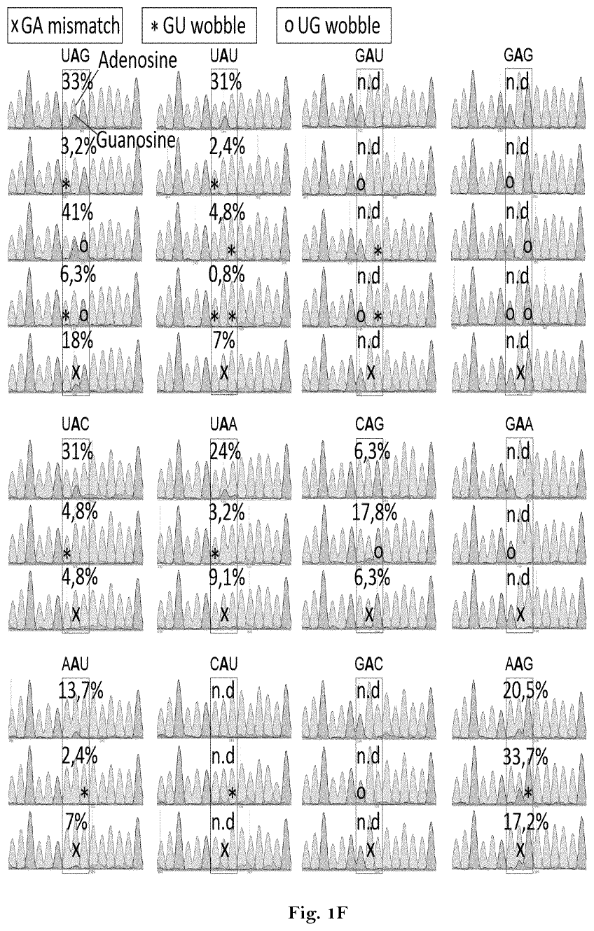

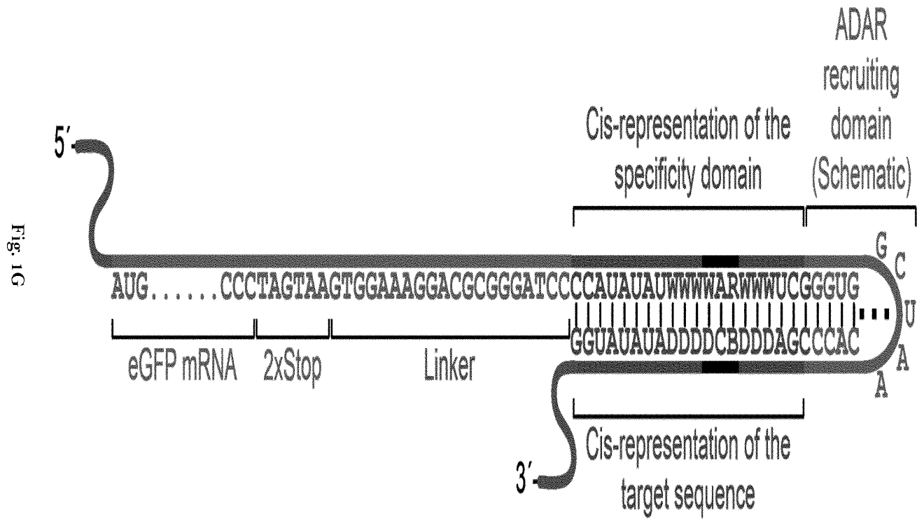

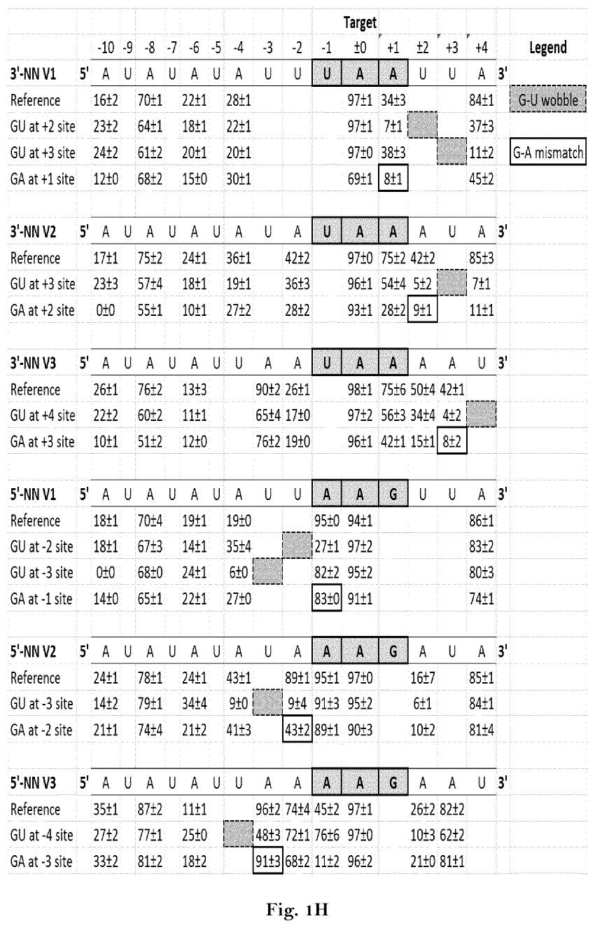

In this context, two types of wobble base-pairs can occur: when the U base in a 5′-UAN-3′ or 5′-NAU-3′ nucleotide triplet (N=U, C, A, G) of the target RNA is base-paired with a G base present in the artificial nucleic acid described herein, this is referred to as a 5′-G·U or 3′-G·U wobble, respectively; when the G base in a 5′-GAN-3′ or 5′-NAG-3′ nucleotide triplet of the target RNA is base-paired with a U base present in the artificial nucleic acid described herein, this is referred to as a 5′-U·G or 3′-U·G wobble, respectively (see also FIG. 1A).

In particular, the inventors found that 5′-G·U wobble base pairs and 3′-G·U wobble base pairs can strongly suppress or reduce bystander/off-target editing in 5′-UAN and 5′-NAU triplets (N=U, C, A, G). This circumstance is preferably exploited in order to reduce bystander/off-target editing of 5′-UAN and 5′-NAU triplets (N=U, C, A, G) in the target RNA (but not at the target site), which are not to be edited. Further, the inventors found an opposite effect for U·G wobble base pairs, which in contrast can be employed for boosting editing in 5′-NAG and 5′-GAN triplets (N=U, C, A, G).

Therefore, according to the invention, the artificial nucleic acid is designed to be complementary or partially complementary to the target RNA, which comprises a target sequence comprising an adenosine as a target nucleotide to be edited and at least one off-target sequence comprising an adenosine that is not to be edited, and in particular is designed to include nucleotide triplets that form U·G wobble base pairs (3′-U·G wobble base pairs and/or 5′-U·G wobble base pairs) with the nucleotides immediately 5′ and/or 3′ to the adenosine nucleotide to be edited, and/or nucleotide triplets that form G·U wobble base pairs (3′-G·U wobble base pairs and/or 5′-G·U wobble base pairs) with the nucleotides immediately 5′ and/or 3′ to an adenosine nucleotide not to be edited.

In other words, the artificial nucleic acid molecule is designed to boost the editing efficiency in the target sequence using U·G wobbling immediately 3′ or 5′ to an adenosine nucleotide to be edited and/or to suppress or reduce off-target editing in an off-target sequence in the target RNA using G·U wobbling immediately 3′ or 5′ to a adenosine nucleotide not to be edited.

Moreover, in a specific case when the target RNA comprises an adenosine nucleotide to be edited (A) adjacent to an adenosine nucleotide not to be edited in a sequence context comprising 5′-UAAN-3′ or 5′-NAAU-3′ (N=U, A, C, G), or 5′-UAAAU-3′, the artificial nucleic acid molecule may comprise a third nucleic acid sequence consisting of 4 or 5 nucleotides which forms a 3′-G·U wobble base pair and/or a 5′-G·U wobble base pair, respectively, with a nucleotide immediately 5′ and/or 3′ to the adenosine nucleotide(s) not to be edited.

Therefore, in this case, the artificial nucleic acid molecule is designed to suppress or reduce off-target editing in the off-target sequence using G·U wobbling immediately 3′ or 5′ to a adenosine nucleotide not to be edited while enabling or even enhancing editing of a neighboring adenosine nucleotide.

As used herein, the phrase ‘artificial nucleic acid (molecule)’ typically refers to a nucleic acid that does not occur naturally. In other words, an artificial nucleic acid molecule may be a non-natural nucleic acid. Such an artificial nucleic acid molecule may be non-natural due to its individual nucleotide sequence (which does not occur naturally) and/or due to other modifications, e.g. structural modifications of nucleotides, which do not occur naturally in that context. An artificial nucleic acid as used herein preferably differs from a naturally occurring nucleic acid by at least one nucleotide or by at least one modification of a nucleotide. An artificial nucleic acid may be a DNA molecule, an RNA molecule or a hybrid-molecule comprising DNA and RNA portions. In preferred embodiments, the artificial nucleic acid is an RNA molecule. In particular, an artificial nucleic acid as used herein may comprise unmodified or modified ribonucleotides and/or unmodified or modified deoxynucleotides and preferably comprises unmodified ribonucleotides and/or deoxynucleotides. Further, the phrase ‘artificial nucleic acid (molecule)’ is not restricted to ‘one single molecule’ but may also refer to an ensemble of identical molecules. Accordingly, the phrase may refer to a plurality of identical molecules contained, for example, in a sample. Throughout the present specification, the terms “artificial nucleic acid (molecule)” and “guide RNA” may be used interchangeably.

In the context of the present invention, the phrase ‘RNA editing’ refers to the reaction by which a nucleotide, preferably an adenosine nucleotide, in a target RNA is transformed by a deamination reaction into another nucleotide. That change typically results in a different gene product, since the changed nucleotide preferably results in a codon change, leading e.g. to incorporation of another amino acid in the polypeptide translated from the RNA or to the generation of a start or stop codon or to the deletion of a stop codon. In particular, an adenosine nucleotide in a target RNA is converted to inosine by deamination, e.g. by an adenosine deaminase as described herein. As used herein, the term ‘target RNA’ typically refers to an RNA, which is subject to an editing reaction, which is supported by the artificial nucleic acid described herein.

The RNA editing achieved by the artificial nucleic acid described herein is further ‘site-directed’, which means that a specific adenosine nucleotide at a target sequence in a target RNA is edited, while (off-target) editing of (an) other adenosine nucleotide(s) in an off-target sequence is suppressed by G·U wobbling of an adjacent guanosine nucleotide, as described herein.

Typically, the adenosine nucleotide at a target sequence is targeted by a first nucleic acid sequence of the artificial nucleic acid described herein. In the context of the present invention, the first nucleic acid sequence of the artificial nucleic acid consists of 3 nucleotides which are (at least partially) complementary to a target sequence which comprises a target adenosine nucleotide to be edited and the nucleotides immediately 5′ and 3′ of said adenosine nucleotide. In some embodiments, a target RNA may comprise two or more adenosine nucleotides to be edited, wherein these nucleotides are preferably separated from each other by at least one other nucleotide. In this case, the artificial nucleic acid may comprise two or more “first nucleic acid sequences”.

As used herein, the terms ‘complementary’ or ‘partially complementary’ preferably refer to nucleic acid sequences, which due to their complementary nucleotides are capable of specific intermolecular base-pairing, preferably Watson-Crick and/or wobble base pairing, preferably under physiological conditions. The term ‘complementary’ as used herein may also refer to reverse complementary sequences. In the context of the present invention, the term ‘guide RNA’ may also be used in order to refer to the artificial nucleic acid described herein, which preferably guides the deaminase function to the target site.

As mentioned above, a first nucleotide sequence of the artificial nucleic acid consists of 3 nucleotides which are (at least partially) complementary to a target sequence in the target RNA which comprises an adenosine nucleotide to be edited (A) and the nucleotides immediately 5′ and 3′ of said target adenosine nucleotide. Preferably, the nucleotide triplet of a first nucleotide sequence is complementary to a 5′-NAG or 5′-GAN nucleotide triplet in the target sequence (N=U, C, A, G), wherein a uridine nucleotide immediately 3′ or 5′ to the central nucleotide of the first nucleotide sequence of the artificial nucleic acid preferably forms an U·G wobble base pair with the guanosine nucleotide immediately 3′ or 5′ of the central (target) adenosine nucleotide in the target RNA triplet, while the central nucleotide of the nucleotide triplet of the first nucleotide sequence is preferably a uridine (U) which matches with the target adenosine (A), or a cytosine (C) which mismatches with the target adenosine (A) of the target sequence.

### Concretely:

If the target sequence of the target RNA comprises an adenosine as a target nucleotide to be edited as a central adenosine (A) in a nucleotide triplet context 5′-UAG-3′, the first nucleic acid sequence of the artificial nucleic acid consists of a nucleotide triplet 5′-UUA-3′ or 5′-UCA-3′. (Here and in the following, the U·G wobble nucleotides are represented in bold letters.)

If the target sequence of the target RNA comprises an adenosine as a target nucleotide to be edited in a nucleotide triplet context 5′-GAU-3′, the first nucleic acid sequence of the artificial nucleic acid consists of a nucleotide triplet 5′-AUU-3′ or 5′-ACU-3′.

If the target sequence of the target RNA comprises an adenosine as a target nucleotide to be edited in a nucleotide triplet context 5′-GAG-3′, the first nucleic acid sequence of the artificial nucleic acid consists of a nucleotide triplet selected from the group consisting of 5′-CUU-3′, 5′-UUC-3′, 5′-UUU-3′, 5′-UUG-3′, 5′-GUU-3′, 5′-UUA-3′, 5′-AUU-3′, 5′-CCU-3′, 5′-UCC-3′, 5′-UCU-3′.

If the target sequence of the target RNA comprises an adenosine as a target nucleotide to be edited in a nucleotide triplet context 5′-GAC-3′, the first nucleic acid sequence of the artificial nucleic acid consists of a nucleotide triplet 5′-GUU-3′ or 5′-GCU-3′.

If the target sequence of the target RNA comprises an adenosine as a target nucleotide to be edited in a nucleotide triplet context 5′-GAA-3′, the first nucleic acid sequence of the artificial nucleic acid consists of a nucleotide triplet selected from the group consisting of 5′-UUU-3′, 5′-AUU-3′, 5′-UCU-3′, and 5′-ACU-3′.

If the target sequence of the target RNA comprises an adenosine as a target nucleotide to be edited in a nucleotide triplet context 5′-CAG-3′, the first nucleic acid sequence of the artificial nucleic acid consists of a nucleotide triplet selected from the group consisting of 5′-UUG-3′, 5′-UCG-3′, 5′-UUA-3′, and 5′-UCA-3′.

If the target sequence of the target RNA comprises an adenosine as a target nucleotide to be edited in a nucleotide triplet context 5′-AAG-3′, the first nucleic acid sequence of the artificial nucleic acid consists of a nucleotide triplet 5′-UUU-3′ or 5′-UCU-3′.

As stated above, the U·G ‘wobbling’ at a position 3′ or 5′ of the central target adenosine enhances (boosts) the A-to-I-editing efficiency in the target RNA.

Further, the artificial nucleic acid may comprise a second nucleic acid sequence which comprises at least 3 nucleotides complementary or partially complementary to a nucleic acid sequence in the at least one off-target sequence, which comprises an adenosine nucleotide not to be edited and the nucleotides immediately 5′ and 3′ of said adenosine nucleotide. Typically, a target RNA may comprise two or more adenosine nucleotides not to be edited in an off-target sequence, wherein these nucleotides are preferably separated from each other by at least one, preferably two other nucleotides. In this case, the second nucleic acid sequence of the artificial nucleic acid may comprise two or more nucleotide triplets, each complementary or partially complementary to a nucleic acid triplet in an off-target sequence of the target RNA which comprises an adenosine nucleotide not to be edited and the nucleotides immediately 5′ and 3′ of said (off-target) adenosine nucleotide.

In particular, a nucleotide triplet of a second nucleotide sequence is complementary to a nucleotide triplet in an off-target sequence of the target RNA comprising a 5′-UAN and/or 5′-NAU nucleotide triplet (N=U, C, A, G), respectively, wherein a guanosine nucleotide 3′ or 5′ to the central nucleotide of the nucleotide triplet of the artificial nucleic acid forms a G·U wobble base pair with the uridine nucleotide immediately 3′ and/or 5′ of the central adenosine nucleotide (not to be edited) in the off-target RNA triplet. At the same time, the central nucleotide of the nucleotide triplet of the second nucleotide sequence is a uridine (U) which matches with the central adenosine (A) not to be edited, or a guanosine which mismatches with the central adenosine (A) not to be edited.

### Concretely:

If an off-target sequence in the target RNA comprises an adenosine nucleotide that is not to be edited in a nucleotide triplet context 5′-UAU-3′, the second nucleic acid sequence of the artificial nucleic acid does not comprise a nucleotide triplet 5′-AUA-3′ (complementary to the 5′-UAU-3′ triplet), but rather comprises a nucleotide triplet selected from the group consisting of 5′-GUG-3′, 5′-AUG-3′, 5′-GUA-3′, 5′-GGG-3′, 5′-AGG-3′, and 5′-GGA-3′.

If an off-target sequence in the target RNA comprises an adenosine nucleotide that is not to be edited in a nucleotide triplet context 5′-UAG-3′, the second nucleic acid sequence of the artificial nucleic acid does not comprise a nucleotide triplet 5′-CUA-3′ (complementary to the 5′-UAG-3′ triplet), but rather comprises a nucleotide triplet selected from the group consisting of 5′-CUG-3′, 5′-UUG-3′, 5′-CGG-3′, and 5′-UGG-3′.

If an off-target sequence of the target RNA comprises an adenosine nucleotide that is not to be edited in a nucleotide triplet context 5′-UAC-3′, the second nucleic acid sequence of the artificial nucleic acid does not comprise a nucleotide triplet 5′-GUA-3′ (complementary to the 5′-UAC-3′ triplet), but rather comprises a nucleotide triplet selected from the group consisting of 5′-GUG-3′, and 5′-GGG-3′.

If an off-target sequence in the target RNA comprises an adenosine nucleotide that is not to be edited in a nucleotide triplet context 5′-UAA-3′, the second nucleic acid sequence of the artificial nucleic acid does not comprise a nucleotide triplet 5′-UUA-3′ (complementary to the 5′-UAA-3′ triplet), but rather comprises a nucleotide triplet selected from the group consisting of 5′-UUG-3′, 5′-UGG-3′, 5′-GUG-3′, and 5′-GGG-3′.

If an off-target sequence in the target RNA comprises an adenosine nucleotide that is not to be edited in a nucleotide triplet context 5′-GAU-3′, the second nucleic acid sequence of the artificial nucleic acid does not comprise a nucleotide triplet 5′-AUC-3′ (complementary to the 5′-GAU-3′ triplet), but rather comprises a nucleotide triplet selected from the group consisting of 5′-GUC-3′, 5′-GUU-3′, 5′-GGC-3′, and 5′-GGU-3′.

If an off-target sequence in the target RNA comprises an adenosine nucleotide that is not to be edited in a nucleotide triplet context 5′-CAU-3′, the second nucleic acid sequence of the artificial nucleic acid does not comprise a nucleotide triplet 5′-AUG-3′ (complementary to 5′-CAU-3′ triplet), but rather comprises a nucleotide triplet selected from the group consisting of 5′-GUG-3′, and 5′-GGG-3′.

If an off-target sequence of the target RNA comprises an adenosine nucleotide that is not to be edited in a nucleotide triplet context 5′-AAU-3′ (wherein the central adenosine is the adenosine not to be edited), the second nucleic acid sequence of the artificial nucleic acid does not comprise a nucleotide triplet 5′-AUU-3′ (complementary to 5′-AAU-3′ triplet), but rather comprises a nucleotide triplet selected from the group consisting of 5′-GUU-3′, 5′-GGU-3′, 5′-GUG-3′, and 5′-GGG-3′.

As stated above, the G·U wobbling at a position 3′ or 5′ of the central off-target adenosine suppresses or reduces off-target A-to-I-editing in the off-target sequence of the target RNA.

Further, the artificial nucleic acid may comprise a third nucleic acid sequence consisting of 4 or 5 nucleotides that is complementary or partially complementary to a nucleic acid sequence in the target RNA which comprises an adenosine nucleotide to be edited, at least one neighboring adenosine that is not to be edited and the nucleotides immediately 5′ and 3′ of said adenosine nucleotides in the target RNA.

In particular, a nucleotide sequence of a third nucleotide sequence of the artificial nucleic acid is complementary to a nucleotide sequence in the target RNA comprising an adenosine nucleotide to be edited (A) and a neighboring adenosine nucleotide not to be edited (A) in a 5′-UAAN or 5′-NAAU or 5′-UAAAU-3′ context (N=U, C, A, G), respectively, wherein a guanosine nucleotide of the third nucleotide sequence of the artificial nucleic acid forms a G·U wobble base pair with the uridine nucleotide immediately 3′ or 5′ to the adenosine nucleotide not to be edited in the target RNA sequence.

### Concretely:

If the target RNA comprises an adenosine nucleotide that is not to be edited (A) immediately adjacent to an adenosine nucleotide to be edited (A) in a nucleotide context 5′-UAAA-3′, the third nucleic acid sequence of the artificial nucleic acid consists of a nucleotide sequence 5′-UCUG-3′ or 5′-GCUG-3′.

If the target RNA comprises an adenosine nucleotide that is not to be edited (A) immediately adjacent to an adenosine nucleotide to be edited (A) in a nucleotide context 5′-UAAU-3′, the third nucleic acid sequence of the artificial nucleic acid consists of a nucleotide sequence 5′-ACUG-3′.

If the target RNA comprises an adenosine nucleotide that is not to be edited (A) immediately adjacent to an adenosine nucleotide to be edited (A) in a nucleotide context 5′-UAAU-3′, the third nucleic acid sequence of the artificial nucleic acid consists of a nucleotide sequence 5′-GUCA-3′.

If the target RNA comprises an adenosine nucleotide that is not to be edited (A) immediately adjacent to an adenosine nucleotide to be edited (A) in a nucleotide context 5′-UAAC-3′, the third nucleic acid sequence of the artificial nucleic acid consists of a nucleotide sequence 5′-GCUG-3′.

If the target RNA comprises an adenosine nucleotide that is not to be edited (A) immediately adjacent to an adenosine nucleotide to be edited (A) in a nucleotide context 5′-UAAG-3′, the third nucleic acid sequence of the artificial nucleic acid consists of a nucleotide sequence 5′-CCUG-3′, 5′-UCUG-3′ or 5′-UUUG-3′.

If the target RNA comprises an adenosine nucleotide that is not to be edited (A) immediately adjacent to an adenosine nucleotide to be edited (A) in a nucleotide context 5′-AAAU-3′, the third nucleic acid sequence of the artificial nucleic acid consists of a nucleotide sequence 5′-GUCU-3′, or 5′-GUCG-3′.

If the target RNA comprises an adenosine nucleotide that is not to be edited (A) immediately adjacent to an adenosine nucleotide to be edited (A) in a nucleotide context 5′-CAAU-3′, the third nucleic acid sequence of the artificial nucleic acid consists of a nucleotide sequence 5′-GUCG-3′.

If the target RNA comprises an adenosine nucleotide that is not to be edited (A) immediately adjacent to an adenosine nucleotide to be edited (A) in a nucleotide context 5′-GAAU-3′, the third nucleic acid sequence of the artificial nucleic acid consists of a nucleotide sequence 5′-GUCC-3′, 5′-GUCU-3′, or 5′-GUUU-3′.

If the target RNA comprises adenosine nucleotides that are not to be edited (A) immediately 3′ and 5′ to an adenosine nucleotide to be edited (A) in a nucleotide context 5′-UAAAU-3′, the third nucleic acid sequence of the artificial nucleic acid consists of a nucleotide sequence 5′-GUCUG-3′.

Again, G·U wobbling at positions immediately 3′ and/or 5′ of the adenosine not to be edited suppresses bystander A-to-I-editing in the target RNA. At the same time, editing of the target adenosine nucleotide is preferably enhanced by a C-A mismatch at the target site.

It was shown recently that both ADAR isoforms, ADAR1 and ADAR2, prefer similar nearest neighbors (U>A>C>G) at the 5′ position relative to the target adenosine and G>C/A>U at the 3′ position (see FIG. 1B). Consequently, bystander (off-target) editing is particularly dominated (and a problem) e.g. in all four 5′-UAN-3′ triplets (5′-UAU-3′, 5′-UAG-3′, 5′-UAC-3′, 5′-UAA-3′), while 5′-GAU-3′ and 5′-CAU-3′ triplets are less prone to bystander editing and are rather unproblematic.

Therefore, in a preferred embodiment of the present invention, the second nucleic acid sequence of the artificial nucleic acid molecule comprising at least 3 nucleotides that are complementary or partially complementary to a nucleic acid sequence in the at least one off-target sequence, which comprises an adenosine nucleotide not to be edited and the nucleotides immediately 5′ and 3′ of said adenosine nucleotide,
does not comprise a nucleotide triplet 5′-AUA-3′ and comprises one or more of a nucleotide triplet selected from the group consisting of 5′-GUG-3′, 5′-AUG-3′, 5′-GUA-3′, 5′-GGG-3′, 5′-AGG-3′, and 5′-GGA-3′, preferably if an adenosine nucleotide not to be edited in the off-target sequence is the central adenosine nucleotide in a nucleotide triplet 5′-UAU-3′;
does not comprise a nucleotide triplet 5′-CUA-3′ and comprises one or more of a nucleotide triplet selected from the group consisting of 5′-CUG-3′, 5′-UUG-3′, 5′-CGG-3′, and 5′-UGG-3′, preferably if an adenosine nucleotide not to be edited in the off-target sequence is the central adenosine nucleotide in a nucleotide triplet 5′-UAG-3′;
does not comprise a nucleotide triplet 5′-GUA-3′ and comprises one or more of a nucleotide triplet selected from the group consisting of 5′-GUG-3′, and 5′-GGG-3′, preferably if an adenosine nucleotide not to be edited in the off-target sequence is the central adenosine nucleotide in a nucleotide triplet 5′-UAC-3′;
does not comprise a nucleotide triplet 5′-UUA-3′ and comprises one or more of a nucleotide triplet selected from the group consisting of 5′-UUG-3′, 5′-UGG-3′, 5′-GUG-3′, and 5′-GGG-3′, preferably if an adenosine nucleotide not to be edited in the off-target sequence is the central adenosine nucleotide in a nucleotide triplet 5′-UAA-3′; and/or
does not comprise a nucleotide triplet 5′-AUU-3′ and comprises one or more of a nucleotide triplet selected from the group consisting of 5′-GUU-3′, and 5′-GGU-3′, preferably if an adenosine nucleotide not to be edited in the off-target sequence is the central adenosine nucleotide in a nucleotide triplet 5′-AAU-3′.

Some nucleotide triplets comprised in the second nucleic acid sequence of the artificial nucleic acid have been shown as particularly effective in inhibiting unwanted off-target or bystander editing. In particular, it has been found that, in comparison to the G-A mismatch (at the central adenosine nucleotide not to be edited), the suppressive effect of the G·U wobble immediately 3′ or 5′ to the central adenosine is typically stronger and superior to the conventional used G-A mismatch. Moreover, a G-A mismatch destabilizes the double strand binding strength between the target RNA and the artificial nucleic acid molecule, and often reduces editing efficiency. Thus, in the second nucleic acid sequence of the artificial nucleic acid, nucleotide triplets are preferred which avoid a G-A mismatch at the central adenosine nucleotide (not to be edited), but enable a G·U wobble immediately 3′ and/or 5′ to the central (off-target) adenosine.

Therefore, in a further preferred embodiment of the present invention, the second nucleic acid sequence of the artificial nucleic acid molecule comprising at least 3 nucleotides that are complementary or partially complementary to a nucleic acid sequence in the at least one off-target sequence, which comprises an adenosine nucleotide not to be edited and the nucleotides immediately 5′ and 3′ of said adenosine nucleotide,
does not comprise a nucleotide sequence 5′-AUA-3′ and comprises one or more of a nucleotide triplet selected from the group consisting of 5′-GUG-3′, 5′-AUG-3′, and 5′-GUA-3′, preferably if an adenosine nucleotide not to be edited in the off-target sequence is the central adenosine nucleotide in a nucleotide triplet 5′-UAU-3′;
does not comprise a nucleotide triplet 5′-CUA-3′ and comprises one or more of a nucleotide triplet selected from the group consisting of 5′-CUG-3′, and 5′-UUG-3′, preferably if an adenosine nucleotide not to be edited in the off-target sequence is the central adenosine nucleotide in a nucleotide triplet 5′-UAG-3′;
does not comprise a nucleotide triplet 5′-GUA-3′ and comprises a nucleotide triplet 5′-GUG-3′, preferably if an adenosine nucleotide not to be edited in the off-target sequence is the central adenosine nucleotide in a nucleotide triplet 5′-UAC-3′;
does not comprise a nucleotide triplet 5′-UUA-3′ and comprises a nucleotide triplet 5′-UUG-3′, preferably if an adenosine nucleotide not to be edited in the off-target sequence is the central adenosine nucleotide in a nucleotide triplet 5′-UAA-3′; and/or
does not comprise a nucleotide triplet 5′-AUU-3′ and comprises a nucleotide triplet 5′-GUU-3′, preferably if an adenosine nucleotide not to be edited in the off-target sequence is the central adenosine nucleotide in a nucleotide triplet 5′-AAU-3′.

Further, the inventors found that the boosting effect of U·G wobbles immediately 3′ and/or 5′ of the target adenosine in the target RNA cannot further enhanced—or is even reduced—by a C-A mismatch at the target site which is conventionally used to enhance RNA editing at the target adenosine. Therefore, in the first nucleic acid sequence of the artificial nucleic acid, nucleotide triplets are preferred which avoid a C-A mismatch with the target adenosine nucleotide to be edited, but enable a U·G wobble immediately 3′ or 5′ to the central (target) adenosine.

Therefore, in a preferred embodiment of the present invention, the first nucleic acid sequence
consists of nucleotide triplet 5′-UUA-3′, preferably if the target adenosine nucleotide to be edited in the target sequence is the central adenosine nucleotide in a nucleotide triplet 5′-UAG-3′;
consists of nucleotide triplet 5′-AUU-3′, preferably if the target adenosine nucleotide to be edited in the target sequence is the central adenosine nucleotide in a nucleotide triplet 5′-GAU-3′;
consists of a nucleotide triplet selected from the group consisting of 5′-CUU-3′, 5′-UUC-3′, 5′-UUU-3′, 5′-UUG-3′, 5′-GUU-3′, 5′-UUA-3′, and 5′-AUU-3′, preferably if the target adenosine nucleotide to be edited in the target sequence is the central adenosine nucleotide in a nucleotide triplet 5′-GAG-3′;
consists of nucleotide triplet 5′-GUU-3′, preferably if the target adenosine nucleotide to be edited in the target sequence is the central adenosine nucleotide in a nucleotide triplet 5′-GAC-3′;
consists of nucleotide triplet 5′-UUU-3′, or 5′-AUU-3′, preferably if the target adenosine nucleotide to be edited in the target sequence is the central adenosine nucleotide in a nucleotide triplet 5′-GAA-3′;
consists of nucleotide triplet 5′-UUG-3′, or 5′-UUA-3′, preferably if the target adenosine nucleotide to be edited in the target sequence is the central adenosine nucleotide in a nucleotide triplet 5′-CAG-3′; and/or
consists of nucleotide triplet 5′-UUU-3′, preferably if the target adenosine nucleotide to be edited in the target sequence is the central adenosine nucleotide in a nucleotide triplet 5′-AAG-3′.

As G·U wobbles require a uridine as nearest neighbor to an off-target adenosine, there is a sequence limitation for the use of G·U wobble base pairs in the second nucleic acid sequence of the artificial nucleic acid. In order to avoid or at least reduce unwanted off-target editing of adenosines in the target RNA without a neighboring uridine, the second nucleic acid sequence preferably comprises nucleotide triplets comprising a central guanosine which mismatches with an adenosine not to be edited in the off-target sequence of the target. In another words, in a preferred embodiment of the present invention, the second nucleic acid sequence of the artificial nucleic acid is designed to form G·U wobble base pair(s) with uridine as nearest neighbor to an off-target adenosine, and, if no uridine is present as nearest neighbor to an off-target adenosine in the target RNA, the second nucleic acid sequence of the artificial nucleic acid includes nucleotide triplets comprising a central guanosine which forms a G-A-mismatch with the central adenosine of a nucleotide triplet of the off-target sequence of the target RNA, thereby preventing or at least reducing unwanted off-target editing of the off-target adenosine which is unamenable to G·U wobbling.

Therefore, in a preferred embodiment of the present invention, the second nucleic acid sequence further
does not comprise a nucleotide triplet 5′-CUC-3′ and comprises one or more of a nucleotide triplet 5′-CGC-3′, preferably if an adenosine nucleotide not to be edited in the off-target sequence is the central adenosine nucleotide in a nucleotide triplet 5′-GAG-3′;
does not comprise a nucleotide triplet 5′-GUC-3′ and comprises one or more of a nucleotide triplet 5′-GGC-3′, preferably if an adenosine nucleotide not to be edited in the off-target sequence is the central adenosine nucleotide in a nucleotide triplet 5′-GAC-3′;
does not comprise a nucleotide triplet 5′-UUC-3′ and comprises one or more of a nucleotide triplet selected from the group consisting of 5′-UGC-3′, and 5′-GGC-3′, preferably if an adenosine nucleotide not to be edited in the off-target sequence is the central adenosine nucleotide in a nucleotide triplet 5′-GAA-3′;
does not comprise a nucleotide triplet 5′-CUG-3′ and comprises one or more of nucleotide triplet 5′-CGG-3′, preferably if an adenosine nucleotide not to be edited in the off-target sequence is the central adenosine nucleotide in a nucleotide triplet 5′-CAG-3′;
does not comprise a nucleotide triplet 5′-GUG-3′ and comprises one or more of nucleotide triplet 5′-GGG-3′, preferably if an adenosine nucleotide not to be edited in the off-target sequence is the central adenosine nucleotide in a nucleotide triplet 5′-CAC-3′;
does not comprise a nucleotide triplet 5′-UUG-3′ and comprises one or more of a nucleotide triplet selected from the group consisting of 5′-UGG-3′ and 5′-GGG-3′, preferably if an adenosine nucleotide not to be edited in the off-target sequence is the central adenosine nucleotide in a nucleotide triplet 5′-CAA-3′;
does not comprise a nucleotide triplet 5′-CUU-3′ and comprises one or more of a nucleotide triplet selected from the group consisting of 5′-CGU-3′ and 5′-CGG-3′, preferably if an adenosine nucleotide not to be edited in the off-target sequence is the central adenosine nucleotide in a nucleotide triplet 5′-AAG-3′;
does not comprise a nucleotide triplet 5′-GUU-3′ and comprises one or more of a nucleotide triplet selected from the group consisting of 5′-GGU-3′ and 5′-GGG-3′, preferably if an adenosine nucleotide not to be edited in the off-target sequence is the central adenosine nucleotide in a nucleotide triplet 5′-AAC-3′; and/or
does not comprise a nucleotide triplet 5′-UUU-3′ and comprises one or more of a nucleotide triplet selected from the group consisting of 5′-UGU-3′, 5′-GGU-3′, 5′-UGG-3′, and 5′-GGG-3′, preferably if an adenosine nucleotide not to be edited in the off-target sequence is the central adenosine nucleotide in a nucleotide triplet 5′-AAA-3′.

The individual nucleic acid sequences of the first, second and/or third nucleic acid sequences of the artificial nucleic acid for editing a given target RNA typically depend on the sequence of a specific target RNA to be edited. The first, second and/or third nucleic acid sequences of the artificial nucleic acid are thus preferably complementary or at least partially complementary to the target RNA to be edited, wherein specific nucleotide triplets, as described herein, are preferably used in the artificial nucleic acid in order to boost editing of a target adenosine in the target sequence via U·G wobbling, and/or in order to reduce editing of (an) off-target adenosine(s) in the off-target sequence via G·U wobbling, if necessary in combination with the use of G-A mismatches and/or uridine depletion at off-target adenosine(s) not amenable for G·U wobbling. In the context of the present specification, the term “uridine depletion” refers to the partial or complete removal of pairing(s) of uridine(s) in the artificial nucleic acid described herein with off-target adenosine(s) in the target RNA. The term thus typically refers to the deletion of one or more uridine nucleotides in the artificial nucleic acid or to the substitution of one or more uridine nucleotides in the artificial nucleic acid by another nucleotide.

The artificial nucleic acid of the present invention does not necessarily provide for U·G wobbling in the target sequence to boost editing of a target adenosine and, at the same time, G·U wobbling in the off-target sequence to reduce editing of at least one off-target adenosine.

Thus, in a particular embodiment, the artificial nucleic acid comprises a first nucleic acid sequence which is suitable for boosting editing of a target adenosine via U·G wobbling in the target sequence, and a second nucleic acid sequence which reduces editing of at least one off-target adenosine in the off-target sequence not via G·U wobbling but rather by the use of other means which are suitable to reduce editing of (an) off-target adenosine(s) in the off-target sequence, such as G-A mismatches and/or uridine depletion, as described above.

In another embodiment, the artificial nucleic acid comprises a first nucleic acid sequence which is suitable to boost editing of a target adenosine via U·G wobbling, and a second nucleic acid sequence which is suitable to reduce editing of at least one off-target adenosine in the off-target sequence via G·U wobbling.

In a further preferred embodiment, the artificial nucleic acid comprises a first nucleic acid sequence which promotes editing of a target adenosine not via U·G wobbling, but rather by the use of other means which are suitable to promote editing of a target adenosine as described above, such as a C-A mismatch, and a second nucleic acid sequence which is suitable to reduce editing of at least one off-target adenosine in the off-target sequence via G·U wobbling.

Thus, in a preferred embodiment, the artificial nucleic acid comprises a first nucleic acid sequence which is suitable for promoting editing of a target adenosine via a C-A mismatch, and a second nucleic acid sequence which is suitable to reduce editing of at least one off-target adenosine in the off-target sequence via G·U wobbling, if necessary in combination with the use of G-A mismatches and/or uridine depletion at off-target adenosine(s), as described above.

In a preferred embodiment, the first nucleic acid sequence of the artificial nucleic acid is not identical with the second nucleic acid sequence and/or the third nucleic acid sequence of the artificial nucleic acid. Preferably, each of the first, second and third nucleic acid sequences of the artificial nucleic acid described herein is characterized by a distinct sequence.

In addition to the first nucleic acid sequence and the second nucleic acid sequence and/or a third nucleic acid sequence, the artificial nucleic acid of the present invention may comprise (a) further nucleic acid sequence(s). In a preferred embodiment, the artificial nucleic acid of the present invention comprises a nucleic acid sequence which is capable of recruiting an adenosine deaminase.

The term adenosine ‘deaminase’ as used herein refers to any compound, preferably a peptide, a protein or a protein domain, which is capable of catalysing the deamination of an adenosine nucleotide or a variant thereof in a target RNA. The term thus not only refers to full-length and wild type deaminases, such as ADAR1, ADAR2, but also to a fragment or variant of a deaminase, preferably a functional fragment or a functional variant. In particular, the term also refers to mutants and variants of a deaminase, such as mutants of ADAR1, ADAR2, preferably as described herein. Furthermore, the term deaminase as used herein also comprises any deaminase fusion protein (e.g. based on Cas9, Cas13, MS2 Coat Protein or the Lambda-N-peptide, TAR binding protein). In the context of the present invention, the term ‘deaminase’ also refers to tagged variants of a deaminase, as described herein. The deaminase, as described herein, is preferably derived from human, mouse or rat.

In a preferred embodiment, the artificial nucleic acid comprises a nucleic acid sequence capable of binding to an adenosine deaminase, preferably to the dsRNA binding domain of an adenosine deaminase.

In a more preferred embodiment, the artificial nucleic acid comprises a nucleic acid sequence capable of binding to an adenosine deaminase fusion protein.

In this context, any ADAR fusion protein that engages a specific RNA protein interaction for site-directed RNA editing can be envisaged. For example, the adenosine deaminase fusion protein may be selected from the group consisting of Cas9-ADAR, Cas13-ADAR, MS2 Coat Protein-ADAR, λN-ADAR, CIRTS-ADAR, and TAR binding protein-ADAR.

In certain embodiments, the artificial nucleic acid comprises at least one coupling agent capable of recruiting a deaminase, wherein the deaminase comprises a moiety that binds to said coupling agent. The coupling agent, which recruits a deaminase is typically covalently linked to the 5′-terminus or to the 3′-terminus of the artificial nucleic acid. The coupling agent may alternatively also be linked to an internal nucleotide (i.e. not a 5′- or 3′-terminal nucleotide) of the artificial nucleic acid, for example via linkage to a nucleotide variant or a modified nucleotide, preferably as described herein, such as amino-thymidine.

In some embodiments, the artificial nucleic acid does not comprise a coupling agent capable of recruiting a deaminase, wherein the deaminase preferably comprises a moiety that binds to said coupling agent.

The coupling agent may e.g. be selected from the group consisting of O6-benzylguanine, O2-benzylcytosine, chloroalkane, 1×BG, 2×BG, 4×BG, and a variant of any of these. According to a particular embodiment, the coupling agent is a branched molecule, such as 2×BG or 4×BG, each of which is preferably capable of recruiting a deaminase molecule, thus preferably amplifying the editing reaction. Exemplary structures of suitable branched coupling agents are depicted below:

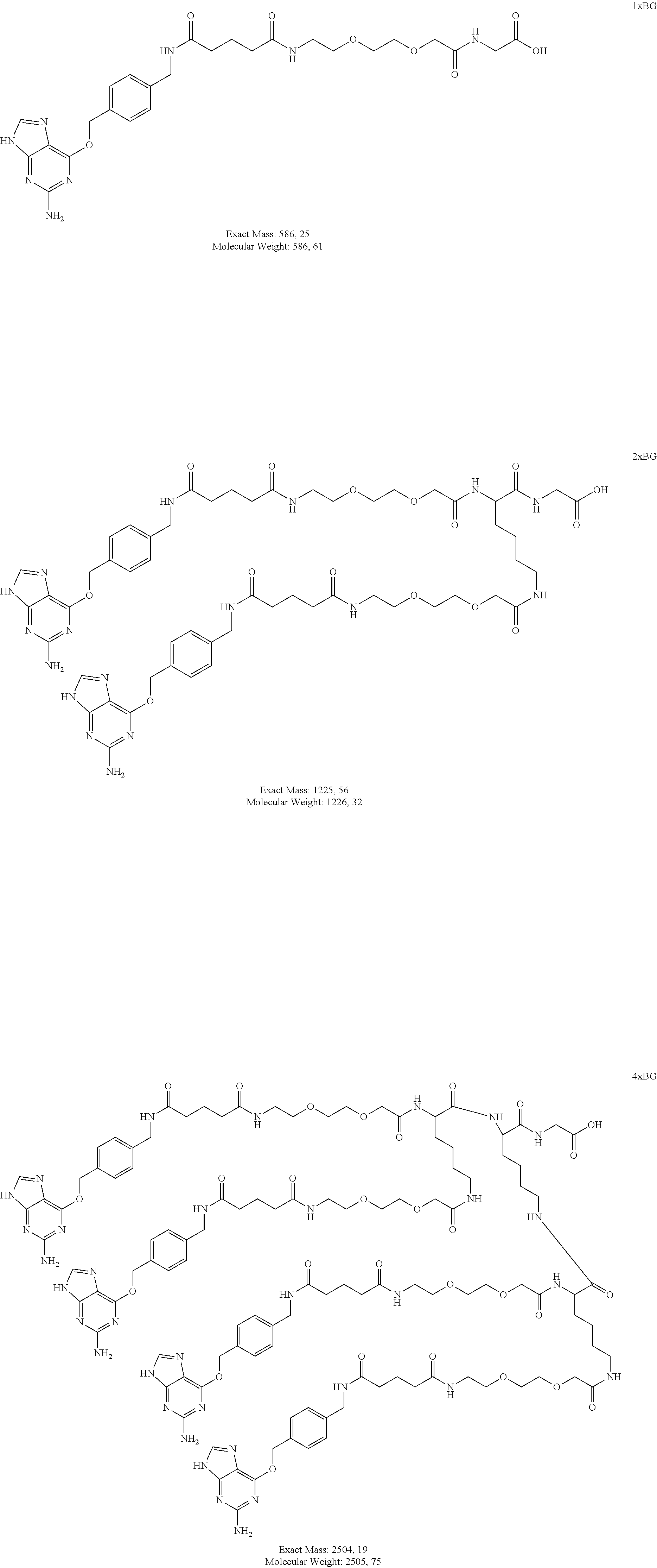 [SMILES: Nc1nc(OCc2ccc(CNC(=O)CCCC(=O)NCCOCCOCC(=O)NCC(=O)O)cc2)c2nc[nH]c2n1 ; Nc1nc(OCc2ccc(CNC(=O)CCCC(=O)NCCOCCOCC(=O)NCCCCC(NC(=O)COCCOCCNC(=O)CCCC(=O)NCc3ccc(COc4nc(N)nc5[nH]cnc45)cc3)C(=O)NCC(=O)O)cc2)c2nc[nH]c2n1 ; Nc1nc(OCc2ccc(CNC(=O)CCCC(=O)NCCOCCOCC(=O)NCCCCC(NC(=O)COCCOCCNC(=O)CCCC(=O)NCc3ccc(COc4nc(N)nc5[nH]cnc45)cc3)C(=O)CNC(CCCCNC(=O)C(CCCCNC(=O)COCCOCCNC(=O)CCCC(=O)NCc3ccc(COc4nc(N)nc5[nH]cnc45)cc3)NC(=O)COCCOCCNC(=O)CCCC(=O)NCc3ccc(COc4nc(N)nc5[nH]cnc45)cc3)C(=O)NCC(=O)O)cc2)c2nc[nH]c2n1]

The coupling agent is preferably capable of specifically binding to a moiety in a deaminase. Said moiety in a deaminase is preferably a tag, which is linked to an adenosine deaminase as described herein. Said tag may e.g. be selected from the group consisting of a SNAP-tag, a CLIP-tag, a HaloTag, and a fragment or variant of any one of these. In an alternative embodiment, the tag is not a SNAP-tag or a fragment or a variant thereof.

In the context of the present invention, a ‘variant’ of a nucleic acid sequence or of an amino acid sequence is at least 40%, preferably at least 50%, more preferably at least 60%, more preferably at least 70%, even more preferably at least 80%, even more preferably at least 90%, most preferably at least 95% identical to the sequence, the variant is derived from. Preferably, the variant is a functional variant.

As used herein, a ‘fragment’ of a nucleic acid sequence or of an amino acid sequence consists of a continuous stretch of nucleotides or amino acid residues corresponding to a continuous stretch of nucleotides or amino acid residues in the full-length sequence, which represents at least 5%, 10%, 20%, preferably at least 30%, more preferably at least 40%, more preferably at least 50%, even more preferably at least 60%, even more preferably at least 70%, even more preferably at least 80%, and most preferably at least 90% of the full-length sequence, the fragment is derived from. Such a fragment, in the sense of the present invention, is preferably a functional fragment.

Accordingly, the deaminases bound by the coupling agent in these embodiments are preferably artificial versions of endogenous deaminases, preferably of a deaminase as described herein.

As mentioned above, the deaminase may be selected from a SNAP-tagged deaminase, a Halo-tagged deaminase, and a Clip-tagged deaminase, wherein the SNAP-tagged deaminase may be selected from the group consisting of SNAP-ADAR1, SNAP-ADAR2, SNAPf-ADAR1, and SNAPf-ADAR2, the Halo-tagged deaminase may be selected from the group consisting of Halo-ADAR1, and Halo-ADAR2, and the Clip-tagged deaminase may be selected from the group consisting of Clip-ADAR1, Clip-ADAR2, Clipf-ADAR1 and Clipf-ADAR2, or a fragment or variant of any of these, wherein the deaminase is derived from human. However, in a preferred embodiment, the deaminase is not selected from a SNAP-tagged deaminase, preferably not from a tagged deaminase.

In some embodiments, the deaminase is not a recombinant deaminase. Preferably, the deaminase is not selected from a SNAP-tagged deaminase, a Halo-tagged deaminase, and a Clip-tagged deaminase, wherein the SNAP-tagged deaminase is preferably selected from the group consisting of SNAP-ADAR1, SNAP-ADAR2, SNAPf-ADAR1, and SNAPf-ADAR2, the Halo-tagged deaminase is preferably selected from the group consisting of Halo-ADAR1, and Halo-ADAR2, and/or the Clip-tagged deaminase is preferably selected from the group consisting of Clip-ADAR1, Clip-ADAR2, Clipf-ADAR1 and Clipf-ADAR2, or a fragment or variant of any of these, wherein the deaminase is preferably derived from a mammalian, more preferably from mouse or human. Even more preferably, the deaminase is an endogenous deaminase.

In a preferred embodiment, the deaminase is a hyperactive mutant of any of the deaminases mentioned herein, preferably a hyperactive Q mutant, more preferably a hyperactive Q mutant of an ADAR1 deaminase, an ADAR2 deaminase (e.g. human ADAR1p150, E1008Q; human ADAR1p110, E713Q; human ADAR2, E488Q) or a tagged version thereof, most preferably as described herein, or a fragment or variant of any of these.

Tagged deaminases, preferably as described herein, (e.g. SNAP-, SNAPf-, Clip-, Clipf-, Halo-tagged deaminases or fragments or variants thereof) are preferably overexpressed for RNA editing, for example by transient transfection of a cell with a vector encoding said tagged deaminase or by stable expression in a transgenic cell, tissue or organism.

The SNAP-ADAR approach and related approaches which apply a self-labeling protein tag like the SNAP-, Clip- or Halo-tag typically apply comparably short (ca. 13-25 nt) and chemically synthesized and modified guide RNAs. Due to the small length and the chemical modification the respective guide RNA typically induce comparably little bystander/off-target editing in the mRNA/guide RNA duplex. It was shown for example that 2′-O-methylation suppresses bystander/off-target editing relatively efficiently if placed at least two nucleosides away from the cytosine opposite the targeted adenosine (Vogel et al., Angew. Chemie 2014, Nature Methods 2018). In adenosine-rich sequences or if a target adenosine is placed in an adenosine-rich codon, e.g. 5′-AAG or 5′-CAA or others, then the suppression of bystander/off-target editing is more difficult without affecting on-target editing. Subtle chemical modification, e.g. with 2′-F, has been shown effective to some extent (Vogel et al., Nat. Meth. 2018), but are typically not able to suppress bystander editing entirely. Another issue is the enhancement of editing yields in difficult to edit 5′-GAN codons (target nucleotide A underlined). Editing yields can be increased by using the hyperactive ADAR mutants referred to as E>Q mutants (see above), however, they also induce notable transcriptome-wide off-target effects (Vogel at al., Nat. Meth. 2018). Consequently, solutions with wildtype deaminases are desirable. Strategic mismatching of the guanosine specifically in the 5′-GAG codon was shown, but only in vitro and not in cell culture, to foster editing with wildtype SNAP-ADAR enzymes (Schneider et al., Nucl. Acids Res 2014).

In certain embodiments, the artificial nucleic acid comprises at least one RNA motif (e.g. MS2-loop(s), direct repeats of trans-activating crRNA(s), BoxB motif(s), HIV trans-activation response (TAR) hairpin(s)) capable of recruiting a deaminase or another effector fusion-protein that was developed for a tethering approach, like MCP-ADAR (Azad, M. T. A., et al.: Site-directed RNA editing by adenosine deaminase acting on RNA for correction of the genetic code in gene therapy. Gene Ther 24(12): 779-786(2017), and D. Katrekar et al. (In vivo RNA editing of point mutations via RNA-guided adenosine deaminases. Nat. Methods 16(3), 239-242(2019)); or like dCas-ADAR (Cox, D. B. T., et al., supra; Omar O. Abudayyeh, et al., supra), or like LambdaN-ADAR (Montiel-Gonzalez, M. F., et al. (Correction of mutations within the cystic fibrosis transmembrane conductance regulator by site-directed RNA editing. Proc Natl Acad Sci USA 110(45): 18285-18290(2013)), or like TBP-ADAR (S. Rauch et al.: Programmable RNA-Guided RNA Effector Proteins Built from Human Parts. Cell 178, 122-134.e12(2019)).

The artificial nucleic acid is suitable for site-directed editing of an RNA by an adenosine deaminase or a fragment or variant thereof, preferably an ADAR (adenosine deaminase acting on dsRNA) enzyme or a fragment or variant thereof, more preferably selected from the group consisting of ADAR1, ADAR2 and a fragment or variant thereof, e.g. a peptide or protein comprising an adenosine deaminase domain.

In preferred embodiments of the present invention, the artificial nucleic acid molecule comprises a nucleic acid sequence capable of specifically binding to a double-stranded (ds) RNA binding domain of an adenosine deaminase. Advantageously, the nucleic acid sequence capable of binding to a deaminase binds to endogenous deaminases. The artificial nucleic acid according to the invention thus may promote site-directed RNA editing employing an endogenous (or heterologously expressed) deaminase.

In a preferred embodiment, the endogenous adenosine deaminase is ADAR1, or ADAR2, preferably ADAR1, more preferably a eukaryotic adenosine deaminase, more preferably a vertebrate adenosine deaminase, even more preferably a mammalian adenosine deaminase, most preferably a human adenosine deaminase, such as hADAR1 or hADAR2, or a fragment or variant of any of these, in particular ADAR1p110 or ADAR1p150, preferably ADAR1p110.

Preferably, the nucleic acid sequence of the artificial nucleic acid molecule capable of recruiting an adenosine deaminase is further capable of intramolecular base pairing. Therefore, the nucleic acid sequence of the artificial nucleic acid molecule preferably comprises a nucleic acid sequence that is capable of forming a stem-loop structure. In certain embodiments, said stem-loop structure comprises or consists of a double-helical stem comprising at least one mismatch. In a preferred embodiment, the stem loop structure comprises a loop consisting of from 3 to 8, preferably from 4 to 6, more preferably 5, nucleotides.

In a preferred embodiment, the loop comprises the nucleic acid sequence GCUAA or GCUCA, and the nucleic acid sequence preferably comprises a nucleotide sequence selected from the group consisting of 5′-GGUGUCGAGAAGAGGAGAACAAUAUGCUAAAUGUUGUUCUCGUCUCCUCGACACC-3′ (SEQ ID NO: 2), and 5′-GUGGAAUAGUAUAACAAUAUGCUAAAUGUUGUUAUAGUAUCCCAC-3′ (SEQ ID NO: 1).

In another preferred embodiment, the loop comprises the nucleic acid sequence CUUUC, and the nucleic acid sequence preferably comprises a nucleotide sequence selected from the group consisting of 5′-GGUGUCGAGAAGAGGAGAACAAUAUCUUUCAUGUUGUUCUCCUCUCCUCGACACC-3′ (SEQ ID NO: 3) and 5′-GUGGAAUAGUAUAACAAUAUCUUUCAUGUUGUUAUACUAUCCCAC-3′ (SEQ ID NO: 4).

Since the above sequences are adapted from a well-known ADAR2 target site in glutamate receptor 2 mRNA, they are also known as R/G motif.

Therefore, the artificial nucleic acid of the present invention is preferably a single-stranded (ss) nucleic acid molecule. In a preferred embodiment, the artificial nucleic acid is a single-stranded nucleic acid, which at physiological conditions comprises double-stranded (ds) regions. Preferably, the artificial nucleic acid is a single-stranded nucleic acid comprising (a) double-stranded region(s), that is/are not intended to bind to the target mRNA, and is/are capable of binding a deaminase.

As mentioned above, the artificial nucleic acid of the present invention can be used in a number of approaches to guide an adenosine deaminase to a target adenosine to be edited while avoiding or reducing off-target editing of non-target adenosines. As an example, the artificial nucleic acid of the present invention can be used in the LEAPER approach (leveraging endogenous ADAR for programmable editing of RNA) that employs short engineered ADAR-recruiting RNAs delivered by a plasmid or viral vector or as a synthetic oligonucleotide (Qu, L., et al., Programmable RNA editing by recruiting endogenous ADAR using engineered RNAs. Nat Biotechnol, 2019).

Another approach for site-directed editing of a target RNA, where the artificial nucleic acid of the present invention can be suitably be used, are CLUSTER guide RNAs as e.g. described in WO2022078995A1. CLUSTER guide RNAs comprise a cluster of recruitment sequences which bind to regions in the target RNA which do not comprise any editable adenosine nucleotides or contain adenosine nucleotide(s) in a 5′-GAN-3′ or 5′-CAN-3′ context, which are less prone to unwanted off-target editing. The artificial nucleic acids of the present invention can be integrated into the CLUSTER approach by using G·U wobble base pairing in the recruitment sequence/target RNA duplex thereby enlarging the options with respect to the regions of the target RNA which are bound by the recruiting sequences. In other words, by using the G·U wobbling strategy, it is not necessary to select regions of the target RNA (to be bound by the recruitment sequences) which are totally free from editable adenosines or contain adenosine nucleotides in a specific context (5′-GAN-3′ or 5′-CAN-3′), since unwanted off-target editing of an adenosine included in the target RNA can be reduced or even avoided by integrating into the recruiting moiety of the artificial nucleic acid an appropriate nucleotide triplet which forms a wobble base pair 3′ or 5′ to said (off-target) adenosine nucleotide. This further enlarges the sequence space, from which CLUSTER guide RNA sequences may be selected. This is a clear improvement as it allows creating CLUSTER guide RNAs that work more efficiently. On the one hand, it allows for the CLUSTER guide RNA to bind closer to the on-target site with all antisense sequences. On the other hand, it allows for selection of sequences that are less prone to misfolding. Furthermore, a larger sequence space for CLUSTER guide RNAs will enable to reduce potentially adverse effects of guide RNA binding to the target transcript, like sequestering of elements that modulate the splicing process.

Therefore, the artificial nucleic acid of the present invention may comprise a further recruiting moiety capable of recruiting an adenosine deaminase, preferably an endogenous adenosine deaminase, by binding to the target RNA, wherein said recruiting moiety comprises a recruitment sequence that is complementary or partially complementary to a nucleic acid sequence in the target RNA which does not contain any adenosine nucleotides to be edited.

In a preferred embodiment, the further recruiting moiety comprises a cluster of recruitment sequences comprising at least two recruitment sequences, which are linked via a nucleotide linker. The linker comprises at least 1 nucleotide, and may comprise 1 to 100, e. g. 1 to 90, 1 to 80, 1 to 70, 1 to 60, 1 to 50, 1 to 40, 1 to 30 1 to 20 or 1 to 10 nucleotides, and preferably comprises 2 to 6, e.g. 2, 3, 4, 5 or 6 nucleotides, preferably adenosine nucleotides.

In this context, the cluster of recruitment sequences may comprise at least three recruitment sequences, preferably 3 to 10, e.g. 3, 4, 5, 6, 7, 8, 9, 10 recruitment sequences, more preferably 3 to 6 recruitment sequences, wherein the cluster recruitment sequences each comprises 10 to 200, preferably 10 to 100, more preferably 15 to 100, or 20 to 100, e.g. 20 to 90, 20 to 80, 20 to 70, 20 to 60, 20 to 50, 20 to 40, or 20 to 30 nucleotides.

With respect to cluster recruitment sequences, it is referred to WO2022078995A1, which is hereby incorporated by reference.

In a preferred embodiment, the recruitment sequence(s), which is/are complementary or partially complementary to a nucleic acid sequence in the target RNA which does not contain adenosine nucleotides to be edited, comprises nucleotide triplets as defined above with respect to the second nucleic acid sequence of the artificial nucleic acid enabling G·U wobbling immediately 3′ or 5′ of the off-target adenosine (not to be edited) in the target RNA.

### Concretely:

If the region of the target RNA which is to be bound by a (cluster) recruitment sequence of the artificial nucleic acid comprises an adenosine nucleotide that is not to be edited in a nucleotide triplet context 5′-UAU-3′, the second nucleic acid sequence of the artificial nucleic acid does not comprise a nucleotide triplet 5′-AUA-3′ (complementary to the 5′-UAU-3′ triplet), but comprises a nucleotide triplet selected from the group consisting of 5′-GUG-3′, 5′-AUG-3′, 5′-GUA-3′, 5′-GGG-3′, 5′-AGG-3′, and 5′-GGA-3′, preferably selected from the group consisting of 5′-GUG-3′, 5′-AUG-3′, and 5′-GUA-3′.

If the region of the target RNA which is to be bound by a (cluster) recruitment sequence of the artificial nucleic acid comprises an adenosine nucleotide that is not to be edited in a nucleotide triplet context 5′-UAG-3′, the second nucleic acid sequence of the artificial nucleic acid does not comprise a nucleotide triplet 5′-CUA-3′ (complementary to the 5′-UAG-3′ triplet), but comprises a nucleotide triplet selected from the group consisting of 5′-CUG-3′, 5′-UUG-3′, 5′-CGG-3′, and 5′-UGG-3′, preferably selected from the group consisting of 5′-CUG-3′, and 5′-UUG-3′.

If the region of the target RNA which is to be bound by a (cluster) recruitment sequence of the artificial nucleic acid comprises an adenosine nucleotide that is not to be edited in a nucleotide triplet context 5′-UAC-3′, the second nucleic acid sequence of the artificial nucleic acid does not comprise a nucleotide triplet 5′-GUA-3′ (complementary to the 5′-UAC-3′ triplet), but comprises a nucleotide triplet selected from the group consisting of 5′-GUG-3′, and 5′-GGG-3′, preferably a nucleotide triplet 5′-GUG-3′.

If the region of the target RNA which is to be bound by a (cluster) recruitment sequence of the artificial nucleic acid comprises an adenosine nucleotide that is not to be edited in a nucleotide triplet context 5′-UAA-3′, the second nucleic acid sequence of the artificial nucleic acid does not comprise a nucleotide triplet 5′-UUA-3′ (complementary to the 5′-UAG-3′ triplet), but comprises a nucleotide triplet selected from the group consisting of 5′-UUG-3′, 5′-UGG-3′, 5′-GUG-3′, and 5′-GGG-3′, preferably a nucleotide triplet 5′-UUG-3′.

If the region of the target RNA which is to be bound by a (cluster) recruitment sequence of the artificial nucleic acid comprises an adenosine nucleotide that is not to be edited in a nucleotide triplet context 5′-AAU-3′ (wherein the central adenosine is the adenosine not to be edited), the second nucleic acid sequence of the artificial nucleic acid does not comprise a nucleotide triplet 5′-AUU-3′ (complementary to 5′-AAU-3′ triplet), but rather comprises a nucleotide triplet selected from the group consisting of 5′-GUU-3′, and 5′-GGU-3′, preferably a nucleotide triplet 5′-GUU-3′.

In this context, it is preferred that a region of a target RNA which is to be bound by a (cluster) recruitment sequence of the artificial nucleic acid, as defined above, comprises no adenosine nucleotides which are not neighbored by a uridine nucleotide (which can be G·U wobbled) or which are not present in a 5′-GAN-3′ or 5′-CAN-3′ context (where editing of adenosine nucleotides rarely occurs).

Therefore, in a preferred embodiment of the present invention, the region(s) of the target RNA bound by the further recruiting moieties does not include an adenosine nucleotide in a 5′-AAG-3′, 5′-AAC-3′ or 5′-AAA-3′ context.

In this context, if a region of the target RNA which is to be bound by a (cluster) recruitment sequence of the artificial nucleic acid comprises an adenosine nucleotide in a triplet context which is not amenable for G·U wobbling, alternatively or additionally G-A mismatches and/or uridine depletion can be applied in order to reduce or even eliminate editing of the off-target adenosine(s), as described above.

The artificial nucleic acid according to the present invention is not limited in its length and may be, for example, an oligonucleotide. As used herein, the term ‘oligonucleotide’ may refer to short nucleic acid molecules (e.g. a 6-mer or a 10-mer) as well as to longer oligonucleotides (e.g. nucleic acid molecules comprising 100 or even 200 nucleotides), wherein the oligonucleotide may comprise (unmodified or modified) ribonucleotides and/or (unmodified or modified) deoxynucleotides. According to a preferred embodiment, the artificial nucleic acid comprises at least about 15, preferably at least about 20, more preferably at least about 25, even more preferably at least about 30, even more preferably at least about 35, most preferably at least about 40, nucleotides. Alternatively, the length of the artificial nucleic acid is in the range from about 15 to about 1000 nucleotides, e.g. from about 15 to about 400 nucleotides, from about 15 to about 300 nucleotides, from about 15 to about 200 nucleotides, preferably from about 20 to about 150 nucleotides, more preferably from about 20 to about 100 nucleotides, most preferably from about 20 to about 80 nucleotides.

The artificial nucleic acid according to the present invention may be a linear or a circular nucleic acid molecule. The artificial nucleic acid may be a circular RNA, for example a circularized RNA, which may be circularized using known methods, for example the Tornado expression system (Litke, J. L. and S. R. Jaffrey (2019). Highly efficient expression of circular RNA aptamers in cells using autocatalytic transcripts. Nat Biotechnol 37(6): 667-675).

In particular embodiments of the present invention, the artificial nucleic acid may be a circular CLUSTER guide RNA. In a preferred embodiment the order of recruitment sequences within the cluster of recruitment sequences of a circular CLUSTER guide RNA is altered to position the 5′- and 3′-exit-points of its target transcript distal of the targeting sequence (the part of the guide RNA that forms a duplex with the section of the target transcript that contains the target adenosine). In a preferred embodiment of the circular CLUSTER guide RNA, the cluster of recruitment sequences comprises 1-20, preferably 2-10, more preferably 3-6, most preferably 4 recruitment sequences of 10-100 nt, preferably 13-50 nt, more preferably 15-30 nt, most preferably 20 nt length each. These recruitment sequences are preferably connected via a nucleotide linker. The linker comprises at least 1 nucleotide, and may comprise 1 to 100, e. g. 1 to 90, 1 to 80, 1 to 70, 1 to 60, 1 to 50, 1 to 40, 1 to 30 1 to 20 or 1 to 10 nucleotides, and preferably comprises 2 to 6, e.g. 2, 3, 4, 5 or 6 nucleotides, preferably adenosine nucleotides.

The circular nucleic acid sequence of the artificial nucleic acid molecule is capable of recruiting an adenosine deaminase preferably by intermolecular base pairing with one or more recruitment sequences (i.e. a cluster of recruitment sequences), more preferably by both inter- and intramolecular base pairing via one or more recruitment sequences and by simultaneously forming a stem-loop structure and even more preferably by both inter- and intramolecular base pairing via one or more recruitment sequences and by simultaneously forming an internal duplex structure. In certain embodiments, said intramolecular duplex structure comprises or consists of a double-helical stem comprising at least one mismatch and at least one bulge. In a preferred embodiment, the duplex structure comprises one mismatch and one bulge consisting of from 3 to 8, preferably from 4 to 6, more preferably 5, nucleotides. In a preferred embodiment, the bulge comprises the nucleic acid sequence UUUC in one strand and the nucleotide C in the other.

The duplex structure preferably comprises a nucleotide sequence selected from the group consisting of 5′-GGUGUCGAGAAGAGGAGAACAAUAUGCUACUGCCAUCAGUCGGCGUGGACUGUAGAA CCAUGCCGACUGAUGGCAGAAUGUUGUUCUCGUCUCCUCGACACC-3′ (SEQ ID NO: 5), 5′-GGUGUCGAGAAGAGGAGAACAAUAUCUUUCUGCCAUCAGUCGGCGUGGACUGUAGAA CCAUGCCGACUGAUGGCAGCAUGUUGUUCUCGUCUCCUCGACACC-3′ (SEQ ID NO: 6) and 5′-GGUGUCGAGAAGAGGAGAACAAUAUCUUUCUGCCAUCAGUCGGCGUGGACUGUAGAA CCAUGCCGACUGAUGGCAGCAUGUUGUUCUCCUCUCCUCGACACC-3′ (SEQ ID NO: 7).

In a preferred embodiment, the targeting sequence of the circular artificial nucleic acid is positioned next to its intramolecular duplex structure or hairpin structure that is capable of recruiting an adenosine deaminase.

In certain embodiments, the artificial nucleic acid comprises at least one G-A mismatch and/or uridine depletion in addition to at least one G·U wobble in order to control off-target editing.

In certain embodiments, the artificial nucleic acid comprises at least one G-A mismatch and/or uridine depletions in addition to at least one G·U wobble in order to control off-target editing and at least one U·G wobble in order to increase on-target editing.

In particular embodiments of the present invention, the artificial nucleic acid may comprise nucleotides which are chemically modified. As used herein, the term ‘chemical modification’ preferably refers to a chemical modification selected from backbone modifications, sugar modifications or base modifications, including abasic sites. A ‘chemically modified nucleic acid’ in the context of the present invention may refer to a nucleic acid comprising at least one chemically modified nucleotide. Particularly, the artificial nucleic acid may comprise a plurality of chemically modified nucleotides, which may result in specific modification patterns which are e.g. disclosed in WO/2020/001793.

Generally, the artificial nucleic acid molecule of the present invention may comprise native (=naturally occurring) nucleotides as well as chemically modified nucleotides. As used herein, the term ‘nucleotide’ generally comprises (unmodified and modified) ribonucleotides as well as (unmodified and modified) deoxynucleotides. The term ‘nucleotide’ thus preferably refers to adenosine, deoxyadenosine, guanosine, deoxyguanosine, inosine, deoxyinosine, 5-methoxyuridine, thymidine, uridine, deoxyuridine, cytidine, deoxycytidine or to a variant thereof. Moreover, where reference is made herein to a ‘nucleotide’, the respective nucleoside is preferably comprised as well.

In this respect, a ‘variant’ of a nucleotide is typically a naturally occurring or an artificial variant of a nucleotide. Accordingly, variants are preferably chemically derivatized nucleotides with non-natively occurring functional groups, which are preferably added to or deleted from the naturally occurring nucleotide or which substitute the naturally occurring functional groups of a nucleotide. Accordingly, in such a nucleotide variant each component of the naturally occurring nucleotide, preferably a ribonucleotide or a deoxynucleotide, may be modified, namely the base component, the sugar (ribose) component and/or the phosphate component forming the backbone of the artificial nucleic acid, preferably by a modification as described herein. The term ‘variant (of a nucleotide, ribonucleotide, deoxynucleotide, etc.)’ thus also comprises a chemically modified nucleotide, preferably as described herein.

A chemically modified nucleotide as used herein is preferably a variant of guanosine, uridine, adenosine, thymidine and cytidine including, without implying any limitation, any natively occurring or non-natively occurring guanosine, uridine, adenosine, thymidine or cytidine that has been altered chemically, for example by acetylation, methylation, hydroxylation, etc., including 1-methyl-adenosine, 1-methyl-guanosine, 1-methyl-inosine, 2,2-dimethyl-guanosine, 2,6-diaminopurine, 2′-amino-2′-deoxyadenosine, 2′-amino-2′-deoxycytidine, 2′-amino-2′-deoxyguanosine, 2′-amino-2′-deoxyuridine, 2-amino-6-chloropurineriboside, 2-aminopurine-riboside, 2′-araadenosine, 2′-aracytidine, 2′-arauridine, 2′-azido-2′-deoxyadenosine, 2′-azido-2′-deoxycytidine, 2′-azido-2′-deoxyguanosine, 2′-azido-2′-deoxyuridine, 2-chloroadenosine, 2′-fluoro-2′-deoxyadenosine, 2′-fluoro-2′-deoxycytidine, 2′-fluoro-2′-deoxyguanosine, 2′-fluoro-2′-deoxyuridine, 2′-fluorothymidine, 2-methyl-adenosine, 2-methyl-guanosine, 2-methyl-thio-N6-isopenenyl-adenosine, 2′-O-methyl-2-aminoadenosine, 2′-O-methyl-2′-deoxyadenosine, 2′-O-methyl-2′-deoxycytidine, 2′-O-methyl-2′-deoxyguanosine, 2′-O-methyl-2′-deoxyuridine, 2′-O-methyl-5-methyluridine, 2′-O-methylinosine, 2′-O-methylpseudouridine, 2-thiocytidine, 2-thio-cytidine, 3-methyl-cytidine, 4-acetyl-cytidine, 4-thiouridine, 5-(carboxyhydroxymethyl)-uridine, 5,6-dihydrouridine, 5-aminoallylcytidine, 5-aminoallyl-deoxyuridine, 5-bromouridine, 5-carboxymethylaminomethyl-2-thio-uracil, 5-carboxymethylamonomethyl-uracil, 5-chloro-ara-cytodine, 5-fluoro-uridine, 5-iodouridine, 5-methoxycarbonylmethyl-uridine, 5-methoxy-uridine, 5-methyl-2-thio-uridine, 6-Azacytidine, 6-azauridine, 6-chloro-7-deaza-guanosine, 6-chloropurineriboside, 6-mercapto-guanosine, 6-methyl-mercaptopurine-riboside, 7-deaza-2′-deoxy-guanosine, 7-deazaadenosine, 7-methyl-guanosine, 8-azaadenosine, 8-bromo-adenosine, 8-bromo-guanosine, 8-mercapto-guanosine, 8-oxoguanosine, benzimidazole-riboside, beta-D-mannosyl-queosine, dihydro-uridine, inosine, N1-methyladenosine, N6-([6-aminohexyl]carbamoylmethyl)-adenosine, N6-isopentenyl-adenosine, N6-methyl-adenosine, N7-methyl-xanthosine, N-uracil-5-oxyacetic acid methyl ester, puromycin, queosine, uracil-5-oxyacetic acid, uracil-5-oxyacetic acid methyl ester, wybutoxosine, xanthosine, and xylo-adenosine. The preparation of such variants is known to the person skilled in the art, for example from US patents U.S. Pat. Nos. 4,373,071, 4,401,796, 4,415,732, 4,458,066, 4,500,707, 4,668,777, 4,973,679, 5,047,524, 5,132,418, 5,153,319, 5,262,530 or 5,700,642.

In some embodiments, the artificial nucleic acid as described herein comprises at least one chemically modified nucleotide selected from 2-amino-6-chloropurineriboside-5′-triphosphate, 2-aminopurine-riboside-5′-triphosphate, 2-aminoadenosine-5′-triphosphate, 2′-amino-2′-deoxycytidine-triphosphate, 2-thiocytidine-5′-triphosphate, 2-thiouridine-5′-triphosphate, 2′-fluorothymidine-5′-triphosphate, 2′-O-methyl-inosine-5′-triphosphate, 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-bromo-2′-deoxycytidine-5′-triphosphate, 5-bromo-2′-deoxyuridine-5′-triphosphate, 5-iodocytidine-5′-triphosphate, 5-iodo-2′-deoxycytidine-5′-triphosphate, 5-iodouridine-5′-triphosphate, 5-iodo-2′-deoxyuridine-5′-triphosphate, 5-methylcytidine-5′-triphosphate, 5-methyluridine-5′-triphosphate, 5-propynyl-2′-deoxycytidine-5′-triphosphate, 5-propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, O6-methylguanosine-5′-triphosphate, pseudouridine-5′-triphosphate, puromycin-5′-triphosphate, or xanthosine-5′-triphosphate.

In some embodiments, the artificial nucleic acid as described herein comprises at least one chemically modified nucleotide selected from pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine.

In some embodiments, the artificial nucleic acid as described herein comprises at least one chemically modified nucleotide selected from 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine.

In other embodiments, the artificial nucleic acid as described herein comprises at least one chemically modified nucleotide selected from 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl) adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine.

In other embodiments, the artificial nucleic acid as described herein comprises at least one chemically modified nucleotide selected from inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2, N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

In certain embodiments, the artificial nucleic acid as described herein comprises at least one chemically modified nucleotide selected from 6-aza-cytidine, 2-thio-cytidine, alpha-thio-cytidine, pseudo-iso-cytidine, 5-aminoallyl-uridine, 5-iodo-uridine, N1-methyl-pseudouridine, 5,6-dihydrouridine, alpha-thio-uridine, 4-thio-uridine, 6-aza-uridine, 5-hydroxy-uridine, deoxy-thymidine, 5-methyl-uridine, pyrrolo-cytidine, inosine, alpha-thio-guanosine, 6-methyl-guanosine, 5-methyl-cytdine, 8-oxo-guanosine, 7-deaza-guanosine, N1-methyl-adenosine, 2-amino-6-chloro-purine, N6-methyl-2-amino-purine, pseudo-iso-cytidine, 6-chloro-purine, N6-methyl-adenosine, alpha-thio-adenosine, 8-azido-adenosine, 7-deaza-adenosine.

In certain embodiments, the artificial nucleic acid comprises at least one chemically modified nucleotide, which is chemically modified at the 2′ position. Preferably, the chemically modified nucleotide comprises a substituent at the 2′ carbon atom, wherein the substituent is selected from the group consisting of a halogen, an alkoxy group, a hydrogen, an aryloxy group, an amino group and an aminoalkoxy group, preferably from 2′-hydrogen (2′-deoxy), 2′-O-methyl, 2′-O-methoxyethyl and 2′-fluoro. In the context of the artificial nucleic acid, in particular if the artificial nucleic acid is an RNA or a molecule comprising ribonucleotides, a 2′-deoxynucleotide (comprising hydrogen as a substituent at the 2′ carbon atom), such as deoxycytidine or a variant thereof, may also be referred to as ‘chemically modified nucleotide’.

Another chemical modification that involves the 2′ position of a nucleotide as described herein is a locked nucleic acid (LNA) nucleotide, an ethylene bridged nucleic acid (ENA) nucleotide and an (S)-constrained ethyl cEt nucleotide. These backbone modifications lock the sugar of the modified nucleotide into the preferred northern conformation. It is believed that the presence of that type of modification in the targeting sequence of the artificial nucleic acid allows for stronger and faster binding of the targeting sequence to the target RNA.

According to some embodiments, the artificial nucleic acid comprises at least one chemically modified nucleotide, wherein the phosphate backbone, which is incorporated into the artificial nucleic acid molecule, is modified. The phosphate groups of the backbone can be modified, for example, by replacing one or more of the oxygen atoms with a different substituent. Further, the modified nucleotide can include the full replacement of an unmodified phosphate moiety with a modified phosphate as described herein. Examples of modified phosphate groups include, but are not limited to, the group consisting of a phosphorothioate, a stereopure phosphorothioate, a phosphoroselenate, a borano phosphate, a borano phosphate ester, a hydrogen phosphonate, a phosphoroamidate, an alkyl phosphonate, an aryl phosphonate and a phosphotriester. The phosphate linker can also be modified by the replacement of a linking oxygen with nitrogen (bridged phosphoroamidates), sulfur (bridged phosphorothioates) and carbon (bridged methylene-phosphonates).

According to a further preferred embodiment, the artificial nucleic acid comprises an abasic site. As used herein, an ‘abasic site’ is a nucleotide lacking the organic base. In preferred embodiments, the abasic nucleotide further comprises a chemical modification as described herein at the 2′ position of the ribose. Preferably, the 2′ C atom of the ribose is substituted with a substituent selected from the group consisting of a halogen, an alkoxy group, a hydrogen, an aryloxy group, an amino group and an aminoalkoxy group, preferably from 2′-hydrogen (2′-deoxy), 2′-O-methyl, 2′-O-methoxyethyl and 2′-fluoro. In the context of the present invention, a ‘chemically modified nucleotide’ may therefore also be an abasic site.

In a preferred embodiment, artificial nucleic acids are 25-59 nt long, comprised of at least 20% 2′-F nucleosides, 20% 2′-O-methyl nucleosides and 15% phosphorothioate linkages, while uniform blocks of more than 6 consecutive 2′-O-methyl modifications are avoided. In the central base triplet opposite the targeted adenosine at least one, more preferably two and most preferably all three nucleosides are 2′-deoxy nucleosides. Given the effect of chemical modifications, in particular of 2′-O-methyl, to suppress or reduce bystander/off-target editing, the need for G·U wobble base pairing to suppress or reduce bystander/off-target editing may be less important. Still, in highly editable codon contexts, or in partially modified artificial nucleic acids, or in cases where target adenosines (underlined) reside in adenosine-rich codons, like 5′-CAA or 5′-AAG or similar, G·U wobble base pairing might be well applicable. This holds also with regard to the need to enhance editing yields in difficult to editing codons like 5′-GAN (N=A, U, G, C) where U·G wobbling might become applicable.

According to another embodiment, the artificial nucleic acid molecule can be modified by the addition of a so-called 5′ CAP structure. A 5′-cap is an entity, typically a modified nucleotide entity, which generally ‘caps’ the 5′-end of a mature mRNA. A 5′-cap may typically be formed by a modified nucleotide, particularly by a derivative of a guanine nucleotide. Preferably, the 5′-cap is linked to the 5′-terminus of the artificial nucleic acid via a 5′-5′-triphosphate linkage. A 5′-cap may be methylated, e.g. m7GpppN, wherein N is the terminal 5′ nucleotide of the nucleic acid carrying the 5′-cap, typically the 5′-end of an RNA. Further examples of 5′ cap structures include glyceryl, inverted deoxy abasic residue (moiety), 4′,5′ methylene nucleotide, 1-(beta-D-erythrofuranosyl) nucleotide, 4′-thio nucleotide, carbocyclic nucleotide, 1,5-anhydrohexitol nucleotide, L-nucleotides, alpha-nucleotide, modified base nucleotide, threo-pentofuranosyl nucleotide, acyclic 3′,4′-seco nucleotide, acyclic 3,4-dihydroxybutyl nucleotide, acyclic 3,5 dihydroxypentyl nucleotide, 3′-3′-inverted nucleotide moiety, 3′-3′-inverted abasic moiety, 3′-2′-inverted nucleotide moiety, 3′-2′-inverted abasic moiety, 1,4-butanediol phosphate, 3′-phosphoramidate, hexylphosphate, aminohexyl phosphate, 3′-phosphate, 3′phosphorothioate, phosphorodithioate, or bridging or non-bridging methylphosphonate moiety. Particularly preferred modified 5′-CAP structures are CAP1 (methylation of the ribose of the adjacent nucleotide of m7G), CAP2 (methylation of the ribose of the 2nd nucleotide downstream of the m′7G), CAP3 (methylation of the ribose of the 3rd nucleotide downstream of the m7G), CAP4 (methylation of the ribose of the 4th nucleotide downstream of the m7G), ARCA (anti-reverse CAP analogue, modified ARCA (e.g., phosphothioate modified ARCA), inosine, N1-methyl-guanosine, 2′-fluoro-guanosine, 7-deaza-guanosine, 8-oxo-guanosine, 2-amino-guanosine, LNA-guanosine, and 2-azido-guanosine.

In some embodiments, the artificial nucleic acid comprises a moiety, which enhances cellular uptake of the artificial nucleic acid. Preferably, the moiety enhancing cellular uptake is a triantennary N-acetyl galactosamine (GalNAc3), which is preferably conjugated with the 3′ terminus or with the 5′ terminus of the artificial nucleic acid.

In a preferred embodiment of the present invention, the artificial nucleic acid is an RNA or an RNA analog which may comprise modifications as defined above. More preferably, the artificial nucleic acid according to the present invention is an endogenously expressible RNA. Therefore, in a more preferred embodiment, the artificial nucleic acid comprises unmodified (ribo) nucleotides. In a more preferred embodiment, the artificial nucleic acid is a genetically encodable nucleic acid, preferably a genetically encodable RNA. In a most preferred embodiment, the artificial nucleic acid does not include any (chemically) modified nucleotides. Preferably, the artificial nucleic acid consists of naturally occurring nucleotides. More preferably, the artificial nucleic acid consists of nucleotides naturally occurring in mammalians, preferably in mouse or human.

The artificial nucleic acid as described herein may be synthesized by a method known in the art. The artificial nucleic acid may be synthesized chemically or by in vitro transcription from a suitable vector, preferably as described herein. Preferably, the artificial nucleic acid of the present invention is synthesized in vivo from a suitable vector, as described herein, which has previously been transfected in a cell or organism.

In a further aspect, the present invention relates to a method for providing an artificial nucleic acid for site-directed editing of a target RNA, wherein the target RNA comprises a target sequence comprising an adenosine as a target nucleotide to be edited and at least one off-target sequence comprising an adenosine that is not to be edited, the method comprising generating a nucleic acid sequence of the artificial nucleic acid that is complementary or partially complementary to a nucleic acid sequence in the target RNA, and,
(i) if the target sequence comprises an adenosine nucleotide to be edited and nucleotides immediately 5′ and 3′ of the adenosine nucleotide in a triplet context of 5′-UAG-3′, 5′-GAU-3′, 5′-GAG-3′, 5′-GAC-3′, 5′-GAA-3′, 5′-CAG-3′, and/or 5′-AAG-3′, replacing, in a first nucleic acid sequence of the artificial nucleic acid that is complementary or partially complementary to the nucleotide acid sequence in the target sequence,
   one or more of a nucleotide triplet 5′-CUA-3′ by a nucleotide triplet selected from the group consisting of 5′-UUA-3′, and 5′-UCA-3′, preferably if the target adenosine nucleotide to be edited in the target sequence is the central adenosine nucleotide in a nucleotide triplet 5′-UAG-3′;
   one or more of a nucleotide triplet 5′-AUC-3′ by a nucleotide triplet selected from the group consisting of 5′-AUU-3′, and 5′-ACU-3′, preferably if the target adenosine nucleotide to be edited in the target sequence is the central adenosine nucleotide in a nucleotide triplet 5′-GAU-3′;
   one or more of a nucleotide triplet 5′-CUC-3′ by a nucleotide triplet selected from the group consisting of 5′-CUU-3′, 5′-UUC-3′, 5′-UUU-3′, 5′-UUG-3′, 5′-GUU-3′, 5′-UUA-3′, 5′-AUU-3′, 5′-CCU-3′, 5′-UCC-3′, and 5′-UCU-3′, preferably if the target adenosine nucleotide to be edited in the target sequence is the central adenosine nucleotide in a nucleotide triplet 5′-GAG-3′;
   one or more of a nucleotide triplet 5′-GUC-3′ by a nucleotide triplet selected from the group consisting of 5′-GUU-3′, and 5′-GCU-3′, preferably if the target adenosine nucleotide to be edited in the target sequence is the central adenosine nucleotide in a nucleotide triplet 5′-GAC-3′;
   one or more of a nucleotide triplet 5′-UUC-3′ by a nucleotide triplet selected from the group consisting of 5′-UUU-3′, 5′-AUU-3′, 5′-UCU-3′ and 5′-ACU-3′, preferably if the target adenosine nucleotide to be edited in the target sequence is the central adenosine nucleotide in a nucleotide triplet 5′-GAA-3′;
   one or more of a nucleotide triplet 5′-CUG-3′ by a nucleotide triplet selected from the group consisting of 5′-UUG-3′, 5′-UCG-3′, 5′-UUA-3′, and 5′-UCA-3′, preferably if the target adenosine nucleotide to be edited in the target sequence is the central adenosine nucleotide in a nucleotide triplet 5′-CAG-3′; and/or
   one or more of a nucleotide triplet 5′-CUU-3′ by a nucleotide triplet selected from the group consisting of 5′-UUU-3′, and 5′-UCU-3′, preferably if the target adenosine nucleotide to be edited in the target sequence is the central adenosine nucleotide in a nucleotide triplet 5′-AAG-3′,
and/or
(ii) if the off-target sequence comprises an adenosine nucleotide not to be edited and nucleotides immediately 5′ and 3′ of the adenosine nucleotide in a triplet context of 5′-UAU-3′, 5′-UAG-3′, 5′-UAC-3′, 5′-UAA-3′, 5′-GAU-3′, 5′-CAU-3′, and/or 5′-AAU-3′,
replacing, in a second nucleic acid sequence of the artificial nucleic acid that is complementary or partially complementary to the nucleotide acid sequence in the off-target sequence,
   one or more of a nucleotide triplet 5′-AUA-3′ by a nucleotide triplet selected from the group consisting of 5′-GUG-3′, 5′-AUG-3′, 5′-GUA-3′, 5′-GGG-3′, 5′-AGG-3′, and 5′-GGA-3′, preferably if an adenosine nucleotide not to be edited in the off-target sequence is the central adenosine nucleotide in a nucleotide triplet 5′-UAU-3′;
   one or more of a nucleotide triplet 5′-CUA-3′ by a nucleotide triplet selected from the group consisting of 5′-CUG-3′, 5′-UUG-3′, 5′-CGG-3′, and 5′-UGG-3′, preferably if an adenosine nucleotide not to be edited in the off-target sequence is the central adenosine nucleotide in a nucleotide triplet 5′-UAG-3′;
   one or more of a nucleotide triplet 5′-GUA-3′ by a nucleotide triplet selected from the group consisting of 5′-GUG-3′, and 5′-GGG-3′, preferably if an adenosine nucleotide not to be edited in the off-target sequence is the central adenosine nucleotide in a nucleotide triplet 5′-UAC-3′;
   one or more of a nucleotide triplet 5′-UUA-3′ by a nucleotide triplet selected from the group consisting of 5′-UUG-3′, 5′-GUG-3′, 5′-UGG-3′, and 5′-GGG-3′ preferably if an adenosine nucleotide not to be edited in the off-target sequence is the central adenosine nucleotide in a nucleotide triplet 5′-UAA-3′;
   one or more of a nucleotide triplet 5′-AUC-3′ by a nucleotide triplet selected from the group consisting of 5′-GUC-3′, 5′-GUU-3′, 5′-GGC-3′, and 5′-GGU-3′, preferably if an adenosine nucleotide not to be edited in the off-target sequence is the central adenosine nucleotide in a nucleotide triplet 5′-GAU-3′;
   one or more of a nucleotide triplet 5′-AUG-3′ by a nucleotide triplet selected from the group consisting of 5′-GUG-3′, and 5′-GGG-3′, preferably if an adenosine nucleotide not to be edited in the off-target sequence is the central adenosine nucleotide in a nucleotide triplet 5′-CAU-3′; and/or
   one or more of a nucleotide triplet 5′-AUU-3′ by a nucleotide triplet selected from the group consisting of 5′-GUU-3′, 5′-GGU-3′, 5′-GUG-3′, and 5′-GGG-3′, preferably if an adenosine nucleotide not to be edited in the off-target sequence is the central adenosine nucleotide in a nucleotide triplet 5′-AAU-3′,
and/or
(iii) if the target RNA comprises an adenosine nucleotide to be edited (A) and at least one neighboring adenosine nucleotide (A) not to be edited and nucleotides immediately 5′ and 3′ of the adenosine nucleotides in a sequence context of 5′-UAAA-3′, 5′-UAAU-3′, 5′-UAAC-3′, 5′-UAAG-3′, 5′-AAAU-3′, 5′-UAAU-3′, 5′-CAAU-3′, 5′-GAAU-3′, and/or 5′-UAAAU-3′, wherein A represents the adenosine to be edited,
replacing, in a third nucleic acid sequence of the artificial nucleic acid that is complementary or partially complementary to the nucleotide acid sequence in the target RNA,
   one or more of a nucleotide sequence 5′-UUUA-3′ by a nucleotide sequence 5′-UCUG-3′, or 5′-GCUG-3′, preferably if the adenosine nucleotides in the target sequence are present in the context of the nucleic acid sequence 5′-UAAA-3′;
   one or more of a nucleotide sequence 5′-AUUA-3′ by a nucleotide sequence 5′-ACUG-3′, preferably if the adenosine nucleotides in the target sequence are present in the context of the nucleic acid sequence 5′-UAAU-3′;
   one or more of a nucleotide sequence 5′-GUUA-3′ by a nucleotide sequence 5′-GCUG-3′, preferably if the adenosine nucleotides in the target sequence are present in the context of the nucleic acid sequence 5′-UAAC-3′;
   one or more of a nucleotide sequence 5′-CUUA-3′ by a nucleotide sequence 5′-CCUG-3′, 5′-UCUG-3′ or 5′-UUUG-3′, preferably if the adenosine nucleotides in the target sequence are present in the context of the nucleic acid sequence 5′-UAAG-3′;
   one or more of a nucleotide sequence 5′-AUUU-3′ by a nucleotide sequence 5′-GUCU-3′, or 5′-GUCG-3′, preferably if the adenosine nucleotides in the target sequence are present in the context of the nucleic acid sequence 5′-AAAU-3′;
   one or more of a nucleotide sequence 5′-AUUA-3′ by a nucleotide sequence 5′-GUCA-3′, preferably if the adenosine nucleotides in the target sequence are present in the context of the nucleic acid sequence 5′-UAAU-3′;
   one or more of a nucleotide sequence 5′-AUUG-3′ by a nucleotide sequence 5′-GUCG-3′, preferably if the adenosine nucleotides in the target sequence are present in the context of the nucleic acid sequence 5′-CAAU-3′;
   one or more of a nucleotide sequence 5′-AUUC-3′ by a nucleotide sequence 5′-GUCC-3′, 5′-GUCU-3′, or 5′-GUUU-3′, preferably if the adenosine nucleotides in the target sequence are present in the context of the nucleic acid sequence 5′-GAAU-3′; and/or
   one or more of a nucleotide sequence 5′-AUUUA-3′ by a nucleotide sequence 5′-GUCUG-3′, preferably if the adenosine nucleotides in the target sequence are present in the context of the nucleic acid sequence 5′-UAAAU-3′.

In particular, in the method of the present invention, a nucleic acid sequence complementary to a target RNA sequence which comprises at least one target adenosine nucleotide to be edited and at least one off-target adenosine not to be edited, may be modified such that a guanosine nucleotide 3′ or 5′ to a target adenosine is wobble base paired with a uridine nucleotide of the artificial nucleic acid thereby promoting or increasing editing of the neighboring target adenosine.

On the other hand, in the method of the present invention, the nucleic acid sequence of the artificial nucleic acid may be modified such that a uridine nucleotide immediately 3′ and/or 5′ to an off-target adenosine is wobble base paired with a guanosine nucleotide of the artificial nucleic acid thereby reducing or inhibiting off-target editing of the neighboring target adenosine. In other words, with the method of the present invention, an optimized artificial nucleic acid for site-directed editing of a target RNA can be provided which increases editing of a target adenosine by U·G wobbling with a guanosine nucleotide immediately 3′ and/or 5′ to the target adenosine, and/or decreases unwanted editing of an off-target adenosine by G·U wobbling with a uridine nucleotide immediately 3′ and/or 5′ to the off-target adenosine.

As mentioned above, in a target RNA, some adenosines, e.g. those which are in a 5′-GAN-3′ or 5′-CAN-3′ context (N=A, C, G, U), are less prone to unwanted off-target editing than e.g. adenosines which are present in a 5′-UAN-3′ or 5′-AAU-3′ context. Therefore, in a preferred embodiment of the present invention, in the off-target sequence of the artificial nucleic acid, all 5′-NUA-3′ (N=A, C, G, U) triplets as well as the 5′-UUA-3′ triplet are replaced by a nucleotide triplet which enables G·U wobbling 3′ or 5′ to the central off-target adenosine thereby reducing its off-target editing.

Thus, in a preferred embodiment of the present invention, the method comprises replacing, in the second nucleic acid sequence of the artificial nucleic acid that is complementary or partially complementary to the nucleotide acid sequence in the off-target sequence,
all of the nucleotide triplets 5′-AUA-3′ (complementary to nucleotide triplet 5′-UAU-3′ in the target RNA) by a nucleotide triplet selected from the group consisting of 5′-GUG-3′, 5′-AUG-3′, 5′-GUA-3′, 5′-GGG-3′, 5′-AGG-3′, and 5′-GGA-3′;
all of the nucleotide triplets 5′-CUA-3′ (complementary to nucleotide triplet 5′-UAG-3′ in the target RNA) by a nucleotide triplet selected from the group consisting of 5′-CUG-3′, 5′-UUG-3′, 5′-CGG-3′, and 5′-UGG-3′;
all of the nucleotide triplets 5′-GUA-3′ (complementary to nucleotide triplet 5′-UAC-3′ in the target RNA) by a nucleotide triplet selected from the group consisting of 5′-GUG-3′, and 5′-GGG-3′;
all of the nucleotide triplets 5′-UUA-3′ (complementary to nucleotide triplet 5′-UAA-3′ in the target RNA) by a nucleotide triplet selected from the group consisting of 5′-UUG-3′, and 5′-UGG-3′; and/or
all of the nucleotide triplets 5′-AUU-3′ (complementary to nucleotide triplet 5′-AAU-3′ in the target RNA) by a nucleotide triplet selected from the group consisting of 5′-GUU-3′, and 5′-GGU-3′.

As mentioned above, it has been found by the present inventors that off-target editing of an adenosine is even more effectively reduced by enabling G·U wobbling immediately 5′ and/or 3′ of the off-target adenosine rather than by introducing a G-A mismatch at the central off-target adenosine. Moreover a G-A mismatch may destabilize the double strand binding strength between the target RNA and the artificial nucleic acid molecule, and may reduce editing efficiency. Therefore, in the second nucleic acid sequence of the artificial nucleic acid, nucleotide triplets are preferred which avoid a G-A mismatch at the central adenosine nucleotide (not to be edited), but enables a G·U wobble immediately 3′ and/or 5′ to the central adenosine.

Thus, in a more preferred embodiment of the present invention, the method comprises replacing, in the second nucleic acid sequence of the artificial nucleic acid that is complementary or partially complementary to the nucleotide acid sequence in the off-target sequence,
all of the nucleotide triplets 5′-AUA-3′ (complementary to nucleotide triplet 5′-UAU-3′ in the target RNA) by a nucleotide triplet selected from the group consisting of 5′-GUG-3′, 5′-AUG-3′, and 5′-GUA-3′;
all of the nucleotide triplets 5′-CUA-3′ (complementary to nucleotide triplet 5′-UAG-3′ in the target RNA) by a nucleotide triplet selected from the group consisting of 5′-CUG-3′, and 5′-UUG-3′;
all of the nucleotide triplets 5′-GUA-3′ (complementary to nucleotide triplet 5′-UAC-3′ in the target RNA) by a nucleotide triplet 5′-GUG-3′;
all of the nucleotide triplets 5′-UUA-3′ 3′ (complementary to nucleotide triplet 5′-UAA-3′ in the target RNA) by a nucleotide triplet 5′-UUG-3′; and/or
all of the nucleotide triplets 5′-AUU-3′ (complementary to nucleotide triplet 5′-AAU-3′ in the target RNA) by a nucleotide triplet 5′-GUU-3′.

As mentioned above, the inventors surprisingly found that the boosting effect of U·G wobbles immediately 3′ and/or 5′ of the target adenosine in the target RNA cannot further enhanced—or is even reduced—by a C-A mismatch at the target site which is conventionally used to enhance RNA editing at the target adenosine. Therefore, in a preferred embodiment, the first nucleic acid sequence of the artificial nucleic acid comprises a uridine nucleotide which is complementary and forms a base pair with the target adenosine in the target RNA, thereby avoiding a C-A mismatch with the target adenosine in the target RNA while enabling a U·G wobble immediately 3′ and/or 5′ to the target adenosine.

Thus, in a preferred embodiment, the method of the present invention comprises:
replacing, in the first nucleic acid sequence of the artificial nucleic acid that is complementary or partially complementary to the nucleotide acid sequence in the target sequence,
   one or more of a nucleotide triplet 5′-CUA-3′ (complementary to nucleotide triplet 5′-UAG-3′ in the target RNA) by the nucleotide triplet 5′-UUA-3′;
   one or more of a nucleotide triplet 5′-AUC-3′ (complementary to nucleotide triplet 5′-GAU-3′ in the target RNA) by a nucleotide triplet 5′-AUU-3′;
   one or more of a nucleotide triplet 5′-CUC-3′ (complementary to nucleotide triplet 5′-GAG-3′ in the target RNA) by a nucleotide triplet selected from the group consisting of 5′-CUU-3′, 5′-UUC-3′, 5′-UUU-3′, 5′-UUG-3′, 5′-GUU-3′, 5′-UAA-3′, and 5′-AUU-3′;
   one or more of a nucleotide triplet 5′-GUC-3′ (complementary to nucleotide triplet 5′-GAC-3′ in the target RNA) by a nucleotide triplet 5′-GUU-3′;
   one or more of a nucleotide triplet 5′-UUC-3′ (complementary to nucleotide triplet 5′-GAA-3′ in the target RNA) by a nucleotide triplet 5′-UUU-3′;
   one or more of a nucleotide triplet 5′-CUG-3′ (complementary to nucleotide triplet 5′-CAG-3′ in the target RNA) by a nucleotide triplet 5′-UUG-3′ or 5′-UUA-3′; and/or
   one or more of a nucleotide triplet 5′-CUU-3′ (complementary to nucleotide triplet 5′-AAG-3′ in the target RNA) by a nucleotide triplet 5′-UUU-3′.

In this way, C-A mismatches at the target adenosine(s) are avoided.

In a particular embodiment, the method of the present invention comprises replacing in the first nucleic acid sequence one or more of a nucleotide triplet as described above by one or more nucleotide triplet as described above thereby introducing U·G wobble base pair(s) in the target sequence to boost editing of target adenosine(s) via U·G wobbling, while editing of an off-target adenosine in the second nucleic acid sequence is not reduced via G·U wobbling by replacing the respective nucleotide triplets as described above, but, if necessary by other appropriate means, such as G-A mismatch and/or uridine depletion, for example. That is, in a particular embodiment, the method comprises step (i), and does not comprise step (ii), as described above.

In another embodiment, the method of the present invention comprises replacing in the first nucleic acid sequence one or more of a nucleotide triplet as described above by one or more nucleotide triplet as described above thereby introducing U·G wobble base pair(s) in the target sequence to boost editing of target adenosine(s) via U·G wobbling, and comprises reducing editing of at least one off-target adenosine in the second nucleic acid sequence via G·U wobbling by replacing the respective nucleotide triplet(s) as described above. That is, in another embodiment, the method comprises step (i), and also comprises step (ii), as described above.

In a further and preferred embodiment, the method of the present invention does not comprise boosting editing of a target adenosine via U·G wobbling by replacing in the first nucleic acid sequence one or more of a nucleotide triplet as described above by one or more nucleotide triplet as described above, but comprises reducing editing of at least one off-target adenosine in the second nucleic acid sequence via G·U wobbling by replacing the respective nucleotide triplet(s) as described above. That is, in a preferred embodiment, the method does not comprise step (i), but comprises step (ii), as described above.

Thus, the method of the present invention does not necessarily provide an artificial nucleic acid wherein editing is boosted by U·G wobbling in the target sequence. Rather, in a preferred embodiment, the method provides an artificial nucleic acid wherein editing of at least one off-target adenosine in the off-target sequence is reduced via G·U wobbling (by replacing nucleotide triplets as described above), while editing of a target adenosine may be promoted by the use of other means, e.g. via introducing a C-A mismatch.

As mentioned above, there is a limitation for the use of G·U wobble base pairs in the second nucleic acid sequence of the artificial nucleic acid since G·U wobbles require a uridine as nearest neighbor to an off-target adenosine. To avoid unwanted off-target editing of adenosines in the target RNA without a neighboring uridine, the second nucleic acid sequence of the artificial nucleic acid is preferably designed to comprise nucleotide triplets including a central guanosine which mismatches with an adenosine not to be edited in the off-target sequence of the target RNA.

Thus, if the target RNA comprises an adenosine nucleotide not to be edited and nucleotides immediately 5′ and 3′ of the adenosine nucleotide in a triplet context of 5′-GAG-3′, 5′-GAC-3′, 5′-GAA-3′, 5′-CAG-3′, 5′-CAC-3′, 5′-CAA-3′, 5′-AAG-3′, 5′-AAC-3′, and/or 5′-AAA-3′, in a preferred embodiment, the method of the present invention comprises:
replacing, in a second nucleic acid sequence of the artificial nucleic acid complementary or partially complementary to a nucleotide sequence in the off-target sequence,
   one or more of a nucleotide triplet 5′-CUC-3′ (complementary to nucleotide triplet 5′-GAG-3′ in the off-target sequence of the target RNA) by a nucleotide triplet 5′-CGC-3′;
   one or more of a nucleotide triplet 5′-GUC-3′ (complementary to nucleotide triplet 5′-GAC-3′ in the off-target sequence of the target RNA) by a nucleotide triplet 5′-GGC-3′;
   one or more of a nucleotide triplet 5′-UUC-3′ (complementary to nucleotide triplet 5′-GAA-3′ in the off-target sequence of the target RNA) by a nucleotide triplet selected from the group consisting of 5′-UGC-3′, and 5′-GGC-3′;
   one or more of a nucleotide triplet 5′-CUG-3′ (complementary to nucleotide triplet 5′-CAG-3′ in the off-target sequence of the target RNA) by a nucleotide triplet 5′-CGG-3′;
   one or more of a nucleotide triplet 5′-GUG-3′ (complementary to nucleotide triplet 5′-CAC-3′ in the off-target sequence of the target RNA) by a nucleotide triplet 5′-GGG-3′;
   one or more of a nucleotide triplet 5′-UUG-3′ (complementary to nucleotide triplet 5′-CAA-3′ in the off-target sequence of the target RNA) by a nucleotide triplet selected from the group consisting 5′-UGG-3′ and 5′-GGG-3′;
   one or more of a nucleotide triplet 5′-CUU-3′ (complementary to nucleotide triplet 5′-AAG-3′ in the off-target sequence of the target RNA) by a nucleotide triplet selected from the group consisting of 5′-CGU-3′ and 5′-CGG-3′;
   one or more of a nucleotide triplet 5′-GUU-3′ (complementary to nucleotide triplet 5′-AAC-3′ in the off-target sequence of the target RNA) by a nucleotide triplet selected from the group consisting of 5′-5′-GGU-3′ and 5′-GGG-3′; and/or
   one or more of a nucleotide triplet 5′-UUU-3′ (complementary to nucleotide triplet 5′-AAA-3′ in the off-target sequence of the target RNA) by a nucleotide triplet selected from the group consisting of 5′-UGU-3′, 5′-GGU-3′, 5′-UGG-3′, and 5′-GGG-3′.

In this way, G-A mismatches at the off-target adenosine(s) are created at positions where unwanted off-target editing cannot be prevented by G·U wobbling.

In this context, it is in particular desirable to reduce off-target editing of adenosine nucleotides in a 5′-AAG-3′, 5′-AAC-3′ and 5′-AAA-3′ context, since, as mentioned above, adenosine nucleotides in a 5′-GAN-3′ or 5′-CAN-3′ context are typically less prone to unwanted off-target editing,

Therefore, in a more preferred embodiment, the method of the present invention comprises:
replacing one or more of a nucleotide triplet 5′-CUU-3′ (complementary to nucleotide triplet 5′-AAG-3′ in the off-target sequence of the target RNA) by a nucleotide triplet selected from the group consisting of 5′-CGU-3′ and 5′-CGG-3′;
replacing one or more of a nucleotide triplet 5′-GUU-3′ (complementary to nucleotide triplet 5′-AAC-3′ in the off-target sequence of the target RNA) by a nucleotide triplet selected from the group consisting of 5′-5′-GGU-3′ and 5′-GGG-3′; and/or
replacing one or more of a nucleotide triplet 5′-UUU-3′ (complementary to nucleotide triplet 5′-AAA-3′ in the off-target sequence of the target RNA) by a nucleotide triplet selected from the group consisting of 5′-UGU-3′, 5′-GGU-3′, 5′-UGG-3′, and 5′-GGG-3′.

In a more preferred embodiment, all of the nucleotide triplets 5′-CUU-3′, 5′-GUU-3′ and 5′-UUU-3′ (complementary to 5′-AAG-3′, 5′-AAC-3′, and 5′-AAA-3′, respectively), are replaced by the nucleotide triplets as stated above to reduce unwanted off-target editing at positions where off-target editing cannot be prevented by G·U wobbling.

In a further aspect, the present invention relates to an artificial nucleic acid which is provided by the method of the present invention. The artificial nucleic acid provided by the method of the present invention is preferably as described above and comprises
a first nucleic acid sequence consisting of 3 nucleotides which is complementary or partially complementary to a nucleic acid sequence in a target sequence of a target RNA, which comprises a target adenosine nucleotide to be edited and the nucleotides immediately 5′ and 3′ of said target adenosine nucleotide, wherein a uridine nucleotide may form a U·G wobble base pair with a guanine nucleotide 3′ or 5′ of the central adenosine of the target sequence to enhance editing of the target adenosine, and
a second nucleic acid sequence comprising at least 3 nucleotides that is complementary or partially complementary to a nucleic acid sequence in at least one off-target sequence of a target RNA, which comprises an adenosine nucleotide not to be edited and the nucleotides immediately 5′ and 3′ of said adenosine nucleotide, wherein a guanosine nucleotide of the second nucleic acid sequence preferably forms a G·U wobble base pair with a uridine nucleotide 3′ or 5′ of the central adenosine of the off-target sequence to reduce editing of the off-target target adenosine, and/or
a third nucleic acid sequence consisting of 4 or 5 nucleotides that is complementary or partially complementary to a nucleic acid sequence in the target RNA which comprises an adenosine nucleotide to be edited, at least one neighboring adenosine that is not to be edited and the nucleotides immediately 5′ and 3′ of said adenosine nucleotides, wherein a guanosine nucleotide of the third nucleic acid sequence preferably forms a G·U wobble base pair with a uridine nucleotide adjacent to an adenosine in the target RNA that is not to be edited to reduce editing of the off-target target adenosine, and a cytosine nucleotide of the third nucleic acid sequence preferably forms a C-A mismatch with a target adenosine adjacent to the off-target adenosine in the target RNA, to enhance editing of the target adenosine while reducing bystander editing of the neighboring adenosine nucleotide.

Preferably, the artificial nucleic acid provided by the method of the present invention comprises a second nucleic acid sequence wherein a guanosine nucleotide forms a G·U wobble base pair with a uridine nucleotide in the target RNA which is in a 5′-UAN-3′ or 5′-AAU-3′ context.

Preferably, the second nucleic acid sequence of the artificial nucleic acid provided by the method of the present invention comprises a uridine nucleotide which is complementary and forms a base pair with the off-target adenosine in the target RNA, thereby avoiding a G-A mismatch with the off-target adenosine in the target RNA.

In a particular embodiment, the first nucleic acid sequence of the artificial nucleic acid provided by the method of the present invention comprises a uridine nucleotide which is complementary and forms a base pair with the target adenosine in the target RNA, thereby avoiding a C-A mismatch with the target adenosine in the target RNA. This is particularly useful, when the U·G wobble is used to promote editing yields of adenosines placed in a 5′-GAN or 5′-NAG (N=T, A, C, G) triplet context. Here, the target adenosine is preferably base-paired with a uridine.

In another embodiment, the first nucleic acid sequence of the artificial nucleic acid provided by the method of the present invention comprises a cytosine nucleotide which forms a C-A mismatch with the target adenosine in the target RNA, and avoids a U·G wobble base pair with a neighboring guanosine nucleotide.

In a further preferred embodiment, the second nucleic acid sequence of the artificial nucleic acid provided by the method of the present invention comprises nucleotide triplets having a central guanosine nucleotide which forms a G-A mismatch with the off-target adenosine in the target RNA which is not neighbored by a uridine nucleotide, i.e. in a 5′-GAG-3′, 5′-GAC-3′, 5′-GAA-3′, 5′-CAG-3′, 5′-CAC-3′, 5′-CAA-3′, 5′-AAG-3′, 5′-AAC-3′, and/or 5′-AAA-3′ context, where unwanted bystander editing cannot be prevented by G·U wobbling. Since, as mentioned above, adenosine nucleotides in a 5′-GAN-3′ or 5′-CAN-3′ context are typically less prone to unwanted off-target editing, the second nucleic acid sequence of the artificial nucleic acid provided by the method of the present invention preferably comprises nucleotide triplets which forms a G-A mismatch with the central adenosine in a 5′-AAG-3′, 5′-AAC-3′ and/or 5′-AAA-3′ triplet of the target RNA.

In the method for providing an artificial nucleic acid for site-directed RNA editing according to the present invention, it is therefore necessary to analyse the RNA to be edited, in particular to analyse the target RNA with respect to adenosines (to be edited) and off-target adenosines (not to be edited) and the neighboring nucleotides thereof. In particular it has to be checked, which adenosines of the target RNA may prone to unwanted bystander editing (off-target editing) and whether unwanted off-target editing of those adenosines can be prevented by G·U wobbling of an adjacent uridine nucleotide.

Thereupon, in a sequence complementary or partially complementary to the target RNA sequence, nucleotide triplets complementary to the off-target adenosine and the nucleotides immediately 3′ and 5′ of said adenosine may be replaced by nucleotide triplets, as specified herein, which enable a G·U wobble base pair adjacent to the off-target adenosine thereby reducing (off-target) editing thereof.

If the nucleotide context of an off-target adenosine prone to off-target editing does not enable a G·U wobbling of an adjacent uridine nucleotide, nucleotide triplets complementary to the off-target adenosine and the nucleotides immediately 3′ and 5′ of said adenosine may be replaced by nucleotide triplets, as specified herein, enabling a G-A mismatch of the off-target adenosine with the corresponding nucleotide of the artificial nucleic acid.

On the other hand, in the course of the analysis of the target RNA, it is checked whether target adenosines to be edited are present in the target RNA in a 5′-GAN-3′ or 5′-NAG-3′ context so that editing may be enhanced by U·G wobbling of an adjacent guanosine nucleotide. In that case, in a sequence complementary or partially complementary to the target RNA sequence, nucleotide triplets complementary to the target adenosine and the nucleotides immediately 3′ and 5′ of said adenosine may be replaced by nucleotide triplets, as specified herein, which enable a U·G wobble base pair adjacent to the target adenosine thereby enhancing editing thereof.

The above analysis and replacement steps may be computer implemented.

The artificial nucleic acid provided by the method of the present invention may comprise further nucleic acid sequences capable of recruiting an adenosine deaminase, which may be an endogenous adenosine deaminase, e.g. ADAR1 or ADAR2, preferably human ADAR1, in particular ADARp110, or an adenosine deaminase fusion protein, such as Cas9-ADAR, Cas13-ADAR, MS2 Coat Protein-ADAR, λN-ADAR, CIRTS-ADAR, and TAR binding protein-ADAR, or a tagged deaminase, such as a SNAP-tagged deaminase, a Halo-tagged deaminase or a Clip-tagged deaminase as described above. Preferably, the adenosine deaminase recruited by the artificial nucleic acid of the present invention is not a SNAP-tagged deaminase. More preferably, the adenosine deaminase recruited by the artificial nucleic acid of the present invention is not a tagged deaminase, preferably as described herein. Most preferably, the adenosine deaminase recruited by the artificial nucleic acid of the present invention is an endogenous adenosine deaminase, preferably an endogenous adenosine deaminase naturally occurring in mammalian, more preferably mouse or human, e.g. ADAR1 or ADAR2, preferably human ADAR1, in particular ADARp110.

The further nucleic acid sequences capable of recruiting an adenosine deaminase may be capable of binding to the adenosine deaminase (adenosine deaminase fusion protein, tagged adenosine deaminase), preferably to the dsRNA binding domain of the adenosine deaminase.

For example, the artificial nucleic acid molecule provided by the method of the present invention may comprise a R/G motif, as described above, which forms an intramolecular imperfect hairpin structure which binds to the dsRNA binding domain of an adenosine deaminase.

As mentioned above, the artificial nucleic acid of the present invention which is provided by the inventive method can be integrated into a variety of engineered RNA base editing systems, for example the λN-ADAR, e.g. the λN-BoxB-ADAR system, and the Cas13-ADAR approaches which apply a hyperactive ADAR mutant and use rather short guide RNAs.

Moreover, the artificial nucleic acid provided by the method according the present invention may be useful in LEAPER guide RNAs. Basic LEAPER guide RNAs are 70 to 200 nt, typically 111 nt long unstructured guide RNAs that are reverse complementary to their target mRNA, except for a C-A mismatch at the target adenosine. They are known for their strong and widespread off-target editing which can be prevented to some degree by introduction of G-A mismatches at problematic sites. The introduction of all the necessary G-A mismatches to completely prevent off-target events does, however, often drastically lower on target editing yields. By integrating an artificial nucleic acid provided by the method of the present invention into a LEAPER guide RNA, i.e. by using G·U wobble base pairing, editing precision of LEAPER guide RNAs can strongly be improved, i.e. bystander editing can be reduced and on target editing can be increased, as shown in the Examples.

Another approach which may take advantage of the artificial nucleic acid provided by the method of the present invention is the CLUSTER approach which bypasses unwanted bystander editing by designing the guide RNA such that the number of editable adenosine bases is minimized. The design builds on the in silico optimization of guide RNAs that contain several (e.g. 3-9) recruitment sequences of e.g. 15 to 20 nt length that bind to the target RNA in a multivalent fashion. The sequence space for the recruitment sequences is usually selected so that they do not cover editable adenosines. By integrating the artificial nucleic acid of the present invention into the cluster approach, the available sequence space for the recruitment sequences can be greatly increased by also allowing 5′-UAN-3′ and 5′-NAU-3′, in particular UAU, UAG, UAC, UAA and AAU triplets, at which off-target editing can be controlled with the G·U wobble strategy thereby expanding the sequence space for CLUSTER guide RNAs.

Therefore, the artificial nucleic acid of the present invention may be integrated and used in a variety of approaches for site-directed RNA editing.

The artificial nucleic acid of the present invention which is provided by the method of the present invention can be a circular RNA, e.g. a circular RNA which is circularized by using the Tornado expression system adopted for guide RNAs (Litke, J. L. and S. R. Jaffrey. Highly efficient expression of circular RNA aptamers in cells using autocatalytic transcripts. Nat Biotechnol 37(6): 667-675(2019)).

In a preferred embodiment, the artificial nucleic acid of the present invention which is provided by the inventive method is circular CLUSTER guide RNA as described above with respect to the artificial nucleic acid of the present invention.

The artificial nucleic acid of the present invention which is provided by the inventive method is preferably an RNA, or an RNA analog comprising nucleotide modifications as described herein. Preferably, the artificial nucleic acid of the present invention, which is provided by the inventive method, consists of naturally occurring nucleotides, more preferably of nucleotides naturally occurring in mammalian, preferably mouse or human. Even more preferably, the artificial nucleic acid of the present invention which is provided by the inventive method does not comprise any (chemically) modified nucleotides. Most preferably, the artificial nucleic acid of the present invention provided by the inventive method is a genetically encodable RNA.

More preferably, the artificial nucleic acid of the present invention which is provided by the inventive method is an RNA which can be expressed endogenously, wherein the RNA is preferably encoded by a vector.

Therefore, in a further aspect, the present invention provides a vector encoding the artificial nucleic acid described herein.

The term ‘vector’ as used herein typically refers to a nucleic acid molecule, preferably to an artificial nucleic acid molecule. A vector in the context of the present invention is suitable for incorporating or harbouring a desired nucleic acid sequence, such as the nucleic acid sequence of the artificial nucleic acid or a fragment thereof. Such vectors may be storage vectors, expression vectors, cloning vectors, transfer vectors etc. A cloning vector may be, e.g., a plasmid vector or a bacteriophage vector. A transfer vector may be a vector, which is suitable for transferring nucleic acid molecules into cells or organisms, for example, viral vectors. Preferably, a vector in the sense of the present application comprises a cloning site, a selection marker, such as an antibiotic resistance factor, and a sequence suitable for multiplication of the vector, such as an origin of replication.

The vector may be an RNA vector or a DNA vector. Preferably, the vector is a DNA vector. The vector may be any vector known to the skilled person, such as a viral vector or a plasmid vector. Preferably, the vector is a plasmid vector, preferably a DNA plasmid vector. In certain embodiments, the vector is a viral vector, which is preferably selected from the group consisting of lentiviral vectors, retroviral vectors, adenoviral vectors, adeno-associated viral (AAV) vectors and hybrid vectors.

Preferably, the vector according to the present invention is suitable for producing the artificial nucleic acid molecule, preferably an RNA, according to the present invention. Thus, preferably, the vector comprises elements needed for transcription, such as a promoter, e.g. an RNA polymerase promoter. Preferably, the vector is suitable for transcription using eukaryotic, prokaryotic, viral or phage transcription systems, such as eukaryotic cells, prokaryotic cells, or eukaryotic, prokaryotic, viral or phage in vitro transcription systems. Thus, for example, the vector may comprise a promoter sequence, which is recognized by a polymerase, such as by an RNA polymerase, e.g. by a eukaryotic, prokaryotic, viral, or phage RNA polymerase. In a preferred embodiment, the vector comprises a phage RNA polymerase promoter such as an SP6, T3 or T7, preferably a T7 promoter. Preferably, the vector is suitable for in vitro transcription using a phage based in vitro transcription system, such as a T7 RNA polymerase based in vitro transcription system.

In some embodiments, the vector is designed for transcription of the artificial nucleic acid upon transfection into a eukaryotic cell, preferably upon transfection into a mammalian cell, or upon administration to a subject, preferably as described herein. In a preferred embodiment, the vector is designed for transcription of the artificial nucleic acid by a eukaryotic RNA polymerase, preferably RNA polymerase II or III, more preferably RNA polymerase III. In certain embodiments, the vector may comprise a U6 snRNA promoter or a H1 promoter and, optionally, a selection marker, e.g. a reporter gene (such as GFP) or a resistance gene (such as a puromycin or a hygromycin resistance gene).

According to one aspect of the present invention, a cell is provided that comprises the artificial nucleic acid or the vector described herein. The cell may be any cell, such as a bacterial cell or a eukaryotic cell, preferably an insect cell, a plant cell, a vertebrate cell, such as a mammalian cell (e.g. a human cell or a murine cell). The cell may be, for example, used for replication of the vector of the present invention, for example, in a bacterial cell. Furthermore, the cell, preferably a eukaryotic cell, may be used for synthesis of the artificial nucleic acid molecule according to the present invention.

The cells according to the present invention are, for example, obtainable by standard nucleic acid transfer methods, such as standard transfection, transduction or transformation methods. The term ‘transfection’ as used herein generally refers to the introduction of nucleic acid molecules, such as DNA or RNA (e.g. mRNA) molecules, into cells, preferably into eukaryotic cells. In the context of the present invention, the term ‘transfection’ encompasses any method known to the skilled person for introducing nucleic acid molecules into cells, preferably into eukaryotic cells, e.g. into mammalian cells. Such methods encompass, for example, electroporation, lipofection, e.g. based on cationic lipids and/or liposomes, calcium phosphate precipitation, nanoparticle based transfection, virus based transfection, or transfection based on cationic polymers, such as DEAE-dextran or polyethylenimine etc. In this context, the artificial nucleic acid or the vector as described herein may be introduced into the cell in a transient approach or in order to maintain the artificial nucleic acid or the vector stably in the cell (e.g. in a stable cell line).

Preferably, the cell is a mammalian cell, such as a cell of human subject, a domestic animal, a laboratory animal, such as a mouse or rat cell. Preferably, the cell is a human cell. The cell may be a cell of an established cell line, such as a CHO, BHK, 293T, COS-7, HELA, HEK, Jurkat cell line etc., or the cell may be a primary cell, such as a human dermal fibroblast (HDF) cell etc., preferably a cell isolated from an organism. In a preferred embodiment, the cell is an isolated cell of a mammalian subject, preferably of a human subject.

In a further aspect, the present invention concerns a composition comprising the artificial nucleic acid, the vector or the cell as described herein and, optionally, an additional excipient, preferably a pharmaceutically acceptable excipient. The composition described herein is preferably a pharmaceutical composition. The composition described herein may be used in treatment or prophylaxis of a subject, such as in a gene therapy approach. Alternatively, the composition can also be used for diagnostic purposes or for laboratory use, e.g. in in vitro experiments.

Preferably, the composition further comprises one or more vehicles, diluents and/or excipients, which are preferably pharmaceutically acceptable. In the context of the present invention, a pharmaceutically acceptable vehicle typically includes a liquid or non-liquid basis for the composition described herein. In one embodiment, the composition is provided in liquid form. In this context, preferably, the vehicle is based on water, such as pyrogen-free water, isotonic saline or buffered (aqueous) solutions, e.g. phosphate, citrate etc. buffered solutions. The buffer may be hypertonic, isotonic or hypotonic with reference to the specific reference medium, i.e. the buffer may have a higher, identical or lower salt content with reference to the specific reference medium, wherein preferably such concentrations of the afore mentioned salts may be used, which do not lead to damage of mammalian cells due to osmosis or other concentration effects. Reference media are, for instance, liquids occurring in in vivo methods, such as blood, lymph, cytosolic liquids, or other body liquids, or e.g. liquids, which may be used as reference media in in vitro methods, such as common buffers or liquids. Such common buffers or liquids are known to a skilled person. Ringer-Lactate solution is particularly preferred as a liquid basis.

One or more compatible solid or liquid fillers or diluents or encapsulating compounds suitable for administration to a subject may be used as well for the inventive pharmaceutical composition. The term “compatible” as used herein preferably means that these components of the (pharmaceutical) composition are capable of being mixed with the artificial nucleic acid, the vector or the cells as defined herein in such a manner that no interaction occurs which would substantially reduce the pharmaceutical effectiveness of the composition under typical use conditions.

The composition according to the present invention may optionally further comprise one or more additional pharmaceutically active components. A pharmaceutically active component in this context is a compound that exhibits a therapeutic effect to heal, ameliorate or prevent a particular indication or disease. Such compounds include, without implying any limitation, peptides or proteins, nucleic acids, (therapeutically active) low molecular weight organic or inorganic compounds (molecular weight less than 5000, preferably less than 1000), sugars, antigens or antibodies, or other therapeutic agents already known in the prior art.

Furthermore, the composition may comprise a carrier for the artificial nucleic acid molecule or the vector. Such a carrier may be suitable for mediating dissolution in physiological acceptable liquids, transport and cellular uptake of the pharmaceutical active artificial nucleic acid molecule or the vector. Accordingly, such a carrier may be a component, which is suitable for depot and delivery of an artificial nucleic acid molecule or vector described herein. Such components may be, for example, cationic or polycationic carriers or compounds, which may serve as transfection or complexation agent. Particularly preferred transfection or complexation agents, in this context, are cationic or polycationic compounds,

The term ‘cationic compound’ typically refers to a charged molecule, which is positively charged (cation) at a pH value typically from 1 to 9, preferably at a pH value of or below 9 (e.g. from 5 to 9), of or below 8 (e.g. from 5 to 8), of or below 7 (e.g. from 5 to 7), most preferably at a physiological pH, e.g. from 7.3 to 7.4. Accordingly, a cationic compound may be any positively charged compound or polymer, preferably selected from a cationic peptide or protein or a cationic lipid, which is positively charged under physiological conditions, particularly under physiological conditions in vivo. A ‘cationic peptide or protein’ may contain at least one positively charged amino acid, or more than one positively charged amino acid, e.g. selected from Arg, His, Lys or Orn. Accordingly, ‘polycationic compounds’ are also within the scope exhibiting more than one positive charge under the conditions given.

The composition as described herein preferably comprises the artificial nucleic acid or the vector in naked form or in a complexed form. In a preferred embodiment, the composition comprises the artificial nucleic acid or the vector in the form of a nanoparticle, preferably a lipid nanoparticle or a liposome.

According to a further aspect, the invention relates to a kit or kit of parts comprising the artificial nucleic acid molecule, the vector, the cell, and/or the (pharmaceutical) composition according to the invention.

Preferably, the kit additionally comprises instructions for use, cells for transfection, means for administration of the composition, a (pharmaceutically acceptable) carrier or vehicle and/or a (pharmaceutically acceptable) solution for dissolution or dilution of the artificial nucleic acid molecule, the vector, the cells or the composition. In preferred embodiments, the kit comprises the artificial nucleic acid or the vector described herein, either in liquid or in solid form (e.g. lyophilized), and a (pharmaceutically acceptable) vehicle for administration. For example, the kit may comprise the artificial nucleic acid or the vector and a vehicle (e.g. water, PBS, Ringer-Lactate or another suitable buffer), which are mixed prior to administration to a subject.

In a further aspect, the present invention concerns the use of the artificial nucleic acid, the vector, the cell, the composition or the kit described herein.

In particular, the invention comprises the use of the artificial nucleic acid, the vector, the cell, the composition or the kit for site-directed editing of a target RNA. Therein, the artificial nucleic acid, the vector, the cell, the composition or the kit described herein is preferably used to promote site-specific editing of a target RNA, preferably by specifically binding to the target RNA, thereby recruiting to the target site a deaminase as described herein. That reaction may take place in vitro or in vivo.

In a preferred embodiment, the artificial nucleic acid, the vector or the composition is administered or introduced into a cell comprising a target RNA to be edited. Said cell comprising a target RNA preferably further comprises a deaminase, preferably as described herein. Said deaminase is preferably an endogenous adenosine deaminase, or a recombinant deaminase (such as a tagged deaminase or a mutant deaminase, preferably as described herein), which is either stably expressed in said cell or introduced into said cell, preferably prior or concomitantly with the artificial nucleic acid, the vector or the composition. Alternatively, the cell comprising the artificial nucleic acid or the vector described herein is used for site-directed editing of a target RNA by bringing into contact the cell and the target RNA or by introducing the target RNA into the cell, e.g. by transfection, preferably as described herein.

In a further preferred embodiment, the invention provides a method for site-directed editing of a target RNA, which comprises contacting a target RNA with the artificial nucleic acid and which essentially comprises the steps as described herein with respect to the use of the artificial nucleic acid, the vector, the composition or the cell for site-directed editing of an RNA.

The editing reaction is preferably monitored or controlled by sequence analysis of the target RNA.

The use and the method described herein may further be employed for in vitro diagnosis of a disease or disorder. Therein, the disease or disorder is preferably selected from the group consisting of infectious diseases, tumour diseases, cardiovascular diseases, autoimmune diseases, allergies and neurological diseases or disorders, and is more preferably selected from genetic diseases or genetic disorders, which are preferably selected from the group consisting of metabolic diseases, tumour diseases, autoimmune diseases, cardiovascular diseases and neurological diseases.

In a further aspect, the artificial nucleic acid, the vector, the cell, the composition, or the kit described herein is provided for use as a medicament, e.g. in gene therapy. Preferably, the artificial nucleic acid, the vector, the composition, the cell or the kit described herein is provided for use in the treatment or prophylaxis of a disease or disorder selected from the group consisting of infectious diseases, tumour diseases, cardiovascular diseases, autoimmune diseases, allergies and neurological diseases or disorders. According to a preferred embodiment, the artificial nucleic acid, the vector, the cell, the composition, or the kit described herein is provided for use as a medicament or for use in the treatment or prophylaxis of a disease or disorder, preferably as defined herein, wherein the use as a medicament or the treatment or prophylaxis comprises a step of site-directed editing of a target RNA.

In one aspect, the present invention further provides a method for treating a subject suffering from a disease or a disorder, the method comprising administering an effective amount of the artificial nucleic acid, the vector, the cell or the composition described herein to the subject. An effective amount in the context of the present disclosure is typically understood to be an amount that is sufficient to trigger the desired therapeutical effect, i.e. to achieve editing of a target RNA.

The disease or the disorder may be selected from the group consisting of infectious diseases, tumour diseases, cardiovascular diseases, autoimmune diseases, allergies and neurological diseases or disorders, wherein the disease or the disorder is preferably selected from a genetic disease or genetic disorder, which is preferably selected from the group consisting of metabolic diseases, tumour diseases, autoimmune diseases, cardiovascular diseases and neurological diseases.

The artificial nucleic acid, the vector, the cell, or the (pharmaceutical) composition described herein may be administered orally, parenterally, by inhalation spray, topically, rectally, nasally, buccally, vaginally, via an implanted reservoir or via jet injection. The term parenteral as used herein includes intra-vitreal, sub-retinal, subcutaneous, intravenous, intramuscular, intra-articular, intra-synovial, intrasternal, intrathecal, intrahepatic, intralesional, intracranial, transdermal, intradermal, intrapulmonal, intraperitoneal, intracardial, intraarterial, and sublingual injection or infusion techniques. In a preferred embodiment, the artificial nucleic acid molecule, the vector, the cell or the (pharmaceutical) composition described herein is administered via needle-free injection (e.g. jet injection).

Preferably, the artificial nucleic acid, the vector, the cell, or the (pharmaceutical) composition described herein is administered parenterally, e.g. by parenteral injection, more preferably by intra-vitreal, sub-retinal, subcutaneous, intravenous, intramuscular, intra-articular, intra-synovial, intrasternal, intrathecal, intrahepatic, intralesional, intracranial, transdermal, intradermal, intrapulmonal, intraperitoneal, intracardial, intraarterial, sublingual injection or via infusion techniques. Particularly preferred is intradermal and intramuscular injection. Sterile injectable forms of the inventive pharmaceutical composition may be aqueous or oleaginous suspension. These suspensions may be formulated according to techniques known in the art using suitable dispersing or wetting agents and suspending agents.

The artificial nucleic acid, the vector, the cell, or the (pharmaceutical) composition described herein may also be administered orally in any orally acceptable dosage form including, but not limited to, capsules, tablets, aqueous suspensions or solutions.

The artificial nucleic acid, the vector, the cell, or the (pharmaceutical) composition described herein may also be administered topically, especially when the target of treatment includes areas or organs readily accessible by topical application, e.g. including diseases of the skin or of any other accessible epithelial tissue. Suitable topical formulations are readily prepared for each of these areas or organs. For topical applications, the artificial nucleic acid, the vector, the cell, or the (pharmaceutical) composition described herein may be formulated in a suitable ointment suspended or dissolved in one or more carriers.

In one embodiment, the use as a medicament comprises the step of transfection of mammalian cells, preferably in vitro or ex vivo transfection of mammalian cells, more preferably in vitro transfection of isolated cells of a subject to be treated by the medicament. If the use comprises the in vitro transfection of isolated cells, the use as a medicament may further comprise the re-administration of the transfected cells to the patient. The use of the artificial nucleic acid or the vector as a medicament may further comprise the step of selection of successfully transfected isolated cells. Thus, it may be beneficial if the vector further comprises a selection marker.

According to another aspect of the present invention, the artificial nucleic acid, the vector, the cell, or the (pharmaceutical) composition described herein is provided for use in the diagnosis of a disease or disorder, which is preferably selected from the group consisting of infectious diseases, tumour diseases, cardiovascular diseases, autoimmune diseases, allergies and neurological diseases or disorders, in particular from a genetic disease or genetic disorder, which is preferably selected from the group consisting of metabolic diseases, tumour diseases, autoimmune diseases, cardiovascular diseases and neurological diseases.

### DESCRIPTION OF THE FIGURES

The figures shown in the following are merely illustrative and shall describe the present invention in a further way. These figures shall not be construed to limit the present invention thereto.

FIG. 1: Modulation of RNA editing by adjacent GU- and UG-wobbles or G-A mismatches characterized using cis-acting guide RNA constructs (Examples 1-3)
1A: Effect of adjacent GU- and UG-wobbles or GA mismatches and compared to regular UA matches at an editing site. Compared to an UA match a GA mismatch or an adjacent GU wobble reduces editing, while an adjacent UG wobble increases editing.
1B: 5′-nearest neighbor (5′-NN), 3′-nearest neighbor (3′-NN) and counterbase positions in the context of an edited adenosine. ADAR1 prefers U>A>C>G at its 5′-NN side, G>A˜C>U at its 3′-NN site and C>U>A˜G as a counterbase for the edited adenosine for high editing yields. N stands for A, G, C or U and represents the continuation of both RNA stands 5′ and 3′ of the target triplet (in this case 5′-UAG) and the antisense triplet (in this case 5′-CCA).
1C: Structure of the cis-acting guide RNA reporter constructs that were used to generate the datasets seen in FIGS. 1D-F. Their guide RNA part was located in the 3′-UTR of an eGFP reporter and was based on an earlier guide RNA design, comprising a double-stranded ADAR recruiting domain and a single-stranded 20 nt specificity domain. They allowed to screen though all possible combinations of 5′-nearest neighbor and 3′-nearest neighbor base pairs, including wobbles. N stands for A, G, C or U. K stands for G or U.
1D: UG-wobbles increase the RNA editing yield at adjacent adenosines. Reference designs do not contain UG wobbles. The target adenosine is underlined (A). Uridines in the antisense triplet that form the UG wobble with the guanosine in the target triplet are bold. The given editing yields represents the mean of n=3 biological replicates performed in ADAR1 p110 Flp-In T-REx cells.
1E: GU-wobbles reduce the RNA editing yield at adjacent adenosines. Reference designs do not contain GU wobbles or GA mismatches. The target adenosine is underlined (A). Guanosines in the antisense triplet that form a UG-wobble or a GA mismatch with the target triplet are bold. The given editing yields represents the mean of n=3 biological replicates performed in ADAR1 p110 Flp-In T-REx cells.
1F: Sequencing read examples from the cis-acting guide RNA reporter constructs for all triplets that can contain either an adjacent GU-(5′-NN or 3′-NN uridine) and/or UG-wobble (5′-NN or 3′-NN guanosine) or a guanosine as counterbase (GA mismatch). For the wobble solutions the counterbase at the target site is always a uridine. GU-wobbles sites are highlighted with an asterisk (*), UG wobbles with a 0 and GA mismatches with a X.
1G: Structure of the cis-acting guide RNA reporter constructs that were used to generate the dataset seen in FIG. 1H. Their guide RNA part was located in the 3′-UTR of an eGFP reporter and was based on an earlier guide RNA design, comprising a double-stranded ADAR recruiting domain and a single-stranded 20 nt specificity domain. To increase the editing yield compared to the construct in FIG. 1C the specificity and recruiting domains were structured as one continuous duplex. They allowed to analyse the effect of GU wobbles and GA mismatches close to off-target sites at 5′-nearest neighbor and 3′-nearest neighbor positions. B stands for C, G or U. D stands for A, G or U. R stands for A or G. W stands for A or U. The exact sequence of each construct is listed in detail in FIG. 1H.
1H: GU-wobbles can reduce off-target editing at adenosines located directly 5′ or 3′ of a target adenosine without reduction of the on-target editing yield in cis-acting guide RNA reporter constructs. The given editing yields represents the mean±SD of n=3 biological replicates. The target triplets (either 5′-UAA or 5′-AAG. On-target adenosine underlined. Nearest neighbor off-target adenosine in bold.) are highlighted in dark grey. All constructs contain a CA mismatch as counterbase of the target adenosine at position ±0, as these constructs were designed to characterized the effect GU-wobbles and GA mismatches in an on-target situation.
1I: UG-wobbles increase the RNA editing yield at adjacent adenosines. Reference designs do not contain UG wobbles. The target adenosine is underlined (A). Uridines in the antisense triplet that form the UG wobble with the guanosine in the target triplet are bold. The given editing yields represents the mean of n=3 biological replicates performed in either ADAR1 p110, ADAR1 p150 or ADAR2 Flp-In T-REX cells.
1J: GU-wobbles reduce the RNA editing yield at adjacent adenosines. Reference designs do not contain GU wobbles or GA mismatches. The target adenosine is underlined (A). Guanosines in the antisense triplet that form a UG-wobble or a GA mismatch with the target triplet are bold. The given editing yields represents the mean of n=3 biological replicates performed in either ADAR1 p110, ADAR1 p150 or ADAR2 Flp-In T-REx cells.

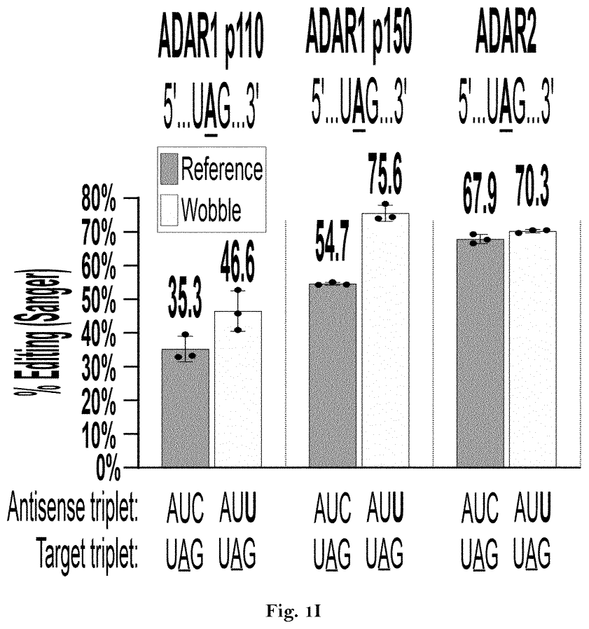

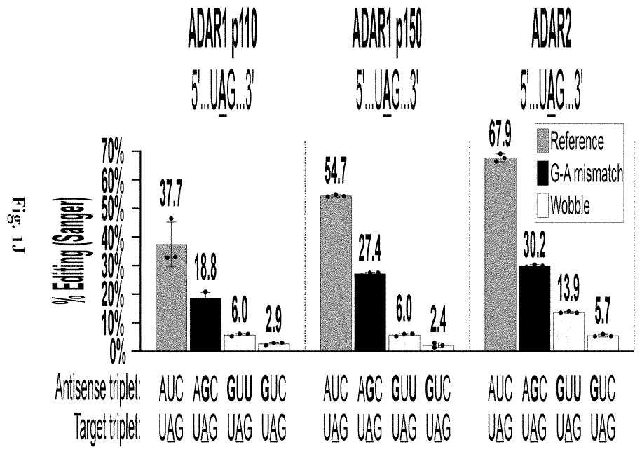

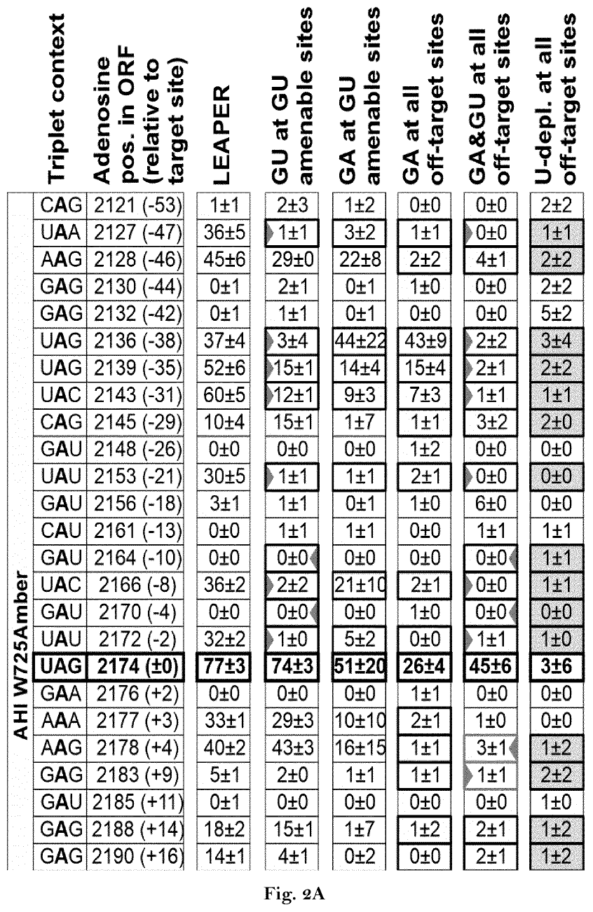

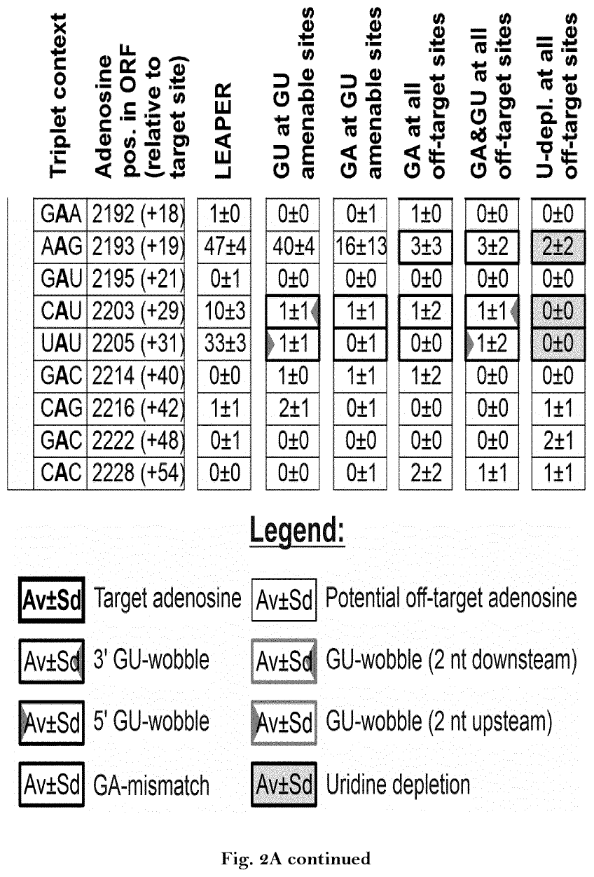

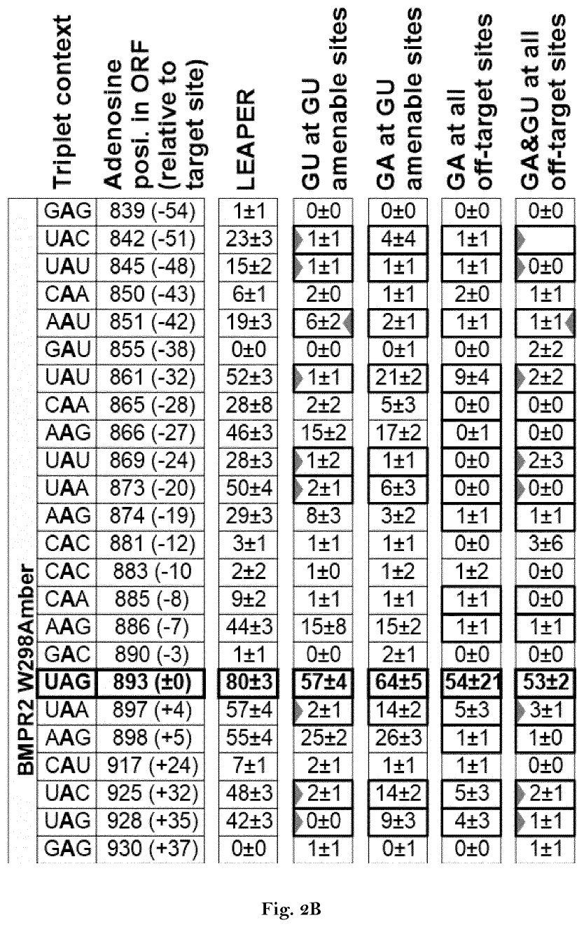

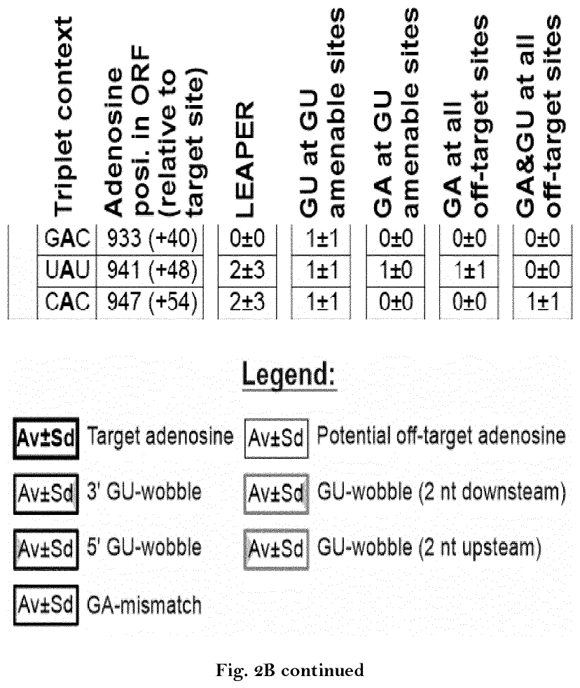

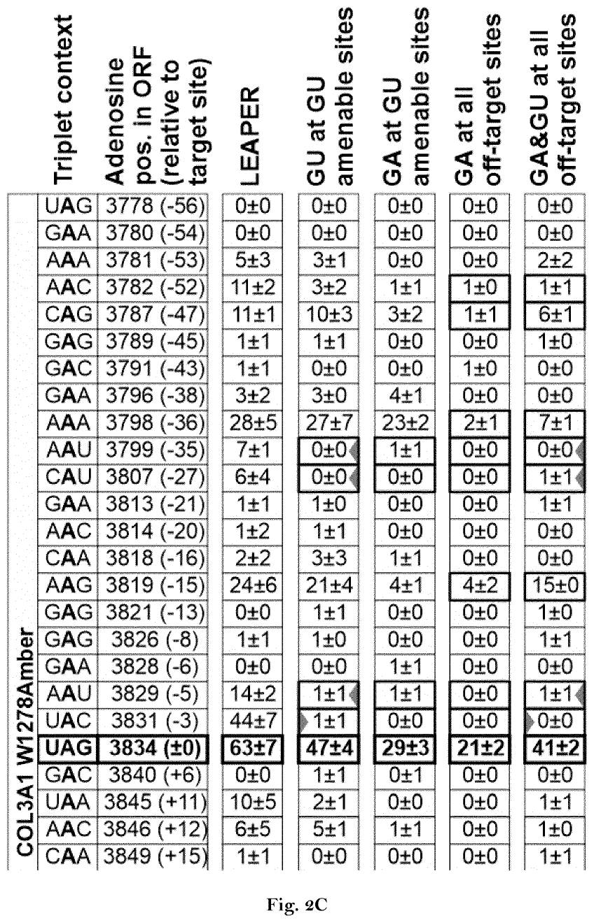

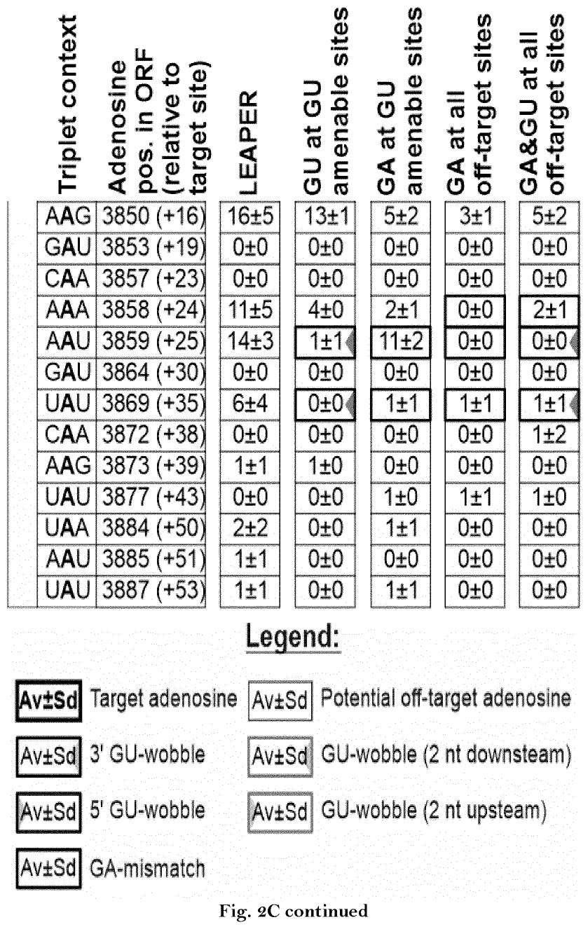

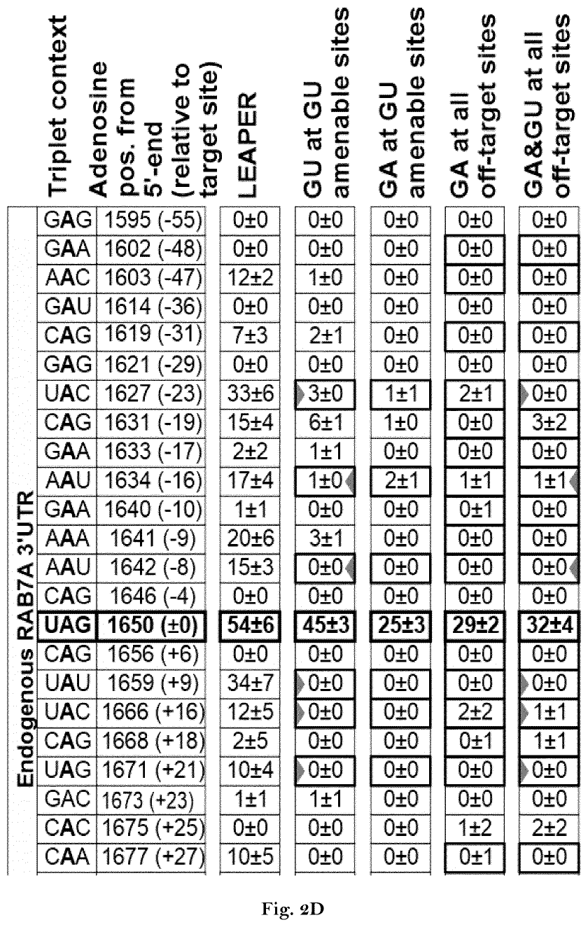

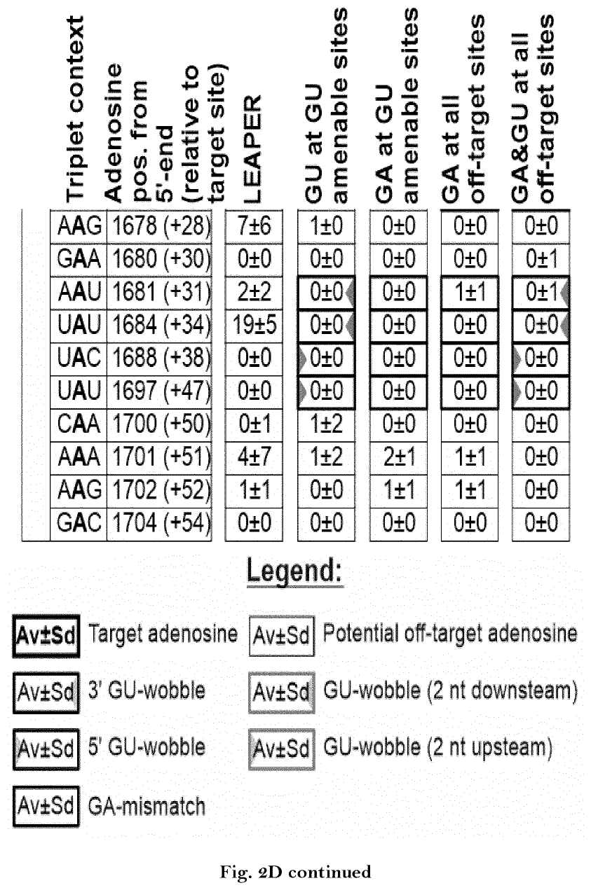

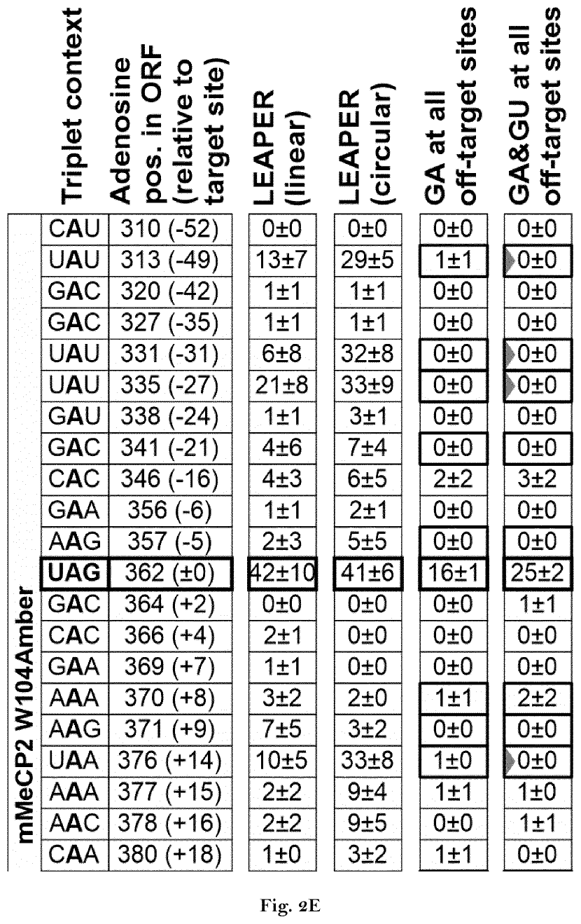

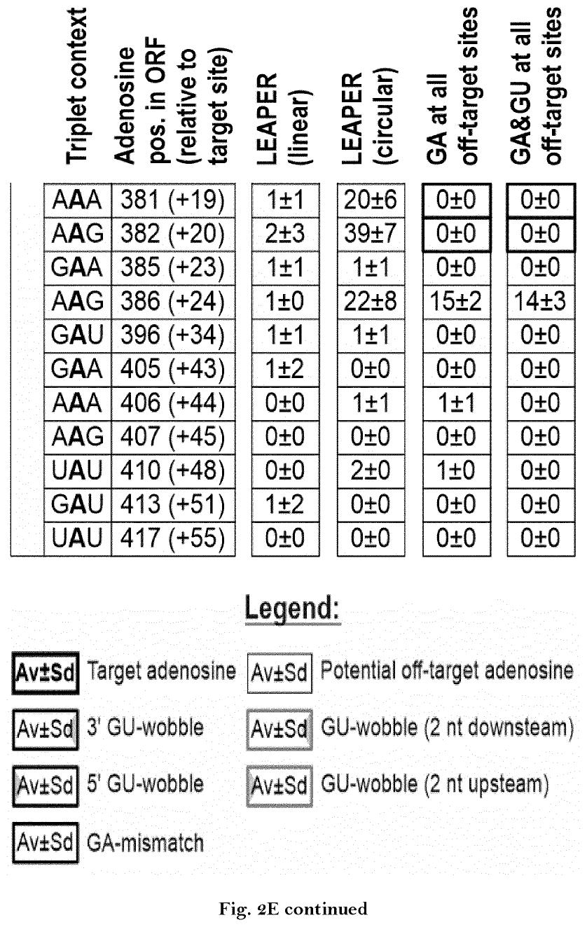

FIG. 2: Off-target editing suppression via GU-wobbles, GA mismatches and Uridine depletion characterized using trans-acting 111 nt long LEAPER guide RNAs (Examples 4-8)
2A: Editing yields of all adenosines within the duplex of the LEAPER guide RNA and the overexpressed AHI W725Amber target mRNA. The row containing the target adenosine is written in bold and highlighted with a wider edge. The column “triplet context” shows the 5′-NN (left base) and the 3′-NN (right base) around the adenosine at this position (centre base, bold). The column “Adenosine pos. in ORF” shows the position of this adenosine counted starting from the first base in the open reading frame (ORF) of the target transcript. The number in brackets in the same column shows the distance of this adenosine relative to target site which is defined as ±0. A negative value means 5′ of the target adenosine in the mRNA. A positive value means 3′ of the target adenosine on the target mRNA. The column “LEAPER” shows the editing results of a symmetric (centred target adenosine) 111 nt long LEAPER guide RNA. The column “GU at GU amenable sites” shows the editing results of a symmetric 111 nt long LEAPER guide RNA containing GU-wobbles 5′ or 3′ of all sites that caused off-target editing in the regular LEAPER guide RNA and are amenable to GU-wobbles. The column “GA at GU amenable sites” shows the editing results of a symmetric 111 nt long LEAPER guide RNA containing GA mismatches at all sites that caused off-target editing in the regular LEAPER guide RNA and would be amenable to GU-wobbles. The column “GA at all off-target sites” shows the editing results of a symmetric 111 nt long LEAPER guide RNA containing GA mismatches at all sites that caused off-target editing in the regular LEAPER guide RNA. The column “GA&GU at all off-target sites” shows the editing results of a symmetric 111 nt long LEAPER guide RNA containing GU-wobbles 5′ or 3′ of all sites that caused off-target editing in the regular LEAPER guide RNA and are amenable to GU-wobbles, and GA mismatches at all sites that caused off-target editing in the regular LEAPER guide RNA but are not amenable to GU-wobbles. The column “U-depl. at all off-target sites” shows the editing results of a symmetric 111 nt long LEAPER guide RNA depleted from uridine counterbases at all sites that caused off-target editing in the regular LEAPER guide RNA. The given editing yields in % represent the mean±SD of n=3 biological replicates.
2B: Editing yields of all adenosines within the duplex of the LEAPER guide RNA and the overexpressed BMPR2 W298Amber target mRNA. For a detailed description of the rows and columns reference is made to FIG. 2A. The given editing yields in % represent the mean±SD of n=3 biological replicates.
2C: Editing yields of all adenosines within the duplex of the LEAPER guide RNA and the overexpressed COL3A1 W1278Amber target mRNA. For a detailed description of the rows and columns reference is made to FIG. 2A. The given editing yields in % represent the mean±SD of n=3 biological replicates.
2D: Editing yields of all adenosines within the duplex of the LEAPER guide RNA and the endogenous RAB7A target mRNA. The column “Adenosine pos. from 5′-end” shows the position of this adenosine counted starting from the first 5′-base of the target transcript. The number in brackets in the same column shows the distance of this adenosine relative to target site which is defined as to. A negative value means 5′ of the target adenosine in the mRNA. A positive value means 3′ of the target adenosine on the target mRNA. The target adenosine of these LEAPER guide RNAs is located within the 3′-UTR of RAB7A. For further descriptions of the rows and columns reference is made to FIG. 2A. The given editing yields in % represent the mean±SD of n=3 biological replicates.
2E: Editing yields of all adenosines within the duplex of the LEAPER guide RNA and the overexpressed murine MeCP2 W104Amber target mRNA. The column “LEAPER (linear)” shows the editing results of a regular not circularized and symmetric (centred target adenosine) 111 nt long LEAPER guide RNA. The column “LEAPER (circular)” shows the editing results of a symmetric (centred target adenosine) 111 nt long LEAPER guide RNA that was circularized using the Tornado expression system adopted for guide RNAs from Litke, J. L. and S. R. Jaffrey (supra). The column “GA at all off-target sites” shows the editing results of a circularized and symmetric 111 nt long LEAPER guide RNA containing GA mismatches at all sites that caused off-target editing in the regular linear or circular LEAPER guide RNA. The column “GA&GU at all off-target sites” shows the editing results of a circularized and symmetric 111 nt long LEAPER guide RNA containing GU-wobbles 5′ or 3′ of all sites that caused off-target editing in the regular linear or circular LEAPER guide RNA and are amenable to GU-wobbles, and GA mismatches at all sites that caused off-target editing in the regular linear or circular LEAPER guide RNA but are not amenable to GU-wobbles. For a detailed description of the remaining rows and columns reference is made to FIG. 2A. The given editing yields in % represent the mean±SD of n=3 biological replicates.

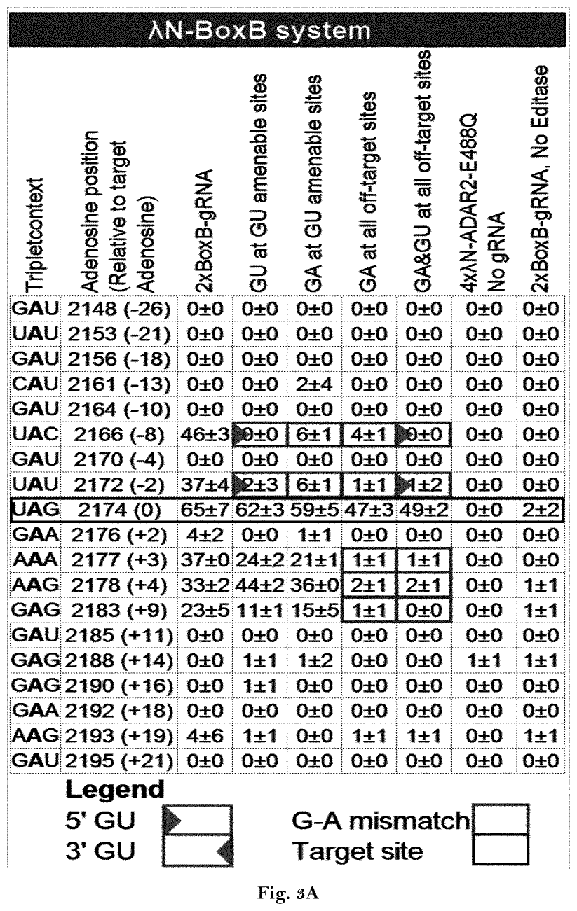

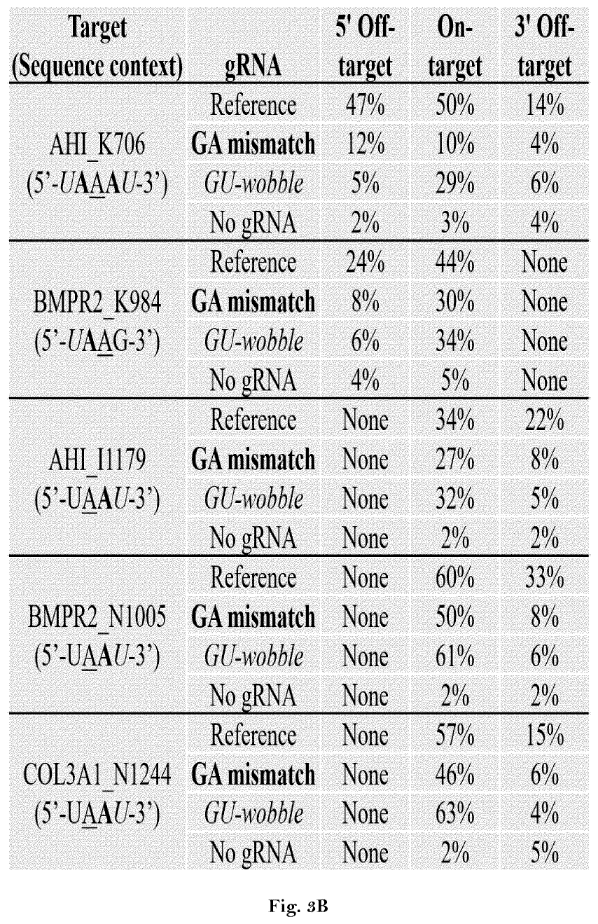

FIG. 3: Off-target editing suppression via GU-wobbles and GA mismatches characterized using trans-acting 2×BoxB guide RNAs (Example 9 and 10)
3A: Editing yields of all adenosines within the duplex of the 2×BoxB guide RNAs and the overexpressed AHI W725Amber target mRNA. The row containing the target adenosine is highlighted with a black box. The column “triplet context” shows the 5′-NN (left base) and the 3′-NN (right base) around the adenosine at this position (centre base, bold). The column “Adenosine position” shows the position of this adenosine counted starting from the first base in the open reading frame (ORF) of the target transcript. The number in brackets in the same column shows the distance of this adenosine relative to target site which is defined as ±0. A negative value means 5′ of the target adenosine in the mRNA. A positive value means 3′ of the target adenosine on the target mRNA. The column “2×BoxB-gRNA” shows the editing results of a 2×BoxB guide RNA co-overexpressed with the 4×λN-ADAR2-DD-E488Q editase and the AHI target transcript. The column “GU at GU amenable sites” shows the editing results of a 4×λN-ADAR2-DD-E488Q editase and the AHI target transcript co-overexpressed with a 2×BoxB guide RNA containing GU-wobbles 5′ or 3′ of all sites that caused off-target editing in the regular 2×BoxB guide RNA and are amenable to GU-wobbles. The column “GA at GU amenable sites” shows the editing results of a 4×λN-ADAR2-DD-E488Q editase and the AHI target transcript co-overexpressed with a 2×BoxB guide RNA containing GA mismatches at all sites that caused off-target editing in the regular 2×BoxB guide RNA and would be amenable to GU-wobbles. The column “GA at all off-target sites” shows the editing results of a 4×λN-ADAR2-DD-E488Q editase and the AHI target transcript co-overexpressed with a 2×BoxB guide RNA containing GA mismatches at all sites that caused off-target editing in the regular 2×BoxB guide RNA. The column “GA&GU at all off-target sites” shows the editing results of a 4×λN-ADAR2-DD-E488Q editase and the AHI target transcript co-overexpressed with a 2×BoxB guide RNA containing GU-wobbles 5′ or 3′ of all sites that caused off-target editing in the regular 2×BoxB guide RNA and are amenable to GU-wobbles, and GA mismatches at all sites that caused off-target editing in the regular 2×BoxB guide RNA but are not amenable to GU-wobbles. The column “4×λN-ADAR2-DD-E488Q, No gRNA” shows the editing results of a 4×λN-ADAR2-DD-E488Q editase and the AHI target transcript co-overexpressed without a 2×BoxB guide RNA. The column “2×BoxB guide RNA, No Editase” shows the editing results of the AHI target transcript co-overexpressed with a 2×BoxB guide RNA without the 4×λN-ADAR2-DD-E488Q editase. The given editing yields in % represent the mean±SD of n=3 biological replicates.
3B: Off-target editing suppression using GU-wobbles or GA mismatches at both 5′ and 3′, only 5′- or only 3′-nearest neighbor sites next to the target adenosine demonstrated using 2×BoxB guide RNAs targeting the AHI W725X, BMPR2 W298X and COL3A1 W1278X transcripts at the amino acid AHI K706, AHI 11179, BMPR2 K984, BMPR2 N1005 and COL3A1 N1244. Overall the GA mismatch solution reduces the on-target editing stronger and the off-target editing weaker than the GU-wobble solution. The given editing yields represent n=1 biological replicate.

FIG. 4: Off-target editing suppression via GU-wobbles and GA mismatches characterized using trans-acting Directed-Repeat (DR) guide RNAs (Example 11)
Editing yields of all adenosines within the duplex of the DR guide RNAs and the overexpressed AHI W725Amber target mRNA. The row containing the target adenosine is highlighted with a black box. The column “triplet context” shows the 5′-NN (left base) and the 3′-NN (right base) around the adenosine at this position (centre base, bold). The column “Adenosine position” shows the position of this adenosine counted starting from the first base in the open reading frame (ORF) of the target transcript. The number in brackets in the same column shows the distance of this adenosine relative to target site which is defined as to. A negative value means 5′ of the target adenosine in the mRNA. A positive value means 3′ of the target adenosine on the target mRNA. The column “DR gRNA” shows the editing results of a DR guide RNA co-overexpressed with the dPspCas13b-ADAR2-DD-E488Q editase and the AHI target transcript. The column “GU at GU amenable sites” shows the editing results of a dPspCas13b-ADAR2-DD-E488Q editase and the AHI target transcript co-overexpressed with a DR guide RNA containing GU-wobbles 5′ or 3′ of all sites that caused off-target editing in the regular DR guide RNA and are amenable to GU-wobbles. The column “GA at GU amenable sites” shows the editing results of a dPspCas13b-ADAR2-DD-E488Q editase and the AHI target transcript co-overexpressed with a DR guide RNA containing GA mismatches at all sites that caused off-target editing in the regular DR guide RNA and would be amenable to GU-wobbles. The column “GA at all off-target sites” shows the editing results of a dPspCas13b-ADAR2-DD-E488Q editase and the AHI target transcript co-overexpressed with a DR guide RNA containing GA mismatches at all sites that caused off-target editing in the regular DR guide RNA. The column “GA&GU at all off-target sites” shows the editing results of a dPspCas13b-ADAR2-DD-E488Q editase and the AHI target transcript co-overexpressed with a DR guide RNA containing GU-wobbles 5′ or 3′ of all sites that caused off-target editing in the regular DR guide RNA and are amenable to GU-wobbles, and GA mismatches at all sites that caused off-target editing in the regular DR guide RNA but are not amenable to GU-wobbles. The column “dPspCas13b-ADAR2-DD-E488Q, No gRNA” shows the editing results of a dPspCas13b-ADAR2-DD-E488Q editase and the AHI target transcript co-overexpressed without a DR guide RNA. The column “DR guide RNA, No Editase” shows the editing results of the AHI target transcript co-overexpressed with a DR guide RNA without the dPspCas13b-ADAR2-DD-E488Q editase. The given editing yields in % represent the mean±SD of n=3 biological replicates.

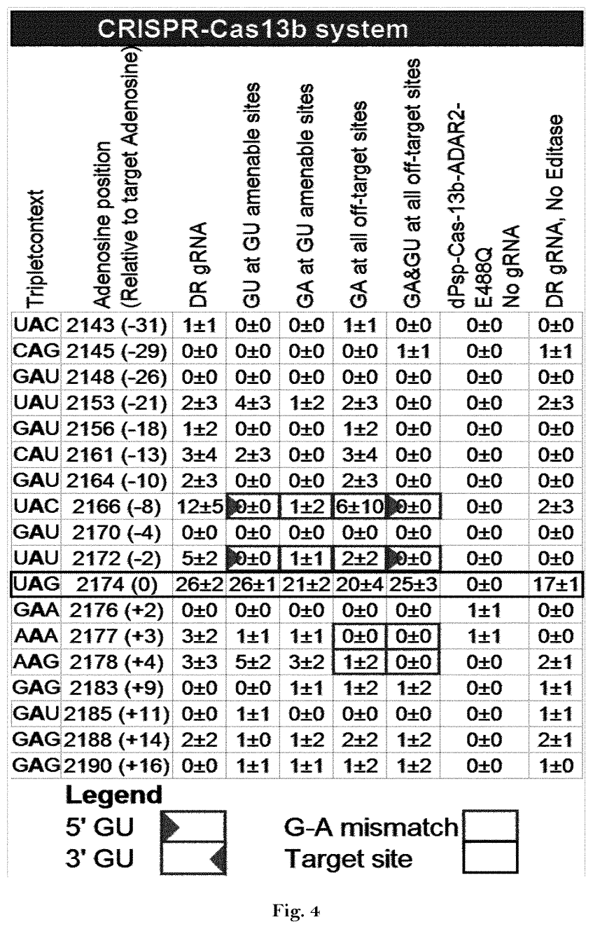

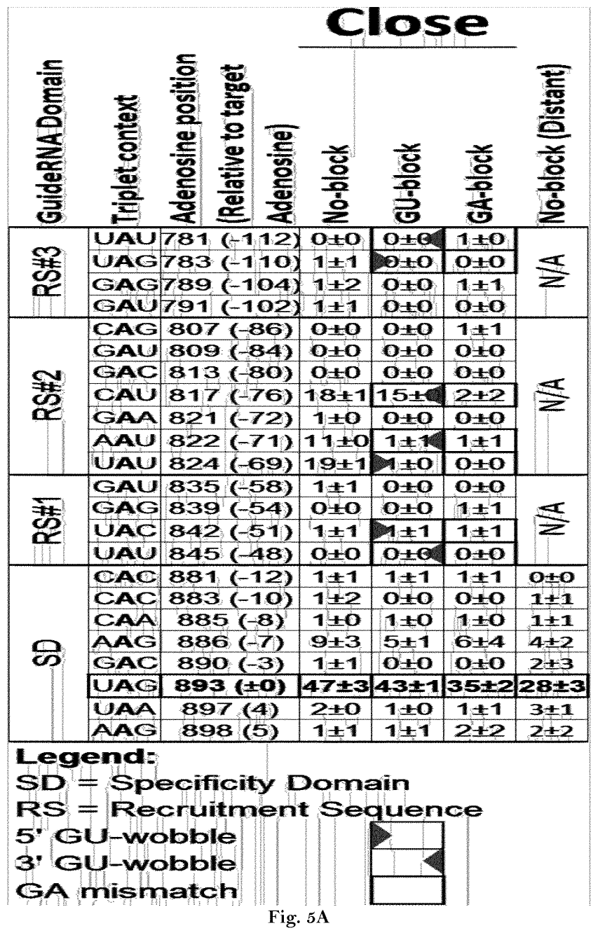

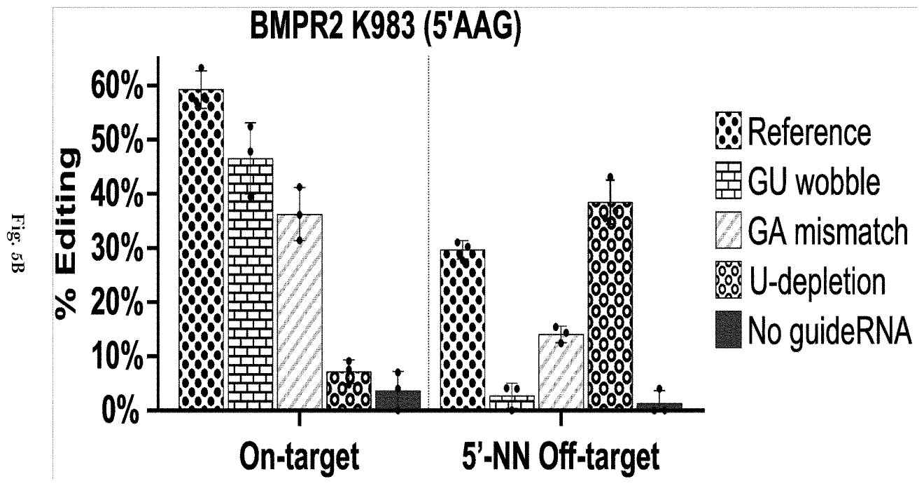

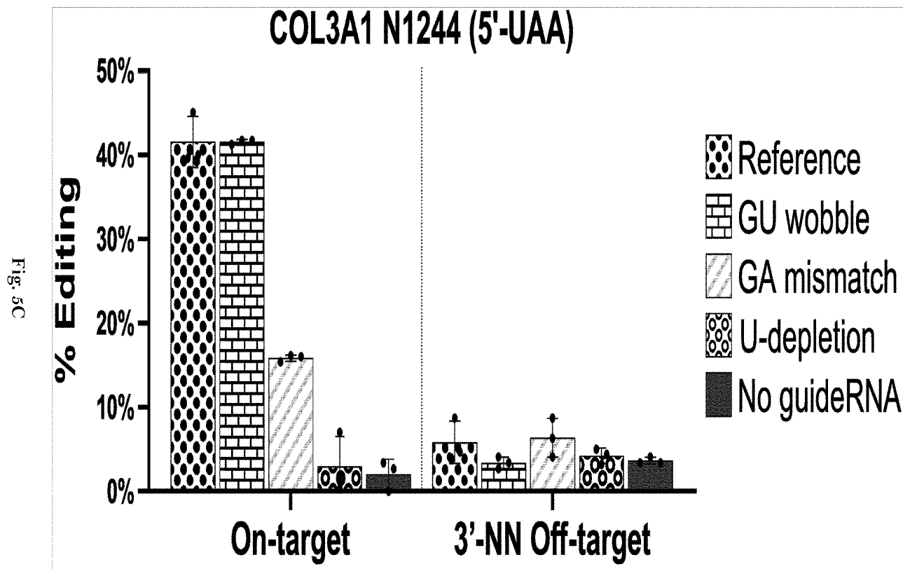

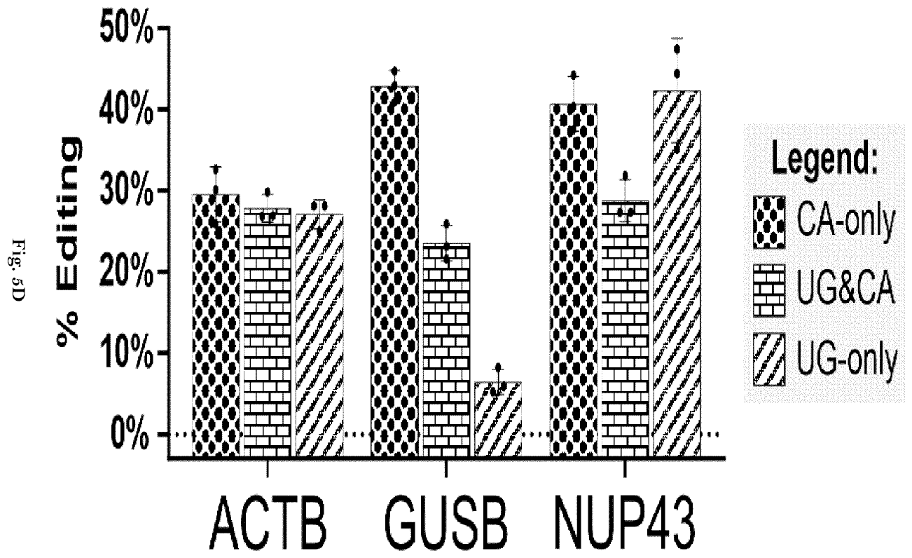

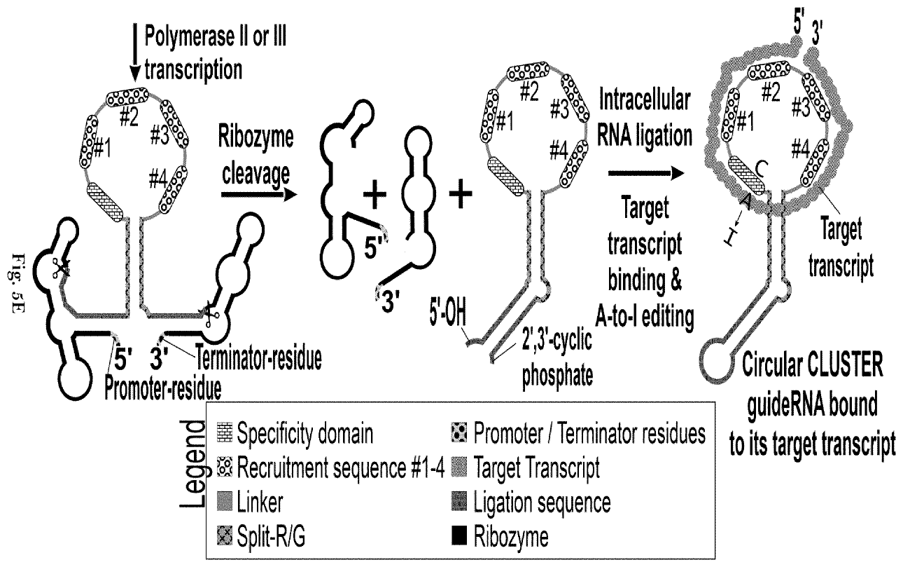

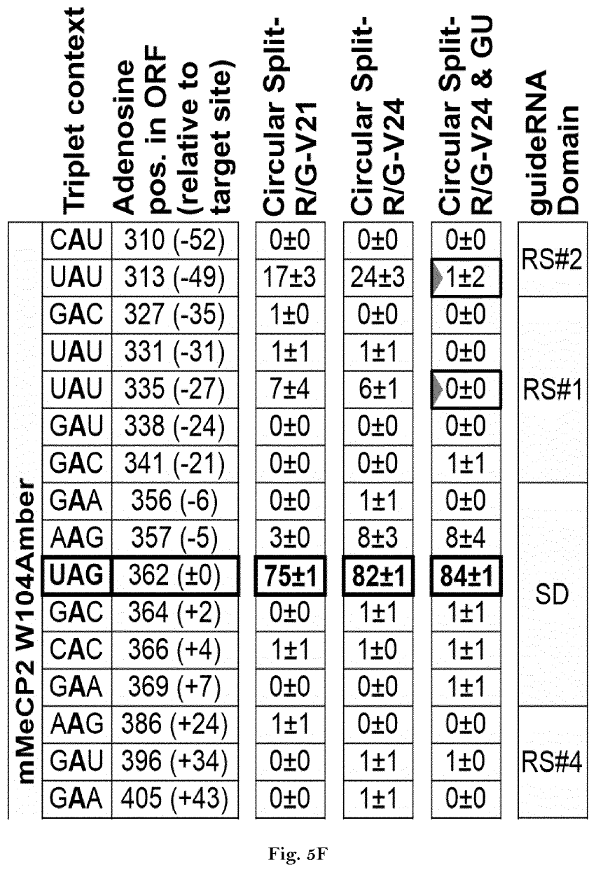

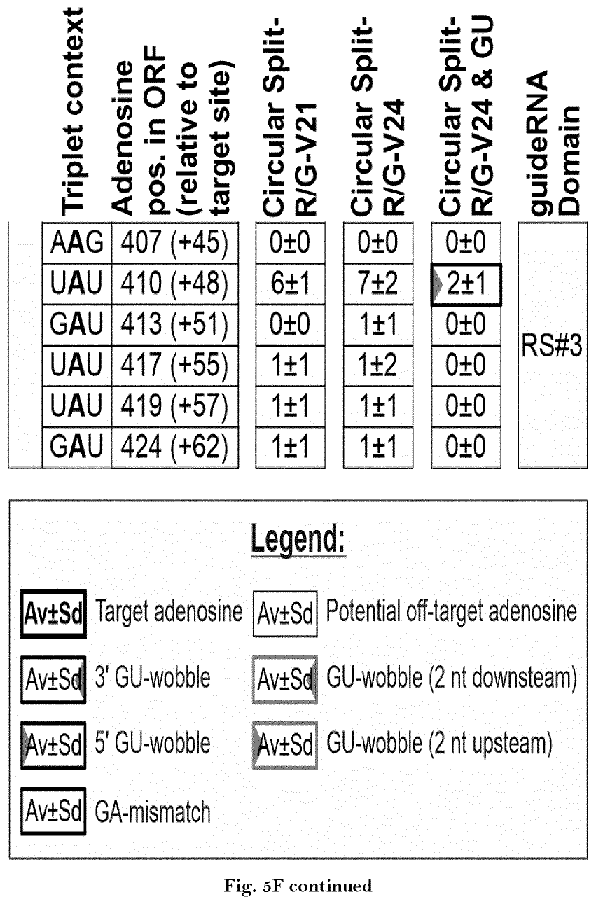

FIG. 5: Off-target editing suppression via GU-wobbles, GA mismatches or Uridine depletion or a boost of on-target editing via UG-wobbles characterized in trans-acting CLUSTER or 2×BoxB guide RNAs (Examples 12-16).
5A: Editing yields of all adenosines within the duplex of the CLUSTER guide RNA and the overexpressed BMPR2 W298Amber target mRNA. The column “Guide RNA Domain” shows which section of the CLUSTER guide RNA (SD or RS) forms the duplex containing the adenosines at these positions with the BMPR2 target transcript. RS stands for Recruitment Sequence. SD stands for Specificity Domain and contains the target adenosine. The row containing the target adenosine is written in bold and highlighted with a wider edge. The column “triplet context” shows the 5′-NN (left base) and the 3′-NN (right base) around the adenosine at this position (centre base, bold). The column “Adenosine position” shows the position of this adenosine counted starting from the first base in the open reading frame (ORF) of the target transcript. The number in brackets in the same column shows the distance of this adenosine relative to target site which is defined as to. A negative value means 5′ of the target adenosine in the mRNA. A positive value means 3′ of the target adenosine on the target mRNA. The CLUSTER guide RNAs that are labelled as “Close” cover a distance of 88 nt between the two binding sites on the target transcript that are most remote to each other (SD and RS #3). They were in-silico optimized with rules allowing for adenosines in a 5′-GAN, 5′-UAB or 5′-BAU (B=C, G, U) triplet contexts within the binding sites. The CLUSTER guide RNAs that is labelled as “No-block (Distant)” covers a distance of 841 nt on the target transcript. It was in-silico optimized with rules allowing for adenosines only in a 5′-GAN triplet contexts within the binding sites as described in Reautschnig, P., et al. (supra). The column “No-block (Close)” and “No-block (Distant)” shows the editing results of CLUSTER guide RNAs that do not contain GU-wobbles or GA mismatches to prevent off-target editing. The editing yields within the recruitment sequence binding sites of the “No-block (Distant)” CLUSTER guide RNA were not analysed (N/A), as a 5′-GAN triplet context is not prone to off-target editing. The column “GU-block (Close)” shows the editing results of a CLUSTER guide RNA that does contain GU-wobbles at sites within the RS predicted to show high off-target editing. The column “GA-block (Close)” shows the editing results of a CLUSTER guide RNA that does contain GA mismatches at sites within the RS predicted to show high off-target editing. The given editing yields in % represent the mean±SD of n=3 biological replicates.
5B: Off-target editing suppression using GU-wobbles, GA mismatches, or Uridine depletion at a 5′-nearest neighbor site next to the target adenosine demonstrated using a CLUSTER guide RNA targeting the BMPR2 W298Amber transcript. The Uridine depletion solution shows the lowest on-target editing yield and simultaneously the highest off-target editing yield in this experiment. The GA mismatch solution reduces the on-target editing at the 5′-AAG (BMPR2 K983) target triplet stronger and the off-target editing weaker than the GU-wobble solution. The given editing yields represents the mean of n=3 biological replicates.
5C: Off-target editing suppression using GU-wobbles, GA mismatches, or Uridine depletion at a 3′-nearest neighbor site next to the target adenosine demonstrated using a CLUSTER guide RNA targeting the COL3A1 W1278X transcript. The Uridine depletion solution obliterates the on-target editing yield close to that of the negative control without guide RNA. The GA mismatch solution reduces the on-target editing at the 5′-UAA (COL3A1 N1244) target triplet stronger and the off-target editing weaker than the GU-wobble solution. The given editing yields represents the mean of n=3 biological replicates.
5D: Evaluation of the UG-wobble boost effect in trans using CLUSTER guide RNAs targeting the endogenous housekeeping gens ACTB, GUSB and NUP43. A UG-wobble next to the target adenosine is compared to the prior art CA mismatch and with a combination of CA mismatch and UG-wobble.
5E: Processing of circular CLUSTER guide RNAs using the Tornado expression system (Litke, J. L. and S. R. Jaffrey (2019). Highly efficient expression of circular RNA aptamers in cells using autocatalytic transcripts. Nat Biotechnol 37(6): 667-675). The Tornado system employs two ribozymes flanking the guide RNA to excise it and create overhangs that allow for ligation via an endogenous RNA ligase. The resulting circular guide RNA is more stable due to its resistance against exonucleases. The constructs are expressed either from polymerase II (e.g. CAG) or III (e.g. U6) promoters, after which the 5′ (e.g. P3 Twister U2A ribozyme) and 3′ (e.g. P1 Twister ribozyme) terminal ribozyme cleavage takes place. The ADAR enzyme recruiting R/G motif is split in half so that instead of sitting at the 5′ end of the guide RNA as a hairpin it is formed via an internal duplex. After the 5′ and 3′ ribozymes are cleaved off the remaining 5′ and 3′ lig. seq. (ligation sequences) form a duplex and allow the ligation of both RNA ends via an endogenous ligase (e.g. the ubiquitous endogenous RNA ligase RtcB). The circularized CLUSTER guide RNA then binds to its target mRNA via its SD (specificity domain) and its cluster of four recruitments sequences (RS), which are interrupted by Linkers (L). The exit of the target mRNAs 5′ and 3′ ends is located between RS #2 and RS #3 on the guide RNA. At all other linkers of the guide RNA the target mRNA form loops, which sizes depend on the binding position of the guide RNA on its target mRNA. Finally, Adenosine (A)-to-Inosine (I) RNA editing takes place at the target adenosine, which is positioned in a C-A mismatch, to increase editing yields.
5F: Editing yields of all adenosines within the duplex of circular CLUSTER guide RNAs and the overexpressed murine MeCP2 W104Amber target mRNA. The column “guide RNA Domain” indicates to which domain of the guide RNA (Specificity Domain (SD), Recruitment Sequence #1, 2, 3 or 4 (RS #1, 2, 3, 4) the listed adenosine positions belong to. The column “Circular Split-R/G-V21” shows the editing results of a Tornado expression system circularized CLUSTER guide RNA consisting of one specificity domain, four recruitment sequences, and the version 21 of the ADAR recruitment motif (R/G motif). The column “Circular Split-R/G-V24” shows the editing results of a Tornado expression system circularized CLUSTER guide RNA consisting of one specificity domain, four recruitment sequences, and the version 24 of the ADAR recruitment motif (R/G motif). The column “Circular Split-R/G-V24 & GU” shows the editing results of a Tornado expression system circularized CLUSTER guide RNA consisting of one specificity domain, four recruitment sequences, the version 24 of the ADAR recruitment motif (R/G motif) and three GU wobbles preventing off-target events at the adenosine positions 313, 335 and 410. For a detailed description of the rows and columns reference is made to FIG. 5A. The given editing yields in % represent the mean±SD of n=3 biological replicates.

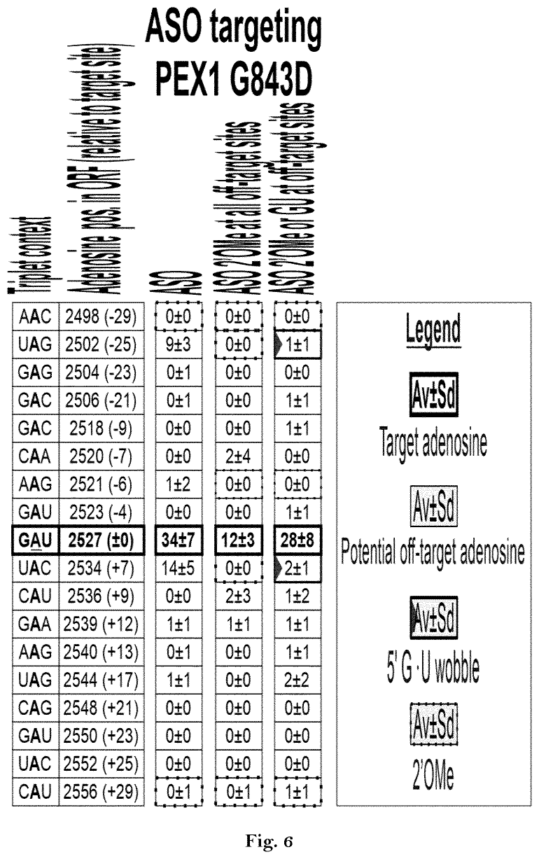

FIG. 6: Off-target editing suppression via GU-wobbles or 2′OMe using 59 nt long ADAR recruiting antisense oligonucleotides (Examples 17)
Editing yields of all adenosines within the duplex of the ASO and the overexpressed PEX1 G843D target mRNA. The row containing the target adenosine is written in bold and highlighted with a wider edge. The column “triplet context” shows the 5′-NN (left base) and the 3′-NN (right base) around the adenosine at this position (centre base, bold). The column “Adenosine pos. in ORF” shows the position of this adenosine counted starting from the first base in the open reading frame (ORF) of the target transcript. The number in brackets in the same column shows the distance of this adenosine relative to target site which is defined as ±0. A negative value means 5′ of the target adenosine in the mRNA. A positive value means 3′ of the target adenosine on the target mRNA. The column “ASO” shows the editing results of a symmetric (centred target adenosine) 59 nt long ASO. The column “ASO 2′OMe at all off-target sites” shows the editing results of a symmetric 59 nt long ASO containing 2′OMe modifications at all sites that caused off-target editing in the regular ASO. The column “ASO 2′OMe or GU at off-target sites” shows the editing results of a symmetric 59 nt long ASO containing GU-wobbles 5′ or 3′ of all sites that caused off-target editing in the regular ASO and are amenable to GU-wobbles, and 2′OMe modifications at all sites that caused off-target editing in the regular ASO but are not amenable to GU-wobbles. The given editing yields in % represent the mean±SD of n=3 biological replicates.

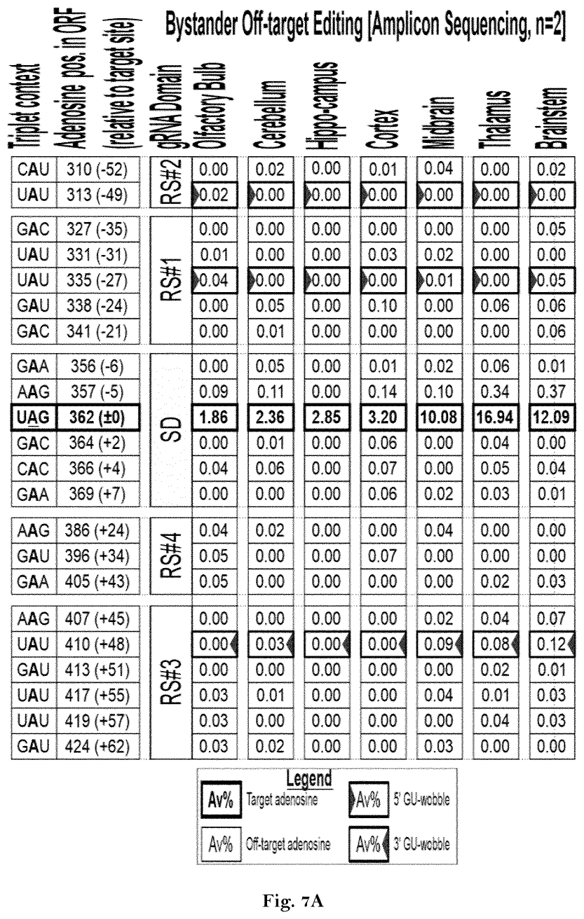

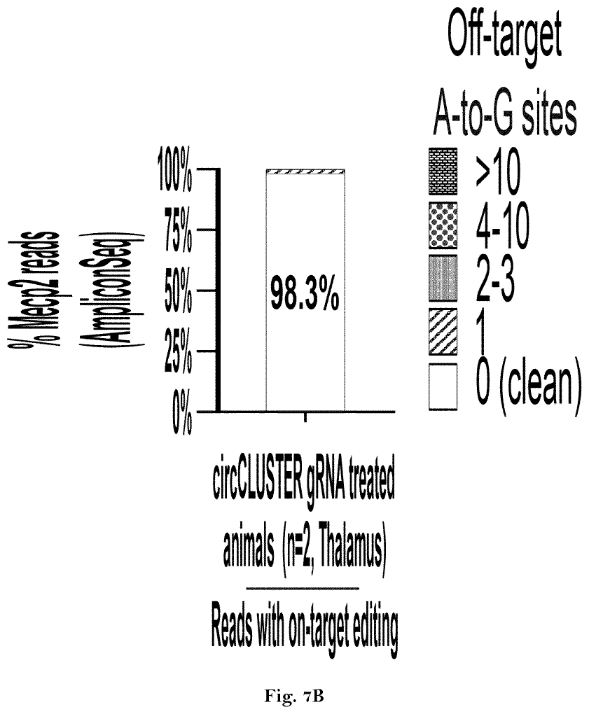

FIG. 7: Off-target editing suppression via GU-wobbles using trans-acting circular CLUSTER gRNAs targeting a murine disease model of Rett syndrome (Examples 18)
7A: Editing yields of all adenosines within the duplex of circular CLUSTER guide RNAs and the endogenous murine MeCP2 W104Amber target mRNA as determined by Amplicon sequencing. The column “gRNA Domain” indicates to which domain of the guide RNA (Specificity Domain (SD), Recruitment Sequence #1, 2, 3 or 4 (RS #1, 2, 3, 4)) the listed adenosine positions belong to. The columns Olfactory Bulb, Cerebellum, Hippocampus, Cortex, Midbrain, Thalamus and Brainstem, show the editing results achieved in the indicated brain region using the Tornado expression system circularized CLUSTER guide RNA “Circular Split-R/G-V24 & GU”. It consists of one specificity domain, four recruitment sequences, and the version 24 of the ADAR recruitment motif (R/G motif) and three GU wobbles preventing off-target events at the adenosine positions 313, 335 and 410. For a detailed description of the rows and columns reference is made to FIG. 5A. The given editing yields in % represent the mean of n=2 animals.
7B: Percentage of mMeCP2 reads from the Thalamus of n=2 circular CLUSTER guide RNA treated Rett animals as determined by Amplicon sequencing that are successfully edited at the target site and additionally contain either 0 (clean), 1, 2-3, 4-10 or >10 bystander off-target events.

### EXAMPLES

The examples shown in the following are merely illustrative and shall describe the present invention in a further way. These examples shall not be construed to limit the present invention thereto.

If not stated otherwise, the nucleic acid sequences provided herein are printed from 5′ to 3′. In other terms, the first nucleotide residue in a nucleic acid sequence printed herein is—if not stated otherwise—the 5′-terminus of said nucleic acid sequence. Amino acid sequences—if not stated otherwise—are printed from the N-terminus to the C-terminus.

### Example 1: UG-Wobbles Increase the RNA Editing Yield at Adjacent Adenosines

In order to investigate the effects of UG wobbles (see FIG. 1A) at nearest neighbor sites (see FIG. 1B) cis acting guide RNAs located within the 3′-UTR of an eGFP reporter (see FIG. 1C) were encoded on plasmids and transfected into 293 cells containing one Flp-In copy of either ADAR1 p110, ADAR1 p150, or ADAR2 under control of a doxycycline inducible CMV promoter.

Experimental settings: Editing of cis-acting reporter constructs using one integrated copy of ADAR in Flp-In T-REX cells. 2.5×10⁵ ADAR1 (p110 or p150) or 3×10⁵ ADAR2 Flp-In T-REx cells were seeded on poly-d-lysine (PDL)-coated 24-well plates in 500 μl of DMEM, 10% FBS and 10 ng/ml doxycycline. After 24 h, cells were transfected with 1,600 ng of cis-acting reporter construct plasmid (NucleoSpin Plasmid Transfection-grade, Macherey Nagel, no. 740490) using a 1:3 ratio of Lipofectamine-2000 (ThermoFisher, no. 11668019). Cells were harvested 72 h after transfection. After RNA-isolation, DNase-I digestion and RT-PCR this was followed by sanger-sequencing.

As shown in FIGS. 1D, IF (ADAR1 p110 Flp-In T-REx cells), and 1I (ADAR1 p110, p150 and ADAR2 Flp-In T-REX cells), cis acting guide RNAs containing an UG-wobble adjacent to an adenosine increase its editing yield when compared to the same triplet without a wobble. This was exemplary shown for 5′-UAG triplet for all three ADAR proteins and for the 5′-AAG and 5′-CAG triplets for the ADAR1 p110 isoform.

### Example 2: GU-Wobbles Reduce the RNA Editing Yield at Adjacent Adenosines

In order to investigate the effects of UG wobbles (see FIG. 1A) at nearest neighbor sites (see FIG. 1B) cis acting guide RNAs located within the 3′-UTR of an eGFP reporter (see FIG. 1C) were encoded on plasmids and transfected into 293 cells containing one Flp-In copy of either ADAR1 p110, ADAR1 p150, or ADAR2 under control of a doxycycline inducible CMV promoter.

Experimental settings: Editing of cis-acting reporter constructs using one integrated copy of ADAR in Flp-In T-REx cells. 2.5×10⁵ ADAR1 (p110 or p150) or 3×10⁵ ADAR2 Flp-In T-REx cells were seeded on poly-d-lysine (PDL)-coated 24-well plates in 500 μl of DMEM, 10% FBS and 10 ng/ml doxycycline. After 24 h, cells were transfected with 1,600 ng of cis-acting reporter construct plasmid (NucleoSpin Plasmid Transfection-grade, Macherey Nagel, no. 740490) using a 1:3 ratio of Lipofectamine-2000 (ThermoFisher, no. 11668019). Cells were harvested 72 h after transfection. After RNA-isolation, DNase-I digestion and RT-PCR this was followed by sanger-sequencing.

As shown in FIGS. 1E, 1F (both ADAR1 p110 Flp-In T-REx cells), and 1J ((ADAR1 p110, p150 and ADAR2 Flp-In T-REX cells) cis acting guide RNAs containing an GU-wobble adjacent to an adenosine reduce its editing yield when compared to the same triplet without a wobble. This reduction is stronger than the one caused by GA mismatches, which are the prior art solution to reduce RNA editing yields. This was shown for the 5′-UAG triplet for all three ADAR proteins and for the 5′-UAU, 5′-UAC, 5′-UAA and 5′-AAU triplets for the ADAR1 p110 isoform. This list contains the majority of triplet contexts that cause the strongest off-target editing events in site-directed RNA editing approaches. The datasets in FIGS. 1E and 1J do also show that the reducing effect of GU-wobbles overrules the increasing effect of UG-wobbles, when comparing the 5′-UUG to the 5′-CUG solutions for the 5′-UAG triplet. In addition, 5′-GU-wobbles seem to show a stronger reduction of the RNA editing yield, compared to 3′-GU-wobbles.

### Example 3: GU-Wobbles can Reduce Off-Target Editing at Adenosines Located Directly 5′ or 3′ of a Target Adenosine without Reduction of the On-Target Editing Yield in Cis-Acting Guide RNA Reporter Constructs

The off-target editing suppression close to the target adenosine, especially of adenosines at nearest neighbor sites next to the target adenosine, is a substantial problem for all encodable SDRE systems, as the on-target yield is in most cases considerably reduced by such attempts when utilizing GA mismatches. The inventor noticed that the natural R/G site within the human GRIA2 mRNA contains a GU wobble that seems to prevent off-target editing at the 5′ nearest neighbor site, without affecting the R/G on-target editing site, which is usually edited with yields of ˜90%. To properly evaluate GU wobbles and GA mismatches for off-target editing suppression at the 5′ and 3′ nearest neighbor sites of a target adenosine, another set of cis-acting constructs was designed. To increase the editing yields and thus make off-target suppression even harder, the previous cis-acting design from two individual duplexes (see FIG. 1C, individual ADAR recruiting moiety duplex and target sequence duplex), which resembles the previously published R/G gRNA design, was changed to one long continuous duplex (see FIG. 1G, ADAR recruiting moiety duplex and target sequence as one long duplex). The two best edible triplets with an adenosine at the nearest neighbor position, which are the 5′-UAA and 5′-AAG triplets were chosen (FIG. 1H, 5′ and 3′ NN V1 constructs). Then, additional versions were created by adding more adenosines 5′ and 3′ of the target triplet (FIG. 1H, 5′ and 3′ NN V2 and V3 constructs), to evaluate also the distance, if any, under which editing would be affected. Due to the GU wobbles sequence restriction both the GU-wobbles and GA mismatches were shifted further away from the target triplet in these constructs. Again the cis acting guide RNA was located within the 3′-UTR of an eGFP reporter (see FIG. 1G) and was encoded on a plasmid for transfection into 293 cells containing one Flp-In copy of ADAR1 p110 under control of a doxycycline inducible CMV promoter.

Experimental settings: Editing of cis-acting reporter constructs using one integrated copy of ADAR1 p110 in Flp-In T-REX cells. 2.5×10⁵ ADAR1 p110 Flp-In T-REx cells were seeded on poly-d-lysine (PDL)-coated 24-well plates in 500 μl of DMEM, 10% FBS and 10 ng/ml doxycycline. After 24 h, cells were transfected with 1,600 ng of cis-acting reporter construct plasmid (NucleoSpin Plasmid Transfection-grade, Macherey Nagel, no. 740490) using a 1:3 ratio of Lipofectamine-2000 (ThermoFisher, no. 11668019). Cells were harvested 72 h after transfection. After RNA-isolation, DNase-I digestion and RT-PCR this was followed by sanger-sequencing.

As shown in FIG. 1H, directly adjacent GU-wobbles seem to be much better suited then GA mismatches to prevent off-target editing at nearest neighbor sites. Adjacent to the 5′ nearest neighbor position a GU-wobble could reduce the off-target editing much more thoroughly then a GA mismatch (FIG. 1H, 5′ NN V1 construct, pos. −1, “Reference” 95±0% vs. “GU at −2 site” 27±1% vs. “GA at −1 site” 83±0), while the editing at the actual target sites stayed basically unchanged for all evaluated 5′ NN constructs, including the GA mismatch containing one. At the 3′ nearest neighbor position both GA mismatch and an adjacent GU wobble suppressed off-target editing to similar levels (FIG. 1H, 3′ NN V1 construct, pos. +1, “Reference” 34±3% vs. “GU at +2 site” 7±1% vs. “GA at +1 site” 8±1%). However, the on-target editing yield dropped considerably for the GA mismatch containing construct, but not the GU wobble containing one (FIG. 1H, 3′ NN V1 construct, pos. +0, “Reference” 97±1% vs. “GU at +2 site” 97±1% vs. “GA at +1 site” 69±1%). When shifting the GU-wobble one nucleotide further away from the nearest neighbor site the suppressing effect was reduced or completely abolished in both 5′ (FIG. 1H, 5′ NN V1 construct, pos. −1, “Reference” 95±0% vs. “GU at −2 site” 27±1% vs. “GU at −3 site” 82±2%) and 3′ direction (FIG. 1H, 3′ NN V1 construct, pos. +1, “Reference” 34±3% vs. “GU at +2 site” 7±1% vs. “GU at +3 site” 38±3%). When comparing GU-wobbles and GA mismatches at the same distance of 1 nt to the nearest neighbor position at which editing should be suppressed, the GU wobbles were superior at both 5′ (FIG. 1H, pos. −1, 5′ NN V1 construct “GU at −2 site” 27±1% vs. 5′ NN V2 construct “GA at −2 site” 89±1%) and 3′ nearest neighbor sites (FIG. 1H, pos. +1, 3′ NN V1 construct “GU at +2 site” 7±1% vs. 3′ NN V2 construct “GA at +2 site” 28±2%). At 2 nt distance however, GA mismatches showed a higher suppression at both the 5′ (FIG. 1H, pos. −1, 5′ NN V2 construct “GU at −3 site” 91±3% vs. 5′ NN V3 construct “GA at −3 site” 11±2%) and the 3′ nearest neighbor sites (FIG. 1H, pos. +1, 3′ NN V2 construct “GU at +3 site” 54±4% vs. 3′ NN V3 construct “GA at +3 site” 42±1%). This might explain why GU-wobbles are superior at nearest neighbor sites. Their suppressing effect is stronger, but seems to descend more rapidly than the one of GA mismatches over distance.

### Example 4: Off-Target Editing Reduction Via GU-Wobbles, GA Mismatches and Uridine Depletion Characterized Using Trans-Acting LEAPER Guide RNAs Targeting the Disease Relevant Transcript AHI W725Amber

To determine if the RNA editing reducing effect of GU-wobbles could be applied in trans, the LEAPER guide RNA system was utilized (Qu, L., et al. (2019). “Programmable RNA editing by recruiting endogenous ADAR using engineered RNAs.” Nat Biotechnol.) The 111 nt long unstructured LEAPER guide RNAs are reverse complementary to their target mRNA, except for a C-A mismatch at the centered target adenosine. As this gRNA design is known for its strong and widespread off-target editing, it represented an optimal test environment to compare GU-wobbles and GA mismatches in trans. First, LEAPER gRNAs containing the same number of GU-wobbles and GA mismatches were compared, and their effect on the on-target yield was evaluated. GU wobbles that prevent RNA editing can only be applied if there is a nearest neighbor uridine next to the off-target adenosine. Thus, all off-target sites caused by LEAPER gRNAs for the disease relevant target AHI W725Amber were characterized and all of the GU amenable off-target sites (GU at GU amenable sites) were used also for the corresponding GA mismatch containing LEAPER gRNA (GA at GU amenable sites). Finally, the prior art solution that uses GA mismatches at all off-target sites and the prior art solution that uses Uridine depletion at all off-target sites were compared with the novel GU&GA solution that combines GU-wobble at amenable sites with GA mismatches at not GU-wobble amenable sites. The target transcript AHI1 encodes the Jouberin protein, and mutations in the expression of the gene are known to cause specific forms of Joubert syndrome.

Experimental settings: Editing of an exogenous human disease relevant transcripts using trans-acting LEAPER guide RNAs and endogenous ADAR1 in Hela cells. HeLa cells (8×10⁴) were seeded in 24-well scale in 500 μl of DMEM and 10% FBS. Cells were transfected 24 h after seeding with 800 ng gRNA plasmid and 200 ng of target-encoding plasmid per well using a plasmid:Lipofectamine-3000 ratio of 1:1.5. Cells were harvested 72 h after transfection. After RNA-isolation, DNase-I digestion and RT-PCR this was followed by sanger-sequencing.

Thus it could be shown that the off-target preventing effect of GU wobbles could indeed be transferred from reporter editing in cis to site-directed RNA editing in trans (FIG. 2A). The on-target editing was clearly increased (˜1.4-fold) when using LEAPER gRNAs containing GU-wobbles compared to GA mismatches at GU-wobble amenable sites (FIG. 2A, LEAPER 77±3%, GU at GU 74±3%, GA at GU 51±20%). The ability of GU wobbles to suppress RNA editing simultaneously at their 5′ and 3′ nearest neighbor positions, allowed in some instances to prevent two off-target events with only one wobble (FIG. 2A, pos. +29 and +31). This allows to minimizes the total number of mismatches/wobbles between mRNA and gRNA that are required to completely prevent off-target editing, which can only benefit the final on-target editing yield. When comparing the prior art solution for LEAPER gRNAs, which is the use of GA mismatches at all off-target sites (GA at all off-target sites), with gRNAs containing a combination of GA mismatches and GU wobbles (GU&GA at all off-target sites) an increased on-target editing for the GU wobble containing gRNA was found (FIG. 2A, GA at all off-target site 26±4%, GU&GA at all off-target sites 45±6%). Interestingly the GU wobble containing gRNAs were also able to prevent off-target editing at sites that could not or only partially be suppressed by GA mismatches (FIG. 2A, GU&GA at all off-target sites vs. GA at all off-target site at pos. −38, −35 and −31). Thus the addition of GU wobbles to the prior art solution simultaneously suppressed off-target editing events strongly and nearly doubled the on-target editing yield. The prior art solution that uses Uridine depletion at all off-target sites (U-depl. at all off-target sites) showed editing yields close to Sanger sequencing background levels (<5%) and was thus clearly outcompeted by the other solutions in this instance.

### Example 5: Off-Target Editing Reduction Via GU-Wobbles and GA Mismatches Characterized Using Trans-Acting LEAPER Guide RNAs Targeting the Disease Relevant Transcript BMPR2 W298Amber

For the same reasons already presented in example 4 and to verify applicability of GU-wobbles in multiple targets in trans, LEAPER guide RNAs were used to target BMPR2 W298Amber. Mutations in bone morphogenetic protein receptor type II (BMPR2) are the most common genetic cause of pulmonary arterial hypertension.

Experimental settings: Editing of an exogenous human disease relevant transcripts using trans-acting LEAPER guide RNAs and endogenous ADAR1 in Hela cells. HeLa cells (8×10⁴) were seeded in 24-well scale in 500 μl of DMEM and 10% FBS. Cells were transfected 24 h after seeding with 800 ng gRNA plasmid and 200 ng of target-encoding plasmid per well using a plasmid:Lipofectamine-3000 ratio of 1:1.5. Cells were harvested 72 h after transfection. After RNA-isolation, DNase-I digestion and RT-PCR this was followed by sanger-sequencing.

Again it could be shown that the off-target preventing effect of GU wobbles could be transferred from reporter editing in cis to site-directed RNA editing in trans (FIG. 2B). The on-target editing was in this example similar for LEAPER gRNAs that contained the same number of GA mismatches or GU wobbles (FIG. 2B, LEAPER 80±3%, GU at GU 57±4%, GA at GU 64±5%). When comparing the prior art solution for LEAPER gRNAs, which is the use of GA mismatches at all off-target sites (GA at all off-target sites), with gRNAs containing a combination of GA mismatches and GU wobbles (GU&GA at all off-target sites), a similar on-target editing for the GU wobble containing gRNA was found (FIG. 2B, GA at all off-target site 54±21%, GU&GA at all off-target sites 53±2%). Again the GU wobble containing gRNAs were also able to prevent off-target editing at sites that could not or only partially be suppressed by GA mismatches (FIG. 2B, BMPR2 GA&GU at all off-target sites vs. GA at all off-target site at pos. −32, +32, +35). Thus the addition of GU wobbles to the prior art solution simultaneously suppressed off-target editing events strongly while achieving the same level of on-target editing.

### Example 6: Off-Target Editing Reduction Via GU-Wobbles and GA Mismatches Characterized Using Trans-Acting LEAPER Guide RNAs Targeting the Disease Relevant Transcript COL3A1 W1278Amber

For the same reasons already presented in example 4 and to verify applicability of GU-wobbles in multiple targets in trans, LEAPER guide RNAs were used to target COL3A1 W1278Amber. Mutations in COL3A1 have been identified to underlie the Ehlers-Danlos syndrome type IV which is an autosomal dominant connective tissue disease.

Experimental settings: Editing of an exogenous human disease relevant transcripts using trans-acting LEAPER guide RNAs and endogenous ADAR1 in Hela cells. HeLa cells (8×10⁴) were seeded in 24-well scale in 500 μl of DMEM and 10% FBS. Cells were transfected 24 h after seeding with 800 ng gRNA plasmid and 200 ng of target-encoding plasmid per well using a plasmid:Lipofectamine-3000 ratio of 1:1.5. Cells were harvested 72 h after transfection. After RNA-isolation, DNase-I digestion and RT-PCR this was followed by sanger-sequencing.

Again it could be shown that the off-target preventing effect of GU wobbles could be transferred from reporter editing in cis to site-directed RNA editing in trans (FIG. 2C). The on-target editing was again clearly increased (˜1.6-fold) when using LEAPER gRNAs containing GU-wobbles compared to GA mismatches at GU-wobble amenable sites (FIG. 2C, LEAPER 63±7%, GU at GU 47±4%, GA at GU 29±3%). The ability of GU wobbles to suppress RNA editing simultaneously at their 5′ and 3′ nearest neighbor positions, allowed in some instances to prevent two off-target events with only one wobble (FIG. 2C, pos. −5 and −3). This allows to minimizes the total number of mismatches/wobbles between mRNA and gRNA that are required to completely prevent off-target editing, which can only benefit the final on-target editing yield. When comparing the prior art solution for LEAPER gRNAs, which is the use of GA mismatches at all off-target sites (GA at all off-target sites), with gRNAs containing a combination of GA mismatches and GU wobbles (GU&GA at all off-target sites), an increased on-target editing for the GU wobble containing gRNA was found (FIG. 2C, GA at all off-target site 21±2%, GA&GU at all off-target sites 41±2%). Thus the addition of GU wobbles to the prior art solution again nearly doubled the on-target editing yield.

### Example 7: Off-Target Editing Reduction Via GU-Wobbles and GA Mismatches Characterized Using Trans-Acting LEAPER Guide RNAs Targeting the Transcript of the Endogenous Housekeeping Gene RAB7A

For the same reasons already presented in example 4 and to verify applicability of GU-wobbles also for endogenous transcripts, LEAPER guide RNAs were used to target an adenosine within the 3′-UTR of RAB7A. Mutations in the RAB7A gene are associated with several diseases including e.g. Charcot-Marie-Tooth Disease

Experimental settings: Editing of the 3′ UTR of the endogenous housekeeping gene RAB7A using trans-acting LEAPER guide RNAs and endogenous ADAR1 in HEK293FT cells. HEK293FT cells (6×10⁴) were seeded in 24-well scale in 450 μl of DMEM and 10% FBS. After 24 h, cells were transfected with 1,200 ng of gRNA plasmid (NucleoSpin Plasmid Transfection-grade, Macherey Nagel, no. 740490) using a 1:3 ratio of FuGene6 (Promega, no. E2691). Forty-eight hours after transfection, cells were harvested. After RNA-isolation, DNase-I digestion and RT-PCR this was followed by sanger-sequencing.

Again it could be shown that the off-target preventing effect of GU wobbles could be transferred from reporter editing in cis to site-directed RNA editing in trans, this time for an endogenous transcript (FIG. 2D). The on-target editing was again increased (˜1.2-fold) when using LEAPER gRNAs containing GU-wobbles compared to GA mismatches at GU-wobble amenable sites (FIG. 2D, LEAPER 54±6%, GU at GU 45±3%, GA at GU 25±3%). In this experiment already the use of GU-wobbles at the GU amenable sites alone prevented all off-target editing and achieved with 45±3% much higher editing then all other evaluated guide RNAs including the prior art solution containing GA mismatches at all off-target sites (FIG. 2D, GU at GU 45±3% vs. GA at all off-target sites 29±2%).

### Example 8: Off-Target Editing Reduction Via GU-Wobbles and GA Mismatches Using Trans-Acting Circular LEAPER Guide RNAs Targeting the Murine Transcript MeCP2 W104Amber

For the same reasons already presented in example 4 and to verify applicability of GU-wobbles in multiple targets in trans, LEAPER guide RNAs were used to target mMeCP2 W104Amber. The mMeCP2 W104Amber mutation is disease causing in a mouse model of Rett syndrome. To verify applicability of GU-wobbles also in systems that use circular designs, most LEAPER guide RNAs used in this example were circularized using the Tornado expression system adopted for guide RNAs from Litke, J. L. and S. R. Jaffrey (Highly efficient expression of circular RNA aptamers in cells using autocatalytic transcripts. Nat Biotechnol 37(6): 667-675″ (2019), similar to Katrekar, D., et al. (Efficient in vitro and in vivo RNA editing via recruitment of endogenous ADARs using circular guide RNAs. Nat Biotechnol (2022)), and Yi, Z., et al. (Engineered circular ADAR-recruiting RNAs increase the efficiency and fidelity of RNA editing in vitro and in vivo. Nat Biotechnol. (2022)). The Tornado system employs two ribozymes flanking the guide RNA to excise it and create overhangs that allow for ligation via the endogenous RNA ligase RtcB. The resulting circular guide RNA is more stable due to its resistance against exonucleases.

Experimental settings: Editing of an exogenous murine transcript using trans-acting circular LEAPER guide RNAs and endogenous ADAR1 in Hela cells. HeLa cells (8×10⁴) were seeded in 24-well scale in 500 μl of DMEM and 10% FBS. Cells were transfected 24 h after seeding with 800 ng gRNA plasmid and 200 ng of target-encoding plasmid per well using a plasmid:Lipofectamine-3000 ratio of 1:1.5. Cells were harvested 72 h after transfection. After RNA-isolation, DNase-I digestion and RT-PCR this was followed by sanger-sequencing.

Thus it could be shown that the off-target preventing effect of GU wobbles could be transferred from reporter editing in cis to site-directed RNA editing using circular guide RNAs in trans (FIG. 2E). Although the circularization did in this example only increased the off-target editing (LEAPER linear vs. LEAPER circular) the latter could still be reduced by GU-wobbles. The on-target editing was again clearly increased (˜1.6-fold) when using LEAPER gRNAs containing GU-wobbles at amenable sites compared to prior art guide RNAs containing exclusively GA mismatches (FIG. 2D, LEAPER 54±6%, GU at GU 45±3%, GA at GU 25±3%).

### Example 9: Off-Target Editing Reduction Via GU-Wobbles and GA Mismatches Using Trans-Acting 2×BoxB Guide RNAs and the λN-BoxB-ADAR System Targeting the Disease Relevant Transcript AHI W725Amber

To verify applicability of GU-wobbles not only in trans and for multiple targets, but also for multiple different RNA editing systems, 2×BoxB guide RNAs of the λN-BoxB-ADAR system were used to target the disease relevant transcript AHI W725Amber. 2×BoxB guide RNAs are explained in detail in Montiel-Gonzalez, M. F., et al. (An efficient system for selectively altering genetic information within mRNAs. Nucleic Acids Res 44(21): e157(2016)). The target transcript AHI1 encodes the Jouberin protein, and mutations in the expression of the gene are known to cause specific forms of Joubert syndrome. The λN-BoxB-ADAR system applied here utilizes an overexpressed engineered fusion protein consisting of an ADAR2 deaminase domain (DD) with a E488Q hyperactive mutant and four λN-peptides (4×λN-ADAR2-DD-E488Q). Its guide RNA consists of three antisense parts of 10 nt, 29 nt and again 10 nt length interrupted by two BoxB motifs for recruitment of the artificial editase 4×λN-ADAR2-DD-E488Q. The centered antisense part contains the counterbase for the target adenosine placing it in a CA mismatch at position 15 counted from the 5′ end of this antisense part. The other two 10 nt long antisense parts bind the target transcript 5′ and 3′ of the binding site of the central antisense part (29 nt) but with two 3 nt spacers in between to considering the space required for the BoxB motifs. First, 2×BoxB gRNAs containing the same number of GU-wobbles and GA mismatches were compared. GU wobbles that prevent RNA editing can only be applied if there is a nearest neighbor uridine next to the off-target adenosine. Thus, all off-target sites caused by 2×BoxB guide RNA for the disease relevant target AHI W725Amber were characterized and all of the GU amenable off-target sites (GU at GU amenable sites) were also used for the corresponding GA mismatch containing 2×BoxB guide RNA (GA at GU amenable sites). Finally, the prior art solution that uses GA mismatches at all off-target sites was compared with the novel GU&GA solution that combines GU-wobble at amenable sites with GA mismatches at not GU-wobble amenable sites.

Experimental settings: Editing of AHI W725Amber using trans-acting 2×BoxB guide RNAs and overexpressed 4×λN-ADAR2-E488Q in Hela cells. HeLa cells (8×10⁴) were seeded in 24-well scale in 500 μl of DMEM and 10% FBS. Cells were transfected 24 h after seeding with 800 ng gRNA plasmid (2×BoxB-guide RNA), 200 ng of target-encoding plasmid (AHI) and 200 ng editase-encoding plasmid (4×λN-ADAR2-DD-E488Q) per well using a plasmid:Lipofectamine-3000 ratio of 1:1.5. Cells were harvested 72 h after transfection. After RNA-isolation, DNase-I digestion and RT-PCR this was followed by sanger-sequencing.

It could be shown that the off-target preventing effect of GU wobbles could be applied to the trans acting λN-BoxB site-directed RNA editing system (FIG. 3A). The off-target editing suppression of GU wobbles seems again stronger than the one caused by GA mismatches (GU at GU vs. GA at GU at pos. −2 and −8, GA at all off-target sites vs. GU&GA at all off-target sites at pos. −8). However, this effect is less pronounced in this example compared to the LEAPER guide RNA system. The “4×λN-ADAR2-DD-E488Q, No gRNA” and “2×BoxB-gRNA, No Editase” negative controls show that the λN-BoxB-ADAR system requires all of its components to function. Its editing is thus indeed caused by the overexpressed 4×λN-ADAR2-DD-E488Q editase and not by endogenous ADAR. As the engineered λN-BoxB RNA base editing approach is lacking ADAR's dsRNA binding domains, it can be assumed that the substrate interaction of the deaminase domain alone is sufficient for wobbles to function as editing modulators. Thus wobbles should be widely applicable in the RNA editing field, even for systems that utilize only the deaminase domain of a deaminase.

### Example 10: Off-Target Editing Reduction at Nearest Neighbor Adenosines Via GU-Wobble or GA Mismatch Using Trans-Acting 2×BoxB Guide RNAs Targeting the Disease Relevant Transcripts AHI W725X, BMPR2 W298X and COL3A1 W1278Amber

The suppression of bystander off-target editing in closest proximity to an on-target adenosine is a common problem for all fully encoded RNA base editing systems. In particular, the GA mismatch strategy can lead to substantial loss in on-target editing yield. Here, it was tested if the GU-wobble base pair might help to suppress bystander editing at 5′, 3′ or both 5′ and 3′ nearest neighbor sites, without affecting the on-target editing site. After testing for this in cis (see example 3) and showing its successful in the 5′ or 3′ direction in trans using CLUSTER guide RNAs (see example 13 and 14), 2×BoxB guide RNAs were designed to target a 5′-UAAAU site at AHI K706, a 5′-UAAU site at AHI I1179, a 5′-UAAU site at BMPR2 N1005, a 5′-UAAU site at COL3A1 N1244 and a 5′-UAAG site at BMPR2 K984 (on-target adenosine underlined, GA mismatch in bold, GU wobble in italic) by harnessing exogenous 4×λN-ADAR2-E488Q.

Experimental settings: Editing of AHI W725Amber, BMPR2 W298X Amber or COL3A1 W1278 Amber transcripts using trans-acting 2×BoxB guide RNAs and overexpressed 4×λN-ADAR2-E488Q in HeLa cells. HeLa cells (8×10⁴) were seeded in 24-well scale in 500 μl of DMEM and 10% FBS. Cells were transfected 24 h after seeding with 800 ng gRNA plasmid (2×BoxB-guide RNA), 200 ng of target-encoding plasmid (AHI, BMPR2, COL3A1) and 200 ng editase-encoding plasmid (4×λN-ADAR2-DD-E488Q) per well using a plasmid:Lipofectamine-3000 ratio of 1:1.5. Cells were harvested 72 h after transfection. After RNA-isolation, DNase-I digestion and RT-PCR this was followed by sanger-sequencing.

As expected, the reference guide RNAs gave good on-target yields (FIG. 3B, Reference, On-target AHI K706 50%, BMPR2 K984 44%, AHI 11179 34%, BMPR2 N1005 60%, COL3A1 N1244 57%) but contaminated with extensive bystander editing at the proximal adenosine(s) (FIG. 3B, Reference, AHI K706 5′-NN off-target 47%, AHI K706 3′-NN off-target 14%, BMPR2 K984 5′-NN off-target 24%, AHI 11179 3′-NN off-target 22%, BMPR2 N1005 3′-NN off-target 33%, COL3A1 N1244 3′-NN off-target 15%). Consistently the GU wobble solution could suppress more off-target editing (FIG. 3B, GA mismatch vs. GU-wobble, AHI K706 5′-NN off-target 12% vs. 5%, AHI K706 3′-NN off-target 4% vs. 6%, BMPR2 K984 5′-NN off-target 8% vs. 6%, AHI 11179 3′-NN off-target 8% vs. 5%, BMPR2 N1005 3′-NN off-target 8% vs. 6%, COL3A1 N1244 3′-NN off-target 6% vs. 4%) while simultaneously sustaining higher on-target editing compared to the GA mismatch solution (FIG. 3B, GA mismatch vs. GU-wobble, AHI K706 on-target 10% vs. 29%, BMPR2 K984 on-target 30% vs. 34%, AHI 11179 on-target 27% vs. 32%, BMPR2 N1005 on-target 50% vs. 61%, COL3A1 N1244 on-target 46% vs. 63%). In many cases (AHI 11179, BMPR2 N1005 and COL3A1 N1244) the on-target editing yield was not negatively affected by GU-wobbles, while off-target editing was still successfully suppressed. In sharp contrast to GA mismatches, which drastically reduced on-target editing in the 5′-UAAAU-3′ context of the AHI K706 target site, GU wobbles still achieved a good editing yield of 29%. These results highlight again the strength of the GU-wobble strategy to suppress bystander editing precisely in A-rich triplets.

### Example 11: Off-Target Editing Reduction Via GU-Wobbles and GA Mismatches Using Trans-Acting DR Guide RNAs and the Cas13b-ADAR System Targeting the Disease Relevant Transcript AHI W725Amber

To verify applicability of GU-wobbles not only in trans and for multiple targets, but also for multiple different RNA editing systems, direct repeat (DR) guide RNAs of the Cas13b system were used to target the disease relevant transcript AHI W725Amber. DR guide RNAs are explained in detail in Cox, D. B. T., et al. (RNA editing with CRISPR-Cas13. Science (2017)). The target transcript AHI1 encodes the Jouberin protein, and mutations in the expression of the gene are known to cause specific forms of Joubert syndrome. The Cas13b-ADAR system applied here utilizes an overexpressed engineered fusion protein consisting of an ADAR2 deaminase domain (DD) with a E488Q hyperactive mutant and a Cas13b protein (dPspCas13b-ADAR2-DD-E488Q). Its guide RNA consists of a 3′ terminal 36 nt long DR for recruitment of the artificial editase dPspCas13b-ADAR2-DD-E488Q and a 5′ terminal 51 nt long antisense part that contains the counterbase for the target adenosine placed in a CA mismatch at position 18 counted from the 5′ end of this antisense part. First, DR gRNAs containing the same number of GU-wobbles and GA mismatches were compared. GU wobbles that prevent RNA editing can only be applied if there is a nearest neighbor uridine next to the off-target adenosine. Thus, all off-target sites caused by DR guide RNA for the disease relevant target AHI W725Amber were characterized, and all of the GU amenable off-target sites (GU at GU amenable sites) were also used for the corresponding GA mismatch containing DR guide RNA (GA at GU amenable sites). Finally, the prior art solution that uses GA mismatches at all off-target sites was compared with the novel GU&GA solution that combines GU-wobble at amenable sites with GA mismatches at not GU-wobble amenable sites.

Experimental settings: Editing of AHI W725Amber using trans-acting Directed-Repeat (DR) guide RNAs and overexpressed dPsp-Cas13b-ADAR2-E488Q in Hela cells. Hela cells (8×10±) were seeded in 24-well scale in 500 μl of DMEM and 10% FBS. Cells were transfected 24 h after seeding with 800 ng gRNA plasmid (Direct-Repeat-guide RNA), 200 ng of target-encoding plasmid (AHI) and 200 ng editase-encoding plasmid (dPspCas13b-ADAR2-DD-E488Q) per well using a plasmid:Lipofectamine-3000 ratio of 1:1.5. Cells were harvested 72 h after transfection. After RNA-isolation, DNase-I digestion and RT-PCR this was followed by sanger-sequencing.

It could be shown that the off-target preventing effect of GU wobbles could be applied to the trans acting Cas13b-ADAR site-directed RNA editing system (FIG. 4). The off-target editing suppression of GU wobbles seems again stronger than the one caused by GA mismatches (GA at all off-target sites vs. GU&GA at all off-target sites at pos. −8). However, this effect is less pronounced in this example compared to the LEAPER guide RNA system. The “dPspCas13b-ADAR2-DD-E488Q, No gRNA” negative control shows that the overexpressed editase does require a guide RNA for specific site-directed RNA editing of its AHI W725Amber target transcript. However, the “DR gRNA, No Editase” negative control shows that the DR guide RNA alone, without overexpression of an editase, seems to be sufficient to cause most of the detected on-target editing seen in the other samples. The DR guide RNAs are probably recruiting endogenous ADARs to achieve this editing. Nevertheless, GU-wobbles were applicable to the Cas13b-ADAR site-directed RNA editing system.

### Example 12: Off-Target Editing Reduction Via GU-Wobbles and GA Mismatches Using Trans-Acting CLUSTER Guide RNAs Targeting the Disease Relevant Transcript BMPR2 W298Amber

To verify applicability of GU-wobbles not only in trans and for multiple targets, but also for multiple different RNA editing systems, endogenous ADAR recruiting CLUSTER guide RNAs were used to target the disease relevant transcript BMPR2 W298Amber. CLUSTER guide RNA are explained in detail in Reautschnig, P., et al. (CLUSTER guide RNAs enable precise and efficient RNA editing with endogenous ADAR enzymes in vivo. Nat Biotechnol 40(5): 759-768(2022). Mutations in bone morphogenetic protein receptor type II (BMPR2) are the most common genetic cause of pulmonary arterial hypertension. The CLUSTER approach minimizes the presence of editable adenosines within the duplex of the CLUSTER guide RNA and the target transcript by fragmenting the guide RNA into several parts which bind the target in areas selected for the absence of editable adenosine bases. Beside avoidance of bystander editing, this fragmentation of binding sequences in CLUSTER guide RNAs furthermore improves editing efficiency by avoiding misfolding of the guide RNA into a nonproductive secondary structure. Finally, a high degree of freedom to choose from the sequence of CLUSTER guide RNAs will help to create CLUSTER guide RNAs to avoid interference with target transcript processing like splicing. This is a potentially severe disadvantage of the LEAPER approach, where guide RNAs can interfere with splicing and other posttranscriptional procession of the target transcript by sequestering certain signals, like splice sites and other cis-acting splice-modulating sequences. A CLUSTER guide RNA consist of a 5′ ADAR recruiting domain of 55 nt length called R/G motif and a structured and in-silico optimized antisense part. The antisense part consists of a specificity domain and a cluster of 3-9 recruitment sequences (RS) of 15-20 nt length each connected by triple-adenosine linkers. The 20 nt long specificity domain contains the counterbase for the target adenosine placed in a CA mismatch at position 8 counted from the 5′ end of the antisense part. The recruitment sequences (RS) are in-silico optimized to minimize the presence of editable adenosines. By allowing only adenosines in a 5′-GAN triplet context within the binding sites of the recruitment sequences off-target editing can be avoided. However, the available sequence space for in-silico optimization could be greatly increased by also allowing for 5′-UAB and 5′-BAU triplets (B=C, G, U), at which off-target editing could be easily controlled with GU wobble base pairs. This is highly desired as CLUSTER guide RNAs often give higher editing yields if the recruitment sequences bind within a window of a few hundred nucleotides. To test this, a regular CLUSTER gRNA with 20 nt specificity domain and a cluster of three RS (each 20 nt) targeting BMPR2 was generated. Due to the A-rich sequence context, a 840 nt space is needed to place the TS and all three RS on the BMPR2 transcript. This guide RNA was called “distant” (FIG. 5A, No-block (distant). Next, 5′-UAB and 5′-BAU triplets were allowed in the in-silico optimization process, and a guide RNA of equal design that could be placed within 88 nt on the BMPR2 transcript was generated. All guide RNAs using this cluster of recruitment sequences were called “close”. Three variants of this guide RNA were then evaluated (FIG. 5A, No-block close, GU-block close, GA-block close). The No-block guide RNA does contain adenosines in 5′-UAB and 5′-BAU triplet context within the RS binding sites, but does not prevent off-target editing via GU-wobbles or GA mismatches. The GU-block guide RNA uses GU-wobbles to prevent off-target editing at some of these sites that were suspected to be particularly prone to off-target editing. The GA-block guide RNA uses GA mismatches to prevent off-target editing at the same sites as the GU-block guide RNA does.

Experimental settings: Editing of an exogenous human disease relevant transcripts using trans-acting CLUSTER guide RNAs and endogenous ADAR1 in Hela cells. HeLa cells (8×10⁴) were seeded in 24-well scale in 500 μl of DMEM and 10% FBS. Cells were transfected 24 h after seeding with 800 ng gRNA plasmid and 200 ng of target-encoding plasmid per well using a plasmid:Lipofectamine-3000 ratio of 1:1.5. Cells were harvested 72 h after transfection. After RNA-isolation, DNase-I digestion and RT-PCR this was followed by sanger-sequencing.

The new close guide RNA design gave clearly better on-target editing then the old, “distant” design (FIG. 5A, No-block close 47±3% vs. No-block distant 28±3%). However, as expected, the RS now induced some bystander editing, which could be controlled by GU wobble base pairs. Compared to the GA-block, the GU-block gave better on-target editing yields (43% versus 35%).

### Example 13: Off-Target Editing Reduction at a 5′ Nearest Neighbor Adenosine Via GU-Wobble, GA Mismatch or Uridine Depletion Using Trans-Acting CLUSTER Guide RNAs Targeting the Disease Relevant Transcript BMPR2 W298Amber at K983 (Target Triplet: 5′-AAG)

The suppression of bystander off-target editing in closest proximity to an on-target adenosine is a common problem for all fully encoded RNA base editing systems. The GA mismatch strategy as well as the Uridine depletion strategy can both lead to substantial loss in on-target editing yield. Here, it was tested if the GU-wobble base pair might help to suppress bystander editing at the 5′ nearest neighbor site, without affecting the on-target editing. After testing for this in cis (see example 3), CLUSTER guide RNAs (3×20 nt recruitment sequence and 20 nt targeting sequence) were designed to target a 5′-UAAG site in BMPR2 (K984) by harnessing endogenous HeLa ADAR. The on-target adenosine is located in a 5′-AAG triplet context (target adenosine underlined), with a 5′ off-target adenosine in a 5′-UAAtriplet context (target adenosine underlined, GU-wobble in italic, GA mismatch or Uridine depletion in bold). The GA mismatch or the Uridine depletion site are located directly at the off-target Adenosine. The GU-wobble is located one basepair 5′ of the off-target adenosine (5′-UAAG, on-target adenosine underlined, GU-wobble position at the 5′-terminal uridine in italic, GA mismatch or Uridine depletion in bold). Editing at the on-target site results in a K983R mutation (5′-AAG to 5′-AIG). Editing at both sites results in a K983G mutation (5′-AAG to 5′-IIG). The CLUSTER approach is explained in Examples 11 and 12.

Experimental settings: Editing of an exogenous human disease relevant transcripts using trans-acting CLUSTER guide RNAs and endogenous ADAR1 in HeLa cells. HeLa cells (8×10⁴) were seeded in 24-well scale in 500 μl of DMEM and 10% FBS. Cells were transfected 24 h after seeding with 800 ng gRNA plasmid and 200 ng of target-encoding plasmid per well using a plasmid:Lipofectamine-3000 ratio of 1:1.5. Cells were harvested 72 h after transfection. After RNA-isolation, DNase-I digestion and RT-PCR this was followed by sanger-sequencing.

As expected, the reference guide RNAs gave good on-target yields (FIG. 5B, Reference, On-target 59.2%) but contaminated with strong bystander editing at the proximal adenosine (FIG. 5B, Reference, 5′-NN Off-target 29.6%). While a strategically placed GU wobble base pair was able to fully suppress bystander editing (FIG. 5B, GU-wobble, 5′-NN Off-target 2.7%), this was not the case with the GA mismatch strategy where only a partial suppression of bystander editing was achieved (FIG. 5B, GA mismatch, 5′-NN Off-target 14%). The Uridine depletion solution strongly increased bystander editing (FIG. 5B, U-depletion, 5′-NN Off-target 38.4%). Notably, not only the bystander control, but also the on-target editing yield was overall much better with the GU wobble base pair (FIG. 5B, GU-wobble vs. GA mismatch vs. U-depletion, On-target, 45.6% vs. 36.2% vs. 7.1%) highlighting the strength of the strategy to suppress bystander editing precisely in A-rich triplets.

### Example 14: Off-Target Editing Reduction at a 3′ Nearest Neighbor Adenosine Via GU-Wobble, GA Mismatch, or Uridine Depletion Using Trans-Acting CLUSTER Guide RNAs Targeting the Disease Relevant Transcript COL3A1 W1278Amber at N1244 (Target Triplet: 5′-UAA)

The suppression of bystander off-target editing in closest proximity to an on-target adenosine is a common problem for all fully encoded RNA base editing systems. The GA mismatch strategy as well as the Uridine depletion strategy can both lead to substantial loss in on-target editing yield. Here we tested if the GU-wobble base pair might help to suppress bystander editing at the 5′ nearest neighbor site, without affecting the on-target editing site. After testing for this in cis (see example 3) and showing its successful in the 5′ direction in trans (see example 13), CLUSTER guide RNAs (3×20 nt recruitment sequence and 20 nt targeting sequence) were designed to target a 3′-UAAU site in COL3A1 (N1244) by harnessing endogenous HeLa ADAR. The on-target adenosine is located in a 5′-UAA triplet context (target adenosine underlined), with a 3′ off-target adenosine in a 5′-AAU triplet context (target adenosine underlined, GU-wobble in italic, GA mismatch or Uridine depletion in bold). The GA mismatch or the Uridine depletion site are located directly at the off-target Adenosine. The GU-wobble is located one basepair 3′ of the off-target adenosine (5′-UAAU, on-target adenosine underlined, GU-wobble in italic, GA mismatch or Uridine depletion in bold). Editing at the on-target site results in a N1244D mutation (5′-AAU to 5′-IAU). Editing at both sites results in a N1244G mutation (5′-AAU to 5′-IIG). The CLUSTER approach is explained in example 11 and 12.

Experimental settings: Editing of an exogenous human disease relevant transcripts using trans-acting CLUSTER guide RNAs and endogenous ADAR1 in HeLa cells. HeLa cells (8×10⁴) were seeded in 24-well scale in 500 μl of DMEM and 10% FBS. Cells were transfected 24 h after seeding with 800 ng gRNA plasmid and 200 ng of target-encoding plasmid per well using a plasmid:Lipofectamine-3000 ratio of 1:1.5. Cells were harvested 72 h after transfection. After RNA-isolation, DNase-I digestion and RT-PCR this was followed by sanger-sequencing.

As expected, the reference guide RNAs gave good on-target yields (FIG. 5C, Reference, On-target 41.5%) but contaminated with some bystander editing at the proximal adenosine (FIG. 5C, Reference, 3′-NN Off-target 5.8%). While a strategically placed GU wobble base pair was able to fully suppress bystander editing (FIG. 5C, GU-wobble, 3′-NN Off-target 3.4%), this was not the case with the GA mismatch strategy where no suppression of bystander editing was achieved (FIG. 5C, GA mismatch, 3′-NN Off-target 6.5%). The Uridine depletion solution showed bystander editing close to the negative control (FIG. 5C, U-depletion, Off-target 4.2%), but simultaneously obliterated nearly all on-target editing. Notably, not only the bystander control, but also the on-target editing yield was overall much better with the GU wobble base pair (FIG. 5C, GU-wobble vs. GA mismatch vs. U-depletion, On-target, 41.5% vs. 15.8% vs. 2.9%) highlighting the strength of the strategy to suppress bystander editing precisely in A-rich triplets.

### Example 15: On-Target Editing Boost Via UG-Wobbles, CA Mismatches, or a Combination of Both Using Trans-Acting CLUSTER Guide RNAs Targeting the Endogenous Housekeeping Genes ACTB, GUSB and NUP43 at Silent Editing Sites

It is intriguing to speculate that the boosting effect of the UG-wobbles, previously seen in cis in Example 1, could be combined with the activating effect of the CA mismatch at the on-target site in trans. Thus, CLUSTER guide RNAs (3×20 nt recruitment sequence and 20 nt targeting sequence) were designed to target 5′-UAG sites in the endogenous transcripts ACTB 3′UTR, GUSB L456L and NUP43 V233V by harnessing endogenous HeLa ADAR. The CLUSTER approach is explained in Examples 11 and 12.

Experimental settings: Editing of the endogenous housekeeping genes ACTB, GUSB and NUP43 using trans-acting CLUSTER guide RNAs and endogenous ADAR1 in HEK293FT cells. HEK293FT cells (6×10⁴) were seeded in 24-well scale in 450 μl of DMEM and 10% FBS. After 24 h, cells were transfected with 1,200 ng of gRNA plasmid (NucleoSpin Plasmid Transfection-grade, Macherey Nagel, no. 740490) using a 1:3 ratio of FuGene6 (Promega, no. E2691). Forty-eight hours after transfection, cells were harvested. After RNA-isolation, DNase-I digestion and RT-PCR this was followed by sanger-sequencing.

While a UG wobbles could in case of the ACTB and NUP43 targets indeed result in on-target editing yields similar to a C-A mismatch (FIG. 5D, ACTB CA-only 30.1% vs. UG-only 27.1%, NUP43 CA-only 40.7% vs. UG-only 42.3%), this was not true for GUSB (FIG. 5D, GUSB CA-only 42.8% vs. UG-only 6.4%), and a combination of UG wobble and CA mismatch even reduced the on-target editing yields of GUSB and NUP43 compared to a situation with a C-A mismatch only (FIG. 5D, GUSB CA&UG 23.5% vs. UG-only 42.8%, NUP43 CA&UG 28.8% vs. UG-only 42.3%). While a combination of UG-wobble and CA mismatch seems not to be productive, the fact that UG-wobbles can replace CA mismatches in some instances makes them very interesting e.g. for sequence optimization of guide RNAs.

### Example 16: Off-Target Editing Reduction Via GU-Wobbles Using Trans-Acting Circular CLUSTER Guide RNAs Targeting the Murine Transcript MeCP2 W104Amber

To verify applicability of GU-wobbles not only in trans and for multiple targets, but also for multiple different RNA editing systems that utilize guide RNA circularization, endogenous ADAR recruiting circular CLUSTER guide RNAs were used to target mMeCP2 W104Amber. The mMeCP2 W104Amber mutation is disease causing in a mouse model of Rett syndrome. CLUSTER guide RNA are explained in detail in Reautschnig, P., et al. (CLUSTER guide RNAs enable precise and efficient RNA editing with endogenous ADAR enzymes in vivo. Nat Biotechnol 40(5): 759-768(2022)).

The control of bystander off-target editing via GU wobbles extends the list of allowed triplet contexts for in-silico optimization of the cluster of recruitment sequences from 5′-GAN only to 5′-GAN, 5′-UAB or 5′-BAU (B=C, G, U). Due to this extended sequence space the chances of finding clusters of recruitment sequences that bind close to the specificity domain and simultaneously have a weak secondary structure and thus a higher affinity for their target transcript increases. Both of these factors have a strong impact on the editing yield. The circularization in combination with the modular design principle of CLUSTER guide RNAs further allows to control the exit position of the 5′- and 3′-ends of the target transcript by changing the order of the recruitment sequences within the guide RNA. In the shown example (FIG. 5E) the RS #2 and RS #3 are positioned next to each other within the guide RNA, while their binding sites within the target transcript are far away from each other. Only the circularization brings the recruitments sequences back to the correct order of binding sites within the target transcripts.

The CLUSTER guide RNAs used in this example were circularized using the Tornado expression system adopted for guide RNAs from Litke, J. L. and S. R. Jaffrey (Highly efficient expression of circular RNA aptamers in cells using autocatalytic transcripts. Nat Biotechnol 37(6): 667-675″ (2019). The Tornado system employs two ribozymes flanking the guide RNA to excise it and create overhangs that allow for ligation via the endogenous RNA ligase RtcB. The resulting circular guide RNA is more stable due to its resistance against exonucleases.

Experimental settings: Editing of an exogenous murine transcript using trans-acting circular CLUSTER guide RNAs and endogenous ADAR1 in Hela cells. HeLa cells (8×10⁴) were seeded in 24-well scale in 500 μl of DMEM and 10% FBS. Cells were transfected 24 h after seeding with 800 ng gRNA plasmid and 200 ng of target-encoding plasmid per well using a plasmid:Lipofectamine-3000 ratio of 1:1.5. Cells were harvested 72 h after transfection. After RNA-isolation, DNase-I digestion and RT-PCR this was followed by Sanger-sequencing.

It could be shown that the off-target preventing effect of GU wobbles could be transferred from reporter editing in cis to site-directed RNA editing using circular CLUSTER guide RNAs in trans (FIGS. 5E and 5F). Off-target editing at the adenosine positions 313, 335 and 410 could successfully be reduced to background levels by GU-wobbles (FIG. 5F, pos. 313 “Circular Split-R/G-V24” 24±3% vs. “Circular Split-R/G-V24 & GU” 1±2%, pos. 335 “Circular Split-R/G-V24” 6±1% vs. “Circular Split-R/G-V24 & GU” 0±0%, pos. 410 “Circular Split-R/G-V24” 7±2% vs. “Circular Split-R/G-V24 & GU” 2±1%). At the same time the on-target editing yield was not negatively affected by adding of GU wobbles (FIG. 5F, “Circular Split-R/G-V24” 82±1% vs. “Circular Split-R/G-V24 & GU” 84±1%). In addition, it was shown that ADAR recruitment motifs can form functional substrates even when not being organized as terminal or internal hairpins but instead being formed by split halves of an original motif (e.g. the Split-R/G motif version 21 or 24) that forms an internal duplex but is interrupted by other parts of the guide RNA sequence.

### Example 17: Off-Target Editing Reduction Via GU-Wobbles Using Chemically Modified Antisense Oligonucleotides (ASOs) Targeting the Disease Relevant Transcript PEX1 G843D

To verify applicability of GU-wobbles not only for encodable systems but also for chemically modified ADAR-recruiting antisense oligonucleotides (ASOs) the disease relevant transcript PEX1 G843D was targeted. The concept of RNA editing ASOs is explained in Merkle, T., et al. (2019). “Precise RNA editing by recruiting endogenous ADARs with antisense oligonucleotides.” Nat Biotechnol 37(2): 133-138. PEX1 encodes the peroxisomal biogenesis factor 1 protein, and mutations in the expression of the gene are known to cause Zellweger syndrome. In ADAR-recruiting ASOs, the strategic placement of chemical modifications allows to control bystander events. However, dense chemical modification, for example with 2′-O-methylated ribose (2′-OMe), can interfere strongly with editing. Thus, G·U wobble base pairs were evaluated in a case where additional chemical modifications diminished the on-target efficiency. The ASO applied here is chemically modified (phosphorothioate linkage, 2′-OMe end-blocked), 59 nt long, symmetric, and unstructured. The centered antisense part contains the counterbase for the target adenosine placing it in a CA mismatch at position 30 counted from the 5′ end of this antisense part. To achieve editing the ASO recruits endogenous ADAR enzymes.

Experimental settings: Editing of PEX1 G843D using ASOs and endogenous ADARs in Hela cells. HeLa cells (1×10⁵) were seeded in 24-well scale in 500 μl of DMEM and 10% FBS. Cells were transfected 24 h after seeding with 300 ng G843D-mutated PEX1 encoding plasmid per well using a plasmid to FuGene® 6 ratio of 1:3. 48 h after seeding, the chemically modified guide RNAs were forward transfected with 25 μmol guide RNA and 1.5 μl Lipofectamine RNAiMAX reagent per well. 24 h guide RNA post transfection, cells were harvested. After RNA-isolation, DNase-I digestion, RT-PCR and nested PCR this was followed by sanger-sequencing.

While placement of additional 2′-OMe modifications at the −25, −6 and +7 position did control bystander editing, they also reduced the on-target yield drastically (FIG. 6, 34±7 to 12±3%). In contrast, applying G·U wobble base pairs enabled to control bystander editing while preserving the on-target yield (28±8%). The latter example shows that also ASO based approaches can benefit from wobble base pairs to maintain high on-target yields.

### Example 18: In-Vivo Application of GU-Wobble Containing Circular CLUSTER Guide RNAs in a Murine Disease Model of Rett Syndrome

To show that GU-wobble solution can also be applied in-vivo an endogenous ADAR recruiting circular CLUSTER guide RNA was encoded as AAV and retro-orbitally injected into Rett syndrome mice. In the used model Rett syndrome is caused by the mMeCP2 W104Amber mutation. The Rett syndrome model is described in Sinnamon, J. R., et al. (2022). “Targeted RNA editing in brainstem alleviates respiratory dysfunction in a mouse model of Rett syndrome.” Proc Natl Acad Sci USA 119(33): e2206053119. The PHP.eB serotype was used for AAV encapsulation as it allows cargo delivery to the mouse brain after systemic administration. The control of bystander off-target editing via GU wobbles extends the list of allowed triplet contexts for in-silico optimization of the cluster of recruitment sequences from 5′-GAN only to 5′-GAN, 5′-UAB or 5′-BAU (B=C, G, U). Due to this extended sequence space the chances of finding clusters of recruitment sequences that bind close to the specificity domain and simultaneously have a weak secondary structure and thus a higher affinity for their target transcript increases. Both of these factors have a strong impact on the editing yield. The circularization in combination with the modular design principle of CLUSTER guide RNAs further allows to control the exit position of the 5′- and 3′-ends of the target transcript by changing the order of the recruitment sequences within the guide RNA. In the shown example (FIG. 7A) the RS #2 and RS #3 are positioned next to each other within the guide RNA, while their binding sites within the target transcript are far away from each other. Only the circularization brings the recruitments sequences back to the correct order of binding sites within the target transcripts. The CLUSTER guide RNAs used in this example were circularized using the Tornado expression system adopted for guide RNAs from Litke, J. L. and S. R. Jaffrey (Highly efficient expression of circular RNA aptamers in cells using autocatalytic transcripts. Nat Biotechnol 37(6): 667-675″ (2019)). The Tornado system employs two ribozymes flanking the guide RNA to excise it and create overhangs that allow for ligation via the endogenous RNA ligase RtcB. The resulting circular guide RNA is more stable due to its resistance against exonucleases. The used GU-wobbles should prevent off-target events at position +48, −27 and −49 relative to the on-target site. The −5 position stays unprotected and can thus serve as an internal control for the sensitivity of the used read-out method Amplicon sequencing. The latter should be able to detect even very low-level off-target editing events.

Experimental settings: Mice were treated with 4×10¹² viral genomes by retro-orbital injection and sacrificed four weeks later. After dissection of the individual brain regions their RNA was isolated, followed by reverse transcription, amplicon PCR and indexing PCR. Illumina sequencing was performed with a read depth of ˜30,000 reads per sample. After primer trimming the .fasta files were processed using Seqtk trimfq to trim low quality terminal bases via a Phred algorithm. Then base-calls with QV<30 were masked as N. The base-call accuracy of the remaining bases was thus 99.9%. The alignment was performed using BWA-mem against the GRCm38/mm10 reference genome. The editing yield of all adenosines within the guide-RNA-mRNA binding region was determined using Integrative Genomics Viewer 2.16.2. The editing yields at all positions were background corrected with the editing yields determined from scrambled guide RNA treated negative control animals. While the on-target yield was verified for n=5 animals (data not shown), the Amplicon sequencing was performed for the n=2 animals with the highest on-target editing yield and thus the animals with the highest chance for detectable off-target editing. For the Thalamus samples further in-silico processing allowed to determine the frequency of bystander off-target events in reads with successful on-target editing. For this the following additional steps were performed: Samtools 1.9 was used to remove all unmapped reads and all indel containing reads. In two successive trimming steps first soft-clippings and then all sequences outside the gRNA binding region were removed from all reads. Trimming was performed using a custom Python script. Then all reads with successful on-target editing were selected by writing reads with a Cytosine at index chrX:74.037.048 into a new file individually for each animal. Finally, these two files were merged using Samtools 1.9. The merged file was sorted and indexed using IGV 2.16.2. The reads were displayed in Quick Consensus Mode with the coverage allele-fraction threshold being set to 0.5%.

Editing levels differed between brain regions, with the highest on-target editing yields being detected in midbrain, brainstem, and thalamus (FIG. 7A, midbrain: 10.08%, brainstem: 12.09% and thalamus: 16.94%). Similar to the off-target event that was detected at the unprotected-5 position for the same guide RNA in-vitro (FIG. 5F, Circular Split-R/G-V24 & GU) an off-target yield of e.g. 0.34% editing was detected at the −5 position in the Thalamus of Rett mice. This confirms that the Amplicon sequencing was able to detect even low level off-target events with high precision. At the same time no off-target events beyond background levels could be detected in-vivo at any site protected by a GU-wobble (FIG. 7A, position +48, −27 and −49).

In addition, the analysis of all Amplicon sequencing reads with successful editing at the target site in the Thalamus showed that 98.3% of these reads were free of bystander off-target events (FIG. 7B). Thus, the majority of all on-target editing events were very clean. The remaining 1.7% of reads showed exactly one bystander off-target event. The majority of these off-targets were at position-5. Overall, these results show that GU-wobbles can successfully protect off-target prone adenosines in-vivo with exceptional efficiency.

### List of Applied Guide RNAs (for the Ease of Use, the Guide RNAs are Given as Coding DNA Sequences):

| | | | Used | SEQ |
|---|---|---|---|---|
| | | | in | ID |
| PTS # | Guide RNA Name | Sequence 5′->3′ | Figure | NO |
| pTS1444 | Cis-acting_systematic_ | ggtgtcgagaagaggagaacaatatgctaaatgttgttctcgtct | 1E, | 8 |
| | GU-evaluation_AAT-TTA | cctcgacacccagtagattcaacatgctgtaaaaaacagcatgtt | 1F | |
| | | gaatctactg | | |
| pTS1445 | Cis-acting_systematic_ | ggtgtcgagaagaggagaacaatatgctaaatgttgttctcgtctcctcgacacc | 1E, | 9 |
| | GU-evaluation_AAT-TTG | cagtaggttcaacatgctgtaaaaaacagcatgttgaatctactg | 1F | |
| pTS1446 | Cis-acting_systematic_ | ggtgtcgagaagaggagaacaatatgctaaatgttgttctcgtctcctcgacacc | 1D, | 10 |
| | GU-evaluation_AAG-TTC | cagtagcttcaacatgctgtaaaaaacagcatgttgaagctactg | 1F | |
| pTS1447 | Cis-acting_systematic_ | ggtgtcgagaagaggagaacaatatgctaaatgttgttctcgtctcctcgacacc | 1D, | 11 |
| | GU-evaluation_AAG-TTT | cagtagtttcaacatgctgtaaaaaacagcatgttgaagctactg | 1F | |
| pTS1448 | Cis-acting_systematic_ | ggtgtcgagaagaggagaacaatatgctaaatgttgttctcgtctcctcgacacc | 1E, | 12 |
| | GU-evaluation_TAA-ATT | cagtagttacaacatgctgtaaaaaacagcatgttgtaactactg | 1F | |
| pTS1449 | Cis-acting_systematic_ | ggtgtcgagaagaggagaacaatatgctaaatgttgttctcgtctcctcgacacc | 1E, | 13 |
| | GU-evaluation_TAA-GTT | cagtagttgcaacatgctgtaaaaaacagcatgttgtaactactg | 1F | |
| pTS1450 | Cis-acting_systematic_ | ggtgtcgagaagaggagaacaatatgctaaatgttgttctcgtctcctcgacacc | 1E, | 14 |
| | GU-evaluation_TAT-ATA | cagtagatacaacatgctgtaaaaaacagcatgttgtatctactg | 1F | |
| pTS1451 | Cis-acting_systematic_ | ggtgtcgagaagaggagaacaatatgctaaatgttgttctcgtctcctcgacacc | 1E, | 15 |
| | GU-evaluation_TAT-GTA | cagtagatgcaacatgtgtaaaaaacagcatgttgtatctactg | 1F | |
| pTS1452 | Cis-acting_systematic_ | ggtgtcgagaagaggagaacaatatgctaaatgttgttctcgtctcctcgacacc | 1E, | 16 |
| | GU-evaluation_TAT-ATG | cagtaggtacaacatgctgtaaaaaacagcatgttgtatctactg | 1F | |
| pTS1453 | Cis-acting_systematic_ | ggtgtcgagaagaggagaacaatatgctaaatgttgttctcgtctcctcgacacc | 1E, | 17 |
| | GU-evaluation_TAT-GTG | cagtaggtgcaacatgctgtaaaaaacagcatgttgtatctactg | 1F | |
| pTS1454 | Cis-acting_systematic_ | ggtgtcgagaagaggagaacaatatgctaaatgttgttctcgtctcctcgacacc | 1D, | 18 |
| | GU-evaluation_TAG-ATC | cagtagctacaacatgctgtaaaaaacagcatgttgtagctactg | 1E, | |
| | | | 1F | |
| pTS1455 | Cis-acting_systematic_ | ggtgtcgagaagaggagaacaatatgctaaatgttgttctcgtctcctcgacacc | 1E, | 19 |
| | GU-evaluation_TAG-GTC | cagtagctgcaacatgctgtaaaaaacagcatgttgtagctactg | 1F | |
| pTS1456 | Cis-acting_systematic_ | ggtgtcgagaagaggagaacaatatgctaaatgttgttctcgtctcctcgacacc | 1D, | 20 |
| | GU-evaluation_TAG-ATT | cagtagttacaacatgctgtaaaaaacagcatgttgtagctactg | 1F | |
| pTS1457 | Cis-acting_systematic_ | ggtgtcgagaagaggagaacaatatgctaaatgttgttctcgtctcctcgacacc | 1E, | 21 |
| | GU-evaluation_TAG-GTT | cagtagttgcaacatgctgtaaaaaacagcatgttgtagctactg | 1F | |
| pTS1458 | Cis-acting_systematic_ | ggtgtcgagaagaggagaacaatatgctaaatgttgttctcgtctcctcgacacc | 1E, | 22 |
| | GU-evaluation_TAC-ATG | cagtaggtacaacatgctgtaaaaaacagcatgttgtacctactg | 1F | |
| pTS1459 | Cis-acting_systematic_ | ggtgtcgagaagaggagaacaatatgctaaatgttgttctcgtctcctcgacacc | 1E, | 23 |
| | GU-evaluation_TAC-GTG | cagtaggtgcaacatgctgtaaaaaacagcatgttgtacctactg | 1F | |
| pTS1460 | Cis-acting_systematic_ | ggtgtcgagaagaggagaacaatatgctaaatgttgttctcgtctcctcgacacc | 1F | 24 |
| | GU-evaluation_GAA-CTT | cagtagttccaacatgctgtaaaaaacagcatgttggaactactg | | |
| pTS1461 | Cis-acting_systematic_ | ggtgtcgagaagaggagaacaatatgctaaatgttgttctcgtctcctcgacacc | 1F | 25 |
| | GU-evaluation_GAA-TTT | cagtagtttcaacatgctgtaaaaaacagcatgttggaactactg | | |
| pTS1462 | Cis-acting_systematic_ | ggtgtcgagaagaggagaacaatatgctaaatgttgttctcgtctcctcgacacc | 1F | 26 |
| | GU-evaluation_GAT-CTA | cagtagatccaacatgctgtaaaaaacagcatgttggatctactg | | |
| pTS1463 | Cis-acting_systematic_ | ggtgtcgagaagaggagaacaatatgctaaatgttgttctcgtctcctcgacacc | 1F | 27 |
| | GU-evaluation_GAT-TTA | cagtagattcaacatgctgtaaaaaacagcatgttggatctactg | | |
| pTS1464 | Cis-acting_systematic_ | ggtgtcgagaagaggagaacaatatgctaaatgttgttctcgtctcctcgacacc | 1F | 28 |
| | GU-evaluation_GAT-CTG | cagtaggtccaacatgctgtaaaaaacagcatgttggatctactg | | |
| pTS1465 | Cis-acting_systematic_ | ggtgtcgagaagaggagaacaatatgctaaatgttgttctcgtctcctcgacacc | 1F | 29 |
| | GU-evaluation_GAT-TTG | cagtaggttcaacatgctgtaaaaaacagcatgttggatctactg | | |
| pTS1466 | Cis-acting_systematic_ | ggtgtcgagaagaggagaacaatatgctaaatgttgttctcgtctcctcgacacc | 1F | 30 |
| | GU-evaluation_GAG-CTC | cagtagctccaacatgctgtaaaaaacagcatgttggagctactg | | |
| pTS1467 | Cis-acting_systematic_ | ggtgtcgagaagaggagaacaatatgctaaatgttgttctcgtctcctcgacacc | 1F | 31 |
| | GU-evaluation_GAG-TTC | cagtagcttcaacatgctgtaaaaaacagcatgttggagctactg | | |
| pTS1468 | Cis-acting_systematic_ | ggtgtcgagaagaggagaacaatatgctaaatgttgttctcgtctcctcgacacc | 1F | 32 |
| | GU-evaluation_GAG-CTT | cagtagttccaacatgctgtaaaaaacagcatgttggagctactg | | |
| pTS1469 | Cis-acting_systematic_ | ggtgtcgagaagaggagaacaatatgctaaatgttgttctcgtctcctcgacacc | 1F | 33 |
| | GU-evaluation_GAG-TTT | cagtagtttcaacatgctgtaaaaaacagcatgttggagctactg | | |
| pTS1470 | Cis-acting_systematic_ | ggtgtcgagaagaggagaacaatatgctaaatgttgttctcgtctcctcgacacc | 1F | 34 |
| | GU-evaluation_GAC-CTG | cagtaggtccaacatgctgtaaaaaacagcatgttggacctactg | | |
| pTS1471 | Cis-acting_systematic_ | ggtgtcgagaagaggagaacaatatgctaaatgttgttctcgtctcctcgacacc | 1F | 35 |
| | GU-evaluation_GAC-TTG | cagtaggttcaacatgctgtaaaaaacagcatgttggacctactg | | |
| pTS1472 | Cis-acting_systematic_ | ggtgtcgagaagaggagaacaatatgctaaatgttgttctcgtctcctcgacacc | 1F | 36 |
| | GU-evaluation_CAT-GTA | cagtagatgcaacatgctgtaaaaaacagcatgttgcatctactg | | |
| pTS1473 | Cis-acting_systematic_ | ggtgtcgagaagaggagaacaatatgctaaatgttgttctcgtctcctcgacacc | 1F | 37 |
| | GU-evaluation_CAT-GTG | cagtaggtgcaacatgctgtaaaaaacagcatgttgcatctactg | | |
| pTS1474 | Cis-acting_systematic_ | ggtgtcgagaagaggagaacaatatgctaaatgttgttctcgtctcctcgacacc | 1D, | 38 |
| | GU-evaluation_CAG-GTC | cagtagctgcaacatgctgtaaaaaacagcatgttgcagctactg | 1F | |
| pTS1475 | Cis-acting_systematic_ | ggtgtcgagaagaggagaacaatatgctaaatgttgttctcgtctcctcgacacc | 1D, | 39 |
| | GU-evaluation_CAG-GTT | cagtagttgcaacatgctgtaaaaaacagcatgttgcagctactg | 1F | |
| pTS1484 | Cis-acting_systematic_ | ggtgtcgagaagaggagaacaatatgctaaatgttgttctcgtctcctcgacacc | 1E, | 40 |
| | GU-evaluation_AAT-TGA | cagtagagtcaacatgctgtaaaaaacagcatgttgaatctactg | 1F | |
| pTS1485 | Cis-acting_systematic_ | ggtgtcgagaagaggagaacaatatgctaaatgttgttctcgtctcctcgacacc | 1F | 41 |
| | GU-evaluation_AAG-TGC | cagtagcgtcaacatgctgtaaaaaacagcatgttgaagctactg | | |
| pTS1486 | Cis-acting_systematic_ | ggtgtcgagaagaggagaacaatatgctaaatgttgttctcgtctcctcgacacc | 1E, | 42 |
| | GU-evaluation_TAA-AGT | cagtagtgacaacatgctgtaaaaaacagcatgttgtaactactg | 1F | |
| pTS1487 | Cis-acting_systematic_ | ggtgtcgagaagaggagaacaatatgctaaatgttgttctcgtctcctcgacacc | 1E, | 43 |
| | GU-evaluation_TAT-AGA | cagtagagacaacatgctgtaaaaaacagcatgttgtatctactg | 1F | |
| pTS1488 | Cis-acting_systematic_ | ggtgtcgagaagaggagaacaatatgctaaatgttgttctcgtctcctcgacacc | 1E, | 44 |
| | GU-evaluation_TAG-AGC | cagtagcgacaacatgctgtaaaaaacagcatgttgtagctactg | 1F | |
| pTS1489 | Cis-acting_systematic_ | ggtgtcgagaagaggagaacaatatgctaaatgttgttctcgtctcctcgacacc | 1E, | 45 |
| | GU-evaluation_TAC-AGG | cagtagggacaacatgctgtaaaaaacagcatgttgtacctactg | 1F | |
| pTS1490 | Cis-acting_systematic_ | ggtgtcgagaagaggagaacaatatgctaaatgttgttctcgtctcctcgacacc | 1F | 46 |
| | GU-evaluation_GAA-CGT | cagtagtgccaacatgctgtaaaaaacagcatgttggaactactg | | |
| pTS1491 | Cis-acting_systematic_ | ggtgtcgagaagaggagaacaatatgctaaatgttgttctcgtctcctcgacacc | 1F | 47 |
| | GU-evaluation_GAT-CGA | cagtagagccaacatgctgtaaaaaacagcatgttggatctactg | | |
| pTS1492 | Cis-acting_systematic_ | ggtgtcgagaagaggagaacaatatgctaaatgttgttctcgtctcctcgacacc | 1F | 48 |
| | GU-evaluation_GAG-CGC | cagtagcgccaacatgctgtaaaaaacagcatgttggagctactg | | |
| pTS1493 | Cis-acting_systematic_ | ggtgtcgagaagaggagaacaatatgctaaatgttgttctcgtctcctcgacacc | 1F | 49 |
| | GU-evaluation_GAC-CGG | cagtagggccaacatgctgtaaaaaacagcatgttggacctactg | | |
| pTS1494 | Cis-acting_systematic_ | ggtgtcgagaagaggagaacaatatgctaaatgttgttctcgtctcctcgacacc | 1F | 50 |
| | GU-evaluation_CAT-GGA | cagtagaggcaacatgctgtaaaaaacagcatgttgcatctactg | | |
| pTS1495 | Cis-acting_systematic_ | ggtgtcgagaagaggagaacaatatgctaaatgttgttctcgtctcctcgacacc | 1F | 51 |
| | GU-evaluation_CAG-GGC | cagtagcggcaacatgctgtaaaaaacagcatgttgcagctactg | | |
| pTS1909 | Cis-acting_NN_ttaAgtt- | ccatatatattaagttatcgggtgtcgagaagaggagaacaatatgctaaatgttg | 1H | 52 |
| | aatCcaa | ttctcgtctcctcgacacccgataacctaatatatatgg | | |
| pTS1910 | Cis-acting_NN_ttaAgtt- | ccatatatattaagttatcgggtgtcgagaagaggagaacaatatgctaaatgttg | 1H | 53 |
| | aGtCcaa | ttctcgtctcctcgacacccgataacctgatatatatgg | | |
| pTS1911 | Cis-acting_NN_ttaAgtt- | ccatatatattaagttatcgggtgtcgagaagaggagaacaatatgctaaatgttg | 1H | 54 |
| | GatCcaa | ttctcgtctcctcgacacccgataacctagtatatatgg | | |
| pTS1912 | Cis-acting_NN_ttaAgtt- | ccatatatattaagttatcgggtgtcgagaagaggagaacaatatgctaaatgttg | 1H | 55 |
| | aaGCcaa | ttctcgtctcctcgacacccgataaccgaatatatatgg | | |
| pTS1913 | Cis-acting_NN_tttAatt- | ccatatatatttaattatcgggtgtcgagaagaggagaacaatatgctaaatgttg | 1H | 56 |
| | aaaCtaa | ttctcgtctcctcgacacccgataatcaaatatatatgg | | |
| pTS1914 | Cis-acting_NN_tttAatt- | ccatatatatttaattatcgggtgtcgagaagaggagaacaatatgctaaatgttg | 1H | 57 |
| | aaaCtGa | ttctcgtctcctcgacacccgatagtcaaatatatatgg | | |
| pTS1915 | Cis-acting_NN_tttAatt- | ccatatatatttaattatcgggtgtcgagaagaggagaacaatatgctaaatgttg | 1H | 58 |
| | aaaCtaG | ttctcgtctcctcgacacccgatgatcaaatatatatgg | | |
| pTS1916 | Cis-acting_NN_tttAatt- | ccatatatatttaattatcgggtgtcgagaagaggagaacaatatgctaaatgttg | 1H | 59 |
| | aaaCGaa | ttctcgtctcctcgacacccgataagcaaatatatatgg | | |
| pTS1917 | Cis-acting_2nd- | ccatatatatataaatatcgggtgtcgagaagaggagaacaatatgctaaatgttg | 1H | 60 |
| | NN_tatAaat-ataCtta | ttctcgtctcctcgacacccgatattcatatatatatgg | | |
| pTS1918 | Cis-acting_2nd- | ccatatatatataaatatcgggtgtcgagaagaggagaacaatatgctaaatgttg | 1H | 61 |
| | NN_tatAaat-ataCttG | ttctcgtctcctcgacacccgatgttcatatatatatgg | | |
| pTS1919 | Cis-acting_2nd- | ccatatatatataaatatcgggtgtcgagaagaggagaacaatatgctaaatgttg | 1H | 62 |
| | NN_tatAaat-ataCtGa | ttctcgtctcctcgacacccgatagtcatatatatatgg | | |
| pTS1920 | Cis-acting_2nd- | ccatatatataaagatatcgggtgtcgagaagaggagaacaatatgctaaatgtt | 1H | 63 |
| | NN_taaAgat-attCcta | gttctcgtctcctcgacacccgatatccttatatatatgg | | |
| pTS1921 | Cis-acting_2nd- | ccatatatataaagatatcgggtgtcgagaagaggagaacaatatgctaaatgtt | 1H | 64 |
| | NN_taaAgat-GttCcta | gttctcgtctcctcgacacccgatatccttgtatatatgg | | |
| pTS1922 | Cis-acting_2nd- | ccatatatataaagatatcgggtgtcgagaagaggagaacaatatgctaaatgtt | 1H | 65 |
| | NN_taaAgat-aGtCcta | gttctcgtctcctcgacacccgatatcctgatatatatgg | | |
| pTS1923 | Cis-acting_3nd- | ccatatattaataaaattcgggtgtcgagaagaggagaacaatatgctaaatgttg | 1H | 66 |
| | NN_taatAaaat-attaCttta | ttctcgtctcctcgacacccgaatttcattaatatatgg | | |
| pTS1924 | Cis-acting_3nd- | ccatatattaataaaattcgggtgtcgagaagaggagaacaatatgctaaatgttg | 1H | 67 |
| | NN_taatAaaat-attaCtttG | ttctcgtctcctcgacacccgagtttcattaatatatgg | | |
| pTS1925 | Cis-acting_3nd- | ccatatattaataaaattcgggtgtcgagaagaggagaacaatatgctaaatgttg | 1H | 68 |
| | NN_taatAaaat-attaCttGa | ttctcgtctcctcgacacccgaagttcattaatatatgg | | |
| pTS1926 | Cis-acting_3nd- | ccatatattaaaagaattcgggtgtcgagaagaggagaacaatatgctaaatgtt | 1H | 69 |
| | NN_taaaAgaat-atttCctta | gttctcgtctcctcgacacccgaattcctttaatatatgg | | |
| pTS1927 | Cis-acting_3nd- | ccatatattaaaagaattcgggtgtcgagaagaggagaacaatatgctaaatgtt | 1H | 70 |
| | NN_taaaAgaat-GtttCctta | gttctcgtctcctcgacacccgaattcctttgatatatgg | | |
| pTS1928 | Cis-acting_3nd- | ccatatattaaaagaattcgggtgtcgagaagaggagaacaatatgctaaatgtt | 1H | 71 |
| | NN_taaaAgaat-aGttCctta | gttctcgtctcctcgacacccgaattccttgaatatatgg | | |
| pTS1717 | AHI_LEAPER_gRNA | gtgaacgtcaaactgtcggaccaatatggcagaatcttctctcatctcaactttcca | 2A | 72 |
| | | tatccgtatcatggaatcatagcatcctgtaactactagctctcttacagctgg | | |
| pTS1758 | AHI_LEAPER_GU_at_ | gtgaacgtcaaactgtcggaccaatgtggcagaatcttctctcatctcaactttcca | 2A | 73 |
| | GU_amenable_sites_gRNA | tgtccgtgtcatggaatcatggcatcctgtgactgctggctctcttgcagctgg | | |
| pTS1791 | AHI_LEAPER_GA_at_ | gtgaacgtcaaactgtcggaccaagagggcagaatcttctctcatctcaactttcc | 2A | 74 |
| | GU_amenable_sites_gRNA | agatccggatcatggaatcagagcatcctggaacgacgagctctctgacagctgg | | |
| pTS1757 | AHI_LEAPER_GA_at_all_ | gtgaacgtcaaactgtcggaccaagagggcagaatcgtcgcgcatcgcaacgg | 2A | 75 |
| | off-target_sites_gRNA | tccagatccggatcatggaatcagagcatccgggaacgacgagctctcggacag | | |
| | | ctgg | | |
| pTS1818 | AHI_LEAPER_GU&GA_at_ | gtgaacgtcaaactgtcggaccaatgtggcagaatcgtcgcgcatctcagctttc | 2A | 76 |
| | all_off-target_sites_ | catgtccgtgtcatggaatcatggcatccggtgactgctggctctcgtgcagctg | | |
| | gRNA | g | | |
| pTS1718 | BMPR2_LEAPER_gRNA | gtgaagataagccagtcctctagtaacagaatgagcaagacggcaagagcttac | 2B | 77 |
| | | ccagtcacttgtgtggagacttaaatacttgcataaagatccattgggatagtact | | |
| | | c | | |
| pTS1752 | BMPR2_LEAPER_GU_at_ | gtgaagataagccagtcctctggtgacagaatgagcaagacggcaagagcttgc | 2B | 78 |
| | GU_amenable_sites_gRNA | ccagtcacttgtgtggagacttgaatgcttgcatgaagatccgttgggatggtgc | | |
| | | to | | |
| pTS1753 | BMPR2_LEAPER_GA_at_ | gtgaagataagccagtcctcgaggaacagaatgagcaagacggcaagagctga | 2B | 79 |
| | GU_amenable_sites_gRNA | cccagtcacttgtgtggagactgaaagacttgcagaaagatccagtgggagag | | |
| | | gactc | | |
| pTS1751 | BMPR2_LEAPER_GA_at_ | gtgaagataagccagtcctcgaggaacagaatgagcaagacggcaagagcgg | 2B | 80 |
| | all_off-target_sites_ | acccagtcacgggtgtggagacggaaagacgggcagaaagatccagtgggag | | |
| | gRNA | aggactc | | |
| pTS1827 | BMPR2_LEAPER_GU&GA_at_ | gtgaagataagccagtcctctggtgacagaatgagcaagacggcaagagcgga | 2B | 81 |
| | all_off-target_sites_ | cccagtcacgggtgtggagacgtgaatgcgggcatgaagatccgttgggatgg | | |
| | gRNA | tgctc | | |
| pTS1760 | COL3A1_LEAPER_gRNA | catattacagaataccttgatagcatccaatttgcatccttggttagggtcaaccca | 2C | 82 |
| | | gtattctccactcttgagttcaggatggcagaatttcaggtctctgcagtttct | | |
| pTS1821 | COL3A1_LEAPER_GU_at_ | catattacagaataccttggtagcatccagtttgcatccttggttagggtcaaccca | 2C | 83 |
| | GU_amenable_sites_gRNA | gtgttctccactcttgagttcagggtggcagagtttcaggtctctgcagtttct | | |
| pTS1822 | COL3A1_LEAPER_GA_at_ | catattacagaataccttgagagcatccaagttgcatccttggttagggtcaaccc | 2C | 84 |
| | GU_amenable_sites_gRNA | aggagtctccactcttgagttcaggagggcagaagttcaggtctctgcagtttct | | |
| pTS1823 | COL3A1_LEAPER_GA_at_ | catattacagaataccttgagagcatccaaggtgcatccttggttagggtcaaccc | 2C | 85 |
| | all_off-target_sites_ | aggagtctccactcgtgagttcaggagggcagaaggtcaggtctcggcaggtt | | |
| | gRNA | ct | | |
| pTS1824 | COL3A1_LEAPER_GU&GA_ | catattacagaataccttggtagcatccagtgtgcatccttggttagggtcaaccc | 2C | 86 |
| | at_all_off-target_ | agtgttctccactcgtgagttcagggtggcagagtgtcaggtctctgcaggttct | | |
| | sites_gRNA | | | |
| pTS1193 | RAB7A_LEAPER_gRNA | gtctttgataaaaggcgtacataattcttgtgtctactgtacagaatactgccgcca | 2D | 87 |
| | | gctggatttcccaattctgagtaacactctgcaatccaaacagggttcaaccct | | |
| pTS1801 | RAB7A_LEAPER_GU_at_ | gtctttgatgaaaggcgtgcgtagttcttgtgtctgctgtgcagaatgctgccgc | 2D | 88 |
| | GU_amenable_sites_gRNA | cagctgggtttcccagttctgagtgacactctgcaatccaaacagggttcaaccct | | |
| pTS1802 | RAB7A_LEAPER_GA_at_ | gtctttgagaaaaggcggacagaagtcttgtgtcgactggacagaagactgccg | 2D | 89 |
| | GU_amenable_sites_gRNA | ccagctggagttcccaagtctgaggaacactctgcaatccaaacagggttcaacc | | |
| | | ct | | |
| pTS1216 | RAB7A_LEAPER_GA_at_ | gtctttgagaaaaggcggacagaagtctggtgtcgacgggacagaagacggcc | 2D | 90 |
| | all_off-target_sites_ | gccagctggagggcccaaggcggaggaacactcggcaatccaaacagggg | | |
| | gRNA | aaccct | | |
| pTS1803 | RAB7A_LEAPER_GU&GA_at_ | gtctttgatgaaaggcgtgcgtagttcgggtgtctgctgtgcagaatgcggccg | 2D | 91 |
| | all_off-target_sites_ | ccagctgggtggcccagtgcggagtgacacgcggcaatccaaacagggggca | | |
| | gRNA | accct | | |
| pTS1964 | mMeCP2_LEAPER_Linear_ | tacatcatactttccagcagatcggccagacttcctttgtttaagctttcgtg | 2E | 92 |
| | gRNA | tccaaccttcaggcaaggtgggsrtggggtcatcatacataggtccccggtca | | |
| | | cggataatgga | | |
| pTS2031 | mMeCP2_LEAPER_Circular_ | aaccatgccgactgatggcagtacatcatactttccagcagateggccagacttcc | 2E | 93 |
| | gRNA | tttgtttaagctttcgtgtccaaccttcaggcaaggtggggtcatcatacataggt | | |
| | | ccccggtcacggataatggactgccatcagtcggcgtggactgtag | | |
| pTS2033 | mMeCP2_LEAPER_Circular_ | aaccatgccgactgatggcagtacatcatactttccagcagatcggccagacttcc | 2E | 94 |
| | GA_at_all_off-target_ | ggtgttgaagcggtcgtgtccaaccgtcaggcaaggtgggggcatcagacaga | | |
| | sites_gRNA | ggtccccggtcacggagaatggactgccatcagtcggcgtggactgtag | | |
| pTS2036 | mMeCP2_LEAPER_Circular_ | aaccatgccgactgatggcagtacatcatactttccagcagateggccagacttcc | 2E | 95 |
| | GU&GAat_all_off-target_ | ggtgtttgagcggtcgtgtccaaccgtcaggcaaggtgggggcatcatgcatg | | |
| | sites_gRNA | ggtccccggtcacggatgatggactgccatcagtcggcgtggactgtag | | |
| pTS1885 | AHI_2xBoxB_10(3)-29p15- | gcagaatcttggccctgaaaaagggcctcatctcaactttccatatccgtatcatg | 3A | 96 |
| | (3)10_gRNA | ggccctgaaaaagggcctcatagcatc | | |
| pTS1969 | AHI_2xBoxB_10(3)-29p15- | gcagaatcttggccctgaaaaagggcctcatctcaactttccatgtccgtgtcatg | 3A | 97 |
| | (3)10_GU_at_GU_ | ggccctgaaaaagggcctcatagcatc | | |
| | amenable_sites_gRNA | | | |
| pTS1968 | AHI_2xBoxB_10(3)-29p15- | gcagaatcttggccctgaaaaagggcctcatctcaactttccagatccggatcatg | 3A | 98 |
| | (3)10_GA_at_GU_ | ggccctgaaaaagggcctcatagcatc | | |
| | amenable_sites_gRNA | | | |
| pTS1967 | AHI_2xBoxB_10(3)-29p15- | gcagaatcttggccctgaaaaagggcctcatcgcaacggtccagatccggatcat | 3A | 99 |
| | (3)10_GA_at_all_off- | gggccctgaaaaagggcctcatagcatc | | |
| | target_sites_gRNA | | | |
| pTS1970 | AHI_2xBoxB_10(3)-29p15- | gcagaatcttggccctgaaaaagggcctcatcgcaacggtccatgtccgtgtcat | 3A | 100 |
| | (3)10_GU&GA_at_all_off- | gggccctgaaaaagggcctcatagcatc | | |
| | target_sites_gRNA | | | |
| pTS1973 | AHI_DR_gRNA | ctctcatctcaactttccatatccgtatcatggaatcatagcatcctgtaagtt | 4 | 101 |
| | | gtggaaggtccagttttgaggggctattacaac | | |
| pTS1976 | AHI_DR_GU_at_GU_ | ctctcatctcaactttccatgtccgtgtcatggaatcatagcatcctgtaagtt | 4 | 102 |
| | amenable_sites_gRNA | gtggaaggtccagttttgaggggctattacaac | | |
| pTS1975 | AHI_DR_GA_at_GU_ | ctctcatctcaactttccagatccggatcatggaatcatagcatcctgtaagt | 4 | 103 |
| | amenable_sites_gRNA | tgtggaaggtccagttttgaggggctattacaac | | |
| pTS1974 | AHI_DR_GA_at_all_off- | ctctcatctcaacggtccagatccggatcatggaatcatagcatcctgtaagt | 4 | 104 |
| | target_sites_gRNA | tgtggaaggtccagttttgaggggctattacaac | | |
| pTS1977 | AHI_DR_GU&GA_at_all_ | ctctcatctcaacggtccatgtccgtgtcatggaatcatagcatcctgtaag | 4 | 105 |
| | off-target_sites_gRNA | ttgtggaaggtccagttttgaggggctattacaac | | |
| pTS1795 | BMPR2_W298X_3x20- | ggtgtcgagaagaggagaacaatatctttcatgttgttctcctctcctcgac | 5A | 106 |
| | 20p8_RG-V25_No- | accgcttacccagtcacttgtgtaaaggacgctcatccaaggagccaaacgg | | |
| | block_distant | ccaatcagctccaacagaaaccagggcacccgccagggcc | | |
| pTS1794 | BMPR2_W298X_3x20- | ggtgtcgagaagaggagaacaatatctttcatgttgttctcctctcctcgac | 5A | 107 |
| | 20p8_RG-V25_No-block_ | accgcttacccagtcacttgtgtaaagggatagtactccatcacaaaaaaat | | |
| | close | attccatgcgtccatctaaaatctccaactataaagcggg | | |
| pTS1792 | BMPR2_W298X_3x20- | ggtgtcgagaagaggagaacaatatctttcatgttgttctcctctcctcgacaccg | 5A | 108 |
| | 20p8_RG-V25_GU-block_ | cttacccagtcacttgtgtaaaggggtagtgctccatcacaaaaaaatgttccgtg | | |
| | close | cgtccatctaaaatctccaactgtaaagcggg | | |
| pTS1793 | BMPR2_W298X_3x20- | ggtgtcgagaagaggagaacaatatctttcatgttgttctcctctcctcgacaccg | 5A | 109 |
| | 20p8_RG-V25_GA-block_ | cttacccagtcacttgtgtaaagggagaggactccatcacaaaaaaagagtccag | | |
| | close | gcgtccatctaaaatctccaacgagaaagcggg | | |
| pTS1997 | BMPR2_K983_3x20- | ggtgtcgagaagaggagaacaatatgctaaatgttgttctcgtctcctcgacacc | 5B | 110 |
| | 20p8_RG-V21_Reference | ccaccgcctaagagaataggaaatgctgccatccaggacatttaaactgaaagat | | |
| | | ccagagaattaaaagaacaccctgtgcaagaaca | | |
| pTS2000 | BMPR2_K983_3x20- | ggtgtcgagaagaggagaacaatatgctaaatgttgttctcgtctcctcgacacc | 5B | 111 |
| | 20p8_RG-V21_GU | ccaccgcctgagagaataggaaatgctgccatccaggacatttaaactgaaagat | | |
| | | ccagagaattaaaagaacaccctgtgcaagaaca | | |
| pTS1998 | BMPR2_K983_3x20- | ggtgtcgagaagaggagaacaatatgctaaatgttgttctcgtctcctcgacacc | 5B | 112 |
| | 20p8_RG-V21_GA | ccaccgccgaagagaataggaaatgctgccatccaggacatttaaactgaaagat | | |
| | | ccagagaattaaaagaacaccctgtgcaagaaca | | |
| pTS1999 | BMPR2_K983_3x20-19p8_ | ggtgtcgagaagaggagaacaatatgctaaatgttgttctcgtctcctcgacacc | 5B | 113 |
| | RG-V21_U-depletion | ccaccgccaagagaataggaaatgctgccatccaggacatttaaactgaaagatc | | |
| | | cagagaattaaaagaacaccctgtgcaagaaca | | |
| pTS2002 | COL3A1_N1244_3x20- | ggtgtcgagaagaggagaacaatatgctaaatgttgttctcgtctcctcgacacct | 5C | 114 |
| | 20p8_RG-V21_Reference | gtccatcaacagacttgagaaaacggggcaaaaccgccagctaaatcacctccaa | | |
| | | tcccagcaataaactccaacaccaccacagcaa | | |
| pTS2005 | COL3A1_N1244_3x20- | ggtgtcgagaagaggagaacaatatgctaaatgttgttctcgtctcctcgacacct | 5C | 115 |
| | 20p8_RG-V21_GU | gtccgtcaacagacttgagaaaacggggcaaaaccgccagctaaatcacctccaa | | |
| | | tcccagcaataaactccaacaccaccacagcaa | | |
| pTS2003 | COL3A1_N1244_3x20- | ggtgtcgagaagaggagaacaatatgctaaatgttgttctcgtctcctcgacacct | 5C | 116 |
| | 20p8_RG-V21_GA | gtccagcaacagacttgagaaaacggggcaaaaccgccagctaaatcacctccaa | | |
| | | tcccagcaataaactccaacaccaccacagcaa | | |
| pTS2004 | COL3A1_N1244_3x20- | ggtgtcgagaagaggagaacaatatgctaaatgttgttctcgtctcctcgacacct | 5C | 117 |
| | 20p8_RG-V21_U-depletion | gtccacaacagacttgagaaaacggggcaaaaccgccagctaaatcacctccaat | | |
| | | cccagcaataaactccaacaccaccacagcaa | | |
| pTS1764 | NUP43_V233V_3x20- | ggtgtcgagaagaggagaacaatatctttcatgttgttctcctctcctcgacaccc | 5D | 118 |
| | 20p8_RG-V25_CA-only | agtagccacaacatgctgtaaaggcactcggtcaccagtcagaaaaaaccctcca | | |
| | | tcagagtccaaaaaggcagcggtcgccagcggg | | |
| pTS1766 | NUP43_V233V_3x20- | ggtgtcgagaagaggagaacaatatctttcatgttgttctcctctcctcgacaccc | 5D | 119 |
| | 20p8_RG-V25_UG&CA | agtagtcacaacatgctgtaaaggcactcggtcaccagtcagaaaaaaccctccat | | |
| | | cagagtccaaaaaggcagcggtcgccagcggg | | |
| pTS1805 | NUP43_V233V_3x20- | ggtgtcgagaagaggagaacaatatctttcatgttgttctcctctcctcgacaccc | 5D | 120 |
| | 20p8_RG-V25_UG-only | agtagttacaacatgctgtaaaggcactcggtcaccagtcagaaaaaaccctccat | | |
| | | cagagtccaaaaaggcagcggtcgccagcggg | | |
| pTS1702 | GUSB_L456L_3x20- | ggtgtcgagaagaggagaacaatatctttcatgttgttctcctctcctcgacaccc | 5D | 121 |
| | 20p8_RG-V25_CA-only | agattccaggtgggacgcaaaagccacagaccacatcacgacaaacacgccgg | | |
| | | gacactcatcgaaaagcaccaagccagcgaagcag | | |
| pTS1770 | GUSB_L456L_3x20- | ggtgtcgagaagaggagaacaatatctttcatgttgttctcctctcctcgacaccc | 5D | 122 |
| | 20p8_RG-V25_UG&CA | agatttcaggtgggacgcaaaagccacagaccacatcacgacaaacacgccggg | | |
| | | acactcatcgaaaagcaccaagccagcgaagcag | | |
| pTS1769 | GUSB_L456L_3x20- | ggtgtcgagaagaggagaacaatatctttcatgttgttctcctctcctcgacaccc | 5D | 123 |
| | 20p8_RG-V25_UG-only | agattttaggtgggacgcaaaagccacagaccacatcacgacaaacacgccggg | | |
| | | acactcatcgaaaagcaccaagccagcgaagcag | | |
| pTS1705 | ACTB_3′-UTR_3x20- | ggtgtcgagaagaggagaacaatatctttcatgttgttctcctctcctcgacacca | 5D | 124 |
| | 20p8_RG-V25_CA-only | cgcaaccaagtcatagtccaaaagccgccgatccacacggagaaacctcaggg | | |
| | | agcggaaccgcaaagctcgaagtccagggcgacg | | |
| pTS1774 | ACTB_3′-UTR_3x20- | ggtgtcgagaagaggagaacaatatctttcatgttgttctcctctcctcgacacca | 5D | 125 |
| | V25_UG&CA | cgcaatcaagtcatagtccaaaagccgccgatccacacggagaaacctcagggc | | |
| | 20p8_RG- | agcggaaccgcaaagctcgaagtccagggcgacg | | |
| pTS1773 | ACTB_3′-UTR_3x20- | ggtgtcgagaagaggagaacaatatctttcatgttgttctcctctcctcgacacca | 5D | 126 |
| | 20p8_RG-V25_UG-only | cgcaattaagtcatagtccaaaagccgccgatccacacggagaaacctcagggca | | |
| | | gcggaaccgcaaagctcgaagtccagggcgacg | | |
| pTS2087 | AHI_K706_2xBoxB_10(3)- | actagctctcggccctgaaaaagggcccagctggatggaatctagccgtgtaaac | 3B | 160 |
| | 29p15-(3)10_5′-_and_3′ | aggccctgaaaaagggccgaaggatgag | | |
| | NN_reference | | | |
| pTS2111 | AHI_K706_2xBoxB_10(3)- | actagctctcggccctgaaaaagggcccagctggatggaagcgagccgtgtaaac | 3B | 161 |
| | 29p15-(3)10_5′-_and_3′ | aggccctgaaaaagggccgaaggatgag | | |
| | NN_GA-mismatch | | | |
| pTS2117 | AHI_K706_2xBoxB_10(3)- | actagctctcggccctgaaaaagggcccagctggatggagtctggccgtgtaaac | 3B | 162 |
| | 29p15-(3)10_5′-_and_3′ | aggccctgaaaaagggccgaaggatgag | | |
| | NN_GU-wobble | | | |
| pTS2088 | BMPR2_K984_2xBoxB_ | atgacccaggggccctgaaaaagggccaggggcgccaccgcctaagagaatagg | 3B | 163 |
| | 10(3)-29p15-(3)10_5′ | gaggccctgaaaaagggccttcacacgtt | | |
| | NN_reference | | | |
| pTS2114 | BMPR2_K984_2xBoxB_ | atgacccaggggccctgaaaaagggccaggggcgccaccgccgaagagaatag | 3B | 164 |
| | 10(3)-29p15-(3)10_5′ | ggaggccctgaaaaagggccttcacacgtt | | |
| | NN_GA-mismatch | | | |
| pTS2120 | BMPR2_K984_2xBoxB_ | atgacccaggggccctgaaaaagggccaggggcgccaccgcctgagagaatagg | 3B | 165 |
| | 10(3)-29p15-(3)10_5′ | gaggccctgaaaaagggccttcacacgtt | | |
| | NN_GU-wobble | | | |
| pTS2086 | AHI_11179_2xBoxB_ | gcttgttcttcggccctgaaaaagggccatccgtgtatccatcatgtgtcctt | 3B | 166 |
| | 10(3)-29p15-(3)10_3′ | ggtcggccctgaaaaagggccatggctctgt | | |
| | NN_reference | | | |
| pTS2110 | AHI_11179_2xBoxB_ | gcttgttcttcggccctgaaaaagggccatccgtgtatccagcatgtgtccttg | 3B | 167 |
| | 10(3)-29p15-(3)10_3′ | gtcggccctgaaaaagggccatggctctgt | | |
| | NN_GA-mismatch | | | |
| pTS2116 | AHI_11179_2xBoxB_ | gcttgttcttcggccctgaaaaagggccatccgtgtatccgtcatgtgtccttg | 3B | 168 |
| | 10(3)-29p15-(3)10_3′ | gtcggccctgaaaaagggccatggctctgt | | |
| | NN_GU-wobble | | | |
| pTS2089 | BMPR2_N1005_2xBoxB_ | gtggaatgaacggccctgaaaaagggcccctgttactgccatcattgttgact | 3B | 169 |
| | 10(3)-29p15-(3)10_3′ | tcacggccctgaaaaagggccccagcgattc | | |
| | NN_reference | | | |
| pTS2112 | BMPR2_N1005_2xBoxB_ | gtggaatgaacggccctgaaaaagggcccctgttactgccagcattgttgacttca | 3B | 170 |
| | 10(3)-29p15-(3)10_3′ | cggccctgaaaaagggccccagcgattc | | |
| | NN_GA-mismatch | | | |
| pTS2118 | BMPR2_N1005_2xBoxB_ | gtggaatgaacggccctgaaaaagggcccctgttactgccgtcattgttgact | 3B | 171 |
| | 10(3)-29p15-(3)10_3′ | tcacggccctgaaaaagggccccagcgattc | | |
| | NN_GU-wobble | | | |
| pTS2091 | COL3A1_N1244_2xBoxB_ | gactaatgagggccctgaaaaagggccttctatttgtccatcaacagacttgagtg | 3B | 172 |
| | 10(3)-29p15-(3)10_3′ | ggccctgaaaaagggcctcataatctc | | |
| | NN_reference | | | |
| pTS2113 | COL3A1_N1244_2xBoxB_ | gactaatgagggccctgaaaaagggccttctatttgtccagcaacagacttgagtg | 3B | 173 |
| | 10(3)-29p15-(3)10_3′ | ggccctgaaaaagggcctcataatctc | | |
| | NN_GA-mismatch | | | |
| pTS2119 | COL3A1_N1244_2xBoxB_ | gactaatgagggccctgaaaaagggccttctatttgtccgtcaacagacttgagtg | 3B | 174 |
| | 10(3)-29p15-(3)10_3′ | ggccctgaaaaagggcctcataatctc | | |
| | NN_GU-wobble | | | |
| pTS2013 | mMeCP2_W104Amber_ | aaccatgccgactgatggcagaatgttgttctcgtctcctcgacacctcgtgtcc | 5F | 175 |
| | Circular_4x20- | aaccttcaggcaaaaagtcatcatacataggtccccaaacacggataatggagcg | | |
| | 20p8_SplitRG-V21 | ccgcaaagatcaaatatacatcatactaaatccagcagatcggccagactaaaaa | | |
| | | ggtgtcgagaagaggagaacaatatgctactgccatcagtcggcgtggactgtag | | |
| pTS2051 | mMeCP2_W104Amber_ | aaccatgccgactgatggcagcatgttgttctcgtctcctcgacacctcgtgtccaa | 5F | 176 |
| | Circular_4x20-20p8_ | ccttcaggcaaaaagtcatcatacataggtccccaaacacggataatggagcgcc | | |
| | SplitRG-V24 | gcaaagatcaaatatacatcatactaaatccagcagatcggccagactaaaaagg | | |
| | | tgtcgagaagaggagaacaatatctttctgccatcagtcggcgtggactgtag | | |
| pTS2108 | mMeCP2_W104Amber_ | aaccatgccgactgatggcagcatgttgttctcgtctcctcgacacctcgtgtccaa | 5F | 177 |
| | Circular_4x20- | ccttcaggcaaaaagtcatcatgcataggtccccaaacacggatgatggagcgcc | | |
| | 20p8_SplitRG-V24_ | gcaaagatcaaatatacatcgtactaaatccagcagatcggccagactaaaaagg | | |
| | GU-wobbles | tgtcgagaagaggagaacaatatctttctgccatcagtcggcgtggactgtag | | |
| | mMeCP2_W104Amber_ | aaccatgccgactgatggcagcatgttgttctcctctcctcgacacctcgtgtccaa | | 178 |
| | Circular_4x20- | ccttcaggcaaaaagtcatcatgcataggtccccaaacacggatgatggagcgcc | | |
| | 20p8_SplitRG-V25_ | gcaaagatcaaatatacatcgtactaaatccagcagatcggccagactaaaaagg | | |
| | GU-wobbles | tgtcgagaagaggagaacaatatctttctgccatcagtcggcgtggactgtag | | |
| | AAV-PHP.eB encoded | aaccatgccgactgatggcagcatgttgttctcgtctcctcgacacctcgtgtccaa | 7 | 182 |
| | guide RNA(mMeCP2_ | ccttcaggcaaaaagtcatcatgcataggtccccaaacacggatgatggagcgcc | | |
| | W104Amber_Circular_ | gcaaagatcaaatatacatcgtactaaatccagcagatcggccagactaaaaagg | | |
| | 4x20-20p8_SplitRG- | tgtcgagaagaggagaacaatatctttctgccatcagtcggcgtggactgtag | | |
| | V24_GU-wobbles) | | | |

### Oligo List:

| Oligo # | Oligo Name | Sequence 5′->3′ | SEQ ID NO |
|---|---|---|---|
| 121 | GFP_fw | gcggatccaccatggctagcaaaggagaagaactc | 127 |
| 144 | BGH_bw | ctagaaggcacagtcgaggc | 128 |
| 551 | BGH_long_bw | gctggcaactagaaggcacagtcgaggc | 129 |
| 566 | GFP_seq_fw | gacacgtgctgaagtcaagtttgaaggtg | 130 |
| 1032 | ACTB_fw | cagcagatgtggatcagcaagcaggag | 131 |
| 1033 | ACTB_bw | ggaagggggggcacgaaggctcatc | 132 |
| 2901 | AHI_W725Amber_fw | gccgtatcccaaacaaacacc | 133 |
| 2902 | AHI_W725Amber_bw | ccaaacaacaatcacccctgt | 134 |
| 2903 | BMPR2_W298Amber_fw | ccttggatgagcgtccagtt | 135 |
| 2905 | COL3A1_W1278Amber_fw | tgctgggattggaggtgaaaa | 136 |
| 2919 | GusB_L456L fw | caacaagcatgaggatgcgg | 137 |
| 2920 | GusB_L456L_bw | gtgcccgtagtcgtgatacc | 138 |
| 2933 | NUP43_V233V_fw | tgtgtgcaacaacccagaaat | 139 |
| 2934 | NUP43_V233V_bw | gtactgcttcttcctccttggtg | 140 |
| 2939 | RAB7A_3′UTR_fw | gccccattacaggctcacac | 141 |
| 2940 | RAB7A_3′UTR_bw | ttgaagtgtggagcaggggg | 142 |
| 3041 | RAB7A_Exon5_fw | ccagacgattgcacggaatg | 143 |
| 3241 | mMecP2_fw | aaccttcagcccaccattct | 144 |
| 3243 | GusB_L456L_Meta-Sense-Oligo | gggcttcgactggccgctgctggtgtttttcctgcttcgctggcttggtgc | 145 |
| | | cttttttcgatgagtgtcccggcgtgggcctggcgctgccgctttttcccc | |
| | | gcggtcgtgatgtggtctgtggccttttttgcgtcccacctcgaatgcc | |
| 3244 | NUP43_V233V_Meta-Sense-Oligo | gttggagaggatggtcgatttttctcctgagattctttttttctgactggtg | 146 |
| | | accgagtgcctttttacagcatgttgtcgctagcc | |
| 3454 | NUP43_5′UTR_fw | ctgctgcggccgctttcg | 147 |
| 3569 | ACTB_3′UTR_Sense-Oligo | cgtcgccctggacttcgagctttgcggttccgctgccctgaggtttctccg | 148 |
| | | tgtggatcggcggcttttggactatgacttcgttggca | |
| 4964 | mMeCP2_65° C._bw | acaacaagtttcccagggctcttctcc | 149 |
| 4965 | mMeCP2_65° C._fw | ctagcgctaccggactcagatctcg | 150 |

### List of Applied ASOs:

### ASO Legend:

mN=2′O-methyl
*=Phosphorothioate linkage

| | | Used in | SEQ ID |
|---|---|---|---|
| ASO Name | Sequence 5′->3′ | Figure | NO |
| ASO | mU*mG*mA*G*U*A*U*C*U*G*C*C*U*A*A*C*U*U*C*A | 6 | 179 |
| | *U*G*U*A*A*CCCACGAAU*C*U*U*G*U*C*C*C*A*A* | | |
| | C*C*C*A*G*G*U*C*U*C*U*A*mG*mG*mU | | |
| ASO 2′OMe at | mU*mG*mA*G*U*A*U*C*U*G*C*C*U*A*A*C*U*U* | 6 | 180 |
| all off-target | C*A*U*mG*mU*mA*A*CCCACGAAU*mC*mU*mU*G | | |
| sites | *U*C*C*C*A*A*C*C*C*A*G*G*U*C*U*mC*mU*mA | | |
| | *mG*mG*mU | | |
| ASO 2′OMe or | mU*mG*mA*G*U*A*U*C*U*G*C*C*U*A*A*C*U*U*C*A | 6 | 181 |
| GU at off-target | *U*G*U*G*A*CCCACGAAU*mC*mU*mU*G*U*C*C*C* | | |
| sites | A*A*C*C*C*A*G*G*U*C*U*C*U*G*mG*mG*mU | | |

Note:
The ASO sequences had to be given with ″t″ instead of ″u″ in WIPO for technical reasons, but were labelled with the Molecule Type ″RNA″.
ASO legend:
mN = 2′O-methyl
* = Phosphorothioate linkage

### Oligo List:

| Oligo # | Oligo Name | Sequence 5′->3′ | SEQ ID NO |
|---|---|---|---|
| | 29 +91 Mecp2 E1 Rev | ggaagctttgtcagagccctacc | 183 |
| | -14 Mecp2 E1 Fwd | aacccgtccggaaaatggcc | 184 |
| | TM766 Rev | tgtacaagaaagctgggtcg | 185 |
| | TM764 Fwd | ctttgcgaagtgtcaacct | 186 |
| | TM768 Rev | ttccaggcggaccatacaac | 187 |
| | mMecp2 178 Fwd | catgagccactacaaccttcag | 188 |
| | mMecp2 554 Rev | ctcctggaggggctccctctc | 189 |

### Primer-Pair List:

| Target | Primer Pair | Sense Oligo | Sequencing Primer |
|---|---|---|---|
| ACTB 3′UTR | 1032 + 1033 | 3569 | 1032 |
| AHI K706, I1179 & W725Amber | 2901 + 144 | None | 2901 (K706), |
| | | | 2902 (W725Amber), |
| | | | or 144 (I1179) |
| BMPR2 K983 & W298Amber | 2903 + 144 | None | 144 |
| COL3A1 N1244 | 2905 + 144 | None | 2905 |
| COL3A1 W1278Amber | 2905 + 144 | None | 144 |
| eGFP cis-acting guide RNA reporter | 551 + 121 | None | 566 |
| GUSB L456L | 2919 + 2920 | 3243 | 2919 |
| mMeCP2 W104Amber | 4964 + 4965 | None | 3241 |
| NUP43 V233V | 2934 + 3454 | 3244 | 2933 |
| RAB7A 3′UTR | 3041 + 2940 | 3134 | 2939 |

### Primer-Pair List:

| Target | Primer Pair | Sense Oligo | Sequencing Primer |
|---|---|---|---|
| | TM766 Rev & | None | None |
| | TM764 Fwd | | |
| | TM768 Rev & | None | TM768 Rev |
| | TM764 Fwd | | |
| | −14 Mecp2 E1 | None | Mecp2 E1 743 Rev |
| | Fwd & +91 | | |
| | Mecp2 E1 Rev | | |
| | mMecp2 178 Fwd & | None | None |
| | mMecp2 554 Rev | | |

1. Artificial nucleic acid for site-directed editing of a target RNA which comprises a target sequence comprising an adenosine as a target nucleotide to be edited and at least one off-target sequence comprising an adenosine that is not to be edited, the artificial nucleic acid comprising a first nucleic acid sequence consisting of 3 nucleotides that is complementary or partially complementary to a nucleic acid sequence in the target sequence, which comprises a target adenosine nucleotide to be edited and the nucleotides immediately 5′ and 3′ of said target adenosine nucleotide, and a second nucleic acid sequence comprising at least 3 nucleotides that is complementary or partially complementary to a nucleic acid sequence in the at least one off-target sequence, which comprises an adenosine nucleotide not to be edited and the nucleotides immediately 5′ and 3′ of said adenosine nucleotide, wherein the target sequence comprises at least one nucleotide triplet selected from the group consisting of 5′-UAG-3′, 5′-GAU-3′, 5′-GAG-3′, 5′-GAC-3′, 5′-GAA-3′, 5′-CAG-3′, and 5′-AAG-3′, and/or the off-target sequence comprises at least one nucleotide triplet selected from the group consisting of 5′-UAU-3′, 5′-UAG-3′, 5′-UAC-3′, 5′-UAA-3′, 5′-GAU-3′, 5′-CAU-3′, and 5′-AAU-3′, and/or
a third nucleic acid sequence consisting of 4 or 5 nucleotides that is complementary or partially complementary to a nucleic acid sequence in the target RNA which comprises an adenosine nucleotide to be edited, at least one neighbouring adenosine that is not to be edited and the nucleotides immediately 5′ and 3′ of said adenosine nucleotides, wherein the target RNA comprises at least one nucleotide sequence selected from the group consisting of 5′-UAAA-3′, 5′-UAAU-3′, 5′-UAAC-3′, 5′-UAAG-3′, 5′-AAAU-3′, 5′-CAAU-3′, 5′-GAAU-3′, and 5′-UAAAU-3′, wherein
the first nucleic acid sequence of the artificial nucleic acid
consists of a nucleotide triplet selected from the group consisting of 5′-UUA-3′, and 5′-UCA-3′, preferably if the target adenosine nucleotide to be edited in the target sequence is the central adenosine nucleotide in a nucleotide triplet 5′-UAG-3′;
consists of a nucleotide triplet selected from the group consisting of 5′-AUU-3′, and 5′-ACU-3′, preferably if the target adenosine nucleotide to be edited in the target sequence is the central adenosine nucleotide in a nucleotide triplet 5′-GAU-3′;
consists of a nucleotide triplet selected from the group consisting of 5′-CUU-3′, 5′-UUC-3′, 5′-UUU-3′, 5′-UUG-3′, 5′-GUU-3′, 5′-UUA-3′, 5′-AUU-3′, 5′-CCU-3′, 5′-UCC-3′, and 5′-UCU-3′, preferably if the target adenosine nucleotide to be edited in the target sequence is the central adenosine nucleotide in a nucleotide triplet 5′-GAG-3′;
consists of a nucleotide triplet selected from the group consisting of 5′-GUU-3′, and 5′-GCU-3′, preferably if the target adenosine nucleotide to be edited in the target sequence is the central adenosine nucleotide in a nucleotide triplet 5′-GAC-3′;
consists of a nucleotide triplet selected from the group consisting of 5′-UUU-3′, 5′-AUU-3′, 5′-UCU-3′, and 5′-ACU-3′, preferably if the target adenosine nucleotide to be edited in the target sequence is the central adenosine nucleotide in a nucleotide triplet 5′-GAA-3′;
consists of a nucleotide triplet selected from the group consisting of 5′-UUG-3′, 5′-UCG-3′, 5′-UUA-3′, and 5′-UCA-3′, preferably if the target adenosine nucleotide to be edited in the target sequence is the central adenosine nucleotide in a nucleotide triplet 5′-CAG-3′; and/or
consists of a nucleotide triplet selected from the group consisting of 5′-UUU-3′, and 5′-UCU-3′, preferably if the target adenosine nucleotide to be edited in the target sequence is the central adenosine nucleotide in a nucleotide triplet 5′-AAG-3′, and/or
the second nucleic acid sequence of the artificial nucleic acid
does not comprise a nucleotide triplet 5′-AUA-3′ and comprises one or more of a nucleotide triplet selected from the group consisting of 5′-GUG-3′, 5′-AUG-3′, 5′-GUA-3′, 5′-GGG-3′, 5′-AGG-3′, and 5′-GGA-3′, preferably if an adenosine nucleotide not to be edited in the off-target sequence is the central adenosine nucleotide in a nucleotide triplet 5′-UAU-3′;
does not comprise a nucleotide triplet 5′-CUA-3′ and comprises one or more of a nucleotide triplet selected from the group consisting of 5′-CUG-3′, 5′-UUG-3′, 5′-CGG-3′, and 5′-UGG-3′, preferably if an adenosine nucleotide not to be edited in the off-target sequence is the central adenosine nucleotide in a nucleotide triplet 5′-UAG-3′;
does not comprise a nucleotide triplet 5′-GUA-3′ and comprises one or more of a nucleotide triplet selected from the group consisting of 5′-GUG-3′, and 5′-GGG-3′, preferably if an adenosine nucleotide not to be edited in the off-target sequence is the central adenosine nucleotide in a nucleotide triplet 5′-UAC-3′;
does not comprise a nucleotide triplet 5′-UUA-3′ and comprises one or more of a nucleotide triplet selected from the group consisting of 5′-UUG-3′, 5′-UGG-3′, 5′-GUG-3′, and 5′-GGG-3′, preferably if an adenosine nucleotide not to be edited in the off-target sequence is the central adenosine nucleotide in a nucleotide triplet 5′-UAA-3′;
does not comprise a nucleotide triplet 5′-AUC-3′ and comprises one or more of a nucleotide triplet selected from the group consisting of 5′-GUC-3′, 5′-GUU-3′, 5′-GGC-3′, and 5′-GGU-3′, preferably if an adenosine nucleotide not to be edited in the off-target sequence is the central adenosine nucleotide in a nucleotide triplet 5′-GAU-3′;
does not comprise a nucleotide triplet 5′-AUG-3′ and comprises one or more of a nucleotide triplet selected from the group consisting of 5′-GUG-3′, and 5′-GGG-3′, preferably if an adenosine nucleotide not to be edited in the off-target sequence is the central adenosine nucleotide in a nucleotide triplet 5′-CAU-3′; and/or
does not comprise a nucleotide triplet 5′-AUU-3′ and comprises one or more of a nucleotide triplet selected from the group consisting of 5′-GUU-3′, 5′-GGU-3′, 5′-GUG-3′, and 5′-GGG-3′, preferably if an adenosine nucleotide not to be edited in the off-target sequence is the central adenosine nucleotide in a nucleotide triplet 5′-AAU-3′, and/or
the third nucleic acid sequence of the artificial nucleic acid
consists of a nucleotide sequence 5′-UCUG-3′, or of a nucleotide sequence 5′-GCUG-3′, preferably if the adenosine nucleotides in the target sequence are present in the context of the nucleic acid sequence 5′-UAAA-3′;
consists of a nucleotide sequence 5′-ACUG-3′, or of a nucleotide sequence 5′-GUCA-3′, preferably if the adenosine nucleotides in the target sequence are present in the context of the nucleic acid sequence 5′-UAAU-3′;
consists of a nucleotide sequence 5′-GCUG-3′, preferably if the adenosine nucleotides in the target sequence are present in the context of the nucleic acid sequence 5′-UAAC-3′;
consists of a nucleotide sequence 5′-CCUG-3′, or of a nucleotide sequence 5′-UCUG-3′, or of a nucleotide sequence 5′-UUUG-3′, preferably if the adenosine nucleotides in the target sequence are present in the context of the nucleic acid sequence 5′-UAAG-3′;
consists of a nucleotide sequence 5′-GUCU-3′, or of a nucleotide sequence 5′-GUCG-3′, preferably if the adenosine nucleotides in the target sequence are present in the context of the nucleic acid sequence 5′-AAAU-3′;
consists of a nucleotide sequence 5′-GUCG-3′, preferably if the adenosine nucleotides in the target sequence are present in the context of the nucleic acid sequence 5′-CAAU-3′;
consists of a nucleotide sequence 5′-GUCC-3′, or of a nucleotide sequence 5′-GUCU-3′, or of a nucleotide sequence 5′-GUUU-3′, preferably if the adenosine nucleotides in the target sequence are present in the context of the nucleic acid sequence 5′-GAAU-3′; and/or
consists of a nucleotide sequence 5′-GUCUG-3′, preferably if the adenosine nucleotides in the target sequence are present in the context of the nucleic acid sequence 5′-UAAAU-3′.

2. The artificial nucleic acid according to claim 1, wherein the second nucleic acid sequence
does not comprise a nucleotide triplet 5′-AUA-3′ and comprises one or more of a nucleotide triplet selected from the group consisting of 5′-GUG-3′, 5′-AUG-3′, 5′-GUA-3′, 5′-GGG-3′, 5′-AGG-3′, and 5′-GGA-3′, preferably if an adenosine nucleotide not to be edited in the off-target sequence is the central adenosine nucleotide in a nucleotide triplet 5′-UAU-3′;
does not comprise a nucleotide triplet 5′-CUA-3′ and comprises one or more of a nucleotide triplet selected from the group consisting of 5′-CUG-3′, 5′-UUG-3′, 5′-CGG-3′, and 5′-UGG-3′, preferably if an adenosine nucleotide not to be edited in the off-target sequence is the central adenosine nucleotide in a nucleotide triplet 5′-UAG-3′;
does not comprise a nucleotide triplet 5′-GUA-3′ and comprises one or more of a nucleotide triplet selected from the group consisting of 5′-GUG-3′, and 5′-GGG-3′, preferably if an adenosine nucleotide not to be edited in the off-target sequence is the central adenosine nucleotide in a nucleotide triplet 5′-UAC-3′;
does not comprise a nucleotide triplet 5′-UUA-3′ and comprises one or more of a nucleotide triplet selected from the group consisting of 5′-UUG-3′, 5′-UGG-3′, 5′-GUG-3′, and 5′-GGG-3′, preferably if an adenosine nucleotide not to be edited in the off-target sequence is the central adenosine nucleotide in a nucleotide triplet 5′-UAA-3′; and/or
does not comprise a nucleotide triplet 5′-AUU-3′ and comprises one or more of a nucleotide triplet selected from the group consisting of 5′-GUU-3′, and 5′-GGU-3′, preferably if an adenosine nucleotide not to be edited in the off-target sequence is the central adenosine nucleotide in a nucleotide triplet 5′-AAU-3′.

3. The artificial nucleic acid according to claim 1 or 2, wherein the second nucleic acid sequence
does not comprise a nucleotide sequence 5′-AUA-3′ and comprises one or more of a nucleotide triplet selected from the group consisting of 5′-GUG-3′, 5′-AUG-3′, and 5′-GUA-3′, preferably if an adenosine nucleotide not to be edited in the off-target sequence is the central adenosine nucleotide in a nucleotide triplet 5′-UAU-3′;
does not comprise a nucleotide triplet 5′-CUA-3′ and comprises one or more of a nucleotide triplet selected from the group consisting of 5′-CUG-3′, and 5′-UUG-3′, preferably if an adenosine nucleotide not to be edited in the off-target sequence is the central adenosine nucleotide in a nucleotide triplet 5′-UAG-3′;
does not comprise a nucleotide triplet 5′-GUA-3′ and comprises a nucleotide triplet 5′-GUG-3′, preferably if an adenosine nucleotide not to be edited in the off-target sequence is the central adenosine nucleotide in a nucleotide triplet 5′-UAC-3′;
does not comprise a nucleotide triplet 5′-UUA-3′ and comprises a nucleotide triplet 5′-UUG-3′, preferably if an adenosine nucleotide not to be edited in the off-target sequence is the central adenosine nucleotide in a nucleotide triplet 5′-UAA-3′; and/or
does not comprise a nucleotide triplet 5′-AUU-3′ and comprises a nucleotide triplet 5′-GUU-3′, preferably if an adenosine nucleotide not to be edited in the off-target sequence is the central adenosine nucleotide in a nucleotide triplet 5′-AAU-3′.

4. The artificial nucleic acid according to any one of claims 1 to 3, wherein the first nucleic acid sequence
consists of nucleotide triplet 5′-UUA-3′, preferably if the target adenosine nucleotide to be edited in the target sequence is the central adenosine nucleotide in a nucleotide triplet 5′-UAG-3′;
consists of nucleotide triplet 5′-AUU-3′, preferably if the target adenosine nucleotide to be edited in the target sequence is the central adenosine nucleotide in a nucleotide triplet 5′-GAU-3′;
consists of a nucleotide triplet selected from the group consisting of 5′-CUU-3′, 5′-UUC-3′, 5′-UUU-3′, 5′-UUG-3′, 5′-GUU-3′, 5′-UUA-3′, and 5′-AUU-3′, preferably if the target adenosine nucleotide to be edited in the target sequence is the central adenosine nucleotide in a nucleotide triplet 5′-GAG-3′;
consists of nucleotide triplet 5′-GUU-3′, preferably if the target adenosine nucleotide to be edited in the target sequence is the central adenosine nucleotide in a nucleotide triplet 5′-GAC-3′;
consists of nucleotide triplet 5′-UUU-3′, or 5′-AUU-3′, preferably if the target adenosine nucleotide to be edited in the target sequence is the central adenosine nucleotide in a nucleotide triplet 5′-GAA-3′;
consists of nucleotide triplet 5′-UUG-3′ or 5′-UUA-3′, preferably if the target adenosine nucleotide to be edited in the target sequence is the central adenosine nucleotide in a nucleotide triplet 5′-CAG-3′; and/or
consists of nucleotide triplet 5′-UUU-3′, preferably if the target adenosine nucleotide to be edited in the target sequence is the central adenosine nucleotide in a nucleotide triplet 5′-AAG-3′.

5. The artificial nucleic acid according to any one of claims 1 to 4, wherein the second nucleic acid sequence further
does not comprise a nucleotide triplet 5′-CUC-3′ and comprises one or more of nucleotide triplet 5′-CGC-3′, preferably if an adenosine nucleotide not to be edited in the off-target sequence is the central adenosine nucleotide in a nucleotide triplet 5′-GAG-3′;
does not comprise a nucleotide triplet 5′-GUC-3′ and comprises one or more of nucleotide triplet 5′-GGC-3′, preferably if an adenosine nucleotide not to be edited in the off-target sequence is the central adenosine nucleotide in a nucleotide triplet 5′-GAC-3′;
does not comprise a nucleotide triplet 5′-UUC-3′ and comprises one or more of a nucleotide triplet selected from the group consisting of 5′-UGC-3′, and 5′-GGC-3′, preferably if an adenosine nucleotide not to be edited in the off-target sequence is the central adenosine nucleotide in a nucleotide triplet 5′-GAA-3′;
does not comprise a nucleotide triplet 5′-CUG-3′ and comprises one or more of nucleotide triplet 5′-CGG-3′, preferably if an adenosine nucleotide not to be edited in the off-target sequence is the central adenosine nucleotide in a nucleotide triplet 5′-CAG-3′;
does not comprise a nucleotide triplet 5′-GUG-3′ and comprises one or more of nucleotide triplet 5′-GGG-3′, preferably if an adenosine nucleotide not to be edited in the off-target sequence is the central adenosine nucleotide in a nucleotide triplet 5′-CAC-3′;
does not comprise a nucleotide triplet 5′-UUG-3′ and comprises one or more of a nucleotide triplet selected from the group consisting of 5′-UGG-3′ and 5′-GGG-3′, preferably if an adenosine nucleotide not to be edited in the off-target sequence is the central adenosine nucleotide in a nucleotide triplet 5′-CAA-3′;
does not comprise a nucleotide triplet 5′-CUU-3′ and comprises one or more of a nucleotide triplet selected from the group consisting of 5′-CGU-3′ and 5′-CGG-3′, preferably if an adenosine nucleotide not to be edited in the off-target sequence is the central adenosine nucleotide in a nucleotide triplet 5′-AAG-3′;
does not comprise a nucleotide triplet 5′-GUU-3′ and comprises one or more of a nucleotide triplet selected from the group consisting of 5′-GGU-3′ and 5′-GGG-3′, preferably if an adenosine nucleotide not to be edited in the off-target sequence is the central adenosine nucleotide in a nucleotide triplet 5′-AAC-3′; and/or
does not comprise a nucleotide triplet 5′-UUU-3′ and comprises one or more of a nucleotide triplet selected from the group consisting of 5′-UGU-3′, 5′-GGU-3′, 5′-UGG-3′, and 5′-GGG-3′, preferably if an adenosine nucleotide not to be edited in the off-target sequence is the central adenosine nucleotide in a nucleotide triplet 5′-AAA-3′.

6. The artificial nucleic acid according to any one of claims 1 to 5, which further comprises a nucleic acid sequence capable of recruiting an adenosine deaminase.

7. The artificial nucleic acid according to claim 6, wherein the nucleic acid sequence is capable of binding to the adenosine deaminase, preferably to the dsRNA binding domain of the adenosine deaminase.

8. The artificial nucleic acid according to claim 7, wherein the nucleic acid sequence is capable of binding to an adenosine deaminase fusion protein.

9. The artificial nucleic acid according to claim 8, wherein the adenosine deaminase fusion protein is selected from the group consisting of Cas9-ADAR, Cas13-ADAR, MS2 Coat Protein-ADAR, λN-ADAR, CIRTS-ADAR, and TAR binding protein-ADAR.

10. The artificial nucleic acid according to claim 6 or 7, wherein the nucleic acid sequence is capable of recruiting and preferably binding to a tagged deaminase.

11. The artificial nucleic acid according to claim 6 or 7, wherein the adenosine deaminase is not a SNAP-tagged deaminase, preferably not a tagged deaminase.

12. The artificial nucleic acid according to claim 6 or 7, wherein the nucleic acid sequence is capable of recruiting and preferably binding to an endogenous adenosine deaminase.

13. The artificial nucleic acid according to claim 12, wherein the endogenous adenosine deaminase is ADAR1 or ADAR2, preferably ADAR1, preferably human ADAR1, in particular ADAR1p110.

14. The artificial nucleic acid according to any one of claims 6 to 13, wherein the nucleic acid sequence capable of recruiting an adenosine deaminase is further capable of intramolecular base pairing, preferably capable of forming a stem-loop structure.

15. The artificial nucleic acid according to claim 14, wherein the stem-loop structure comprises a double-helical stem comprising at least one mismatch, and a loop consisting of from 3 to 8, preferably from 4 to 6, more preferably 5 nucleotides.

16. The artificial nucleic acid according to any one of claims 6 to 15, which comprises a further recruiting moiety capable of recruiting an adenosine deaminase, preferably an endogenous adenosine deaminase, by binding to the target RNA, wherein said recruiting moiety comprises a recruitment sequence that is complementary or partially complementary to a nucleic acid sequence in the target RNA which does not contain any adenosine nucleotides to be edited.

17. The artificial nucleic acid according to claim 16, wherein the further recruiting moiety comprises a cluster of recruitment sequences comprising at least two recruitment sequences, which are linked via a nucleotide linker, which comprises at least 1 nucleotide, preferably 2 to 6 nucleotides, preferably adenosine nucleotides.

18. The artificial nucleic acid according to claim 17, wherein the cluster of recruitment sequences comprises 2 to 10, preferably 3 to 6, recruitment sequences, each comprising 10 to 200, preferably 10 to 100, nucleotides.

19. The artificial nucleic acid according to any one of the claims 16 to 18, wherein a recruitment sequence of a further recruiting moiety, which is complementary or partially complementary to a nucleic acid sequence in the target RNA which does not contain adenosine nucleotides to be edited,
does not comprise a nucleotide triplet 5′-AUA-3′ and comprises one or more of a nucleotide triplet selected from the group consisting of 5′-GUG-3′, 5′-AUG-3′, 5′-GUA-3′, 5′-GGG-3′, 5′-AGG-3′, and 5′-GGA-3′, preferably a nucleotide triplet selected from the group consisting of 5′-GUG-3′, 5′-AUG-3′, and 5′-GUA-3′, if an adenosine nucleotide not to be edited in the target RNA sequence is the central adenosine nucleotide in a nucleotide triplet 5′-UAU-3′;
does not comprise a nucleotide triplet 5′-CUA-3′ and comprises one or more of a nucleotide triplet selected from the group consisting of 5′-CUG-3′, 5′-UUG-3′, 5′-CGG-3′, and 5′-UGG-3′, preferably a nucleotide triplet selected from the group consisting of 5′-CUG-3′, and 5′-UUG-3′, if an adenosine nucleotide not to be edited in the target RNA sequence is the central adenosine nucleotide in a nucleotide triplet 5′-UAG-3′;
does not comprise a nucleotide triplet 5′-GUA-3′ and comprises one or more of a nucleotide triplet selected from the group consisting of 5′-GUG-3′, and 5′-GGG-3′, preferably a nucleotide triplet 5′-GUG-3′, if an adenosine nucleotide not to be edited in the target RNA sequence is the central adenosine nucleotide in a nucleotide triplet 5′-UAC-3′;
does not comprise a nucleotide triplet 5′-UUA-3′ and comprises one or more of a nucleotide triplet selected from the group consisting of 5′-UUG-3′, 5′-UGG-3′, 5′-GUG-3′, and 5′-GGG-3′, preferably a nucleotide triplet 5′-UUG-3′, if an adenosine nucleotide not to be edited in the target RNA sequence is the central adenosine nucleotide in a nucleotide triplet 5′-UAA-3′; and/or
does not comprise a nucleotide triplet 5′-AUU-3′ and comprises one or more of a nucleotide triplet selected from the group consisting of 5′-GUU-3′, and 5′-GGU-3′, preferably a nucleotide triplet 5′-GUU-3′, if an adenosine nucleotide not to be edited in the target RNA sequence is the central adenosine nucleotide in a nucleotide triplet 5′-AAU-3′.

20. The artificial nucleic acid according to any one of the claims 16 to 19, wherein the region(s) of the target RNA bound by the further recruiting moieties does not include an adenosine nucleotide in a 5′-AAG-3′, 5′-AAC-3′ or 5′-AAA-3′ context.

21. The artificial nucleic acid according to any one of the preceding claims, which is a circular nucleic acid molecule.

22. The artificial nucleic acid according to claim 21, which comprises a nucleotide sequence selected from the group consisting of 5′-GGUGUCGAGAAGAGGAGAACAAUAUGCUACUGCCAUCAGUCGGCGUGGACUGU AGAACCAUGCCGACUGAUGGCAGAAUGUUGUUCUCGUCUCCUCGACACC-3′ (SEQ ID NO: 5), 5′-GGUGUCGAGAAGAGGAGAACAAUAUCUUUCUGCCAUCAGUCGGCGUGGACUGU AGAACCAUGCCGACUGAUGGCAGCAUGUUGUUCUCGUCUCCUCGACACC-3′ (SEQ ID NO: 6), and 5′-GGUGUCGAGAAGAGGAGAACAAUAUCUUUCUGCCAUCAGUCGGCGUGGACUGU AGAACCAUGCCGACUGAUGGCAGCAUGUUGUUCUCCUCUCCUCGACACC-3′ (SEQ ID NO: 7).

23. The artificial nucleic acid according to any one of claims 14 to 20, wherein the loop comprises the nucleic acid sequence GCUAA or GCUCA, and wherein the nucleic acid sequence preferably comprises a nucleotide sequence selected from the group consisting of 5′-GGUGUCGAGAAGAGGAGAACAAUAUGCUAAAUGUUGUUCUCGUCUCCUCGACA CC-3′ (SEQ ID NO: 2), and 5′-GUGGAAUAGUAUAACAAUAUGCUAAAUGUUGUUAUAGUAUCCCAC-3′ (SEQ ID NO: 1).

24. The artificial nucleic acid according to any one of claims 14 to 20, wherein the loop comprises the nucleic acid sequence CUUUC, and the nucleic acid sequence preferably comprises a nucleotide sequence selected from the group consisting of 5′-GGUGUCGAGAAGAGGAGAACAAUAUCUUUCAUGUUGUUCUCCUCUCCUCGACA CC-3′ (SEQ ID NO: 3), and 5′-GUGGAAUAGUAUAACAAUAUCUUUCAUGUUGUUAUACUAUCCCAC-3′ (SEQ ID NO: 4).

25. The artificial nucleic acid according to any one of the preceding claims, which does not include any chemically modified nucleotides.

26. The artificial nucleic acid according to any one of the preceding claims, which consists of naturally occurring nucleotides, preferably of nucleotides naturally occurring in mammalian, more preferably mouse or human.

27. The artificial nucleic acid according to any one of the preceding claims, which is a genetically encodable RNA, preferably an RNA that can be expressed endogenously.

28. The artificial nucleic acid according to claim 27, wherein the RNA is encoded by a vector.

29. Method for providing an artificial nucleic acid for site-directed editing of a target RNA, wherein the target RNA comprises a target sequence comprising an adenosine as a target nucleotide to be edited and at least one off-target sequence comprising an adenosine that is not to be edited, the method comprising:
generating a nucleic acid sequence of the artificial nucleic acid that is complementary or partially complementary to a nucleic acid sequence in the target RNA, and
(i) if the target sequence comprises an adenosine nucleotide to be edited and nucleotides immediately 5′ and 3′ of the adenosine nucleotide in a triplet context of 5′-UAG-3′, 5′-GAU-3′, 5′-GAG-3′, 5′-GAC-3′, 5′-GAA-3′, 5′-CAG-3′, and/or 5′-AAG-3′,
replacing, in a first nucleic acid sequence of the artificial nucleic acid that is complementary or partially complementary to the nucleotide acid sequence in the target sequence,
one or more of a nucleotide triplet 5′-CUA-3′ by a nucleotide triplet selected from the group consisting of 5′-UUA-3′, and 5′-UCA-3′, preferably if the target adenosine nucleotide to be edited in the target sequence is the central adenosine nucleotide in a nucleotide triplet 5′-UAG-3′;
one or more of a nucleotide triplet 5′-AUC-3′ by a nucleotide triplet selected from the group consisting of 5′-AUU-3′, and 5′-ACU-3′, preferably if the target adenosine nucleotide to be edited in the target sequence is the central adenosine nucleotide in a nucleotide triplet 5′-GAU-3′;
one or more of a nucleotide triplet 5′-CUC-3′ by a nucleotide triplet selected from the group consisting of 5′-CUU-3′, 5′-UUC-3′, 5′-UUU-3′, 5′-UUG-3′, 5′-GUU-3′, 5′-UUA-3′, 5′-AUU-3′, 5′-CCU-3′, 5′-UCC-3′, and 5′-UCU-3′, preferably if the target adenosine nucleotide to be edited in the target sequence is the central adenosine nucleotide in a nucleotide triplet 5′-GAG-3′;
one or more of a nucleotide triplet 5′-GUC-3′ by a nucleotide triplet selected from the group consisting of 5′-GUU-3′, and 5′-GCU-3′, preferably if the target adenosine nucleotide to be edited in the target sequence is the central adenosine nucleotide in a nucleotide triplet 5′-GAC-3′;
one or more of a nucleotide triplet 5′-UUC-3′ by a nucleotide triplet selected from the group consisting of 5′-UUU-3′, 5′-AUU-3′, 5′-UCU-3′, and 5′-ACU-3′, preferably if the target adenosine nucleotide to be edited in the target sequence is the central adenosine nucleotide in a nucleotide triplet 5′-GAA-3′;
one or more of a nucleotide triplet 5′-CUG-3′ by a nucleotide triplet selected from the group consisting of 5′-UUG-3′, 5′-UCG-3′, 5′-UUA-3′, and 5′-UCA-3′, preferably if the target adenosine nucleotide to be edited in the target sequence is the central adenosine nucleotide in a nucleotide triplet 5′-CAG-3′; and/or
one or more of a nucleotide triplet 5′-CUU-3′ by a nucleotide triplet selected from the group consisting of 5′-UUU-3′, and 5′-UCU-3′, preferably if the target adenosine nucleotide to be edited in the target sequence is the central adenosine nucleotide in a nucleotide triplet 5′-AAG-3′,
and/or
(ii) if the off-target sequence comprises an adenosine nucleotide not to be edited and nucleotides immediately 5′ and 3′ of the adenosine nucleotide in a triplet context of 5′-UAU-3′, 5′-UAG-3′, 5′-UAC-3′, 5′-UAA-3′, 5′-GAU-3′, 5′-CAU-3′, and/or 5′-AAU-3′,
replacing, in a second nucleic acid sequence of the artificial nucleic acid that is complementary or partially complementary to the nucleotide acid sequence in the off-target sequence,
one or more of a nucleotide triplet 5′-AUA-3′ by a nucleotide triplet selected from the group consisting of 5′-GUG-3′, 5′-AUG-3′, 5′-GUA-3′, 5′-GGG-3′, 5′-AGG-3′, and 5′-GGA-3′, preferably if an adenosine nucleotide not to be edited in the off-target sequence is the central adenosine nucleotide in a nucleotide triplet 5′-UAU-3′;
one or more of a nucleotide triplet 5′-CUA-3′ by a nucleotide triplet selected from the group consisting of 5′-CUG-3′, 5′-UUG-3′, 5′-CGG-3′, and 5′-UGG-3′, preferably if an adenosine nucleotide not to be edited in the off-target sequence is the central adenosine nucleotide in a nucleotide triplet 5′-UAG-3′;
one or more of a nucleotide triplet 5′-GUA-3′ by a nucleotide triplet selected from the group consisting of 5′-GUG-3′, and 5′-GGG-3′, preferably if an adenosine nucleotide not to be edited in the off-target sequence is the central adenosine nucleotide in a nucleotide triplet 5′-UAC-3′;
one or more of a nucleotide triplet 5′-UUA-3′ by a nucleotide triplet selected from the group consisting of 5′-UUG-3′, 5′-GUG-3′, 5′-UGG-3′, and 5′-GGG-3′, preferably if an adenosine nucleotide not to be edited in the off-target sequence is the central adenosine nucleotide in a nucleotide triplet 5′-UAA-3′;
one or more of a nucleotide triplet 5′-AUC-3′ by a nucleotide triplet selected from the group consisting of 5′-GUC-3′, 5′-GUU-3′, 5′-GGC-3′, and 5′-GGU-3′, preferably if an adenosine nucleotide not to be edited in the off-target sequence is the central adenosine nucleotide in a nucleotide triplet 5′-GAU-3′;
one or more of a nucleotide triplet 5′-AUG-3′ by a nucleotide triplet selected from the group consisting of 5′-GUG-3′, and 5′-GGG-3′, preferably if an adenosine nucleotide not to be edited in the off-target sequence is the central adenosine nucleotide in a nucleotide triplet 5′-CAU-3′; and/or
one or more of a nucleotide triplet 5′-AUU-3′ by a nucleotide triplet selected from the group consisting of 5′-GUU-3′, 5′-GGU-3′, 5′-GUG-3′, and 5′-GGG-3′, preferably if an adenosine nucleotide not to be edited in the off-target sequence is the central adenosine nucleotide in a nucleotide triplet 5′-AAU-3′,
and/or
(iii) if the target RNA comprises an adenosine nucleotide to be edited (A) and at least one neighbouring adenosine nucleotide not to be edited and nucleotides immediately 5′ and 3′ of the adenosine nucleotides in a sequence context of 5′-UAAA-3′, 5′-UAAU-3′, 5′-UAAC-3′, 5′-UAAG-3′, 5′-AAAU-3′, 5′-UAAU-3′5′-CAAU-3′,5′-GAAU-3′, and/or 5′-UAAAU-3′, wherein A represents the adenosine to be edited,
replacing, in a third nucleic acid sequence of the artificial nucleic acid that is complementary or partially complementary to the nucleotide acid sequence in the target RNA,
one or more of a nucleotide sequence 5′-UUUA-3′ by a nucleotide sequence 5′-UCUG-3′, or 5′-GCUG-3′, preferably if the adenosine nucleotides in the target sequence are present in the context of the nucleic acid sequence 5′-UAAA-3′;
one or more of a nucleotide sequence 5′-AUUA-3′ by a nucleotide sequence 5′-ACUG-3′, preferably if the adenosine nucleotides in the target sequence are present in the context of the nucleic acid sequence 5′-UAAU-3′;
one or more of a nucleotide sequence 5′-GUUA-3′ by a nucleotide sequence 5′-GCUG-3′, preferably if the adenosine nucleotides in the target sequence are present in the context of the nucleic acid sequence 5′-UAAC-3′;
one or more of a nucleotide sequence 5′-CUUA-3′ by a nucleotide sequence 5′-CCUG-3′, 5′-UCUG-3′ or 5′-UUUG-3′, preferably if the adenosine nucleotides in the target sequence are present in the context of the nucleic acid sequence 5′-UAAG-3′;
one or more of a nucleotide sequence 5′-AUUU-3′ by a nucleotide sequence 5′-GUCU-3′ or 5′-GUCG-3′, preferably if the adenosine nucleotides in the target sequence are present in the context of the nucleic acid sequence 5′-AAAU-3′;
one or more of a nucleotide sequence 5′-AUUA-3′ by a nucleotide sequence 5′-GUCA-3′, preferably if the adenosine nucleotides in the target sequence are present in the context of the nucleic acid sequence 5′-UAAU-3′;
one or more of a nucleotide sequence 5′-AUUG-3′ by a nucleotide sequence 5′-GUCG-3′, preferably if the adenosine nucleotides in the target sequence are present in the context of the nucleic acid sequence 5′-CAAU-3′;
one or more of a nucleotide sequence 5′-AUUC-3′ by a nucleotide sequence 5′-GUCC-3′, 5′-GUCU-3′, or 5′-GUUU-3′, preferably if the adenosine nucleotides in the target sequence are present in the context of the nucleic acid sequence 5′-GAAU-3′; and/or
one or more of a nucleotide sequence 5′-AUUUA-3′ by a nucleotide sequence 5′-GUCUG-3′, preferably if the adenosine nucleotides in the target sequence are present in the context of the nucleic acid sequence 5′-UAAAU-3′.

30. The method according to claim 29, which comprises, in terms of (ii),
replacing, in the second nucleic acid sequence of the artificial nucleic acid that is complementary or partially complementary to the nucleotide acid sequence in the off-target sequence,
all of the nucleotide triplets 5′-AUA-3′ by a nucleotide triplet selected from the group consisting of 5′-GUG-3′, 5′-AUG-3′, 5′-GUA-3′, 5′-GGG-3′, 5′-AGG-3′, and 5′-GGA-3′;
all of the nucleotide triplets 5′-CUA-3′ by a nucleotide triplet selected from the group consisting of 5′-CUG-3′, 5′-UUG-3′, 5′-CGG-3′, and 5′-UGG-3′;
all of the nucleotide triplets 5′-GUA-3′ by a nucleotide triplet selected from the group consisting of 5′-GUG-3′, and 5′-GGG-3′;
all of the nucleotide triplets 5′-UUA-3′ by a nucleotide triplet selected from the group consisting of 5′-UUG-3′, and 5′-UGG-3′; and/or
all of the nucleotide triplets 5′-AUU-3′ by a nucleotide triplet selected from the group consisting of 5′-GUU-3′, and 5′-GGU-3′.

31. The method according to claim 29 or 30, which comprises, in terms of (ii),
replacing, in the second nucleic acid sequence of the artificial nucleic acid that is complementary or partially complementary to the nucleotide acid sequence in the off-target sequence,
all of the nucleotide triplets 5′-AUA-3′ by a nucleotide triplet selected from the group consisting of 5′-GUG-3′, 5′-AUG-3′, and 5′-GUA-3′;
all of the nucleotide triplets 5′-CUA-3′ by a nucleotide triplet selected from the group consisting of 5′-CUG-3′, and 5′-UUG-3′;
all of the nucleotide triplets 5′-GUA-3′ by a nucleotide triplet 5′-GUG-3′;
all of the nucleotide triplets 5′-UUA-3′ by a nucleotide triplet 5′-UUG-3′; and/or
all of the nucleotide triplets 5′-AUU-3′ by a nucleotide triplet 5′-GUU-3′.

32. The method according to any one of claims 29 to 31, which comprises, in terms of (i), replacing, in the first nucleic acid sequence of the artificial nucleic acid that is complementary or partially complementary to the nucleotide acid sequence in the target sequence,
one or more of a nucleotide triplet 5′-CUA-3′ by the nucleotide triplet 5′-UUA-3′;
one or more of a nucleotide triplet 5′-AUC-3′ by a nucleotide triplet 5′-AUU-3′;
one or more of a nucleotide triplet 5′-CUC-3′ by a nucleotide triplet selected from the group consisting of 5′-CUU-3′, 5′-UUC-3′, 5′-UUU-3′, 5′-UUG-3′, 5′-GUU-3′, 5′-UUA-3′, and 5′-AUU-3′;
one or more of a nucleotide triplet 5′-GUC-3′ by a nucleotide triplet 5′-GUU-3′;
one or more of a nucleotide triplet 5′-UUC-3′ by a nucleotide triplet 5′-UUU-3′;
one or more of a nucleotide triplet 5′-CUG-3′ by a nucleotide triplet 5′-UUG-3′ or 5′-UUA-3′; and/or
one or more of a nucleotide triplet 5′-CUU-3′ by a nucleotide triplet 5′-UUU-3′.

33. The method according to any one of claims 29 to 32, which comprises (iv) if the target RNA comprises an adenosine nucleotide not to be edited and nucleotides immediately 5′ and 3′ of the adenosine nucleotide in a triplet context of 5′-GAG-3′, 5′-GAC-3′, 5′-GAA-3′, 5′-CAG-3′, 5′-CAC-3′, 5′-CAA-3′, 5′-AAG-3′, 5′-AAC-3′, and/or 5′-AAA-3′, replacing, in the second nucleic acid sequence of the artificial nucleic acid complementary or partially complementary to the nucleotide sequence in the off-target sequence,
one or more of a nucleotide triplet 5′-CUC-3′ by a nucleotide triplet 5′-CGC-3′;
one or more of a nucleotide triplet 5′-GUC-3′ by a nucleotide triplet 5′-GGC-3′;
one or more of a nucleotide triplet 5′-UUC-3′ by a nucleotide triplet selected from the group consisting of 5′-UGC-3′, and 5′-GGC-3′;
one or more of a nucleotide triplet 5′-CUG-3′ by a nucleotide triplet 5′-CGG-3′;
one or more of a nucleotide triplet 5′-GUG-3′ by a nucleotide triplet 5′-GGG-3′;
one or more of a nucleotide triplet 5′-UUG-3′ by a nucleotide triplet selected from the group consisting of 5′-UGG-3′ and 5′-GGG-3′;
one or more of a nucleotide triplet 5′-CUU-3′ by a nucleotide triplet selected from the group consisting of 5′-CGU-3′ and 5′-CGG-3′;
one or more of a nucleotide triplet 5′-GUU-3′ by a nucleotide triplet selected from the group consisting of 5′-GGU-3′ and 5′-GGG-3′; and/or
one or more of a nucleotide triplet 5′-UUU-3′ by a nucleotide triplet selected from the group consisting of 5′-UGU-3′, 5′-GGU-3′, 5′-UGG-3′, and 5′-GGG-3′.

34. Vector encoding the artificial nucleic acid according to any one of claims 1 to 28.

35. Cell comprising the artificial nucleic acid according to any one of claims 1 to 28 or the vector according to claim 34.

36. Composition comprising the artificial nucleic acid according to any one of claims 1 to 28, the vector according to claim 34, or the cell according to claim 35, and an additional excipient, preferably a pharmaceutically acceptable excipient.

37. Kit comprising the artificial nucleic acid according to any one of claims 1 to 28, the vector according to claim 34, the cell according to claim 35, or the composition according to claim 36.

38. Use of the artificial nucleic acid according to any one of claims 1 to 28, the vector according to claim 34, the cell according to claim 35, the composition according to claim 36, or the kit according to claim 37 for site-directed editing of a target RNA.

39. The artificial nucleic acid according to any one of claims 1 to 28, the vector according to claim 34, the cell according to claim 35, the composition according to claim 36, or the kit according to claim 37 for use as a medicament.

40. The artificial nucleic acid according to any one of claims 1 to 28, the vector according to claim 34, the cell according to claim 35, the composition according to claim 36, or the kit according to claim 37 for use in the treatment or prophylaxis of a disease or disorder, wherein the treatment or prophylaxis comprises a step of site-directed editing of a target RNA.

41. The artificial nucleic acid according to any one of claims 1 to 28, the vector according to claim 34, the cell according to claim 35, the composition according to claim 36, or the kit according to claim 37 for use in the treatment or prophylaxis of a disease or disorder selected from the group consisting of genetic diseases or genetic disorders, metabolic diseases, tumour diseases, autoimmune diseases, cardiovascular diseases, neurological diseases or disorders, infectious diseases, and allergies.

42. The artificial nucleic acid according to any one of claims 1 to 28, the vector according to claim 34, the cell according to claim 35, the composition according to claim 36, or the kit according to claim 37 for use in the diagnosis of a disease or disorder selected from the group consisting of genetic diseases or genetic disorders, metabolic diseases, tumour diseases, autoimmune diseases, cardiovascular diseases, neurological diseases or disorders, infectious diseases, and allergies.

43. Method for treating a subject suffering from a disease or a disorder, the method comprising administering an effective amount of the artificial nucleic acid according to any one of claims 1 to 28, the vector according to claim 34, the cell according to claim 35, the composition according to claim 36, or the kit according to claim 37 to the subject.

44. The method according to claim 43, wherein the disease or the disorder is selected from the group consisting of genetic diseases or genetic disorders, metabolic diseases, tumour diseases, autoimmune diseases, cardiovascular diseases, neurological diseases or disorders, infectious diseases, and allergies.
